SEQUENCE LISTING (48 sequences)

SEQ ID NO: 1 - DNA, 957 bp, Homo sapiens
atggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaa

SEQ ID NO: 2 - protein, 318 aa, Homo sapiens
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

SEQ ID NO: 3 - DNA, 2306 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcgcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccgccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 4 - DNA, 792 bp, Artificial Sequence
  Description of Artificial Sequence Synthetic polynucleotide
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 5 - DNA, 2244 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgccc

SEQ ID NO: 6 - DNA, 714 bp, Artificial Sequence
  Description of Artificial Sequence Synthetic polynucleotide
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 7 - DNA, 2303 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 5573 bp, Artificial Sequence
  Description of Artificial Sequence Synthetic polynucleotide
gcagctgcgcgctcgctcgctcactgaggccgcccgggcaaagcccgggcgtcgggcgacctttggtcgcccggcctcagtgagcgagcgagcgcgcagagagggagtggggttaaccaattggcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccatcttcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaat

SEQ ID NO: 9 - DNA, 148 bp, Artificial Sequence
  Description of Artificial Sequence Synthetic polynucleotide
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 10 - DNA, 53 bp, Artificial Sequence
  Description of Artificial Sequence Synthetic oligonucleotide
ggccgcaataaaagatctttattttcattagatctgtgtgttggttttttgtg

SEQ ID NO: 11 - DNA, 110 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 12 - DNA, 130 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 1429 bp, Homo sapiens
ctctgtcactcaccgggcgggccaggccgggcagccatggctgagacactcttctggactcctctcctcgtggttctcctggcagggctgggggacaccgaggcccagcagaccacgctacacccacttgtgggccgtgtctttgtgcacaccttggaccatgagacgtttctgagccttcctgagcatgtcgctgtcccacccgctgtccacatcacctaccacgcccacctccagggacacccagacctgccccggtggctccgctacacccagcgcagcccccaccaccctggcttcctctacggctctgccaccccagaagatcgtgggctccaggtcattgaggtcacagcctacaatcgggacagctttgataccactcggcagaggctggtgctggagattggggacccagaaggccccctgctgccataccaagccgagttcctggtgcgcagccacgatgcggaggaggtgctgccctcaacacctgccagccgcttcctctcagccttggggggactctgggagcccggagagcttcagctgctcaacgtcacctctgccttggaccgtgggggccgtgtcccccttcccattgagggccgaaaagaaggggtatacattaaggtgggttctgcctcacctttttctacttgcctgaagatggtggcatcccccgatagccacgcccgctgtgcccagggccagcctccacttctgtcttgctacgacaccttggcaccccacttccgcgttgactggtgcaatgtgaccctggtggataagtcagtgccggagcctgcagatgaggtgcccaccccaggtgatgggatcctggagcatgacccgttcttctgcccacccactgaggccccagaccgtgacttcttggtggatgctctggtcaccctcctggtgcccctgctggtggccctgcttctcaccttgctgctggcctatgtcatgtgctgccggcgggagggaaggctgaagagagacctggctacctccgacatccagatggtccaccactgcaccatccacgggaacacagaggagctgcggcagatggcggccagccgcgaggtgccccggccactctccaccctgcccatgttcaatgtgcacacaggtgagcggctgcctccccgcgtggacagcgcccaggtgcccctcattctggaccagcactgacagcctagccagtggttccaggtccagccctgacttcatcctcccttctctgtccacaccacgagtggcacatcccacctgctgattccagctcctggccctcctggaacccaggctctaaacaagcagggagagggggtggggtggggtgagagtgtgtggagtaaggacattcagaataaatatctgctgctctgctcaccaattgctgctggcagcctctcccgtc

SEQ ID NO: 14 - DNA, 1057 bp, Homo sapiens
ctctgtcactcaccgggcgggccaggccgggcagccatggctgagacactcttctggactcctctcctcgtggttctcctggcagggctgggggacaccgaggcccagcagaccacgctacacccacttgtgggccgtgtctttgtgcacaccttggaccatgagacgtttctgagccttcctgagcatgtcgctgtcccacccgctgtccacatcacctaccacgcccacctccagggacacccagacctgccccggtggctccgctacacccagcgcagcccccaccaccctggcttcctctacggctctgccaccccagaagatcgtgggctccaggtcattgaggtcacagcctacaatcgggacagctttgataccactcggcagaggctggtgctggagattggggacccagaaggccccctgctgccataccaagccgagttcctggtgcgcagccacgatgcggaggaggtgctgccctcaacacctgccagccgcttcctctcagccttggggggactctgggagcccggagagcttcagctgctcaacgtcacctctgccttggaccgtgggggccgtgtcccccttcccattgagggccgaaaagaagggctgaagagagacctggctacctccgacatccagatggtccaccactgcaccatccacgggaacacagaggagctgcggcagatggcggccagccgcgaggtgccccggccactctccaccctgcccatgttcaatgtgcacacaggtgagcggctgcctccccgcgtggacagcgcccaggtgcccctcattctggaccagcactgacagcctagccagtggttccaggtccagccctgacttcatcctcccttctctgtccacaccacgagtggcacatcccacctgctgattccagctcctggccctcctggaacccaggctctaaacaagcagggagagggggtggggtggggtgagagtgtgtggagtaaggacattcagaataaatatctgctgctctgctcaccaattgctgctggcagcctctcccgtc

SEQ ID NO: 15 - protein, 387 aa, Homo sapiens
MAETLFWTPLLVVLLAGLGDTEAQQTTLHPLVGRVFVHTLDHETFLSLPEHVAVPPAVHITYHAHLQGHPDLPRWLRYTQRSPHHPGFLYGSATPEDRGLQVIEVTAYNRDSFDTTRQRLVLEIGDPEGPLLPYQAEFLVRSHDAEEVLPSTPASRFLSALGGLWEPGELQLLNVTSALDRGGRVPLPIEGRKEGVYIKVGSASPFSTCLKMVASPDSHARCAQGQPPLLSCYDTLAPHFRVDWCNVTLVDKSVPEPADEVPTPGDGILEHDPFFCPPTEAPDRDFLVDALVTLLVPLLVALLLTLLLAYVMCCRREGRLKRDLATSDIQMVHHCTIHGNTEELRQMAASREVPRPLSTLPMFNVHTGERLPPRVDSAQVPLILDQH

SEQ ID NO: 16 - protein, 263 aa, Homo sapiens
MAETLFWTPLLVVLLAGLGDTEAQQTTLHPLVGRVFVHTLDHETFLSLPEHVAVPPAVHITYHAHLQGHPDLPRWLRYTQRSPHHPGFLYGSATPEDRGLQVIEVTAYNRDSFDTTRQRLVLEIGDPEGPLLPYQAEFLVRSHDAEEVLPSTPASRFLSALGGLWEPGELQLLNVTSALDRGGRVPLPIEGRKEGLKRDLATSDIQMVHHCTIHGNTEELRQMAASREVPRPLSTLPMFNVHTGERLPPRVDSAQVPLILDQH

SEQ ID NO: 17 - DNA, 4248 bp, Homo sapiens
acagtcgggcggggagctcggcggcggcgggcgcgggaagatggcggcagcggcggcggcggctgcagaacagcaaagttccaatggtcctgtaaagaagtccatgcgtgagaaggctgttgagagaaggagtgtcaataaagagcacaacagtaactttaaagctggatacattccgattgatgaagatcgtctccacaaaacagggttgagaggaagaaagggcaatttagccatctgtgtgattatcctcttgtttatcctggctgtcatcaatttaataataacacttgttatttgggccgtgattcgcattggaccaaatggctgtgatagtatggagtttcatgaaagtggcctgcttcgatttaagcaagtatctgacatgggagtgatccaccctctttataaaagcacagtaggaggaaggcgaaatgaaaatttggtcatcactggcaacaaccagcctattgtttttcagcaagggacaacaaagctcagtgtagaaaacaacaaaacttctattacaagtgacatcggcatgcagttttttgacccgaggactcaaaatatcttattcagcacagactatgaaactcatgagtttcatttgccaagtggagtgaaaagtttgaatgttcaaaaggcatctactgaaaggattaccagcaatgctaccagtgatttaaatataaaagttgatgggcgtgctattgtgcgtggaaatgaaggtgtattcattatgggcaaaaccattgaatttcacatgggtggtaatatggagttaaaggcggaaaacagtatcatcctaaatggatctgtgatggtcagcaccacccgcctacccagttcctccagtggagaccagttgggtagtggtgactgggtacgctacaagctctgcatgtgtgctgatgggacgctcttcaaggtgcaagtaaccagccagaacatgggctgccaaatctcagacaacccctgtggaaacactcattaaaagaaccccagaggtcaccaacatgtttatatcttgacttgacttttttatgcatgcaaatcattgtttttacagagtttgtgataactcataattattttaatggcagagcactgctgtatctgttttatggtctacatagttaaaatcttctcagagagcctaaattctaatacattttattaatttatactaatcttcatatttactgttctctaaaataattatgagaagcaaataaaatcaaaagtcatgtttaaagacgtgtttttaaaattccactatcccttttctaaaggttaaaggtctgaagcagctgtttagattcactgtaagtaaactttggtaactctaatggggatagacccacttaagatatttaaaaaggtatggcatcagcgtttcatgctctgccttttagcttctaaaaggaaagatgcagatttctagtgcattaagcctgagccatattctcacatgcaagtgaagtcattaaagaactttacatatgtgagatagaaacaatggttccttagttttgcactgggaagaaaatattttgtaaaagaatgtttatttgaaataatgataactatcaattgttcacaatgtggtggaaattaaaacaccatctcagctttaacttttaaataataatgataactatctttattgagcatcttctacatcctaggcattgtcctaggcattgcatgtttatatccccaattctcaccacaaccctgcaagtaggtggtattatccaagttttacccattaagaaactgaagatcagagaagttaagaaacttgctcaacatcatatagtaagtagcagagttgggattggaattcaggcatgactcaaacctggatgtacttgattccaaatgccatgttgttttcactctctgcactgactttttaattatttaaaactctagaaagatgaacaaaggttaatttaaacttacctaagaagatgagaatcaaacaaaacagatatgcttactctagttaaaaagaaaataaatctcatgtcagacccagaaaggaccaatcactgtccgattgtaagctatgttgggccaattccaaaatattatacgatggagaggtcaaatttacctacttctgagttacctcagtttcccaacaatggaccttggcacactggagtaacaatacataacagagttgccaagatatttataccctcagcactcggggcaacacagtggaaagtggggaggccatagacccaaacaagttctttgggccaggcatggcctagtaagtacaccatgcctcgaaaataagtccagaagcactggactaaagagtgctaatgcaggaaataatacacataatttttaggtaaggataataatttatctctgctcctaatattactatcccattgtaattatttataaccctcaagccagttgatttttaatatatttgattggaaaagaactctctggtattattaagactcacacagaatcagggacagggcccccaaaggagtttgctgtaaaataggcagtagagttgtggcatgggcccaaccctgcattcaagtgtaacagcattctgtcagggtcactttgaattgtgcacataagaaaaccaatacaaaaaacaatttgtattcaatattgtcacatttctctctggtagaaaaatcaaataccttagagattatgaagtcattaatttatactgaaattggattgacttactacctaacactgagcgctgtttttaaatgaaaagaatgagatttataaccacttgagtgttattgcagtgatatttgaactcatttgaatatattcagtatcatttaatgtctgaattcagaaaaaaatgccgaaatttttattcagatggtccataaattaaattgcatattcattacttatctgctctatttagatttattttaaaagtttatttaagtaaatatttttataaaaaccagaaaacactgtattacaaaatattatttattaaatgtagttcaggaaataatctatttttactcttttttgggaaatacttgtgtttttgatacatctccatgaagttcttttgagaggagaggctattttgatgtttttatacaactgaaggttaacagccatagcattttatgatactttacaggtagtcctggctttttccctgaaacataagcttggaaaatctattagaaagcagaacagggcaaactctccattttacttatggcttttctaatttttaattaattaatttatttttttgagacagagtcttgttctgtcgccaggctggagtgcagtggcacaatctcggctctcggctcactgcaacctctgcctcccaggttcaagcgattctcctgcctcagcctcccgagtacctgggactacaggcatgtgccacagttcccggctaatttttgtatttttagtagagacggggtttcaccatgttggccaggatggtctcaatctcttgacctcgtgatctgcctgccttggcctcccaaagtgctgggattacaggtgtgagccaccacgcctggccggcttatttttatccacagtaaatcttcagcaactcattgtctccaccagatagtatttttctgtaaatgaaatgctgacttcgcctcttcctgctgtatgctcatccctgcactgagcacagatatgacaagcagtagccatgggggaggtgggtgacaaagataggaccccgggagggggcgcaggtacatgctagtttcaattaccacagtattctagagacgggttgcaatgacaaggggggcaaatgaaatcaatgcaagatttcttaataatgggcagacagaaaaatgtaaaaccacacaaaacggactgctgataatattttaaaatatacttatttgtcttctttttgcattgtgaaaaaaacaaaataaattttgtgtgataattttgatgatgaaaggtggaagttctacctagatttgaatgagtgtttttttaagggaatgagaatgtcatggtgctaaacctgacaaataagagatcattgaaatgctgaaaattttaacagtcttcttaaaagtattgagggggcaaaaattaccaattatggtatacaaaaataagcctataaatgtgtttcacattgctaacttgagtttcagttgattcagtttgtaataactagtaatgagcttctgtttacaataaaaattctgtaaattg

SEQ ID NO: 18 - protein, 318 aa, Homo sapiens; identical to SEQ ID NO: 2

SEQ ID NO: 19 - DNA, 2225 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcgcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggtgagggagcagtacaccacagcaaccgagggaatctgcatcgagaggccagagaaccagtacgtgtataagatcggcatctacggctggcggaagagatgtctgtatctgttcgtgctgctgctgctgatcatcctggtggtgaatctggccctgaccatctggatcctgaaagtgatgtggttttccccagcaggaatgggacacctgtgcgtgacaaaggacggactgcggctggagggagagtctgagttcctgtttcccctgtatgccaaggagatccacagcagagtggatagctccctgctgctgcagtccacccagaacgtgacagtgaacgcaaggaatagcgagggagaggtgaccggcagactgaaggtcggccccaagatggtggaggtgcagaatcagcagttccagatcaactccaatgacggcaagcctctgtttacagtggatgagaaggaggtggtggtgggcaccgacaagctgagggtgacaggacctgagggcgccctgttcgagcactctgtggagaccccactggtgcgcgcagacccttttcaggatctgaggctggagagcccaacacgcagcctgtccatggacgcacccagaggcgtgcacatccaggcacacgcaggcaagatcgaggccctgagccagatggatatcctgttccactctagcgacggcatgctggtgctggatgccgagaccgtgtgcctgcctaagctggtgcagggcacatggggcccatctggctcctctcagagcctgtacgagatctgcgtgtgcccagatggcaagctgtatctgtccgtggccggcgtgtctaccacatgccaggagcacaaccacatctgtctgtgactcgagggccgcaataaaagatctttattttcattagatctgtgtgttggttttttgtgtgtcctgcaggggcgcgcctaatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 20 - DNA, 876 bp, Artificial Sequence
  Description of Artificial Sequence Synthetic polynucleotide
atggtgagggagcagtacaccacagcaaccgagggaatctgcatcgagaggccagagaaccagtacgtgtataagatcggcatctacggctggcggaagagatgtctgtatctgttcgtgctgctgctgctgatcatcctggtggtgaatctggccctgaccatctggatcctgaaagtgatgtggttttccccagcaggaatgggacacctgtgcgtgacaaaggacggactgcggctggagggagagtctgagttcctgtttcccctgtatgccaaggagatccacagcagagtggatagctccctgctgctgcagtccacccagaacgtgacagtgaacgcaaggaatagcgagggagaggtgaccggcagactgaaggtcggccccaagatggtggaggtgcagaatcagcagttccagatcaactccaatgacggcaagcctctgtttacagtggatgagaaggaggtggtggtgggcaccgacaagctgagggtgacaggacctgagggcgccctgttcgagcactctgtggagaccccactggtgcgcgcagacccttttcaggatctgaggctggagagcccaacacgcagcctgtccatggacgcacccagaggcgtgcacatccaggcacacgcaggcaagatcgaggccctgagccagatggatatcctgttccactctagcgacggcatgctggtgctggatgccgagaccgtgtgcctgcctaagctggtgcagggcacatggggcccatctggctcctctcagagcctgtacgagatctgcgtgtgcccagatggcaagctgtatctgtccgtggccggcgtgtctaccacatgccaggagcacaaccacatctgtctgtga

SEQ ID NO: 21 - DNA, 1594 bp, Homo sapiens
attctgtaagtcatagaaaagtttgaaacattctgtctgtggtagagctcgggccagctgtagttcattcgccagtgtgcttttcttaatatctaagatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgcttctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgcttgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacaaccacatctgcctctgagctgcctgcgtcctctcggtgagctgtgcagtgccggccccagatcctcacacccagggagcagctgcacatcgtgaaagactgaggcagcgtggatgggaagtaaacgcttccagaggaactcagaaaaaattatgtgccagtgaaagtgtttggacaaaaactacatgatctcaaaatgcacgtggatgtgagacacaaaagttgacaaaatggaaaagcaatgtgtttttccactggattaattttcaccggaacaattgcgaattctctctgcctcgcctccccctatcttgtccgtgtgggcacacactgagtgttgagttgccgtgtggagttaatgtatgacgctccactgtggatatctaatgccctgttgagagtagccttgctcagtactaaaatgccccaaagttctatacagcatttcctttatagcattcaaacctcacatcctcccttcagtttaatgcaagtaagtcaggtttcacaagaaaattttcaagttttgaagggaatttgaggttgatctggttttcaagatgtagttaaaggaataaatcactcaaaattaaactttctgtatatagtcaataagcaataaaaacctcatttttcaga

SEQ ID NO: 22 - DNA, 1636 bp, Homo sapiens
agagtcgtcgctgcggtcgctgaggaaggacggagcagaggcccgcgctgtctggggagaagactgtggtgtcatcccgtcgggaatgaagggaaatgcagcggctgtttgcgccccgggactccaagaagtacagcagatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgcttctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgcttgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacaaccacatctgcctctgagctgcctgcgtcctctcggtgagctgtgcagtgccggccccagatcctcacacccagggagcagctgcacatcgtgaaagactgaggcagcgtggatgggaagtaaacgcttccagaggaactcagaaaaaattatgtgccagtgaaagtgtttggacaaaaactacatgatctcaaaatgcacgtggatgtgagacacaaaagttgacaaaatggaaaagcaatgtgtttttccactggattaattttcaccggaacaattgcgaattctctctgcctcgcctccccctatcttgtccgtgtgggcacacactgagtgttgagttgccgtgtggagttaatgtatgacgctccactgtggatatctaatgccctgttgagagtagccttgctcagtactaaaatgccccaaagttctatacagcatttcctttatagcattcaaacctcacatcctcccttcagtttaatgcaagtaagtcaggtttcacaagaaaattttcaagttttgaagggaatttgaggttgatctggttttcaagatgtagttaaaggaataaatcactcaaaattaaactttctgtatatagtcaataagcaataaaaacctcatttttcaga

SEQ ID NO: 23 - DNA, 1696 bp, Homo sapiens
cctttctccagggacagttgctgaagcttcatcctttgctctcattcttcttattttcagaaactatgaataaatcttctacaccatcttcccgagagctttagtaagacctcagactggagtagagttgaaggagggaatgccagtctgaagaaggaaaggaagaggagagacagcaaggagaacttcagttgtcaagatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgcttctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgcttgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacaaccacatctgcctctgagctgcctgcgtcctctcggtgagctgtgcagtgccggccccagatcctcacacccagggagcagctgcacatcgtgaaagactgaggcagcgtggatgggaagtaaacgcttccagaggaactcagaaaaaattatgtgccagtgaaagtgtttggacaaaaactacatgatctcaaaatgcacgtggatgtgagacacaaaagttgacaaaatggaaaagcaatgtgtttttccactggattaattttcaccggaacaattgcgaattctctctgcctcgcctccccctatcttgtccgtgtgggcacacactgagtgttgagttgccgtgtggagttaatgtatgacgctccactgtggatatctaatgccctgttgagagtagccttgctcagtactaaaatgccccaaagttctatacagcatttcctttatagcattcaaacctcacatcctcccttcagtttaatgcaagtaagtcaggtttcacaagaaaattttcaagttttgaagggaatttgaggttgatctggttttcaagatgtagttaaaggaataaatcactcaaaattaaactttctgtatatagtcaataagcaataaaaacctcatttttcaga

SEQ ID NO: 24 - DNA, 1745 bp, Homo sapiens
attctgtaagtcatagaaaagtttgaaacattctgtctgtggtagagctcgggccagctgtagttcattcgccagtgtgcttttcttaatatctaagtcttattttcagaaactatgaataaatcttctacaccatcttcccgagagctttagtaagacctcagactggagtagagttgaaggagggaatgccagtctgaagaaggaaaggaagaggagagacagcaaggagaacttcagttgtcaagatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgcttctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgcttgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacaaccacatctgcctctgagctgcctgcgtcctctcggtgagctgtgcagtgccggccccagatcctcacacccagggagcagctgcacatcgtgaaagactgaggcagcgtggatgggaagtaaacgcttccagaggaactcagaaaaaattatgtgccagtgaaagtgtttggacaaaaactacatgatctcaaaatgcacgtggatgtgagacacaaaagttgacaaaatggaaaagcaatgtgtttttccactggattaattttcaccggaacaattgcgaattctctctgcctcgcctccccctatcttgtccgtgtgggcacacactgagtgttgagttgccgtgtggagttaatgtatgacgctccactgtggatatctaatgccctgttgagagtagccttgctcagtactaaaatgccccaaagttctatacagcatttcctttatagcattcaaacctcacatcctcccttcagtttaatgcaagtaagtcaggtttcacaagaaaattttcaagttttgaagggaatttgaggttgatctggttttcaagatgtagttaaaggaataaatcactcaaaattaaactttctgtatatagtcaataagcaataaaaacctcatttttcaga

SEQ ID NO: 25 - protein, 291 aa, Homo sapiens
MVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHNHICL

SEQ ID NO: 26 - protein, 291 aa, Homo sapiens; identical to SEQ ID NO: 25

SEQ ID NO: 27 - protein, 309 aa, Homo sapiens
MKGNAAAVCAPGLQEVQQMVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHNHICL

SEQ ID NO: 28 - protein, 291 aa, Homo sapiens; identical to SEQ ID NO: 25

SEQ ID NO: 29 - protein, 291 aa, Homo sapiens; identical to SEQ ID NO: 25

SEQ ID NO: 30 - DNA, 9805 bp, Homo sapiens
agagaggacttatctccagcatctagcactacagagcagaggctgtgtggagaatggctgaaaaatcagaattggttgtagaagcagttttctttctggttgtgagtatgagcccggcagacaccatgagcgctgttgcaggggagtcggcctgtgcttgacacatgtgtttcccattgatagctggagacagcccagtagctgtgagtcggtctgacaaagccatattgaagtacggagtacggtttcaaagcagtcagaaaaagaacgggaatgctgttcaggaaattcttcaggcatgggcagggacttggctgcagttctgcagttggaaaatctgactggggcagcttctgagcgcaggctgggcctgcacacactcagcgggccgagtggccacctccttcagagctgctcagcacgccctgggatcgcgggcggttttcatcggccggtttgtgaaacggacaagagagagacattactgccgggagtgttgagtgaagggaccaggtggagatgatgcctcaggagcagtacactcaccaccggagcaccatgcctggctctgtggggccacaggtatacaaggtggggatttatggctggcggaaacgatgcctgtatttctttgtcctgctcctcatgattttaatactggtgaacttggccatgaccatctggattctcaaagtcatgaacttcacaattgatggaatgggaaacctgaggatcacagaaaaaggtctaaagctagaaggagactctgaattcttacaacctctctacgccaaagaaatccagtcccgaccaggtaatgccctgtacttcaagtctgccagaaatgttacagtgaacattctcaatgaccagactaaagtgctaactcagcttataacaggtccaaaagccgtagaagcttatggtaaaaaatttgaggtaaaaactgtttctggaaaattgctcttctctgcagacaataatgaagtggtagtaggagctgaaagattacgagttttaggagcggagggcacagtgttccctaaatctatagaaacacctaatgtcagggcagaccccttcaaagaactaaggttggagtccccaacccggtctctagtgatggaggccccaaaaggagtggaaatcaatgcagaagctggcaatatggaagccacctgcaggacagagctgagactggaatccaaagatggagagattaagttagatgctgcgaaaatcaggctacctagactgcctcatggatcctacacgcctacaggaacgaggcagaaggtcttcgagatctgcgtctgcgccaatgggagattattcctgtctcaggcaggagctgggtccacttgtcagataaacacaagtgtctgcctctgaaagactatccatagtggacattgttggcagcataaaggccttttttggctttagacactggctgccagctatttttactagaacacagaaagcctatcaaagaccttgtgtgtatgtgtacgtgtgtgtgcgtgcttgagtgtgttcgcgtgtgtgggtggatataaatatatataaatatatataaataaatatatatatcctctgtataaaatgaggtttcagtacaaaaggaaccatgggtgaccctgcgatatccactgtcattttccatcccatccccaccacctgagtgacagaaatctaaacacacatccgtcccaacattccccagaccattcagaatcacacagcgtattaaacactgacagaatcttcatctagatattcgagtagcagcatatcttctcttttagtgtcattacgagggagtgatggcgggaatctcagtcgcactcaagctctgagacctttgtatcaaaaataggcatttgatttcctgttttagctttagtaaggctggctaacttccccctcttcaagctaggaactggcaatgctgtagaagtcagccgtaggaattcaaaatggctggcctaccttggctaccagacatattggggtttttgtagttgaatgaatgaggaggatgaatttcagcaaattttgaactgctcacccaacttctgctatcttgctccctccaaactcacagattctcctacagtcaaattaggagctgtaaatcagcacaaaataagataacagctgttcctcagtgagctggaagctacttaatggcctgatgggcaatgaacaaacgggtgatatgtctctgtttaagggaaaaatggcttaaaagctgttctgttctcacttctgactttaaccaaaagatttcaacccacaatgatcaggtcaatcaaaatccctaagagcagaactcctaccccaaaagaagcctggaagtctaattaagagtagcataaggaacctaattatgtttaccatgtttcttgggattggtgggaaatgtcaaaacatgccctttatttttaaaggcattcacaaactctctgactttgttcttcttatatattttttcagtgccgggatattcatattcctaaagccactattgtgttttctctaagaagcactacatgccaccagaattgtgcactgaaagataatacaaactgagtgtctttatgagaatcactgtgtcccctgaggcccagcagtacctgcttccctgtatgtggaagcagcacctcattcccgccatcagctacctctcatcaccccaccttcatcatcatgctccagggtcaccctggccagctcttgtgctgggacaggggattacaccatctctgttcaaagagggggaaatgtgcctatgccttaagtcaatgcactcagcaaggagaagcacatcttatttatcttgttacctatagtttactttgggtgattggaggggaatgacttagttatgactggacatcttaaaagctgatagacaagccaaatggctggcagatgatgtggatttcaaagagcccagaatgaactcatcactggcttagacagtcctggatgccatttggaaagtagtggccctgcaagcctaaatgaagtagtatttgtaggcctgggtggcatttggatttttcttttccctcaacaaggttttactttttcttactttacaagcaagggaagttttgtgataggagagaaataaaagatttgatattttttgagatgacactcaagcatcaggctgagatttgcacacatgggatgtaaaagcaagctgtgtgttgcttagtcacttacttagaagtagatggtgggggacagcggcgtgggtcctagcctggccagtgatgctgctggcgtccagaccccagactcactccaagcactcttgttcaatatctcatgcagaagagttgggctggtcactcttaggggtgagaccccgtgattggttggtttgtagcactaaggtctaaaaaggaaaaccataaaagacatcagattacgctggatcagtataattaatattccatagggccatgttgccagaatctgtatgtatcaatacagggtttttccaagccaggaaacgccctccttggctactaggagcacctatcccatatcattcagataaacaatgatagctacaaagtcatttgtggctagataggttaagacaggtgattttttaaagtagactgtctttgcattttgccatctgaagttcattaatctttagatgacaaaaaagcaaaaagttcccagaacgtttttgcttagattttgttctaatcaccacgtgaaggaatgagattagcaccacaagtttcatgccaataaaagagactggtgtgatcccacatgcaaaatttaatcctaagggtagtgagatcacaaacagaataaaaataagagcaatcaaccatataaatcaagtacctattgggaacagacataacattcaatttttcatttatgctaagtgaccacagtatacaaagtaataagcaggaaatttgatatgggttaaattatgcattttgttcagattttggaaattggtatgcattataagtttctcaatgtacatctttttatcccaacaccctcaaactagaatatttgtcagtggtcaagagaaaaaattttacctgaattcttgggggccggcggggagcttttacattaaaaatctactaacgcctactttttaaaaaatgagattctttctaatctttatatatgacattttctagacaatcgcacctttgggtatattaaacagctggtatcataacaaagaatccaaatgaaccttcaatatactagaagttctagtaggttaatattgttcagaagatttttacaaattaaaaactgatttccaaatatgttcaacatttacttctatttgatatctgctcaagaagtcatagaagtcttgggaaactattcgagtatcacagaggttttcaaaagcccttatggtgacatctacctaggtaaaagcctgacatgtggctttataattgttatgttacccaagggataaacttgaactggctttgaacatcctttaggtcattttctctttggataattttcatcgcatatccagcaactatagaccaaagtttgcttaaggtttgaccctagagcagaggtgggcaaactatgacctggaaaccaaatctgacttaccaccttttcctgtagttaaagttttattgtcacatagccacatacattcatttacatgttttctgattttacactacagcagcaggatggagtagttgtgtcagagactatgtatcccacaaagcatagattacttactatttagtcctttacaggcaaagtttcctcacccctgacctagaggtttttgtggtatgcattggatatagcaggaaagaaagcacatttccaaacagcagggagtaagcttacatttctgtgtaggtttgggaatatagttacttggcaaagtctttcaggaaggaagcccttccttatgttacatgtggaaagcctgccttccaagacatgtggaagtaattgatccacctgccagagaaacacaggctcagaggatgcctgggaacagggaggatgggattagtggaagcttaatggaaaaggaaagttatgatcctccaagacccttaattgatagaccataccaggttctgcaggtcagcatcattgtgtaatgagagtgaagtaggggaccctgtggttcaaccttagaatctgtttcctgtaggctctttctgctgtctatattcattaaagttttccacttcaccctcccatagtctagagggatgcccattccatggtcctccagagaatagttttgacttaacatgtctgtttagcccacatcacgtcagttatcaacaccgccactgtgcttactgttcctacagccacaccaggcttgaagagttagtgagaccaacaaataattggaagtattggaaaaagcaaaatacatggggacaaaaaaaatacagtgaaattctttttatcaaactgatgctgtgagaaaccagatgaatgccagtttggctttatttctaagaatctgggtcttcattctctggtgtagaaggaatgcaaaaaactataacaacaacaacaaaaacatattttgaaaagacatattctgacatctctgcttgtgtgtggtaaggcaggttcctatcagacatttatccctttggtcaagatcccttttgctcatccagggtttcatactcaatatcgcttaaaaaaaaaaaagtatcagctagggatgactctggaagtatgagtatcatggtggggaggaaggaattttttttaaatgtaaatgacccccatttaccagaccctaatcaaagtcacttaagggaatccctcagccttttatttggaaacagttgaaataaactggcagcagctagatcagagtatcttgctttattttataaaggccaaaggtagtatgaagtttggaccaaaaaggtaaatagatccattccagcacctgatactgatttttcaaggctctatgaaaggtcaaaaatttcattaaacaagaccagttctccctcttccccctgtcccaagaaatcttaggcatgaaaaggataaggaaacagctcctggaatgatacatttgcatagtgccctagtagcaggttgggaaaaagttataatataagaaacaaccttcgaaaacaggccttttatctcaaagataaaatgtctttcttgtggtctttcatcactatctccgtggtggaaggttcccctagttccaacatatttccattaaaatagtcaaagccacggcattgggatgtcagatgcctctctttctttgtggtaatcggaatttaaaattatacagttgcctctgaatttctcatgcacaaagccaaaccactgataagagataaagcagtctgaagcctgctgcttcagccagcacagcacaccacacacgctcgcactttcaaaagcaatgtgatttctatggttcttaaaagctttcttcataagggagtccctgaaatttctcaaggcaggtttgaatggcaaagggaaaataattacttgtgggaggtcctccttttgagtattgttagagcatacatgtaaaagaaaataacctttttggggcaactcatgctcacacatgctgttttctttggttctccccctaccttccttttgtagatattgacagaataggaggaaatgagcatccttatttgagaaagagcaagaatgtcatgagcccttgatgcaatagtaagtgtgatgtcatcatacagtgttaatgatgctatcaaatccatcaataaacagtctcaaaccttccaacaacagtgctcactgctgctcctcaacttcagcccagcaagcagtaatatcatcacccattttgagatatgcaggtggaatagaacaaagaaacagccactgtaatcgagaagcatgtttactgtctaaatccacctgttgcagtaggaagccagagtggggttccaaatgcctcattaagtatgtggacagcctcactagtaagtgagtgaatttggcttcatcactgaacattagctaaggtcagcttaataacacaaatatgaggccgacttctttgcgagaagagaaaagaaaacatctgcttgatttaaaatccaccccacatgcctagagttgtctaatagtccctcactttccaatggcttcacatcctacttctacatttggggttttttggtgaaatcagagatagctcaggatttcataaaacgagaaactccaaactggtctattaggttccatgggaacacttgtagccaaaggattgtctgagggcaggaagacgacacttgtcaacaaggaagacagtgtttctttagttcccatattcatctaattcatggggttctaacattttggggggccatagattcttttgacaatctgagccaatctatgaaacttctccccaaaaagaaccaccccacaaaatcttgcaaaacgtaagagattttcctggatctgaagctacccgaagacatgggaagagttgtattctattattcaattttaagaatatttattaatattcactgagctattgctagccactgtgctaaacattttacatacattctcctatttcatcctcaaaacaatcctttgagttgggttttaatatagttccaatattcggatgtgaaaactgaggcttatagtggctaagaaacttgcccaaagccactacctagaaaatggcagagctggaatgtaggtttaactcctgaccactatgctataaagtcaccacatgtcaaactaattttcaagttgttgggacatgtcccctactaggttttaaactagatcttccttggtagatgaagctatagaacttctattttccctgctttctgtagtctctcacagtgacagcatctatactaaagtatagatacctaaggggaaaatatagaaagcctgcctgaataatagaatctagaacaacaacaaaaatattaattttttctgtgtatgcttaggtcaagcttaaaaaaaaaaaaaaaagaccggaaaatacctgggttgttagcctcacatttaggaaaaaattgtaatactcagttatctgtgtgtgtggctaaacaagtcagcatttctgcacacatacatctctttcctttatacttcccttcaaaagacaaatatcttacttttgatctttgacactatttggtcagtattcttctttacctttacttgtggcaaaactcaaggaagcgatcaaatagagggaagctcatttctatcattgtctctgtttccctataagaaagaactaccagggactcactgactgcattaggcatacaatgtcagagctgagctgaccactctggtcctgtaatgtctttggcctcacaccttggcagccatcattaatgggccatacccttccccaggtgcagaattctcctccccagagcactcaggccgttactaccaatttatctgagttggaaataagactcatttgccagttcttatttttaaagtggcaccctttaactttgaacctgtgtattttacactggcatcctagattcagcaatgaggtttggtggtgtttcaactaggaagggagaaaatgagtgcatctgaagttccttacagcttggtttctttggaatgctttcatcttctaagcaaagggatcagggtttgatctgtaagagttaaaaagacaaagtcattttgaagaattaactcagccagggatcatgcaaaaagattagaaaccataatgcccttgttaaagccctgctgtcaacctgccttcacccagagcttagagggccacagcagcaaagaggttggggtccatccctctctgatgtgctttttccacaacacatatctggtcctctggcaggattgtggatagagctcctcaccatacccaaaagactcagccccagtgccagtgctttcctggttcaacaacccaccacaaaaccttagtaaaaggatgagccaaaaatgaaaaagactcgactctacagtaagtcagtcagggatttcctttttaatggtttaagacatccaaatggcaagccaggaatagataccattaaagggtctcataggactaaccttaccagagccagaaatctagctctctggaagagatgcaagattctagaaaagtaaagggaagtgtcggcacatctaaatttagtgaacacaaaattaatttttatctagtctgtgacggagggaataaagtttttcatgtatcaaccacctcccccagtcaggtttctccctttttgagattatgaagaagctgagacatacttcttaaggaggtcgtgttttagaaggaaaaggcagaggctatccatcattatgctggctagatgcgcttctgaagaagccggattctgatgttcttaaccaaaatggtgaggtcatggaagtcccatttgcttggagattttgaaaaaaaaaaaaaaaaaaaacccattcccataaagtaattgagttcagcctttggattatttttggtttggtttttctctggttttgggtgtgatgtaagaagagctttttagttttgttttgaataacatcaatccttgcacactctatgcaaaaattttgtaagcatttcaataatgctatgaattacaaggaactattttaactttattacactttctgtataaaaaatttgtatttaatattatttcgaccacagtcttgtaaaatatattaataaaaataatgattggtaagaaggaaaaaaaaaaaaaaaaaa

SEQ ID NO: 31 - DNA, 9337 bp, Homo sapiens
agagctgctcagcacgccctgggatcgcgggcggttttcatcggccggtttgtgaaacggacaagagagatgcctcaggagcagtacactcaccaccggagcaccatgcctggctctgtggggccacaggtatacaaggtggggatttatggctggcggaaacgatgcctgtatttctttgtcctgctcctcatgattttaatactggtgaacttggccatgaccatctggattctcaaagtcatgaacttcacaattgatggaatgggaaacctgaggatcacagaaaaaggtctaaagctagaaggagactctgaattcttacaacctctctacgccaaagaaatccagtcccgaccaggtaatgccctgtacttcaagtctgccagaaatgttacagtgaacattctcaatgaccagactaaagtgctaactcagcttataacaggtccaaaagccgtagaagcttatggtaaaaaatttgaggtaaaaactgtttctggaaaattgctcttctctgcagacaataatgaagtggtagtaggagctgaaagattacgagttttaggagcggagggcacagtgttccctaaatctatagaaacacctaatgtcagggcagaccccttcaaagaactaaggttggagtccccaacccggtctctagtgatggaggccccaaaaggagtggaaatcaatgcagaagctggcaatatggaagccacctgcaggacagagctgagactggaatccaaagatggagagattaagttagatgctgcgaaaatcaggctacctagactgcctcatggatcctacacgcctacaggaacgaggcagaaggtcttcgagatctgcgtctgcgccaatgggagattattcctgtctcaggcaggagctgggtccacttgtcagataaacacaagtgtctgcctctgaaagactatccatagtggacattgttggcagcataaaggccttttttggctttagacactggctgccagctatttttactagaacacagaaagcctatcaaagaccttgtgtgtatgtgtacgtgtgtgtgcgtgcttgagtgtgttcgcgtgtgtgggtggatataaatatatataaatatatataaataaatatatatatcctctgtataaaatgaggtttcagtacaaaaggaaccatgggtgaccctgcgatatccactgtcattttccatcccatccccaccacctgagtgacagaaatctaaacacacatccgtcccaacattccccagaccattcagaatcacacagcgtattaaacactgacagaatcttcatctagatattcgagtagcagcatatcttctcttttagtgtcattacgagggagtgatggcgggaatctcagtcgcactcaagctctgagacctttgtatcaaaaataggcatttgatttcctgttttagctttagtaaggctggctaacttccccctcttcaagctaggaactggcaatgctgtagaagtcagccgtaggaattcaaaatggctggcctaccttggctaccagacatattggggtttttgtagttgaatgaatgaggaggatgaatttcagcaaattttgaactgctcacccaacttctgctatcttgctccctccaaactcacagattctcctacagtcaaattaggagctgtaaatcagcacaaaataagataacagctgttcctcagtgagctggaagctacttaatggcctgatgggcaatgaacaaacgggtgatatgtctctgtttaagggaaaaatggcttaaaagctgttctgttctcacttctgactttaaccaaaagatttcaacccacaatgatcaggtcaatcaaaatccctaagagcagaactcctaccccaaaagaagcctggaagtctaattaagagtagcataaggaacctaattatgtttaccatgtttcttgggattggtgggaaatgtcaaaacatgccctttatttttaaaggcattcacaaactctctgactttgttcttcttatatattttttcagtgccgggatattcatattcctaaagccactattgtgttttctctaagaagcactacatgccaccagaattgtgcactgaaagataatacaaactgagtgtctttatgagaatcactgtgtcccctgaggcccagcagtacctgcttccctgtatgtggaagcagcacctcattcccgccatcagctacctctcatcaccccaccttcatcatcatgctccagggtcaccctggccagctcttgtgctgggacaggggattacaccatctctgttcaaagagggggaaatgtgcctatgccttaagtcaatgcactcagcaaggagaagcacatcttatttatcttgttacctatagtttactttgggtgattggaggggaatgacttagttatgactggacatcttaaaagctgatagacaagccaaatggctggcagatgatgtggatttcaaagagcccagaatgaactcatcactggcttagacagtcctggatgccatttggaaagtagtggccctgcaagcctaaatgaagtagtatttgtaggcctgggtggcatttggatttttcttttccctcaacaaggttttactttttcttactttacaagcaagggaagttttgtgataggagagaaataaaagatttgatattttttgagatgacactcaagcatcaggctgagatttgcacacatgggatgtaaaagcaagctgtgtgttgcttagtcacttacttagaagtagatggtgggggacagcggcgtgggtcctagcctggccagtgatgctgctggcgtccagaccccagactcactccaagcactcttgttcaatatctcatgcagaagagttgggctggtcactcttaggggtgagaccccgtgattggttggtttgtagcactaaggtctaaaaaggaaaaccataaaagacatcagattacgctggatcagtataattaatattccatagggccatgttgccagaatctgtatgtatcaatacagggtttttccaagccaggaaacgccctccttggctactaggagcacctatcccatatcattcagataaacaatgatagctacaaagtcatttgtggctagataggttaagacaggtgattttttaaagtagactgtctttgcattttgccatctgaagttcattaatctttagatgacaaaaaagcaaaaagttcccagaacgtttttgcttagattttgttctaatcaccacgtgaaggaatgagattagcaccacaagtttcatgccaataaaagagactggtgtgatcccacatgcaaaatttaatcctaagggtagtgagatcacaaacagaataaaaataagagcaatcaaccatataaatcaagtacctattgggaacagacataacattcaatttttcatttatgctaagtgaccacagtatacaaagtaataagcaggaaatttgatatgggttaaattatgcattttgttcagattttggaaattggtatgcattataagtttctcaatgtacatctttttatcccaacaccctcaaactagaatatttgtcagtggtcaagagaaaaaattttacctgaattcttgggggccggcggggagcttttacattaaaaatctactaacgcctactttttaaaaaatgagattctttctaatctttatatatgacattttctagacaatcgcacctttgggtatattaaacagctggtatcataacaaagaatccaaatgaaccttcaatatactagaagttctagtaggttaatattgttcagaagatttttacaaattaaaaactgatttccaaatatgttcaacatttacttctatttgatatctgctcaagaagtcatagaagtcttgggaaactattcgagtatcacagaggttttcaaaagcccttatggtgacatctacctaggtaaaagcctgacatgtggctttataattgttatgttacccaagggataaacttgaactggctttgaacatcctttaggtcattttctctttggataattttcatcgcatatccagcaactatagaccaaagtttgcttaaggtttgaccctagagcagaggtgggcaaactatgacctggaaaccaaatctgacttaccaccttttcctgtagttaaagttttattgtcacatagccacatacattcatttacatgttttctgattttacactacagcagcaggatggagtagttgtgtcagagactatgtatcccacaaagcatagattacttactatttagtcctttacaggcaaagtttcctcacccctgacctagaggtttttgtggtatgcattggatatagcaggaaagaaagcacatttccaaacagcagggagtaagcttacatttctgtgtaggtttgggaatatagttacttggcaaagtctttcaggaaggaagcccttccttatgttacatgtggaaagcctgccttccaagacatgtggaagtaattgatccacctgccagagaaacacaggctcagaggatgcctgggaacagggaggatgggattagtggaagcttaatggaaaaggaaagttatgatcctccaagacccttaattgatagaccataccaggttctgcaggtcagcatcattgtgtaatgagagtgaagtaggggaccctgtggttcaaccttagaatctgtttcctgtaggctctttctgctgtctatattcattaaagttttccacttcaccctcccatagtctagagggatgcccattccatggtcctccagagaatagttttgacttaacatgtctgtttagcccacatcacgtcagttatcaacaccgccactgtgcttactgttcctacagccacaccaggcttgaagagttagtgagaccaacaaataattggaagtattggaaaaagcaaaatacatggggacaaaaaaaatacagtgaaattctttttatcaaactgatgctgtgagaaaccagatgaatgccagtttggctttatttctaagaatctgggtcttcattctctggtgtagaaggaatgcaaaaaactataacaacaacaacaaaaacatattttgaaaagacatattctgacatctctgcttgtgtgtggtaaggcaggttcctatcagacatttatccctttggtcaagatcccttttgctcatccagggtttcatactcaatatcgcttaaaaaaaaaaaagtatcagctagggatgactctggaagtatgagtatcatggtggggaggaaggaattttttttaaatgtaaatgacccccatttaccagaccctaatcaaagtcacttaagggaatccctcagccttttatttggaaacagttgaaataaactggcagcagctagatcagagtatcttgctttattttataaaggccaaaggtagtatgaagtttggaccaaaaaggtaaatagatccattccagcacctgatactgatttttcaaggctctatgaaaggtcaaaaatttcattaaacaagaccagttctccctcttccccctgtcccaagaaatcttaggcatgaaaaggataaggaaacagctcctggaatgatacatttgcatagtgccctagtagcaggttgggaaaaagttataatataagaaacaaccttcgaaaacaggccttttatctcaaagataaaatgtctttcttgtggtctttcatcactatctccgtggtggaaggttcccctagttccaacatatttccattaaaatagtcaaagccacggcattgggatgtcagatgcctctctttctttgtggtaatcggaatttaaaattatacagttgcctctgaatttctcatgcacaaagccaaaccactgataagagataaagcagtctgaagcctgctgcttcagccagcacagcacaccacacacgctcgcactttcaaaagcaatgtgatttctatggttcttaaaagctttcttcataagggagtccctgaaatttctcaaggcaggtttgaatggcaaagggaaaataattacttgtgggaggtcctccttttgagtattgttagagcatacatgtaaaagaaaataacctttttggggcaactcatgctcacacatgctgttttctttggttctccccctaccttccttttgtagatattgacagaataggaggaaatgagcatccttatttgagaaagagcaagaatgtcatgagcccttgatgcaatagtaagtgtgatgtcatcatacagtgttaatgatgctatcaaatccatcaataaacagtctcaaaccttccaacaacagtgctcactgctgctcctcaacttcagcccagcaagcagtaatatcatcacccattttgagatatgcaggtggaatagaacaaagaaacagccactgtaatcgagaagcatgtttactgtctaaatccacctgttgcagtaggaagccagagtggggttccaaatgcctcattaagtatgtggacagcctcactagtaagtgagtgaatttggcttcatcactgaacattagctaaggtcagcttaataacacaaatatgaggccgacttctttgcgagaagagaaaagaaaacatctgcttgatttaaaatccaccccacatgcctagagttgtctaatagtccctcactttccaatggcttcacatcctacttctacatttggggttttttggtgaaatcagagatagctcaggatttcataaaacgagaaactccaaactggtctattaggttccatgggaacacttgtagccaaaggattgtctgagggcaggaagacgacacttgtcaacaaggaagacagtgtttctttagttcccatattcatctaattcatggggttctaacattttggggggccatagattcttttgacaatctgagccaatctatgaaacttctccccaaaaagaaccaccccacaaaatcttgcaaaacgtaagagattttcctggatctgaagctacccgaagacatgggaagagttgtattctattattcaattttaagaatatttattaatattcactgagctattgctagccactgtgctaaacattttacatacattctcctatttcatcctcaaaacaatcctttgagttgggttttaatatagttccaatattcggatgtgaaaactgaggcttatagtggctaagaaacttgcccaaagccactacctagaaaatggcagagctggaatgtaggtttaactcctgaccactatgctataaagtcaccacatgtcaaactaattttcaagttgttgggacatgtcccctactaggttttaaactagatcttccttggtagatgaagctatagaacttctattttccctgctttctgtagtctctcacagtgacagcatctatactaaagtatagatacctaaggggaaaatatagaaagcctgcctgaataatagaatctagaacaacaacaaaaatattaattttttctgtgtatgcttaggtcaagcttaaaaaaaaaaaaaaaagaccggaaaatacctgggttgttagcctcacatttaggaaaaaattgtaatactcagttatctgtgtgtgtggctaaacaagtcagcatttctgcacacatacatctctttcctttatacttcccttcaaaagacaaatatcttacttttgatctttgacactatttggtcagtattcttctttacctttacttgtggcaaaactcaaggaagcgatcaaatagagggaagctcatttctatcattgtctctgtttccctataagaaagaactaccagggactcactgactgcattaggcatacaatgtcagagctgagctgaccactctggtcctgtaatgtctttggcctcacaccttggcagccatcattaatgggccatacccttccccaggtgcagaattctcctccccagagcactcaggccgttactaccaatttatctgagttggaaataagactcatttgccagttcttatttttaaagtggcaccctttaactttgaacctgtgtattttacactggcatcctagattcagcaatgaggtttggtggtgtttcaactaggaagggagaaaatgagtgcatctgaagttccttacagcttggtttctttggaatgctttcatcttctaagcaaagggatcagggtttgatctgtaagagttaaaaagacaaagtcattttgaagaattaactcagccagggatcatgcaaaaagattagaaaccataatgcccttgttaaagccctgctgtcaacctgccttcacccagagcttagagggccacagcagcaaagaggttggggtccatccctctctgatgtgctttttccacaacacatatctggtcctctggcaggattgtggatagagctcctcaccatacccaaaagactcagccccagtgccagtgctttcctggttcaacaacccaccacaaaaccttagtaaaaggatgagccaaaaatgaaaaagactcgactctacagtaagtcagtcagggatttcctttttaatggtttaagacatccaaatggcaagccaggaatagataccattaaagggtctcataggactaaccttaccagagccagaaatctagctctctggaagagatgcaagattctagaaaagtaaagggaagtgtcggcacatctaaatttagtgaacacaaaattaatttttatctagtctgtgacggagggaataaagtttttcatgtatcaaccacctcccccagtcaggtttctccctttttgagattatgaagaagctgagacatacttcttaaggaggtcgtgttttagaaggaaaaggcagaggctatccatcattatgctggctagatgcgcttctgaagaagccggattctgatgttcttaaccaaaatggtgaggtcatggaagtcccatttgcttggagattttgaaaaaaaaaaaaaaaaaaaacccattcccataaagtaattgagttcagcctttggattatttttggtttggtttttctctggttttgggtgtgatgtaagaagagctttttagttttgttttgaataacatcaatccttgcacactctatgcaaaaattttgtaagcatttcaataatgctatgaattacaaggaactattttaactttattacactttctgtataaaaaatttgtatttaatattatttcgaccacagtcttgtaaaatatattaataaaaataatgattggtaagaaggaaa

SEQ ID NO: 32 - DNA, 1166 bp, Homo sapiens
agagctgctcagcacgccctgggatcgcgggcggttttcatcggccggtttgtgaaacggacaagagagagacattactgccgggagtgttgagtgaagggaccaggtggagatgatgcctcaggagcagtacactcaccaccggagcaccatgcctggctctgtggggccacaggtatacaaggtggggatttatggctggcggaaacgatgcctgtatttctttgtcctgctcctcatgattttaatactggtgaacttggccatgaccatctggattctcaaagtcatgaacttcacaattgatggaatgggaaacctgaggatcacagaaaaaggtctaaagctagaaggagactctgaattcttacaacctctctacgccaaagaaatccagtcccgaccaggtaatgccctgtacttcaagtctgccagaaatgttacagtgaacattctcaatgaccagactaaagtgctaactcagcttataacaggtccaaaagccgtagaagcttatggtaaaaaatttgaggtaaaaactgtttctggaaaattgctcttctctgcagacaataatgaagtggtagtaggagctgaaagattacgagttttaggagcggagggcacagtgttccctaaatctatagaaacacctaatgtcagggcagaccccttcaaagaactaaggttggagtccccaacccggtctctagtgatggaggccccaaaaggagtggaaatcaatgcagaagctggcaatatggaagccacctgcaggacagagctgagactggaatccaaagatggagaggtgagggatgagaaggacagaagttcaaagagctacagcttcaacaggccaacccttcccataactggttgacctcggagttggatcctacagtgtatcaacaaaaggagccaagcaggttttatttctgaaacaattaattgagcagcatgattataagccaaacccacaatccatcaaagtgatgatttcttatttgtaaaatgcagagataatggcatgtattccaagtacagaattatatgaccatgaaaatgaatgctattttcaaattctctcttgtcaccttaaaataagattttgttagccaacataattaagctgtatatattatacacatctggctcaagatgaa

SEQ ID NO: 33 - protein, 290 aa, Homo sapiens
MMPQEQYTHHRSTMPGSVGPQVYKVGIYGWRKRCLYFFVLLLMILILVNLAMTIWILKVMNFTIDGMGNLRITEKGLKLEGDSEFLQPLYAKEIQSRPGNALYFKSARNVTVNILNDQTKVLTQLITGPKAVEAYGKKFEVKTVSGKLLFSADNNEVVVGAERLRVLGAEGTVFPKSIETPNVRADPFKELRLESPTRSLVMEAPKGVEINAEAGNMEATCRTELRLESKDGEIKLDAAKIRLPRLPHGSYTPTGTRQKVFEICVCANGRLFLSQAGAGSTCQINTSVCL

SEQ ID NO: 34 - protein, 289 aa, Homo sapiens
MPQEQYTHHRSTMPGSVGPQVYKVGIYGWRKRCLYFFVLLLMILILVNLAMTIWILKVMNFTIDGMGNLRITEKGLKLEGDSEFLQPLYAKEIQSRPGNALYFKSARNVTVNILNDQTKVLTQLITGPKAVEAYGKKFEVKTVSGKLLFSADNNEVVVGAERLRVLGAEGTVFPKSIETPNVRADPFKELRLESPTRSLVMEAPKGVEINAEAGNMEATCRTELRLESKDGEIKLDAAKIRLPRLPHGSYTPTGTRQKVFEICVCANGRLFLSQAGAGSTCQINTSVCL

SEQ ID NO: 35 - protein, 256 aa, Homo sapiens
MMPQEQYTHHRSTMPGSVGPQVYKVGIYGWRKRCLYFFVLLLMILILVNLAMTIWILKVMNFTIDGMGNLRITEKGLKLEGDSEFLQPLYAKEIQSRPGNALYFKSARNVTVNILNDQTKVLTQLITGPKAVEAYGKKFEVKTVSGKLLFSADNNEVVVGAERLRVLGAEGTVFPKSIETPNVRADPFKELRLESPTRSLVMEAPKGVEINAEAGNMEATCRTELRLESKDGEVRDEKDRSSKSYSFNRPTLPITG

SEQ ID NO: 36 - DNA, 6964 bp, Homo sapiens
  modified_base (432)..(531): a, c, t, g, unknown or other
  modified_base (650)..(650): a, c, t, g, unknown or other
  modified_base (862)..(862): a, c, t, g, unknown or other
  modified_base (916)..(1015): a, c, t, g, unknown or other
  modified_base (1456)..(1555): a, c, t, g, unknown or other
  modified_base (1990)..(2089): a, c, t, g, unknown or other
  modified_base (2328)..(2427): a, c, t, g, unknown or other
  modified_base (2703)..(2802): a, c, t, g, unknown or other
  modified_base (2821)..(2821): a, c, t, g, unknown or other
  modified_base (2896)..(2896): a, c, t, g, unknown or other
  modified_base (3142)..(3241): a, c, t, g, unknown or other
  modified_base (3508)..(3607): a, c, t, g, unknown or other
  modified_base (4034)..(4133): a, c, t, g, unknown or other
  modified_base (4394)..(4493): a, c, t, g, unknown or other
  modified_base (4898)..(4997): a, c, t, g, unknown or other
  modified_base (5445)..(5544): a, c, t, g, unknown or other
  modified_base (5548)..(5548): a, c, t, g, unknown or other
  modified_base (5825)..(5825): a, c, t, g, unknown or other
  modified_base (5905)..(6004): a, c, t, g, unknown or other
  modified_base (6365)..(6464): a, c, t, g, unknown or other
gaagtcataactgatagaagatcatctactttgtttacatgtttgaatcatatagattcaagtcagttaatttcactaaaactcatcaattattattcatcaattagggtaaatgtatttaaaaaattgttttttaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcaggtaattcaggattttactttctaccctcatttttatttacttgttttttccctaacgatacactgtaaactgtaaaggtacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnataaaaatgcagtmttagtaaaaatattctttgcctmaagaactacttagagacatcctttaaacatgggaattgtttttgggcctgtgtttagacataacacaatgatgaattgtgtnaaaagtaatcagcacaccagtaatgccttataacgggtctcgtttcagaatcacatacaaaccctgaagaaatggatggctgaagttgatgtttttctgaaggaggaatggcctgcccttggggattcagaaattctaaaaaagcagctgaaacagtgcagagtaagatttttatatgatgcctttaatatgaataattttgtatgaatatnatttggttagatcagtgttttacagctggggtggattttgctctcctctccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagactgttaggcagtcatctatatcaaatatctctgtatctcaagatcccaaaagacaaaaatccaatatgcaatgccatcagttcccaattttctagctatgtttcatatctatatgtggcagtaatttttttcagctggcttaaattgatttattttcttagcttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtatagacaatctctttcactgaggcttgcctcaacgtacttaactaagatttcctaatgtctcccttcaccgttacttttggttaaggctttgttcctatgtttttgctttaaagcacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcatgcatataagctttttacttctttatcattactatctaagctttctattctttacatactgatgaaataatataataataatgtttcatcactgtcaataatcgtgttttgtttgtttgttttgtggaaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaaggtaaaaaaaaaaaagaaaaactaagtaaaacaaaggaaataaatggaaaaagaaagaaatgcaacaatgcttgaagtcgtatacagtctgctctttcctggttctaagagaagaggttgattcttcattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagtcagttgcttttcttggtctttgtcaattatatgtcaatacatggtcatagttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaagttttaataatgaaatggcaaaatttcacatttacttttctaccataatatttaatctgtgatatatatttctttcttaggaagtttgggcatgttggcatgagttattgtcatacttggagaaagcaaacaagtggctaaatgaagtagaatttaaacttaaaaccactgaaaacattcctggcggagctgaggaaatctctgaggtgctagatgtaagttgtaaattaagccaaatgatgatgatttatatgcagtattaaaagaggtacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgctttaagattatgancttaatgatatgtaaatcagaagatactgagcatttgctgataatccaatgtatttagaaaaaaaaggagaaatngtaattattgcaaatgtgtttcagtcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggtattaagataagtgaaaatctctttaatctaatttgcattaatgtatagcagatacagctctcagatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagctatgaagggtaatcgttttacctgatacagtyttattycttctttttaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccaggcaagtacatctgggaatcagcttccattctttttgtttgtatgacctcacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacgtgtaagaaaatagttattttaaataacagaaatataaaagttccaaataagtggttataacgaaatttgaattaaagagtaaactaaattacatttcataataattcttttcaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggtaaaacctatatcactgaaggttattttgaacatacgtgaaaacacataatatgattttgtaaggaagtattaacatgtagcaataatagcatcataaatattaatattgtttcatattccctttcttaaaattaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaataaatgtatttcttttggtttatgtttcttataaaaagtaattttgatttaaagtagractacctttttttttaggcctccattcctttgaaggaattggagcagtttaactcagatatacaaaaattgcttgaaccactggaggctgaaattcagcagggggtgaatctgaaagaggaagacttcaataaagatatggtaaattggttgtgaatgaactaggagtggaaataaatatttggaaagaacttagataagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggattaaagtgtgtgtttaaataacatgtctaattatctctgttaacaatgtacagctttttaaaaaccaaaatgaagactgtacttgttgtttttgatcagaatgaagacaatgagggtactgtaaaagaattgttgcaaagaggagacaacttacaacaaagaatcacagatgagagaaagcgagaggaaataaagataaaacagcagctgttacagacaaaacataatgctctcaaggtattagagctaaaattataatataccttgcctgtggtttttttttaatatatagggtaatatataatgtgcattaataaaatctgcttcagactcttagtcatcagaaactcactttttctgttcaatgtgtatgcttatttaacatttttgaggtggtatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacctaagttgtacaaaaagatgagggacgcaagttattttgataaataactgcagccagaagtgcactatacatatatattgatattttaataatgtctgcaccatgaacaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggtaatgttgtgttttagaatgtcaataccagattttattatacagtttaattaacctgtgaagatcatatttaaaatgttgatgttcttgtttctattaacgttctctttgagggatgcatttccttgtattgtgtgttgttttcagtaaatgtattgtattaaggtgttattcaattgacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatnacattcatagcttgtttcttttctttrgtaattctgcacatattcttcttcctgctgtcctgtaggacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtaggaaaagatgtggagcaaaaaggccacaaataaatnaaaatggccaaattttcctcattgtcttagcacaagtaactggtatctcacatgtctacgtaaatcatcccaatttcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaccatcagtgtataaataaaagagatagaaattgacctggagtttcataaacaagttctgagcacccaggattaattttgagaagaatgccacaagcctttcttagcacttcttttcatctcatttcacaggccttcaagagggaattgaaaactaaagaacctgtaatcatgagtactcttgagactgtacgaatatttctgacagagcagcctttggaaggactagagaaactctaccaggagcccagaggtaattgaatgtggaactataataacatattgatagaaggatcagtggtgacggagcagcccatccattcttgctgccagggtctggatagctctcatattttcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaacgtcggattgatataaggtagaaactcaggaagatatactttagatgttctgggctgtatcaaaatttatgccaaagtataaaaaagccgttaatcagtaggttaccctcttgttcaactgtactctttctttcttccagtatgacctttttgacaatgtttaaaaaaaaagaatgtggcctaaaaccttgtcatattgccaatttagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggtacggtctacttctttact

SEQ ID NO: 37 - protein, 428 aa, Homo sapiens
ALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACTRFLLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQ

SEQ ID NO: 38 - DNA, 3581 bp, Artificial Sequence
  Description of Artificial Sequence Synthetic polynucleotide
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagc

SEQ ID NO: 39 - protein, 1192 aa, Artificial Sequence
  Description of Artificial Sequence Synthetic polypeptide
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 40 - DNA, 5417 bp, Artificial Sequence
  Description of Artificial Sequence Synthetic polynucleotide
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctaga

SEQ ID NO: 41 - DNA, 5339 bp, Artificial Sequence
  Description of Artificial Sequence Synthetic polynucleotide
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctaga

SEQ ID NO: 42 - DNA, 5462 bp, Artificial Sequence
  Description of Artificial Sequence Synthetic polynucleotide
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatgctcctggactgaccactattggagcctctcctactcagactgttactctggtgacacaacctgtggttactaaggaaactgccatctccaaactagaaatgccatcttccttgatgttggagcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctaga

SEQ ID NO: 43 - DNA, 8689 bp, Artificial Sequence
  Description of Artificial Sequence Synthetic polynucleotide
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaagacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 44 - DNA, 11443 bp, Artificial Sequence
  Description of Artificial Sequence Synthetic polynucleotide
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaagacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 45 - DNA, 1164 bp, Homo sapiens
atggccgagacactgttctggactcctctgctggtggtgctgctggctggactgggagataccgaggctcagcagaccacactgcacccactggtgggccgggtgttcgtgcacaccctggaccatgagacatttctgagtctgccagaacacgtggctgtgccacctgctgtgcatatcacttaccacgcccatctgcagggccatcctgatctgccacggtggctgagatacacccagagatcaccccaccatcctggattcctgtatggaagcgctaccccagaggacaggggactgcaggtgatcgaagtgacagcttacaaccgcgacagttttgatactaccaggcagcgcctggtgctggagattggggatccagaaggacccctgctgccttatcaggccgagttcctggtgcggtcacacgacgctgaggaagtgctgccatcaacacccgccagcagatttctgtccgctctgggaggactgtgggagccaggagaactgcagctgctgaatgtgactagcgctctggataggggaggaagggtgccactgccaatcgagggaaggaaggaaggggtgtacattaaagtgggaagcgcttccccattctccacctgcctgaagatggtggcttctcctgatagtcacgctaggtgcgctcagggacagccaccactgctgtcctgttatgacacactggccccccattttcgcgtggactggtgcaacgtgactctggtggataaatctgtgcctgagccagctgacgaagtgccaacccctggagacggaatcctggagcacgatcctttcttttgtcctccaacagaagccccagacagggatttcctggtggacgctctggtgactctgctggtgcctctgctggtggctctgctgctgaccctgctgctggcttatgtgatgtgctgtcggagagagggacggctgaagagagacctggccacatctgatatccagatggtgcaccattgtactattcacggcaacaccgaggaactgcgccagatggctgcttctagggaggtgccaaggccactgagtacactgcctatgtttaatgtgcacactggcgaacggctgccccctagagtggatagcgcccaggtgccactgattctggaccagcattga

SEQ ID NO: 46 - protein, 387 aa, Homo sapiens; identical to SEQ ID NO: 15

SEQ ID NO: 47 - DNA, 2476 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtaccaccatggccgagacactgttctggactcctctgctggtggtgctgctggctggactgggagataccgaggctcagcagaccacactgcacccactggtgggccgggtgttcgtgcacaccctggaccatgagacatttctgagtctgccagaacacgtggctgtgccacctgctgtgcatatcacttaccacgcccatctgcagggccatcctgatctgccacggtggctgagatacacccagagatcaccccaccatcctggattcctgtatggaagcgctaccccagaggacaggggactgcaggtgatcgaagtgacagcttacaaccgcgacagttttgatactaccaggcagcgcctggtgctggagattggggatccagaaggacccctgctgccttatcaggccgagttcctggtgcggtcacacgacgctgaggaagtgctgccatcaacacccgccagcagatttctgtccgctctgggaggactgtgggagccaggagaactgcagctgctgaatgtgactagcgctctggataggggaggaagggtgccactgccaatcgagggaaggaaggaaggggtgtacattaaagtgggaagcgcttccccattctccacctgcctgaagatggtggcttctcctgatagtcacgctaggtgcgctcagggacagccaccactgctgtcctgttatgacacactggccccccattttcgcgtggactggtgcaacgtgactctggtggataaatctgtgcctgagccagctgacgaagtgccaacccctggagacggaatcctggagcacgatcctttcttttgtcctccaacagaagccccagacagggatttcctggtggacgctctggtgactctgctggtgcctctgctggtggctctgctgctgaccctgctgctggcttatgtgatgtgctgtcggagagagggacggctgaagagagacctggccacatctgatatccagatggtgcaccattgtactattcacggcaacaccgaggaactgcgccagatggctgcttctagggaggtgccaaggccactgagtacactgcctatgtttaatgtgcacactggcgaacggctgccccctagagtggatagcgcccaggtgccactgattctggaccagcattgaggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 48 - DNA, 5749 bp, Artificial Sequence
  Description of Artificial Sequence Synthetic polynucleotide
atgcagctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtaccaccatggccgagacactgttctggactcctctgctggtggtgctgctggctggactgggagataccgaggctcagcagaccacactgcacccactggtgggccgggtgttcgtgcacaccctggaccatgagacatttctgagtctgccagaacacgtggctgtgccacctgctgtgcatatcacttaccacgcccatctgcagggccatcctgatctgccacggtggctgagatacacccagagatcaccccaccatcctggattcctgtatggaagcgctaccccagaggacaggggactgcaggtgatcgaagtgacagcttacaaccgcgacagttttgatactaccaggcagcgcctggtgctggagattggggatccagaaggacccctgctgccttatcaggccgagttcctggtgcggtcacacgacgctgaggaagtgctgccatcaacacccgccagcagatttctgtccgctctgggaggactgtgggagccaggagaactgcagctgctgaatgtgactagcgctctggataggggaggaagggtgccactgccaatcgagggaaggaaggaaggggtgtacattaaagtgggaagcgcttccccattctccacctgcctgaagatggtggcttctcctgatagtcacgctaggtgcgctcagggacagccaccactgctgtcctgttatgacacactggccccccattttcgcgtggactggtgcaacgtgactctggtggataaatctgtgcctgagccagctgacgaagtgccaacccctggagacggaatcctggagcacgatcctttcttttgtcctccaacagaagccccagacagggatttcctggtggacgctctggtgactctgctggtgcctctgctggtggctctgctgctgaccctgctgctggcttatgtgatgtgctgtcggagagagggacggctgaagagagacctggccacatctgatatccagatggtgcaccattgtactattcacggcaacaccgaggaactgcgccagatggctgcttctagggaggtgccaaggccactgagtacactgcctatgtttaatgtgcacactggcgaacggctgccccctagagtggatagcgcccaggtgccactgattctggaccagcattgaggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccatcttcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

(19) United States
(12) Patent Application Publication
(10) Pub. No.: US 2023/0040544 A1
(43) Pub. Date: Feb. 9, 2023

(54) COMPOSITIONS AND METHODS FOR RESTORING AND MAINTAINING THE DYSTROPHIN-ASSOCIATED PROTEIN COMPLEX (DAPC)

(71) Applicants: Research Institute at Nationwide Children's Hospital, Columbus, OH (US); Sarepta Therapeutics, Inc., Cambridge, MA (US)

(72) Inventors: Eric Pozsgai, Cambridge, MA (US); Louise Rodino-Klapac, Cambridge, MA (US)

(21) Appl. No.: 17/785,879

(22) PCT Filed: Dec. 15, 2020
(86) PCT No.: PCT/US2020/065157

§ 371 Date: Jun. 15, 2022

Related U.S. Application Data
(60) Provisional application No. 62/948,586, filed on Dec. 16, 2019.
(60) Provisional application No. 63/023,144, filed on May 11, 2020.

(51) Int. Cl. A61K 38/17; A61K 48/00; A61P 21/00

(52) U.S. Cl. CPC A61K 38/1719; A61K 48/0008; A61K 48/0058; A61P 21/00

(57) ABSTRACT

Disclosed herein are methods of repairing or restoring a sarcoglycan complex or DAPC, stabilizing DAPC, restoring DAPC function, or increasing or enhancing expression of one or more components of a sarcoglycan complex or DAPC in a subject suffering from a muscular dystrophy.

### CROSS REFERENCE TO RELATED APPLICATIONS

This application claims benefit under 35 U.S.C. § 119(e) of U.S. Provisional Application No. 62/948,586 filed Dec. 16, 2019 and U.S. Provisional Application No. 63/023,144 filed May 11, 2020, the contents of both of which are incorporated herein by reference in their entireties.

### TECHNICAL FIELD

This disclosure provides methods of enhancing expression of one or more components of a sarcoglycan complex and/or dystrophin-associated protein complex (DAPC), restoring or stabilizing a DAPC, restoring DAPC function, and localizing components of a sarcoglycan complex and/or DAPC complex to a cell membrane by administering a sarcoglycan, sarcospan, and/or dystrophin transgene to a subject in need thereof.

### BACKGROUND

Limb-girdle muscular dystrophy type 2 (LGMD2) is caused by recessive mutations in a variety of muscle specific genes involved in the structure and function of muscle cells. Duchenne muscular dystrophy (DMD) or Becker Muscular Dystrophy (BMD) is caused by mutations in the dystrophin (DMD) gene. A subset of type 2 LGMDs (sarcoglycanopathies) involve mutations in the sarcoglycan proteins (α-, β-, γ-, and δ-). These mutations ultimately lead to protein deficiency, loss of function in proteins involved in muscle homeostasis and membrane repair, and loss of stabilization of the dystrophin-associated protein complex (DAPC).

There is an urgent need for a therapy that can stabilize the DAPC in type 2 LGMD patients.

### SUMMARY

Without wishing to be bound to theory, the sarcoglycans and a protein called sarcospan along with dystrophin are integral proteins critical for stabilizing the DAPC and providing mechanical support to the sarcolemma.

Disclosed herein are methods of enhancing expression of one or more components of a sarcoglycan complex and/or dystrophin-associated protein complex (DAPC), restoring or stabilizing a DAPC, restoring DAPC function, and localizing components of a sarcoglycan complex and/or DAPC complex to a cell membrane by administering a sarcoglycan, sarcospan, and/or dystrophin transgene or its abbreviated version to a subject in need thereof.

In some embodiments, a method of restoring or stabilizing a dystrophin-associated protein complex (DAPC) in a subject suffering from muscular dystrophy, comprises, or consists essentially of, or yet further consists of administering to the subject a polynucleotide sequence encoding (a) a sarcoglycan and/or (b) dystrophin or abbreviated version thereof. In one aspect, the polynucleotide further comprises a promoter and/or an enhancer element, non-limiting examples of such are provided herein, e.g., in the sequence listing of this disclosure.

In some embodiments, the muscular dystrophy is Duchenne muscular dystrophy (DMD) or Becker Muscular Dystrophy (BMD). In some embodiments, the method comprises, or consists essentially of, or yet further consists of administering to the subject a polynucleotide encoding dystrophin or a fragment of dystrophin. In some embodiments, the abbreviated version of dystrophin is a microdystrophin or mini dystrophin. In one aspect, the polynucleotide further comprises a promoter and/or an enhancer element, non-limiting examples of such are provided herein, e.g., in the sequence listing of this disclosure.

In some embodiments, the muscular dystrophy is LGMD2C. In some embodiments, the method comprises, or consists essentially of, or yet further consists of administering to the subject a polynucleotide encoding the sarcoglycan, wherein the sarcoglycan is SGCG.

In some embodiments, the muscular dystrophy is LGMD2D. In some embodiments, the method comprises, or consists essentially of, or yet further consists of administering to the subject a polynucleotide encoding the sarcoglycan, wherein the sarcoglycan is SGCA.

In some embodiments, the muscular dystrophy is LGMD2E. In some embodiments, the method comprises, or consists essentially of, or yet further consists of administering to the subject a polynucleotide encoding the sarcoglycan, wherein the sarcoglycan is SGCB.

In some embodiments, the muscular dystrophy is LGMD2F. In some embodiments, the method comprises, or consists essentially of, or yet further consists of administering to the subject a polynucleotide encoding the sarcoglycan, wherein the sarcoglycan is SGCD.

In some embodiments, a method of localizing a first sarcoglycan, sarcospan, and/or dystrophin to a muscle cell membrane or sarcolemma in a subject suffering from muscular dystrophy, comprises, or consists essentially of, or yet further consists of administering to the subject a polynucleotide sequence encoding (a) a second sarcoglycan; and/or (b) dystrophin or abbreviated version thereof, wherein the first sarcoglycan is different from the second sarcoglycan. In one aspect, the polynucleotide further comprises a promoter and/or an enhancer element, non-limiting examples of such are provided herein. In one aspect the polynucleotide further comprises a MHCK7 promoter polynucleotide and an alpha heavy chain enhancer.

In some embodiments, a method of increasing or enhancing expression of a first sarcoglycan, sarcospan, and/or dystrophin to a muscle cell membrane or sarcolemma in a subject suffering from muscular dystrophy, comprises, or consists essentially of, or yet further consists of administering to the subject a polynucleotide sequence encoding a second sarcoglycan; and/or (b) dystrophin or abbreviated version thereof, wherein the first sarcoglycan is different from the second sarcoglycan. In one aspect, the polynucleotide further comprises a promoter and/or an enhancer element, non-limiting examples of such are provided herein. In one aspect the polynucleotide further comprises a MHCK7 promoter polynucleotide and an alpha heavy chain enhancer.

In some embodiments, the muscular dystrophy is Duchenne muscular dystrophy (DMD) or BMD. In some embodiments, the method comprises, or consists essentially of, or yet further consists of administering to the subject a polynucleotide encoding dystrophin or an abbreviated version of dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the abbreviated version of dystrophin is a microdystrophin or mini dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In one aspect, the polynucleotide further comprises a promoter and/or an enhancer element, non-limiting examples of such are provided herein. In one aspect the polynucleotide further comprises a MHCK7 promoter polynucleotide and an alpha heavy chain enhancer.

In some embodiments, the muscular dystrophy is LGMD2C. In some embodiments, the method comprises, or consists essentially of, or yet further consists of administering to the subject a polynucleotide encoding the second sarcoglycan, wherein the second sarcoglycan is SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In one aspect, the polynucleotide further comprises a promoter and/or an enhancer element, non-limiting examples of such are provided herein. In one aspect the polynucleotide further comprises a MHCK7 promoter polynucleotide and an alpha heavy chain enhancer.

In some embodiments, the muscular dystrophy is LGMD2D. In some embodiments, the method comprises, or consists essentially of, or yet further consists of administering to the subject a polynucleotide encoding the second sarcoglycan, wherein the second sarcoglycan is SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In one aspect, the polynucleotide further comprises a promoter and/or an enhancer element. In one aspect the polynucleotide further comprises a MHCK7 promoter polynucleotide and an alpha heavy chain enhancer.

In some embodiments, the muscular dystrophy is LGMD2E. In some embodiments, the method comprises, or consists essentially of, or yet further consists of administering to the subject a polynucleotide encoding the second sarcoglycan, wherein the second sarcoglycan is SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In one aspect, the polynucleotide further comprises a promoter and/or an enhancer element, non-limiting examples of such are provided herein, e.g., in the sequence listing of this disclosure.

In some embodiments, the muscular dystrophy is LGMD2F. In some embodiments, the method comprises, or consists essentially of, or yet further consists of administering to the subject a polynucleotide encoding the second sarcoglycan, wherein the second sarcoglycan is SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In one aspect, the polynucleotide further comprises a promoter and/or an enhancer element, non-limiting examples of such are provided herein, e.g., in the sequence listing of this disclosure.

In some embodiments, the method increases or enhances expression of the first sarcoglycan at or increases localization of the first sarcoglycan to the muscle cell membrane or sarcolemma. In some embodiments, the first sarcoglycan is SGCD. In some embodiments, the first sarcoglycan is SGCB. In some embodiments, the first sarcoglycan is SGCA. In some embodiments, the first sarcoglycan is SGCG. In some embodiments, the expression of the first sarcoglycan at or localization of the first sarcoglycan to the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of the first sarcoglycan prior to administering one or more doses of the polynucleotide. In some embodiments, the method further comprises, or consists essentially of, or yet further consists of detecting expression of the first sarcoglycan. In some embodiments, detecting expression of the first sarcoglycan comprises, or consists essentially of, or yet further consists of detecting protein levels of the first sarcoglycan. In some embodiments, detecting expression of the first sarcoglycan comprises, or consists essentially of, or yet further consists of detecting RNA levels of the first sarcoglycan. Any known methods in the art can be used to detect protein and/or RNA levels of the first sarcoglycan. Such methods included, but are not limited to, western blot, PCR, immunofluorescence, and ELISA. In some embodiments, detecting expression of the first sarcoglycan comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

In some embodiments, the method increases or enhances expression of dystrophin to the muscle cell membrane or sarcolemma. In some embodiments, the expression of dystrophin or localization of dystrophin to the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of dystrophin prior to administering one or more doses of the polynucleotide. In some embodiments, the method further comprises detecting expression of dystrophin. In some embodiments, detecting expression of dystrophin comprises, or consists essentially of, or yet further consists of detecting protein levels of dystrophin. In some embodiments, detecting expression of dystrophin comprises, or consists essentially of, or yet further consists of detecting RNA levels of dystrophin. Any known methods in the art can be used to detect protein and/or RNA levels of dystrophin. Such methods included, but are not limited to, western blot, PCR, immunofluorescence, and ELISA. In some embodiments, the dystrophin is the abbreviated dystrophin. In some embodiments, detecting expression of dystrophin comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

In some embodiments, the method increases or enhances expression of sarcospan at or increases localization of the sarcospan to the muscle cell membrane or sarcolemma. In some embodiments, the expression of sarcospan or localization of sarcospan to the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of sarcospan prior to administering one or more doses of the polynucleotide. In some embodiments, the method further comprises detecting expression of sarcospan. In some embodiments, detecting expression of sarcospan comprises, or consists essentially of, or yet further consists of detecting protein levels of sarcospan. In some embodiments, detecting expression of sarcospan comprises, or consists essentially of, or yet further consists of detecting RNA levels of sarcospan. Any known methods in the art can be used to detect protein and/or RNA levels of sarcospan. Such methods included, but are not limited to, western blot, PCR, immunofluorescence, and ELISA. In some embodiments, detecting expression of sarcospan comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein, wherein the first sarcoglycan is selected from α-sarcoglycan (SGCA), γ-sarcoglycan (SGCG) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding a γ-sarcoglycan (SGCG) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a γ-sarcoglycan (SGCG) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a γ-sarcoglycan (SGCG) protein, wherein the first sarcoglycan is selected from α-sarcoglycan (SGCA), β-sarcoglycan (SGCB) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein, wherein the first sarcoglycan is selected from γ-sarcoglycan (SGCG), β-sarcoglycan (SGCB) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

In some embodiments, the polynucleotide is encapsulated in a nanoparticle, liposome, or encapsidated within a viral vector (i.e., viral vector particle). Alternatively, or additionally, the polynucleotide is comprised within a vector, e.g., a plasmid or viral vector. In some embodiments, the viral vector is a vector of retrovirus, adenovirus, adeno-associated virus (AAV), lentivirus, alphavirus, flavivirus, rhabdovirus, measles virus, poxvirus, picornavirus, or herpes simplex virus. In some embodiments, the viral vector is a recombinant viral vector. In some embodiments, the viral vector is a recombinant AAV vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13. In some embodiments, the viral vector is AAVrh.74. In some embodiments, the viral vector is a self-complementary vector, e.g., a self-complementary AAVrh.74. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

In some embodiments, the polynucleotide is administered systemically.

In some embodiments, the polynucleotide is administered locally.

In some embodiments, the polynucleotide is administered intravenously or intramuscularly.

In some embodiments, the polynucleotide further comprises, or consists essentially of, or yet further consists of a promoter. In some embodiments, the promoter is a muscle-specific promoter. In some embodiments, the muscle-specific promoter is selected from an MHCK7 promoter and tMCK promoter. In some embodiments, the promoter comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 4 or 6 across the entire length of SEQ ID NO: 4 or 6.

In some embodiments, the polynucleotide further comprises, or consists essentially of, or yet further consists of an intron. In some embodiments, the intron is a SV40 chimeric intron. In some embodiments, the intron comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 9 across the entire length of SEQ ID NO: 9.

In some embodiments, the polynucleotide further comprises, or consists essentially of, or yet further consists of a polyA sequence. In some embodiments, the polyA sequence comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 10 across the entire length of SEQ ID NO: 10.

In some embodiments, the polynucleotide further comprises, or consists essentially of, or yet further consists of an inverted terminal repeat (ITR). In some embodiments, the ITR comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 11 or 12 across the entire length of SEQ ID NO: 11 or 12.

In some embodiments, a composition for restoring or stabilizing a dystrophin-associated protein complex (DAPC) in a subject suffering from muscular dystrophy, wherein the composition comprises, or consists essentially of, or yet further consists of a polynucleotide sequence encoding (a) a sarcoglycan; and/or (b) dystrophin or abbreviated version thereof.

In some embodiments, the muscular dystrophy is Duchenne muscular dystrophy (DMD) or BMD. In some embodiments, the composition comprises, or consists essentially of, or yet further consists of a polynucleotide encoding dystrophin or abbreviated version thereof. In some embodiments, the abbreviated version of dystrophin is a microdystrophin or mini dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39.

In some embodiments, the muscular dystrophy is LGMD2C. In some embodiments, the composition comprises, or consists essentially of, or yet further consists of a polynucleotide encoding the sarcoglycan, wherein the sarcoglycan is SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29.

In some embodiments, the muscular dystrophy is LGMD2D. In some embodiments, the composition comprises, or consists essentially of, or yet further consists of a polynucleotide encoding the sarcoglycan, wherein the sarcoglycan is SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46.

In some embodiments, the muscular dystrophy is LGMD2E. In some embodiments, the composition comprises, or consists essentially of, or yet further consists of a polynucleotide encoding the sarcoglycan, wherein the sarcoglycan is SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18.

In some embodiments, the muscular dystrophy is LGMD2F. In some embodiments, the composition comprises, or consists essentially of, or yet further consists of a polynucleotide encoding the sarcoglycan, wherein the sarcoglycan is SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35.

In some embodiments, provided herein is a composition for localizing a first sarcoglycan, a sarcospan, and/or a dystrophin to muscle cell membrane or sarcolemma in a subject suffering from muscular dystrophy, wherein the composition comprises, or consists essentially of, or yet further consists of a polynucleotide sequence encoding (a) a second sarcoglycan; or (b) dystrophin or abbreviated version thereof.

In some embodiments, a composition for enhancing expression of a first sarcoglycan, a sarcospan, and/or a dystrophin in a subject suffering from muscular dystrophy, wherein the composition comprises, or consists essentially of, or yet further consists of a polynucleotide sequence encoding (a) a second sarcoglycan; or (b) dystrophin or abbreviated version thereof.

In some embodiments, the muscular dystrophy is Duchenne muscular dystrophy (DMD) or BMD. In some embodiments, the composition comprises, or consists essentially of, or yet further consists of a polynucleotide encoding dystrophin or abbreviated version thereof. In some embodiments, the abbreviated of dystrophin is a microdystrophin or mini dystrophin.

In some embodiments, the muscular dystrophy is LGMD2C. In some embodiments, the composition comprises, or consists essentially of, or yet further consists of a polynucleotide encoding the second sarcoglycan, wherein the second sarcoglycan is SGCG. In some embodiments, the first sarcoglycan is selected from SGCA, SGCB, and SGCD.

In some embodiments, the muscular dystrophy is LGMD2D. In some embodiments, the composition comprises, or consists essentially of, or yet further consists of a polynucleotide encoding the second sarcoglycan, wherein the second sarcoglycan is SGCA. In some embodiments, the first sarcoglycan is selected from SGCD, SGCB, and SGCG.

In some embodiments, the muscular dystrophy is LGMD2E. In some embodiments, the composition comprises, or consists essentially of, or yet further consists of a polynucleotide encoding the second sarcoglycan, wherein the second sarcoglycan is SGCB. In some embodiments, the first sarcoglycan is selected from SGCA, SGCD, and SGCG.

In some embodiments, the muscular dystrophy is LGMD2F. In some embodiments, the composition comprises, or consists essentially of, or yet further consists of a polynucleotide encoding the second sarcoglycan, wherein the second sarcoglycan is SGCD. In some embodiments, the first sarcoglycan is selected from SGCA, SGCB, and SGCG.

In some embodiments, the composition increases or enhances expression of the first sarcoglycan at or increases localization of the first sarcoglycan to the muscle cell membrane or sarcolemma. In some embodiments, the first sarcoglycan is SGCD. In some embodiments, the first sarcoglycan is SGCB. In some embodiments, the first sarcoglycan is SGCA. In some embodiments, the first sarcoglycan is SGCG. In some embodiments, the expression of the first sarcoglycan or localization of the first sarcoglycan at the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of the first sarcoglycan prior to administering one or more doses of the polynucleotide. In some embodiments, the composition further comprises, or consists essentially of, or yet further consists of one or more reagents for detecting expression of the first sarcoglycan. In some embodiments, detecting expression of the first sarcoglycan comprises, or consists essentially of, or yet further consists of detecting protein levels of the first sarcoglycan. In some embodiments, detecting expression of the first sarcoglycan comprises, or consists essentially of, or yet further consists of detecting RNA levels of the first sarcoglycan. Any known methods in the art can be used to detect protein and/or RNA levels of the first sarcoglycan. Such methods included, but are not limited to, western blot, PCR, immunofluorescence, and ELISA. Accordingly, the composition may further comprise antibodies or nucleic acids to detect the first sarcoglycan. In some embodiments, detecting expression of the first sarcoglycan comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

In some embodiments, the composition increases or enhances expression of dystrophin at or increases localization of dystrophin to the muscle cell membrane or sarcolemma after administering the subject the polynucleotide sequence encoding (a) a second sarcoglycan; and/or (b) dystrophin or abbreviated version thereof. In some embodiments, the expression of the first sarcoglycan or localization of dystrophin at the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of dystrophin prior to administering one or more doses of the polynucleotide. In some embodiments, the composition further comprises one or more reagents for detecting expression of dystrophin. In some embodiments, detecting expression of dystrophin comprises, or consists essentially of, or yet further consists of detecting protein levels of dystrophin. In some embodiments, detecting expression of dystrophin comprises, or consists essentially of, or yet further consists of detecting RNA levels of dystrophin. Any known methods in the art can be used to detect protein and/or RNA levels of dystrophin. Such methods included, but are not limited to, western blot, PCR, immunofluorescence, and ELISA. Accordingly, the composition may further comprise antibodies or nucleic acids to detect dystrophin. In some embodiments, the dystrophin is the abbreviated dystrophin. In some embodiments, detecting expression of dystrophin comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

In some embodiments, the composition increases or enhances expression of sarcospan at or increases localization of sarcospan to the muscle cell membrane or sarcolemma. In some embodiments, the expression of sarcospan or localization of sarcospan at the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression or localization of sarcospan prior to administering one or more doses of the polynucleotide. In some embodiments, the composition further comprises one or more reagents for detecting expression of sarcospan. In some embodiments, detecting expression of sarcospan comprises, or consists essentially of, or yet further consists of detecting protein levels of sarcospan. In some embodiments, detecting expression of sarcospan comprises, or consists essentially of, or yet further consists of detecting RNA levels of sarcospan. Any known methods in the art can be used to detect protein and/or RNA levels of sarcospan. Such methods included, but are not limited to, western blot, PCR, immunofluorescence, and ELISA. Accordingly, the composition may further comprise antibodies or nucleic acids to detect sarcospan. In some embodiments, detecting expression of sarcospan comprises, or consists essentially of, or yet further consists of performing one or more histological evaluations. Exemplary histological evaluations include, but are not limited to hematoxylin and eosin staining. In some embodiments, the histological evaluation comprises hematoxylin and eosin staining of skeletal muscle (tibialis anterior [TA] and gastrocnemius [GAS]) and quantification of central nucleation.

In some embodiments, the polynucleotide is encapsulated in a nanoparticle, liposome or encapsidated within a viral vector (i.e., viral vector particle). Alternatively, or additionally, the polynucleotide is comprised within a vector, e.g., a plasmid or viral vector. In some embodiments, the viral vector is a vector of retrovirus, adenovirus, adeno-associated virus (AAV), lentivirus, alphavirus, flavivirus, rhabdovirus, measles virus, poxvirus, picornavirus, or herpes simplex virus. In some embodiments, the viral vector is a recombinant viral vector. In some embodiments, the viral vector is a recombinant AAV vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13. In some embodiments, the viral vector is AAVrh.74. In some embodiments, the viral vector is a self-complementary vector, e.g., a self-complementary AAVrh.74.

In some embodiments, the polynucleotide is administered systemically.

In some embodiments, the polynucleotide is administered locally.

In some embodiments, the polynucleotide is administered intravenously or intramuscularly.

In some embodiments, the polynucleotide further comprises, or consists essentially of, or yet further consists of a promoter. In some embodiments, the promoter is a muscle-specific promoter. In some embodiments, the muscle-specific promoter is selected from an MHCK7 promoter and tMCK promoter. In some embodiments, the promoter comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 4 or 6 across the entire length of SEQ ID NO: 4 or 6.

In some embodiments, the polynucleotide further comprises, or consists essentially of, or yet further consists of an intron. In some embodiments, the intron is a SV40 chimeric intron. In some embodiments, the intron comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 9 across the entire length of SEQ ID NO: 9.

In some embodiments, the polynucleotide further comprises, or consists essentially of, or yet further consists of a polyA sequence. In some embodiments, the polyA sequence comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 10 across the entire length of SEQ ID NO: 10.

In some embodiments, the polynucleotide further comprises, or consists essentially of, or yet further consists of an inverted terminal repeat (ITR). In some embodiments, the ITR comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 11 or 12 across the entire length of SEQ ID NO: 11 or 12.

### BRIEF DESCRIPTION OF THE DRAWINGS

FIG. 1A shows the histological evaluations of skeletal muscle (tibialis anterior [TA] and gastrocnemius [GAS]) in wild type (WT) mice, SGCB+/− (SGCB het) mice, and SGCB−/− (SGCB KO) mice.

FIG. 1B shows the quantification of central nucleation in the TA and GAS in wild type (WT) mice, SGCB+/− (SGCB het) mice, and SGCB−/− (SGCB KO) mice.

FIG. 2A shows the SGCB mRNA levels as measured by qRT-PCR in WT mice, SGCB het mice, and SGCB KO mice.

FIG. 2B shows an immunofluorescence image of SGCB protein production in WT mice, SGCB het mice, and SGCB KO mice.

FIG. 2C shows SGCB protein production as measured by western blot in WT mice, SGCB het mice, and SGCB KO mice.

FIG. 3A shows the absolute force in TA muscle of WT, SGCB het, and SGCB KO mice.

FIG. 3B shows the resistance to eccentric contraction in TA muscle of WT, SGCB het, and SGCB KO mice.

FIG. 4A shows ambulation of WT, SGCB het, and SGCB KO mice.

FIG. 4B shows vertical activity of WT, SGCB het, and SGCB KO mice.

FIG. 5A shows immunofluorescence images of dystrophin and SGCB expression in TA muscle in untreated SGCB KO mice and SGCB KO mice treated with hSGCB gene transfer.

FIG. 5B shows immunofluorescence images of SGCA and SGCB expression in cardiac muscle in untreated SGCB KO mice and SGCB KO mice treated with hSGCB gene transfer.

FIG. 5C shows immunofluorescence images of SGCA and SGCB expression in the diaphragm in untreated SGCB KO mice and SGCB KO mice treated with hSGCB gene transfer.

FIG. 5D shows immunofluorescence images of SGCB and dystrophin expression in TA muscle following scAAV.hSGCB gene transfer using the tMCK promoter.

FIG. 5E shows immunofluorescence images of SGCBA and dystrophin expression in TA muscle following scAAV.hSGCB gene transfer using the tMCK promoter.

FIG. 6 shows immunofluorescence staining of SGCG−/− mouse muscle.

FIG. 7 shows immunofluorescence staining for sarcospan in LGMD2E mice.

FIG. 8A shows western blotting for sarcospan in LGMD2E mice.

FIG. 8B shows normalization of sarcospan western blot quantitation.

FIG. 9A shows immunofluorescence images of α-sarcoglycan (SGCA), β-sarcoglycan (SGCB), γ-sarcoglycan (SGCG) and δ-sarcoglycan (SGCD) expression in wild-type mice, SGCB−/− mice, and SGCB−/− mice treated with scAAV.MHCK7.hSGCB at 1.85e13 vg/kg (low dose) and 7.41e13 vg/kg (high dose).

FIG. 9B shows SGCB expression in various tissues of SGCB−/− mice treated with scAAV.MHCK7.hSGCB at 1.85e13 vg/kg (low dose) and 7.41e13 vg/kg (high dose).

FIG. 9C shows SGCA, SGCD, and SGCG expression in SGCB−/− mice treated with scAAV.MHCK7.hSGCB at 1.85e13 vg/kg (low dose) and 7.41e13 vg/kg (high dose).

### DETAILED DESCRIPTION

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises, or consists essentially of, or yet further consists of components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used herein, the terms “increased”, “decreased”, “high”, “low” or any grammatical variation thereof refer to a variation of about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, 5%, 1%, 0.5%, or even 0.1% of the reference composition, polynucleotide, polypeptide, protein, etc.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal sequence identity while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity across the length of the reference sequence and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

An equivalent of a protein or a polypeptide (referred to herein as the reference) shares at least 50% (or at least 55%, or at least 60%, or at least 65%, or at least 70%, or at least 75%, or at least 80%, or at least 85%, or at least 88%, or at least 90%, or at least 93%, or at least 95%, or at least 97%, or at least 98%, or at least 99%) identity to the reference and retains the reference's function and manufacturability.

As used herein, the terms “function,” “activity,” and “enzymatic activity” are used interchangeably. Loss of sarcoglycan function may lead to protein deficiency of other sarcoglycans, dystropin or sarcospan, loss of formation of the sarcoglycan complex, and/or loss of stabilization of the dystrophin-associated protein complex (DAPC). For instance, loss of SGCB protein also leads to a loss of SGCA protein, sarcospan, and dystrophin. In another example, loss of SGCG protein leads to loss of SGCA protein, SGCB protein, and dystrophin. Examples of activities of sarcoglycans include, but are not limited to, stabilizing DAPC and providing mechanical support to the sarcolemma. Functional assessments of sarcoglycan proteins include, but are not limited to, measurement of force production and resistance to contraction-induced injury in the tibialis anterior (TA) muscle along with laser monitoring of open-field cage activity to assess overall ambulation (movement around the cage) and vertical activity (rearing onto hind limbs).

An equivalent of a polynucleotide (referred to herein as the reference) shares at least 50% (or at least 55%, or at least 60%, or at least 65%, or at least 70%, or at least 75%, or at least 80%, or at least 85%, or at least 88%, or at least 90%, or at least 93%, or at least 95%, or at least 97%, or at least 98%, or at least 99%) identity to the reference, and encodes the same polypeptide as the one encoded by the reference, or encodes an equivalent of the polypeptide encoded by the reference that in one aspect, has the same or similar activity or function.

To arrive at a position or a consecutive segment of a test sequence equivalent to (or corresponding to) an/a amino acid/nucleotide residue or a consecutive segment of a reference sequence, a sequence alignment is performed between the test and reference sequences. The positions or segments aligned to each other are determined as equivalents.

The term “affinity tag” refers to a polypeptide that may be included within a fusion protein to allow detection of the fusion protein and/or purification of the fusion protein from the cellular milieu using a ligand that is able to bind to, i.e., has affinity for, the affinity tag. The ligand may be, but is not limited to, an antibody, a resin, or a complementary polypeptide. An affinity tag may comprise a small peptide, commonly a peptide of approximately 4 to 16 amino acids in length, or it may comprise a larger polypeptide. Commonly used affinity tags include polyarginine, FLAG, V5, polyhistidine, c-Myc, Strep II, maltose binding protein (MBP), N-utilization substance protein A (NusA), thioredoxin (Trx), and glutathione S-transferase (GST), among others (for examples, see GST Gene Fusion System Handbook—Sigma-Aldrich). In an embodiment the affinity tag is a polyhistidine tag, for example a His6 tag. The inclusion of an affinity tag in a fusion protein allows the fusion protein to be purified from the cellular milieu by affinity purification, using an affinity medium that is able to tightly and specifically bind the affinity tag. The affinity medium may comprise, for example, a metal-charged resin or a ligand covalently linked to a stationary phase (matrix) such as agarose or metal beads. For example, polyhistidine tagged fusion proteins (also referred to as His tagged fusion proteins) can be recovered by immobilized metal ion chromatography using Ni²⁺ or Co²⁺ loaded resins, anti-FLAG affinity gels may be used to capture FLAG tagged fusion proteins, and glutathione cross-linked to a solid support such as agarose may be used to capture GST tagged fusion proteins.

As used herein the terms “purification”, “purifying”, or “separating” refer to the process of isolating one or more biomaterials (e.g., polynucleotides, polypeptides, or viral vectors) from a complex mixture, such as a cell lysate or a mixture of polypeptides. The purification, separation, or isolation need not be complete, i.e., some components of the complex mixture may remain with the one or more biomaterials (e.g., polynucleotides, polypeptides, or viral vectors) after the purification process. However, the product of purification should be enriched for the one or more biomaterials (e.g., polynucleotides, polypeptides, or viral vectors) relative to the complex mixture before purification and a significant portion of the other components initially present within the complex mixture should be removed by the purification process.

The term “cell” as used herein may refer to either a prokaryotic or eukaryotic cell, optionally obtained from a subject or a commercially available source.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, Chinese Hamster Ovary (CHO) cells, 293T cells, stem cells, satellite cells, and muscle cells. Examples of muscle cells include, but are not limited to, skeletal muscle cells, cardiac muscle cells, and smooth muscle cells.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or abbreviated version thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality (for example, having a similar function or activity). It should be understood, without being explicitly stated that when referring to an equivalent or biological equivalent to a reference polypeptide, protein, or polynucleotide, that an equivalent or biological equivalent has the recited structural relationship to the reference polypeptide, protein, or polynucleotide and equivalent or substantially equivalent biological activity. For example, non-limiting examples of equivalent polypeptides, proteins, or polynucleotides include a polypeptide, protein or polynucleotide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide, polynucleotide or protein sequences across the length of the reference polypeptide, polynucleotide, or protein. Alternatively, an equivalent polypeptide is one that is encoded by a polynucleotide or its complement that hybridizes under conditions of high stringency to a polynucleotide encoding such reference polypeptide sequences and that have substantially equivalent or equivalent biological activity. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity across the length of the reference polynucleotide to the reference polynucleotide, e.g., the wild-type polynucleotide. Such equivalent polypeptides have the same biological activity as the polypeptide encoded by the reference polynucleotide.

Non-limiting examples of equivalent polynucleotides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide. Such equivalent polynucleotides have the same biological activity as the reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences across the length of the reference polynucleotide. The alignment and the percent homology or sequence identity can be determined using software programs known in the art, for example those described in Current Protocols in Molecular Biology (Ausubel et al., eds. 1987) Supplement 30, section 7.7.18, Table 7.7.1.In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, the term “at least 90% identical” refers to an identity of two compared sequences (polynucleotides or polypeptides) of about 90% to about 100%. It also include an identity of at least at least 91%, at least 92%, at least 93%, at least 94%, at least 95%, at least 96%, at least 97%, at least 98%, at least 99%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

As used herein, the terms “retain” “similar” and “same” are used interchangeably while describing a function, an activity or an functional activity of a polynucleotide, a protein and/or a peptide, referring to a functional activity of at least about 20% (including but not limited to: at least about 30%, at least about 40%, at least about 50%, at least about 60%, at least about 70%, at least about 80%, at least about 90%, at least about 95%, at least about 97%, or about 100%) of the activity of the reference protein, polynucleotide and/or peptide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. In one aspect, an equivalent polynucleotide is one that hybridizes under stringent conditions to a reference polynucleotide or its complement. In another aspect, an equivalent polypeptide is a polypeptide that is encoded by a polynucleotide is one that hybridizes under stringent conditions to a reference polynucleotide or its complement.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, complementary DNA (cDNA), DNA-RNA hybrids, or a polymer comprising purine and pyrimidine bases or other natural, chemically or biochemically modified, non-natural, or derivatized nucleotide bases. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a double-strand (ds) DNA, such as an engineered ds DNA or a ds cDNA synthesized from a single-stranded RNA.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, a consecutive amino acid sequence refers to a sequence having at least two amino acids. However, it is noted that a consecutive amino acid sequence of a first part and a second part does not limit the amino acid sequence to have the first part directly conjugated to the second part. It is also possible that the first part is linked to the second part via a third part, such as a link, thus forming one consecutive amino acid sequence.

As used herein, the terms “conjugate,” “conjugated,” “conjugating,” and “conjugation” refer to the formation of a bond between molecules, and in particular between two amino acid sequences and/or two polypeptides. Conjugation can be direct (i.e. a bond) or indirect (i.e. via a further molecule). The conjugation can be covalent or non-covalent.

As used herein a consecutive amino acid sequence may comprise two or more polypeptides conjugated with each other directly or indirectly (for example via a linker).

As used herein, the term “recombinant expression system” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; lipid nanoparticles; polypeptides; polysaccharides; lipopolysaccharides; artificial viral envelopes; metal particles; and bacteria, or viruses, such as rabies virus, flavivirus, lentivirus, baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer” “mRNA-based delivery”, “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes, including for example protamine complexes, lipid nanoparticles, polymeric nanoparticles, lipid-polymer hybrid nanoparticles, and inorganic nanoparticles, or combinations thereof) as well as techniques facilitating the delivery of “naked” polynucleotides (such as electroporation, “gene gun” delivery and various other techniques used for the introduction of polynucleotides). The introduced polynucleotide can be unmodified or can comprise one or more modifications; for example, a modified mRNA may comprise ARCA capping; enzymatic polyadenylation to add a tail of 100-250 adenosine residues; and substitution of one or both of cytidine with 5-methylcytidine and/or uridine with pseudouridine. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

A “yeast artificial chromosome” or “YAC” refers to a vector used to clone large DNA fragments (larger than 100 kb and up to 3000 kb).It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

As used herein, the term “nanoparticle” refers to a particle having dimensions less than about 500 nm, 400 nm, 300 nm, 200 nm, or 100 nm is size. A nanoparticle may comprise or be engineered from various materials. For instance, a nanoparticle may comprise or be engineered from biological materials, such as phospholipids, lipids, lactic acid, dextran, or chitosan. Alternatively, a nanoparticle may comprise or be engineered from polymers, carbon, silica, and metals. A nanoparticle may be biodegradable. Exemplary nanoparticles and nanoparticle compositions are described in, for example, Jong and Borm, Int. J. Nanomedicine 3(2):133-149 and Xue et al., Curr. Pharm. Des. 21(22):3140-3147, which are incorporated by reference in their entireties.

As used herein, the term “liposome” refers to a nanoparticle composed of a phospholipid bilayer with an aqueous core. A liposome may be prepared with biocompatible lipid/phospholipid ingredients. A liposome may be comprise functionalized lipids, such as PEG-lipids or lipids conjugated with targeting moieties for tissue-specific delivery. Exemplary liposomes are described in, for example, Xue et al., Curr. Pharm. Des. 21(22):3140-3147, which is incorporated by reference in its entirety.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid. The capsid may be a wild-type capsid. Alternatively, the capsid may be a modified capsid. A modified capsid may differ from the wild-type capsid by one or more mutations, substitutions, or deletions in the amino acid or nucleic acid sequence of the wild-type capsid.

As used herein, the term “helper” in reference to a virus or plasmid refers to a virus or plasmid used to provide the additional components necessary for replication and packaging of a viral particle or recombinant viral particle, such as the modified AAV disclosed herein. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

In another aspect, the recombinant AAV vectors described herein may be operably linked to a muscle-specific control element. For example the muscle-specific control element is human skeletal actin gene element, cardiac actin gene element, myocyte-specific enhancer binding factor MEF, muscle creatine kinase (MCK), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE).

In some embodiments, the muscle-specific promoter is MHCK7 (SEQ ID NO: 4). An exemplary rAAV described herein is pAAV.MHCK7.hSCGB which comprises, or consists essentially of, or yet further consists of the nucleotide sequence of SEQ ID NO: 3. Within the nucleotide sequence of SEQ ID NO: 3, the MCHK7 promoter spans nucleotides 130-921, a SV40 chimeric intron (SEQ ID NO: 9) spans nucleotides 931-1078, the β-sarcoglycan sequence (SEQ ID NO: 1) spans nucleotides 1091-2047 and the poly A (SEQ ID NO: 10) spans nucleotides 2054-2106. In some embodiments, the pAAV.MHCK7.hSCGB comprises, or consists essentially of, or yet further consists of a nucleotide sequence of SEQ ID NO: 7. Within the nucleotide sequence of SEQ ID NO: 7, the MCHK7 promoter spans nucleotides 128-919, a SV40 chimeric intron spans nucleotides 929-1076, the β-sarcoglycan sequence spans nucleotides 1086-2042 and the poly A spans nucleotides 2049-2101.

In some embodiments, the pAAV.MHCK7.hSCGB comprises, or consists essentially of, or yet further consists of a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence set forth in any one of SEQ ID NOs: 3, 7, and 8 across the entire length of SEQ ID NOs: 3, 7, and 8. In some embodiments, the pAAV.MHCK7.hSCGB comprises, or consists essentially of, or yet further consists of a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence of any one of SEQ ID NOs: 1 and 17 across the entire length of SEQ ID NOs: 1 and 17. In some embodiments, the pAAV.MHCK7.hSCGB comprises, or consists essentially of, or yet further consists of a nucleotide sequence that encodes a polypeptide that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the amino acid sequence of any one of SEQ ID NOs: 2 and 18 across the entire length of SEQ ID NOs: 2 and 18. In one embodiment, the polynucleotide sequence encodes a protein that retains sarcoglycan activity, including beta- and/or alpha-sarcoglycan activity. In another embodiment, the polynucleotide sequence encodes a protein that retains beta-sarcoglycan activity.

In some embodiments, the muscle-specific promoter is tMCK (SEQ ID NO: 6). An exemplary rAAV described herein is pAAV.tMCK.hSCGB which comprises, or consists essentially of, or yet further consists of the nucleotide sequence of SEQ ID NO: 5. Within the nucleotide sequence of SEQ ID NO: 5, the tMCK promoter spans nucleotides 141-854, an SV40 chimeric intron spans nucleotides 886-1018, the β-sarcoglycan sequence spans nucleotides 1058-2014 and the poly A spans nucleotides 2021-2073. In some embodiments, the polynucleotide sequence encoding a pAAV.tMCK.hSCGB comprises, or consists essentially of, or yet further consists of a sequence e.g. at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% or more identical to the nucleotide sequence set forth in SEQ ID NO: 5, wherein the polynucleotide sequence encodes a protein that retains sarcoglycan activity, including but not limited to, beta- and/or alpha-sarcoglycan activity. In some embodiments, the pAAV.tMCK.hSCGB comprises, or consists essentially of, or yet further consists of a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence of any one of SEQ ID NOs: 1 and 17 across the entire length of SEQ ID NOs: 1 and 17. In some embodiments, the pAAV.tMCK.hSCGB comprises, or consists essentially of, or yet further consists of a nucleotide sequence that encodes a polypeptide that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the amino acid sequence of any one of SEQ ID NOs: 2 and 18 across the entire length of SEQ ID NOs: 2 and 18.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. The cell sample may comprise cells from a tissue or organ. Exemplary organs include, but are not limited to, heart, lungs, liver, eyes, stomach, spleen, kidney, stomach, pancreas, and gallbladder. Exemplary tissues include, but are not limited to, connective tissue, epithelial tissue, muscle tissue, and nervous tissue. The cell sample may comprise a muscle cell or component of a muscle cell. A component of a muscle cell may include a muscle cell membrane or sarcolemma. In some embodiments, the cell membrane is a skeletal muscle cell membrane or sarcolemma. The tissue sample may comprise muscle tissue.

As used herein, the terms “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose6 phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵ I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluor, FITC, TRITC or any other fluorescent dye or hapten.

As used herein, an epitope tag is a biological structure or sequence, such as a protein or carbohydrate, which acts as an antigen that is recognized by an antibody. In certain embodiments, an epitope tag is used interchangeably with a purification marker and/or an affinity tag.

A “composition” is intended to mean a combination of two or more compounds, such as a combination of an active polypeptide, polynucleotide, viral vector, or antibody and another compound or composition, inert (e.g., a detectable label) or active (e.g., a gene delivery vehicle).

A “pharmaceutical composition” is intended to include the combination of an active polypeptide, polynucleotide, vector or antibody with a carrier, inert or active such as a solid support or liquid carrier, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to muscular dystrophies. In one embodiment, the mammals include horses, dogs, and cats. In another embodiment of the present invention, the human is an adult, which is a human over the age of eighteen years of age, an adolescent, which is a human between the age of thirteen to eighteen years of age, a child, which is a human under the age of thirteen years of age, or under the age of 10, or under the age of 8, or under the age of 6, or under the age of 4, or under the age of 2. In some embodiments, the subject or human is a male. In some embodiments, the subject or human is a female.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the term “treatment” excludes prevention or prophylaxis.

The term “suffering” as it related to the term “treatment” refers to a subject who has been diagnosed with or is predisposed to a disease. In one embodiment, a subject is diagnosed with a muscular dystrophy. In one embodiment, a subject is predisposed to muscular dystrophy. A subject predisposed to muscular dystrophy is a subject, individual, or patient having one or more mutations or alterations in genes responsible for healthy muscle structure and function. A subject suffering from muscular dystrophy may exhibit one or more symptoms or signs of muscular dystrophy. Exemplary symptoms or signs of muscular dystrophy include, but are not limited to, enlarged calf muscles, difficulty walking or running, unusual walking gait (like waddling), trouble swallowing, eart problems, such as arrhythmia and heart failure (cardiomyopathy), learning disabilities, stiff or loose joints, muscle pain, curved spine (scoliosis), and breathing problems. Alternatively, a subject suffering from muscular dystrophy may have one or more mutations or alterations in genes responsible for healthy muscle structure and function.

The term “muscular dystrophy” as used herein refers to a disorder in which strength and muscle bulk gradually decline. Non-limiting examples of muscular dystrophy diseases may include Becker muscular dystrophy (BMID), tibial muscular dystrophy, Duchenne muscular dystrophy (DMD), Emery-Dreifuss muscular dystrophy, facioscapulohumeral muscular dystrophy, sarcoglycanopathies, congenital muscular dystrophy such as congenital muscular dystrophy due to partial LAMA2 deficiency, merosin-deficient congenital muscular dystrophy, type ID congenital muscular dystrophy, Fukuyama congenital muscular dystrophy, limb-girdle type 1 A muscular dystrophy, limb-girdle type 2 A muscular dystrophy, limb-girdle type 2B muscular dystrophy, limb-girdle type 2C muscular dystrophy, limb-girdle type 2D muscular dystrophy, limb-girdle type 2E muscular dystrophy, limb-girdle type 2F muscular dystrophy, limb-girdle type 2G muscular dystrophy, limb-girdle type 21H muscular dystrophy, limb-girdle type 21 muscular dystrophy, limb-girdle type 21 muscular dystrophy, limb-girdle type 2J muscular dystrophy, limb-girdle type 2K muscular dystrophy, limb-girdle type IC muscular dystrophy, rigid spine muscular dystrophy with epidermolysis bullosa simplex, oculopharyngeal muscular dystrophy, Ullrich congenital muscular dystrophy, and Ullrich scleroatonic muscular dystrophy. In some embodiments, the subject is suffering from limb-girdle muscular dystrophy. In some embodiments, the subject is suffering from limb-girdle muscular dystrophy type 2E (LGMD2E).

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat muscular dystrophies, e.g., LGMD and DMD, ex vivo, in vitro or in vivo.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, Handbook of Parvoviruses 1:169-228, 1989, and Berns, Virology 1743-1764, 1999. However, it is fully expected that these same principles will be applicable to additional AAV serotypes since it is well known that the various serotypes are quite closely related, both structurally and functionally, even at the genetic level. (See, for example, Blacklowe, Parvoviruses and Human Disease 165-174, 1988, J. R. Pattison, ed.; and Rose, Comprehensive Virology 3:1-61, 1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

As described herein, the term “abbreviated version of dystrophin” refers to a protein that is less than full length of dystrophin protein, while at least partially maintain the function of a dystrophin protein. In one embodiment, the abbreviated version is mini-dystrophin or a micro-dystrophin. In one embodiment, the micro-dystrophin protein is about ⅓ size of a full length dystrophin protein. For example, one embodiment of micro-dystrophin protein can be found at WO2017181015, which is incorporated by reference. In another embodiment, the mini-dystrophin protein sequences can be found at U.S. Pat. No. 6,869,777, which is incorporated by reference.

Without wishing to be bound by theory, in skeletal and cardiac muscles, dystrophin is part of a group of proteins (DAPC) that work together to strengthen muscle fibers and protect them from injury as muscles contract and relax. In some embodiments, the dystrophin protein transfers the force of muscle contraction from inside of the muscle cell outward to the cell membrane. Absence or reduced expression of dystrophin or many of the DAPC components cause the muscular dystrophies, a group of inherited diseases in which repeated bouts of muscle damage lead to atrophy and fibrosis, and eventually muscle degeneration.

An “AAV expression cassette” as used herein refers to a nucleotide sequence comprising, or consisting essentially of, or yet further consisting of one or more polynucleotides of interest (or transgenes) that are flanked by AAV terminal repeat sequences (ITRs). Such AAV expression cassette can be replicated and packaged into infectious viral particles (e.g., AAV vectors) when present in a host cell that has been transfected with a vector encoding and expressing rep and cap gene products.

An “AAV virion” or “AAV vector” or “AAV viral particle” or “AAV vector particle” refers to a viral particle composed of at least one AAV capsid protein and an encapsidated polynucleotide AAV expression cassette. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV expression cassette, as such a cassette is contained within an AAV vector particle. An AAV vector may be a single-stranded AAV (ssAAV) vector or self-complementary AAV (scAAV) vector. For ssAAV vectors, the coding sequence and complementary sequence of the transgene expression cassette are on separate strands and are packaged in separate viral capsids. For scAAV vectors, both the coding and complementary sequence of the transgene expression cassette are present on each plus- and minus-strand genome.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac, et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

Recombinant AAV (rAAV) genomes of the disclosure comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. In some embodiments, to promote skeletal muscle specific expression, AAV1, AAV6, AAV8 or AAVrh.74 is used. In some embodiments, the rAAV genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “recombinant” as used herein with respect to polypeptides or polynucleotides, such as DNA or RNA, refers to molecules formed by laboratory methods of recombination, such as molecular cloning. Molecular cloning techniques are known in the art and may include, but is not limited to, PCR amplification of a polynucleotide, enzymatic digestion of a polynucleotide, ligation of a polynucleotide into an expression cassette (e.g., mammalian expression cassette), transformation, transfection or transduction of a cell with the polynucleotide, and expression of the polynucleotide to produce the polypeptide. See e.g., Green and Sambrook, Molecular Cloning: A Laboratory Manual, 2012. The term “recombinant polynucleotide” is meant to include fragments of protein-encoding polynucleotides. For instance, a recombinant polynucleotide may include a fragment of the polynucleotide that encodes for a human sarcoglycan protein. A recombinant polynucleotide may be produced by PCR amplification of a fragment of a protein-encoding polynucleotide. A recombinant polypeptide may be produced by expression of one or more recombinant polynucleotides.

Modes for Carrying Out the Disclosure

Disclosed herein are methods of restoring or stabilizing a dystrophin-associated protein complex (DAPC) in a subject suffering from muscular dystrophy. In some embodiments, the method comprises, or consists essentially of, or yet further consists of administering to the subject a polynucleotide sequence encoding (a) a sarcoglycan; (b) dystrophin; or (c) an abbreviated version of dystrophin. As described here, the term “abbreviated version of dystrophin” refers to a protein that is less than full length of dystrophin protein, while at least partially maintain the function of a dystrophin protein. In one embodiment, the abbreviated version is mini-dystrophin or a micro-dystrophin. In one embodiment, the micro-dystrophin protein is about ⅓ size of a full length dystrophin protein. For example, one embodiment of micro-dystrophin protein can be found at WO2017181015, which is incorporated by reference. In another embodiment, the mini-dystrophin protein sequences can be found at U.S. Pat. No. 6,869,777, which is incorporated by reference. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Disclosed herein are methods of localizing a first sarcoglycan, sarcospan, or dystrophin to a muscle cell membrane or sarcolemma in a subject suffering from muscular dystrophy. In some embodiments, the method comprises, or consists essentially of, or yet further consists of administering to the subject a polynucleotide sequence encoding (a) a second sarcoglycan; (b) dystrophin; or (c) an abbreviated version of dystrophin, wherein the first sarcoglycan is different from the second sarcoglycan. In some embodiments, the first sarcoglycan is SGCA and the second sarcoglycan is selected from SGCB, SGCD, and SGCG. In some embodiments, the first sarcoglycan is SGCB and the second sarcoglycan is selected from SGCA, SGCD, and SGCG. In some embodiments, the first sarcoglycan is SGCD and the second sarcoglycan is selected from SGCB, SGCA, and SGCG. In some embodiments, the first sarcoglycan is SGCG and the second sarcoglycan is selected from SGCB, SGCD, and SGCA. In some embodiments, the first sarcoglycan is selected from SGCA, SGCD, and SGCG and the second sarcoglycan is SGCB. In some embodiments, the first sarcoglycan is selected from SGCB, SGCD, and SGCG and the second sarcoglycan is SGCA. In some embodiments, the first sarcoglycan is selected from SGCA, SGCB, and SGCG and the second sarcoglycan is SGCD. In some embodiments, the first sarcoglycan is selected from SGCA, SGCB, and SGCD and the second sarcoglycan is SGCG. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Disclosed herein are methods of increasing or enhancing expression of a first sarcoglycan, sarcospan, or dystrophin to a muscle cell membrane or sarcolemma in a subject suffering from muscular dystrophy, In some embodiments, the method comprises, or consists essentially of, or yet further consists of administering to the subject a polynucleotide sequence encoding (a) a second sarcoglycan; (b) dystrophin; or (c) an abbreviated version of dystrophin, wherein the first sarcoglycan is different from the second sarcoglycan. In some embodiments, the first sarcoglycan is SGCA and the second sarcoglycan is selected from SGCB, SGCD, and SGCG. In some embodiments, the first sarcoglycan is SGCB and the second sarcoglycan is selected from SGCA, SGCD, and SGCG. In some embodiments, the first sarcoglycan is SGCD and the second sarcoglycan is selected from SGCB, SGCA, and SGCG. In some embodiments, the first sarcoglycan is SGCG and the second sarcoglycan is selected from SGCB, SGCD, and SGCA. In some embodiments, the first sarcoglycan is selected from SGCA, SGCD, and SGCG and the second sarcoglycan is SGCB. In some embodiments, the first sarcoglycan is selected from SGCB, SGCD, and SGCG and the second sarcoglycan is SGCA. In some embodiments, the first sarcoglycan is selected from SGCA, SGCB, and SGCG and the second sarcoglycan is SGCD. In some embodiments, the first sarcoglycan is selected from SGCA, SGCB, and SGCD and the second sarcoglycan is SGCG. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Disclosed herein are compositions for restoring or stabilizing a dystrophin-associated protein complex (DAPC) in a subject suffering from muscular dystrophy. In some embodiments, the composition comprises, or consists essentially of, or yet further consists of a polynucleotide sequence encoding (a) a sarcoglycan; (b) dystrophin; or (c) an abbreviated version of dystrophin. In one embodiment, the abbreviated version is mini-dystrophin or a micro-dystrophin. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Disclosed herein are compositions for localizing a first sarcoglycan, a sarcospan, and/or a dystrophin to muscle cell membrane or sarcolemma in a subject suffering from muscular dystrophy. In some embodiments, the composition comprises, or consists essentially of, or yet further consists of a polynucleotide sequence encoding (a) a second sarcoglycan; (b) dystrophin; or (c) an abbreviated version of dystrophin. In one embodiment, the abbreviated version is mini-dystrophin or a micro-dystrophin. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Disclosed herein are compositions for enhancing expression of a first sarcoglycan, a sarcospan, and/or a dystrophin in a subject suffering from muscular dystrophy. In some embodiments, the composition comprises, or consists essentially of, or yet further consists of a polynucleotide sequence encoding (a) a second sarcoglycan; (b) dystrophin; or (c) an abbreviated version of dystrophin. In one embodiment, the abbreviated version is mini-dystrophin or a micro-dystrophin. In some embodiments, the method comprises, or consists essentially of, or yet further consists of administering a polynucleotide sequence encoding dystrophin. In some embodiments, the polynucleotide encoding dystrophin comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37. In some embodiments, the method comprises, or consists essentially of, or yet further consists of administering a polynucleotide sequence encoding an abbreviated version of dystrophin. In some embodiments, the polynucleotide encoding the abbreviated version of dystrophin comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44; or (b) a nucleotide sequence that encodes an abbreviated version of a dystrophin protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the method comprises, or consists essentially of, or yet further consists of administering a polynucleotide encoding SGCG. In some embodiments, the polynucleotide encoding SGCG comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 20-24 across the entire length of SEQ ID NOs: 20-24; or (b) a nucleotide sequence that encodes a SGCG protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 25-29 across the entire length of SEQ ID NO: 25-29. In some embodiments, the method comprises, or consists essentially of, or yet further consists of administering a polynucleotide encoding SGCA. In some embodiments, the polynucleotide encoding SGCA comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 13, 14, and 45 across the entire length of SEQ ID NOs: 13, 14, and 45; or (b) a nucleotide sequence that encodes a SGCA protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 15, 16, and 46 across the entire length of SEQ ID NO: 15, 16, and 46. In some embodiments, the method comprises, or consists essentially of, or yet further consists of administering a polynucleotide encoding SGCB. In some embodiments, the polynucleotide encoding SGCB comprises, or consists essentially of, or yet further consists of (a) a nucleotide sequence that is at least 70%, 80%, 85%, 90%, 95%, 97%, 98%, 99%, or 100% identical to the nucleotide sequence of SEQ ID NO: 1 or 17 across the entire length of SEQ ID NO: 1 or 17; or (b) a nucleotide sequence that encodes a SGCB protein comprising, or consisting essentially of, or yet further consisting of an amino acid sequence that is at least 70%, 80%, 85%, 90%, 95%, 97%, 98%, 99%, or 100% identical to the amino acid sequence of SEQ ID NO: 2 or 18 across the entire length of SEQ ID NO: 2 or 18. In some embodiments, the method comprises, or consists essentially of, or yet further consists of administering a polynucleotide encoding SGCD. In some embodiments, the polynucleotide encoding SGCD comprises, or consists essentially of, or yet further consists of (a) a nucleotide sequence that is at least 70%, 80%, 85%, 90%, 95%, 97%, 98%, 99%, or 100% identical to the nucleotide sequence of any one of SEQ ID NOs: 30-32 across the entire length of SEQ ID NOs: 30-32; or (b) a nucleotide sequence that encodes a SGCD protein comprising, or consisting essentially of, or yet further consisting of an amino acid sequence that is at least 70%, 80%, 85%, 90%, 95%, 97%, 98%, 99%, or 100% identical to the amino acid sequence of any one of SEQ ID NOs: 33-35 across the entire length of SEQ ID NOs: 33-35. In some embodiments, the method comprises, or consists essentially of, or yet further consists of administering a viral vector comprising, or consisting essentially of, or yet further consisting of a viral genome comprising, or consisting essentially of, or yet further consisting of the polynucleotide encoding sarcoglycan, dystrophin or an abbreviated version of dystrophin. In some embodiments, the viral vector comprises, or consists essentially of, or yet further consists of a viral genome comprising, or consisting essentially of, or yet further consisting of a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a β-sarcoglycan (SGCB) protein, wherein the first sarcoglycan is selected from α-sarcoglycan (SGCA), γ-sarcoglycan (SGCG) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 3, 5, 7, and 8 across the entire length of SEQ ID NOs: 3, 5, 7, and 8.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding a 7-sarcoglycan (SGCG) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a 7-sarcoglycan (SGCG) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding a γ-sarcoglycan (SGCG) protein, wherein the first sarcoglycan is selected from α-sarcoglycan (SGCA), β-sarcoglycan (SGCB) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 19 across the entire length of SEQ ID NO: 19.

Further disclosed herein is a method of restoring or stabilizing a dystrophin-associated protein complex (DAPC) in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

Further disclosed herein is a method of localizing a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein. In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

Further disclosed herein is a method of increasing or enhancing expression of a first sarcoglycan, sarcospan, and/or dystrophin to a muscle cell membrane or sarcolemma in a subject suffering from muscular dystrophy, comprising, or consisting essentially of, or yet further consisting of administering to the subject a viral vector genome comprising, or consisting essentially of, or yet further consisting of a polynucleotide sequence encoding an α-sarcoglycan (SGCA) protein, wherein the first sarcoglycan is selected from γ-sarcoglycan (SGCG), β-sarcoglycan (SGCB) and δ-sarcoglycan (SGCD). In some embodiments, the virial vector genome comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 47 or 48 across the entire length of SEQ ID NO: 47 or 48.

Sarcoglycan Complex and DAPC

The sarcoglycans and a protein called sarcospan along with dystrophin are integral proteins critical for stabilizing the DAPC and providing mechanical support to the sarcolemma. Autosomal recessive mutations in the sarcoglycans lead to protein deficiency, loss of formation of the sarcoglycan complex, and loss of stabilization of the dystrophin-associated protein complex (DAPC). Disclosed herein are methods of using any of the polynucleotides, expression cassettes viral vectors, and compositions disclosed herein to repair a sarcoglycan complex, restore or increase sarcoglycan expression, restore or increase dystrophin expression, restore or increase sarcospan expression, stabilize a DAPC or sarcolemma, or restore or increase the function of DAPC or sarcolemma. Methods to determine such are known in the art.

Sarcoglycans

Sarcoglycans are transmembrane proteins found as a plasma membrane-associated complex known as the sarcoglycan complex, which is a subcomplex of DAPC. The exact function of the sarcoglycan complex is not known, but it may have both mechanical and nonmechanical roles in stabilizing the plasma membrane in cardiac and skeletal muscle. A variant of the sarcoglycan complex is found in vascular smooth muscle and in some nonmuscle cell and tissue types. Thus, the sarcoglycan complex has important roles in both muscle and nonmuscle tissues.

Examples of sarcoglycans include, but are not limited to, α-sarcoglycan (SGCA), β-sarcoglycan (SGCB), γ-sarcoglycan (SGCG), δ-sarcoglycan (SGCD), ε-sarcoglycan (SGCE), and ζ-sarcoglycan (SGCZ). Autosomal recessive mutations in several sarcoglycan genes, α, β, γ and δ, may lead to disruption of the sarcoglycan complex, which results in sarcoglycanopathies, such as type 2 Limb Girdle Muscular Dystrophies (LGMD2). For instance, mutations in SGCG may lead to LGMD2C, mutations in SGCA may lead to LGMD2D, mutations in SGCB may lead to LGMD2E, and mutations in SGCD may lead to LGMD2F.

In some embodiments, the methods disclosed herein comprise, or consist essentially of, or yet further consist of administering to a subject suffering from a muscular dystrophy a polynucleotide encoding SGCA. In some embodiments, the methods disclosed herein comprise, or consist essentially of, or yet further consist of administering a polynucleotide encoding a SGCA protein to a subject suffering from LGMD2D. In some embodiments, the sarcoglycan is SGCA. Polynucleotides encoding SGCA proteins are known in the art, e.g. polynucleotides encoding the amino acid sequences described below. For instance, the polynucleotide encoding the SGCA protein may comprise, or consist essentially of, or yet further consist of a SGCA nucleotide sequence disclosed in International App. No. PCT/US2020/47339 (corresponding to SEQ ID NO: 45), Genbank Accession No. N. Mex._000023.4 (corresponding to SEQ ID NO: 13), or Genbank Accession No. N. Mex._001135697.3 (corresponding to SEQ ID NO: 14), each of which are incorporated by reference in their entireties. In some embodiments, the polynucleotide encoding the SGCA protein comprises, or consists essentially of, or yet further consists of a nucleotide sequence that is at least 70%, 80%, 85%, 90%, 95%, 97%, 98%, 99%, or 100% identical to a SGCA-encoding nucleotide sequence disclosed in International App. No. PCT/US2020/47339 (corresponding to SEQ ID NO: 45), Genbank Accession No. N. Mex._000023.4 (corresponding to SEQ ID NO: 13), or Genbank Accession No. N. Mex._001135697.3 (corresponding to SEQ ID NO: 14) across the entire length of the SGCA-encoding nucleotide sequence. In some embodiments, the polynucleotide encoding the SGCA protein comprises, or consists essentially of, or yet further consists of a nucleotide sequence that is at least about 80% identical to the nucleotide sequence of any one of SEQ ID NOs: 13, 14, and 45 across the entire length of SEQ ID Nos: 13, 14, and 45. In some embodiments, the polynucleotide encoding the SGCA protein comprises, or consists essentially of, or yet further consists of a nucleotide sequence that is at least about 85% identical to the nucleotide sequence of any one of SEQ ID NOs: 13, 14, and 45 across the entire length of SEQ ID Nos: 13, 14, and 45. In some embodiments, the polynucleotide encoding the SGCA protein comprises, or consists essentially of, or yet further consists of a nucleotide sequence that is at least about 90% identical to the nucleotide sequence of any one of SEQ ID NOs: 13, 14, and 45 across the entire length of SEQ ID Nos: 13, 14, and 45. In some embodiments, the polynucleotide encoding the SGCA protein comprises, or consists essentially of, or yet further consists of a nucleotide sequence that is at least about 95% identical to the nucleotide sequence of any one of SEQ ID NOs: 13, 14, and 45 across the entire length of SEQ ID Nos: 13, 14, and 45. In some embodiments, the polynucleotide encoding the SGCA protein comprises, or consists essentially of, or yet further consists of a nucleotide sequence that is 100% identical to the nucleotide sequence of any one of SEQ ID NOs: 13, 14, and 45 across the entire length of SEQ ID Nos: 13, 14, and 45. SGCA protein sequences are known in the art. For instance, the SGCA protein may comprise, or consist essentially of, or yet further consist of an SGCA amino acid sequence disclosed in International App. No. PCT/US2020/47339 (corresponding to SEQ ID NO: 46), Genbank Accession No. NP_000014.1 (corresponding to SEQ ID NO: 15), or NCBI Reference Sequence NP_0011292169.1 (corresponding to SEQ ID NO: 16), each of which are incorporated by reference in their entireties. In some embodiments, the SGCA protein comprises, or consists essentially of, or yet further consists of an amino acid sequence that is at least 70%, 80%, 85%, 90%, 95%, 97%, 98%, 99%, or 100% identical to an SGCA amino acid sequence disclosed in International App. No. PCT/US2020/47339 (corresponding to SEQ ID NO: 46), Genbank Accession No. NP_000014.1 (corresponding to SEQ ID NO: 15), or NCBI Reference Sequence NP_0011292169.1 (corresponding to SEQ ID NO: 16) across the entire length of the SGCA amino acid sequence. In some embodiments, the SGCA protein comprises, or consists essentially of, or yet further consists of an amino acid sequence that is at least about 80% identical to the SGCA amino acid sequence of any one of SEQ ID NOs: 15, 16, and 46 across the entire length of SEQ ID NOs: 15, 16, and 46. In some embodiments, the SGCA protein comprises, or consists essentially of, or yet further consists of an amino acid sequence that is at least about 85% identical to the SGCA amino acid sequence of any one of SEQ ID NOs: 15, 16, and 46 across the entire length of SEQ ID NOs: 15, 16, and 46. In some embodiments, the SGCA protein comprises, or consists essentially of, or yet further consists of an amino acid sequence that is at least about 90% identical to the SGCA amino acid sequence of any one of SEQ ID NOs: 15, 16, and 46 across the entire length of SEQ ID NOs: 15, 16, and 46. In some embodiments, the SGCA protein comprises, or consists essentially of, or yet further consists of an amino acid sequence that is at least about 95% identical to the SGCA amino acid sequence of any one of SEQ ID NOs: 15, 16, and 46 across the entire length of SEQ ID NOs: 15, 16, and 46. In some embodiments, the SGCA protein comprises, or consists essentially of, or yet further consists of an amino acid sequence that is at least about 100% identical to the SGCA amino acid sequence of any one of SEQ ID NOs: 15, 16, and 46 across the entire length of SEQ ID NOs: 15, 16, and 46. In some embodiments, the polynucleotide encoding the SGCA protein is a codon-optimized.

Disclosed herein are methods of increasing or restoring expression of SGCA in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is Duchenne Muscular Dystrophy (DMD) or Becker Muscular Dystrophy (BMD) and the method comprises, or consists essentially of, or yet further consists of administering to the subject a polynucleotide encoding a dystrophin protein or an abbreviated version of a dystrophin protein.

In some embodiments, the expression level of SGCA is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCA prior to administering a first dose of the polynucleotide encoding SGCG, SGCB, SGCD, or the abbreviated version of the dystrophin protein. In some embodiments, the expression level of SGCA is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCA prior to administering one or more subsequent doses of the polynucleotide encoding SGCG, SGCB, SGCD, or the abbreviated version of the dystrophin protein. In some embodiments, the expression level of SGCA is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCA in a reference sample, wherein the reference sample is from one or more subjects suffering from a muscular dystrophy caused by a mutation in a dystrophin or sarcoglycan gene (e.g., SGCD, SGCB, or SGCG). In some embodiments, the expression level of SGCA is at least 50%, 60%, 70%, 80%, 90%, 100%, 110%, 120%, 130%, 140%, or 150% of the expression level of SGCA in a reference sample, wherein the reference sample is from one or more healthy subjects (e.g., a subject not suffering from a muscular dystrophy caused by a mutation in a dystrophin or sarcoglycan gene). The expression level of SGCA may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, SGCA expression is detected in a sample from the subject. In some embodiments, the sample comprises, or consists essentially of, or yet further consists of a skeletal muscle cell or cardiac muscle cell.

Disclosed herein are methods of localizing SGCA to a cell membrane in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD or Becker Muscular Dystrophy (BMD) and the method comprises, or consists essentially of, or yet further consists of administering to the subject a polynucleotide encoding a dystrophin protein or an abbreviated version of a dystrophin protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

In some embodiments, the methods disclosed herein comprise, or consist essentially of, or yet further consist of administering to a subject suffering from a muscular dystrophy a polynucleotide encoding SGCB. In some embodiments, the methods disclosed herein comprise, or consist essentially of, or yet further consist of administering a polynucleotide encoding a SGCB protein to a subject suffering from LGMD2E. In some embodiments, the sarcoglycan is SGCB. Polynucleotides encoding SGCB proteins are known in the art. For instance, the polynucleotide encoding the SGCB protein may comprise, or consist essentially of, or yet further consist of a SGCB nucleotide sequence disclosed in International App. No. PCT/US2020/19892 (corresponding to SEQ ID NO: 1) or Genbank Accession No. N. Mex._000232.5 (corresponding to SEQ ID NO: 17), each of which are incorporated by reference in their entireties. In some embodiments, the polynucleotide encoding the SGCB protein comprises, or consists essentially of, or yet further consists of a nucleotide sequence that is at least 70%, 80%, 85%, 90%, 95%, 97%, 98%, 99%, or 100% identical to a SGCB-encoding nucleotide sequence disclosed in International App. No. PCT/US2020/19892 (corresponding to SEQ ID NO: 1) or Genbank Accession No. N. Mex._000232.5 (corresponding to SEQ ID NO: 17) across the entire length of the SGCB-encoding nucleotide sequence. In some embodiments, the polynucleotide encoding the SGCB protein comprises, or consists essentially of, or yet further consists of a nucleotide sequence that is at least about 80% identical to the nucleotide sequence of SEQ ID NO: 1 or 17 across the entire length of SEQ ID NO: 1 or 17. In some embodiments, the polynucleotide encoding the SGCB protein comprises, or consists essentially of, or yet further consists of a nucleotide sequence that is at least about 85% identical to the nucleotide sequence of SEQ ID NO: 1 or 17 across the entire length of SEQ ID NO: 1 or 17. In some embodiments, the polynucleotide encoding the SGCB protein comprises, or consists essentially of, or yet further consists of a nucleotide sequence that is at least about 90% identical to the nucleotide sequence of SEQ ID NO: 1 or 17 across the entire length of SEQ ID NO: 1 or 17. In some embodiments, the polynucleotide encoding the SGCB protein comprises, or consists essentially of, or yet further consists of a nucleotide sequence that is at least about 95% identical to the nucleotide sequence of SEQ ID NO: 1 or 17 across the entire length of SEQ ID NO: 1 or 17. In some embodiments, the polynucleotide encoding the SGCB protein comprises, or consists essentially of, or yet further consists of a nucleotide sequence that is at least about 100% identical to the nucleotide sequence of SEQ ID NO: 1 or 17 across the entire length of SEQ ID NO: 1 or 17. SGCB protein sequences are known in the art. For instance, the SGCB protein may comprise, or consist essentially of, or yet further consist of an SGCB amino acid sequence disclosed in International App. No. PCT/US2020/19892 (corresponding to SEQ ID NO: 2) or Genbank Accession No. NP_000223.1 (corresponding to SEQ ID NO: 18), each of which are incorporated by reference in their entireties. In some embodiments, the SGCB protein comprises, or consists essentially of, or yet further consists of an amino acid sequence that is at least 70%, 80%, 85%, 90%, 95%, 97%, 98%, 99%, or 100% identical to an SGCB amino acid sequence disclosed in International App. No. PCT/US2020/19892 (corresponding to SEQ ID NO: 2) or Genbank Accession No. NP_000223.1 (corresponding to SEQ ID NO: 18) across the entire length of the SGCB amino acid sequence. In some embodiments, the SGCB protein comprises, or consists essentially of, or yet further consists of an amino acid sequence that is at least about 80% identical to the amino acid sequence of SEQ ID NO: 2 or 18 across the entire length of SEQ ID NO: 2 or 18. In some embodiments, the SGCB protein comprises, or consists essentially of, or yet further consists of an amino acid sequence that is at least about 85% identical to the amino acid sequence of SEQ ID NO: 2 or 18 across the entire length of SEQ ID NO: 2 or 18. In some embodiments, the SGCB protein comprises, or consists essentially of, or yet further consists of an amino acid sequence that is at least about 90% identical to the amino acid sequence of SEQ ID NO: 2 or 18 across the entire length of SEQ ID NO: 2 or 18. In some embodiments, the SGCB protein comprises, or consists essentially of, or yet further consists of an amino acid sequence that is at least about 95% identical to the amino acid sequence of SEQ ID NO: 2 or 18 across the entire length of SEQ ID NO: 2 or 18. In some embodiments, the SGCB protein comprises, or consists essentially of, or yet further consists of an amino acid sequence that is at least about 100% identical to the amino acid sequence of SEQ ID NO: 2 or 18 across the entire length of SEQ ID NO: 2 or 18. In some embodiments, the polynucleotide encoding the SGCB protein is a codon-optimized.

Disclosed herein are methods of increasing or restoring expression of SGCB in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD or BMD and the method comprises, or consists essentially of, or yet further consists of administering to the subject a polynucleotide encoding a dystrophin protein or an abbreviated version of a dystrophin protein.

In some embodiments, the expression level of SGCB is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCB prior to administering a first dose of the polynucleotide encoding SGCA, SGCG, SGCD, or the abbreviated version of the dystrophin protein. In some embodiments, the expression level of SGCB is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCB prior to administering one or more subsequent doses of the polynucleotide encoding SGCA, SGCG, SGCD, or the abbreviated version of the dystrophin protein. In some embodiments, the expression level of SGCB is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCB in a reference sample, wherein the reference sample is from one or more subjects suffering from a muscular dystrophy caused by a mutation in a dystrophin or sarcoglycan gene (e.g., SGCA, SGCG, or SGCD). In some embodiments, the expression level of SGCB is at least 50%, 60%, 70%, 80%, 90%, 100%, 110%, 120%, 130%, 140%, or 150% of the expression level of SGCB in a reference sample, wherein the reference sample is from one or more healthy subjects (e.g., a subject not suffering from a muscular dystrophy caused by a mutation in a dystrophin or sarcoglycan gene). The expression level of SGCB may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, SGCB expression is detected in a sample from the subject. In some embodiments, the sample comprises, or consists essentially of, or yet further consists of a skeletal muscle cell or cardiac muscle cell.

Disclosed herein are methods of localizing SGCB to a cell membrane in a subject suffering in need thereof, e.g., a subject from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD and the method comprises, or consists essentially of, or yet further consists of administering to the subject a polynucleotide encoding a dystrophin protein or an abbreviated version of a dystrophin protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

In some embodiments, the methods disclosed herein comprise, or consist essentially of, or yet further consist of administering to a subject suffering from a muscular dystrophy a polynucleotide encoding SGCG. In some embodiments, the methods disclosed herein comprise, or consist essentially of, or yet further consist of administering a polynucleotide encoding a SGCG protein to a subject suffering from LGMD2C. In some embodiments, the sarcoglycan is SGCG. Polynucleotides encoding SGCG proteins are known in the art. For instance, the polynucleotide encoding the SGCG protein may comprise, or consists essentially of, or yet further consists of a SGCG nucleotide sequence disclosed in International App. No. PCT/US2019/015779 (corresponding to SEQ ID NO: 20), Genbank Accession No. N. Mex._000231.3 (corresponding to SEQ ID NO: 21), Genbank Accession No. N. Mex._001378244.1 (corresponding to SEQ ID NO: 22), Genbank Accession No. N. Mex._001378245.1 (corresponding to SEQ ID NO: 23), or Genbank Accession No. N. Mex._001378246.1 (corresponding to SEQ ID NO: 24), each of which are incorporated by reference in their entireties. In some embodiments, the polynucleotide encoding the SGCG protein comprises, or consists essentially of, or yet further consists of a nucleotide sequence that is at least 70%, 80%, 85%, 90%, 95%, 97%, 98%, 99%, or 100% identical to a SGCG-encoding nucleotide sequence disclosed in International App. No. PCT/US2019/015779 (corresponding to SEQ ID NO: 20), Genbank Accession No. N. Mex._000231.3 (corresponding to SEQ ID NO: 21), Genbank Accession No. N. Mex._001378244.1 (corresponding to SEQ ID NO: 22), Genbank Accession No. N. Mex._001378245.1 (corresponding to SEQ ID NO: 23), or Genbank Accession No. N. Mex._001378246.1 (corresponding to SEQ ID NO: 24) across the entire length of the SGCG-encoding nucleotide sequence. In some embodiments, the polynucleotide encoding the SGCG protein comprises, or consists essentially of, or yet further consists of a nucleotide sequence that is at least about 80% identical to the nucleotide sequence of any one of SEQ ID NOs: 20-24 across the entire length of SEQ ID NOs: 20-24. In some embodiments, the polynucleotide encoding the SGCG protein comprises, or consists essentially of, or yet further consists of a nucleotide sequence that is at least about 85% identical to the nucleotide sequence of any one of SEQ ID NOs: 20-24 across the entire length of SEQ ID NOs: 20-24. In some embodiments, the polynucleotide encoding the SGCG protein comprises, or consists essentially of, or yet further consists of a nucleotide sequence that is at least about 90% identical to the nucleotide sequence of any one of SEQ ID NOs: 20-24 across the entire length of SEQ ID NOs: 20-24. In some embodiments, the polynucleotide encoding the SGCG protein comprises, or consists essentially of, or yet further consists of a nucleotide sequence that is at least about 95% identical to the nucleotide sequence of any one of SEQ ID NOs: 20-24 across the entire length of SEQ ID NOs: 20-24. In some embodiments, the polynucleotide encoding the SGCG protein comprises, or consists essentially of, or yet further consists of a nucleotide sequence that is at least about 100% identical to the nucleotide sequence of any one of SEQ ID NOs: 20-24 across the entire length of SEQ ID NOs: 20-24. SGCG protein sequences are known in the art. For instance, the SGCG protein may comprise, or consist essentially of, or yet further consist of an SGCG amino acid sequence disclosed in International App. No. PCT/US2019/015779 (corresponding to SEQ ID NO: 25), Genbank Accession No. NP_000222.2 (corresponding to SEQ ID NO: 26), Genbank Accession No. NP_001365173.1 (corresponding to SEQ ID NO: 27), Genbank Accession No. NP_001365174.1 (corresponding to SEQ ID NO: 28), or Genbank Accession No. NP_001365175.1 (corresponding to SEQ ID NO: 29), each of which are incorporated by reference in their entireties. In some embodiments, the SGCG protein comprises, or consists essentially of, or yet further consists of an amino acid sequence that is at least 70%, 80%, 85%, 90%, 95%, 97%, 98%, 99%, or 100% identical to an SGCG amino acid sequence disclosed in International App. No. PCT/US2019/015779 (corresponding to SEQ ID NO: 25), Genbank Accession No. NP_000222.2 (corresponding to SEQ ID NO: 26), Genbank Accession No. NP_001365173.1 (corresponding to SEQ ID NO: 27), Genbank Accession No. NP_001365174.1 (corresponding to SEQ ID NO: 28), or Genbank Accession No. NP_001365175.1 (corresponding to SEQ ID NO: 29) across the entire length of the SGCG amino acid sequence. In some embodiments, the SGCG protein comprises, or consists essentially of, or yet further consists of an amino acid sequence that is at least 80% identical to the amino acid sequence of any one of SEQ ID NOs: 25-29 across the entire length of SEQ ID NOs: 25-29. In some embodiments, the SGCG protein comprises, or consists essentially of, or yet further consists of an amino acid sequence that is at least 85% identical to the amino acid sequence of any one of SEQ ID NOs: 25-29 across the entire length of SEQ ID NOs: 25-29. In some embodiments, the SGCG protein comprises, or consists essentially of, or yet further consists of an amino acid sequence that is at least 90% identical to the amino acid sequence of any one of SEQ ID NOs: 25-29 across the entire length of SEQ ID NOs: 25-29. In some embodiments, the SGCG protein comprises, or consists essentially of, or yet further consists of an amino acid sequence that is at least 95% identical to the amino acid sequence of any one of SEQ ID NOs: 25-29 across the entire length of SEQ ID NOs: 25-29. In some embodiments, the SGCG protein comprises, or consists essentially of, or yet further consists of an amino acid sequence that is at least 100% identical to the amino acid sequence of any one of SEQ ID NOs: 25-29 across the entire length of SEQ ID NOs: 25-29. In some embodiments, the polynucleotide encoding the SGCG protein is a codon-optimized.

Disclosed herein are methods of increasing or restoring expression of SGCG in a subject in need thereof, e.g., suffering from a muscular dystrophy. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD and the method comprises, or consists essentially of, or yet further consists of administering to the subject a polynucleotide encoding a dystrophin protein or an abbreviated version of a dystrophin protein.

In some embodiments, the expression level of SGCG is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCG prior to administering a first dose of the polynucleotide encoding SGCA, SGCB, SGCD, or the abbreviated version of the dystrophin protein. In some embodiments, the expression level of SGCG is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCG prior to administering one or more subsequent doses of the polynucleotide encoding SGCA, SGCB, SGCD, or the abbreviated version of the dystrophin protein. In some embodiments, the expression level of SGCG is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCG in a reference sample, wherein the reference sample is from one or more subjects suffering from a muscular dystrophy caused by a mutation in a dystrophin or sarcoglycan gene (e.g., SGCA, SGCB, or SGCD). In some embodiments, the expression level of SGCG is at least 50%, 60%, 70%, 80%, 90%, 100%, 110%, 120%, 130%, 140%, or 150% of the expression level of SGCG in a reference sample, wherein the reference sample is from one or more healthy subjects (e.g., a subject not suffering from a muscular dystrophy caused by a mutation in a dystrophin or sarcoglycan gene). The expression level of SGCG may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, SGCG expression is detected in a sample from the subject. In some embodiments, the sample comprises, or consists essentially of, or yet further consists of a skeletal muscle cell or cardiac muscle cell.

Disclosed herein are methods of localizing SGCG to a cell membrane in a subject in need thereof, e.g., a subject suffering from a muscular dystrophy. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD and the method comprises, or consists essentially of, or yet further consists of administering to the subject a polynucleotide encoding a dystrophin protein or an abbreviated version of a dystrophin protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

In some embodiments, the methods disclosed herein comprise, or consist essentially of, or yet further consist of administering to a subject suffering from a muscular dystrophy a polynucleotide encoding SGCD. In some embodiments, the methods disclosed herein comprise, or consist essentially of, or yet further consist of administering a polynucleotide encoding a SGCD protein to a subject suffering from LGMD2F. In some embodiments, the sarcoglycan is SGCD. Polynucleotides encoding SGCD proteins are known in the art. For instance, the polynucleotide encoding the SGCD protein may comprise, or consists essentially of, or yet further consists of a SGCD nucleotide sequence disclosed in Genbank Accession No. N. Mex._000337.5 (corresponding to SEQ ID NO: 30), Genbank Accession No. N. Mex._001128209.2 (corresponding to SEQ ID NO: 31), or Genbank Accession No. N. Mex._172244.3 (corresponding to SEQ ID NO: 32), each of which are incorporated by reference in their entireties. In some embodiments, the polynucleotide encoding the SGCD protein comprises, or consists essentially of, or yet further consists of a nucleotide sequence that is at least 70%, 80%, 85%, 90%, 95%, 97%, 98%, 99%, or 100% identical to a SGCD-encoding nucleotide sequence disclosed in Genbank Accession No. N. Mex._000337.5 (corresponding to SEQ ID NO: 30), Genbank Accession No. N. Mex._001128209.2 (corresponding to SEQ ID NO: 31), or Genbank Accession No. N. Mex._172244.3 (corresponding to SEQ ID NO: 32) across the entire length of the SGCD-encoding nucleotide sequence. In some embodiments, the polynucleotide encoding the SGCD protein comprises, or consists essentially of, or yet further consists of a nucleotide sequence that is at least about 80% identical to the nucleotide sequence of any one of SEQ ID NOs: 30-32 across the entire length of SEQ ID NOs: 30-32. In some embodiments, the polynucleotide encoding the SGCD protein comprises, or consists essentially of, or yet further consists of a nucleotide sequence that is at least about 85% identical to the nucleotide sequence of any one of SEQ ID NOs: 30-32 across the entire length of SEQ ID NOs: 30-32. In some embodiments, the polynucleotide encoding the SGCD protein comprises, or consists essentially of, or yet further consists of a nucleotide sequence that is at least about 90% identical to the nucleotide sequence of any one of SEQ ID NOs: 30-32 across the entire length of SEQ ID NOs: 30-32. In some embodiments, the polynucleotide encoding the SGCD protein comprises, or consists essentially of, or yet further consists of a nucleotide sequence that is at least about 95% identical to the nucleotide sequence of any one of SEQ ID NOs: 30-32 across the entire length of SEQ ID NOs: 30-32. In some embodiments, the polynucleotide encoding the SGCD protein comprises, or consists essentially of, or yet further consists of a nucleotide sequence that is at least about 100% identical to the nucleotide sequence of any one of SEQ ID NOs: 30-32 across the entire length of SEQ ID NOs: 30-32. SGCD protein sequences are known in the art. For instance, the SGCD protein may comprise, or consists essentially of, or yet further consists of an SGCD amino acid sequence disclosed in Genbank Accession No. NP_000328.2 (corresponding to SEQ ID NO: 33), Genbank Accession No. NP_001121681.1 (corresponding to SEQ ID NO: 34), or Genbank Accession No. NP_758447.1 (corresponding to SEQ ID NO: 35), each of which are incorporated by reference in their entireties. In some embodiments, the SGCD protein comprises, or consists essentially of, or yet further consists of an amino acid sequence that is at least 70%, 80%, 85%, 90%, 95%, 97%, 98%, 99%, or 100% identical to an SGCD amino acid sequence disclosed in Genbank Accession No. NP_000328.2 (corresponding to SEQ ID NO: 33), Genbank Accession No. NP_001121681.1 (corresponding to SEQ ID NO: 34), or Genbank Accession No. NP_758447.1 (corresponding to SEQ ID NO: 35) across the entire length of the SGCD amino acid sequence. In some embodiments, the SGCD protein comprises, or consists essentially of, or yet further consists of an amino acid sequence that is at least about 80% identical to the amino acid sequence of any one of SEQ ID NOs: 33-35 across the entire length of SEQ ID NOs: 33-35. In some embodiments, the SGCD protein comprises, or consists essentially of, or yet further consists of an amino acid sequence that is at least about 85% identical to the amino acid sequence of any one of SEQ ID NOs: 33-35 across the entire length of SEQ ID NOs: 33-35. In some embodiments, the SGCD protein comprises, or consists essentially of, or yet further consists of an amino acid sequence that is at least about 90% identical to the amino acid sequence of any one of SEQ ID NOs: 33-35 across the entire length of SEQ ID NOs: 33-35. In some embodiments, the SGCD protein comprises, or consists essentially of, or yet further consists of an amino acid sequence that is at least about 95% identical to the amino acid sequence of any one of SEQ ID NOs: 33-35 across the entire length of SEQ ID NOs: 33-35. In some embodiments, the SGCD protein comprises, or consists essentially of, or yet further consists of an amino acid sequence that is at least about 100% identical to the amino acid sequence of any one of SEQ ID NOs: 33-35 across the entire length of SEQ ID NOs: 33-35. In some embodiments, the polynucleotide encoding the SGCD protein is a codon-optimized.

Disclosed herein are methods of increasing or restoring expression of SGCD in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is DMD or BMD and the method comprises, or consists essentially of, or yet further consists of administering to the subject a polynucleotide encoding a dystrophin protein or an abbreviated version of a dystrophin protein.

In some embodiments, the expression level of SGCD is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCD prior to administering a first dose of the polynucleotide encoding SGCA, SGCG, SGCB, or the abbreviated version of the dystrophin protein. In some embodiments, the expression level of SGCD is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCD prior to administering one or more subsequent doses of the polynucleotide encoding SGCA, SGCG, SGCB, or the abbreviated version of the dystrophin protein. In some embodiments, the expression level of SGCD is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of SGCD in a reference sample, wherein the reference sample is from one or more subjects suffering from a muscular dystrophy caused by a mutation in a dystrophin or sarcoglycan gene (e.g., SGCA, SGCB, or SGCG). In some embodiments, the expression level of SGCD is at least 50%, 60%, 70%, 80%, 90%, 100%, 110%, 120%, 130%, 140%, or 150% of the expression level of SGCD in a reference sample, wherein the reference sample is from one or more healthy subjects (e.g., a subject not suffering from a muscular dystrophy caused by a mutation in a dystrophin or sarcoglycan gene). The expression level of SGCD may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, SGCD expression is detected in a sample from the subject. In some embodiments, the sample comprises, or consists essentially of, or yet further consists of a skeletal muscle cell or cardiac muscle cell.

Disclosed herein are methods of localizing SGCD to a cell membrane in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is DMD or BMD and the method comprises, or consists essentially of, or yet further consists of administering to the subject a polynucleotide encoding a dystrophin protein or an abbreviated version of a dystrophin protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

Sarcospan

Sarcospan is a component of the DAPC. Sarcospan expression may be reduced or loss in subjects suffering from a muscular dystrophy. Disclosed herein are methods of increasing or restoring expression of sarcospan in a subject in need thereof, e.g., a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD or BMD and the method comprises, or consists essentially of, or yet further consists of administering to the subject a polynucleotide encoding a dystrophin protein or an abbreviated version of a dystrophin protein.

In some embodiments, the expression level of sarcospan is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of sarcospan prior to administering a first dose of the polynucleotide encoding SGCA, SGCG, SGCB, SGCD, or the abbreviated version of the dystrophin protein. In some embodiments, the expression level of sarcospan is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of sarcospan prior to administering one or more subsequent doses of the polynucleotide encoding SGCA, SGCG, SGCB, SGCD, or the abbreviated version of the dystrophin protein. In some embodiments, the expression level of sarcospan is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of sarcospan in a reference sample, wherein the reference sample is from one or more subjects suffering from a muscular dystrophy caused by a mutation in a dystrophin or sarcoglycan gene. In some embodiments, the expression level of sarcospan is at least 50%, 60%, 70%, 80%, 90%, 100%, 110%, 120%, 130%, 140%, or 150% of the expression level of sarcospan in a reference sample, wherein the reference sample is from one or more healthy subjects (e.g., a subject not suffering from a muscular dystrophy caused by a mutation in a dystrophin or sarcoglycan gene). The expression level of sarcospan may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, sarcospan expression is detected in a sample from the subject. In some embodiments, the sample comprises, or consists essentially of, or yet further consists of a skeletal muscle cell or cardiac muscle cell.

Disclosed herein are methods of localizing sarcospan to a cell membrane in a subject in need thereof, e.g. a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD OR BMD and the method comprises, or consists essentially of, or yet further consists of, or consists essentially of, or yet further consists of administering to the subject a polynucleotide encoding a dystrophin protein or an abbreviated version of a dystrophin protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

Dystrophin

In some embodiments, the methods disclosed herein comprise, or consist essentially of, or yet further consist of administering to a subject in need thereof, e.g., a subject suffering from a muscular dystrophy, a polynucleotide encoding a dystrophin protein or abbreviated version of a dystrophin protein. In some embodiments, the methods disclosed herein comprise, or consist essentially of, or yet further consist of administering a polynucleotide encoding a dystrophin protein or an abbreviated version of a dystrophin protein to a subject suffering from DMD OR BMD. Polynucleotides encoding a dystrophin protein are known in the art. For instance, the polynucleotide encoding the dystrophin may comprise, or consist essentially of, or yet further consist of a nucleotide sequence disclosed in Genbank Accession No. AH003182.2, which is incorporated by reference in its entirety. In some embodiments, the polynucleotide encoding the dystrophin protein comprises, or consists essentially of, or yet further consists of a nucleotide sequence that is at least 70%, 80%, 85%, 90%, 95%, 97%, 98%, 99%, or 100% identical to the nucleotide sequence of Genbank Accession No. AH003182.2 (corresponding to SEQ ID NO: 36) across the entire length of SEQ ID NO: 36. In some embodiments, the polynucleotide encoding the dystrophin protein comprises, or consists essentially of, or yet further consists of a nucleotide sequence that is at least about 80% identical to the nucleotide sequence of SEQ ID NO: 36 across the entire length of SEQ ID NO: 36. In some embodiments, the polynucleotide encoding the dystrophin protein comprises, or consists essentially of, or yet further consists of a nucleotide sequence that is at least about 85% identical to the nucleotide sequence of SEQ ID NO: 36 across the entire length of SEQ ID NO: 36. In some embodiments, the polynucleotide encoding the dystrophin protein comprises, or consists essentially of, or yet further consists of a nucleotide sequence that is at least about 90% identical to the nucleotide sequence of SEQ ID NO: 36 across the entire length of SEQ ID NO: 36. In some embodiments, the polynucleotide encoding the dystrophin protein comprises, or consists essentially of, or yet further consists of a nucleotide sequence that is at least about 95% identical to the nucleotide sequence of SEQ ID NO: 36 across the entire length of SEQ ID NO: 36. In some embodiments, the polynucleotide encoding the dystrophin protein comprises, or consists essentially of, or yet further consists of a nucleotide sequence that is at least about 100% identical to the nucleotide sequence of SEQ ID NO: 36 across the entire length of SEQ ID NO: 36.

Dystrophin protein sequences are known in the art. For instance, the dystrophin protein may comprise, or consist essentially of, or yet further consist of the amino acid sequence of Genbank Accession No. AAA74506.1 (corresponding to SEQ ID NO: 37), which are incorporated by reference in its entirety. In some embodiments, the dystrophin protein comprises, or consists essentially of, or yet further consists of an amino acid sequence that is at least 70%, 80%, 85%, 90%, 95%, 97%, 98%, 99%, or 100% identical to the amino acid sequence of Genbank Accession No. AAA74506.1 (corresponding to SEQ ID NO: 37) across the entire length of SEQ ID NO: 37. In some embodiments, the dystrophin protein comprises, or consists essentially of, or yet further consists of an amino acid sequence that is at least about 80% identical to the amino acid sequence of SEQ ID NO: 37 across the entire length of SEQ ID NO: 37. In some embodiments, the dystrophin protein comprises, or consists essentially of, or yet further consists of an amino acid sequence that is at least about 85% identical to the amino acid sequence of SEQ ID NO: 37 across the entire length of SEQ ID NO: 37. In some embodiments, the dystrophin protein comprises, or consists essentially of, or yet further consists of an amino acid sequence that is at least about 90% identical to the amino acid sequence of SEQ ID NO: 37 across the entire length of SEQ ID NO: 37. In some embodiments, the dystrophin protein comprises, or consists essentially of, or yet further consists of an amino acid sequence that is at least about 95% identical to the amino acid sequence of SEQ ID NO: 37 across the entire length of SEQ ID NO: 37. In some embodiments, the dystrophin protein comprises, or consists essentially of, or yet further consists of an amino acid sequence that is at least about 100% identical to the amino acid sequence of SEQ ID NO: 37 across the entire length of SEQ ID NO: 37. In some embodiments, the polynucleotide encoding the dystrophin protein is a codon-optimized. Polynucleotides encoding an abbreviated versions of a dystrophin protein are known in the art. For instance, the polynucleotide encoding the abbreviated version of a dystrophin protein may comprise, or consist essentially of, or yet further consist of a microdystrophin or mini dystrophin gene as disclosed in Fabb et al., Hum Mol Genet, 11(7):733-741, 2002, Gregorevic et al., Mol Ther, 16(4):657-64, 2008, Zhang and Duan, Hum Gene Ther, 23(1):98-103, 2012, or Decrouy et al., Gene Ther, 5(1):59-64, 1998, each of which are incorporated by reference in their entireties. In some embodiments, the polynucleotide encoding the abbreviated version of a dystrophin protein comprises, or consists essentially of, or yet further consists of a nucleotide sequence that is at least 70%, 80%, 85%, 90%, 95%, 97%, 98%, 99%, or 100% identical to a microdystrophin or mini dystrophin gene disclosed in Fabb et al., Hum Mol Genet, 11(7):733-741, 2002, Gregorevic et al., Mol Ther, 16(4):657-64, 2008, Zhang and Duan, Hum Gene Ther, 23(1):98-103, 2012, or Decrouy et al., Gene Ther, 5(1):59-64, 1998. In some embodiments, the polynucleotide encoding the abbreviated version of a dystrophin protein comprises, or consists essentially of, or yet further consists of a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44. In some embodiments, the polynucleotide encoding the abbreviated version of a dystrophin protein comprises, or consists essentially of, or yet further consists of a nucleotide sequence that is at least about 80% identical to the nucleotide sequence of any one of SEQ ID NOs: 38 and 40-44 across the entire length of SEQ ID NOs: 38 and 40-44. In some embodiments, the polynucleotide encoding the abbreviated version of a dystrophin protein comprises, or consists essentially of, or yet further consists of a nucleotide sequence that is at least about 85% identical to the nucleotide sequence of any one of SEQ ID NOs: 38 and 40-44 across the entire length of SEQ ID NOs: 38 and 40-44. In some embodiments, the polynucleotide encoding the abbreviated version of a dystrophin protein comprises, or consists essentially of, or yet further consists of a nucleotide sequence that is at least about 90% identical to the nucleotide sequence of any one of SEQ ID NOs: 38 and 40-44 across the entire length of SEQ ID NOs: 38 and 40-44. In some embodiments, the polynucleotide encoding the abbreviated version of a dystrophin protein comprises, or consists essentially of, or yet further consists of a nucleotide sequence that is at least about 95% identical to the nucleotide sequence of any one of SEQ ID NOs: 38 and 40-44 across the entire length of SEQ ID NOs: 38 and 40-44. In some embodiments, the polynucleotide encoding the abbreviated version of a dystrophin protein comprises, or consists essentially of, or yet further consists of a nucleotide sequence that is at least about 100% identical to the nucleotide sequence of any one of SEQ ID NOs: 38 and 40-44 across the entire length of SEQ ID NOs: 38 and 40-44. Microdystrophin and mini dystrophin protein sequences are known in the art. For instance, a microdystrophin or mini dystrophin protein sequence may comprise, or consist essentially of, or yet further consist of amino acid sequence disclosed in Fabb et al., Hum Mol Genet, 11(7):733-741, 2002, Gregorevic et al., Mol Ther, 16(4):657-64, 2008, Zhang and Duan, Hum Gene Ther, 23(1):98-103, 2012, or Decrouy et al., Gene Ther, 5(1):59-64, 1998. In some embodiments, the abbreviated version of a dystrophin protein comprises, or consists essentially of, or yet further consists of an amino acid sequence that is at least 70%, 80%, 85%, 90%, 95%, 97%, 98%, 99%, or 100% identical to a microdystrophin or mini dystrophin amino acid sequence disclosed in Fabb et al., Hum Mol Genet, 11(7):733-741, 2002, Gregorevic et al., Mol Ther, 16(4):657-64, 2008, Zhang and Duan, Hum Gene Ther, 23(1):98-103, 2012, or Decrouy et al., Gene Ther, 5(1):59-64, 1998. In some embodiments, the abbreviated version of a dystrophin protein comprises, or consists essentially of, or yet further consists of an amino acid sequence that is at least 70%, 80%, 85%, 90%, 95%, 97%, 98%, 99%, or 100% identical to the amino acid sequence of SEQ ID NO: 39 across the entire length of SEQ ID NO: 39. In some embodiments, the abbreviated version of a dystrophin protein comprises, or consists essentially of, or yet further consists of an amino acid sequence that is at least about 80% identical to the amino acid sequence of SEQ ID NO: 39 across the entire length of SEQ ID NO: 39. In some embodiments, the abbreviated version of a dystrophin protein comprises, or consists essentially of, or yet further consists of an amino acid sequence that is at least about 85% identical to the amino acid sequence of SEQ ID NO: 39 across the entire length of SEQ ID NO: 39. In some embodiments, the abbreviated version of a dystrophin protein comprises, or consists essentially of, or yet further consists of an amino acid sequence that is at least about 90% identical to the amino acid sequence of SEQ ID NO: 39 across the entire length of SEQ ID NO: 39. In some embodiments, the abbreviated version of a dystrophin protein comprises, or consists essentially of, or yet further consists of an amino acid sequence that is at least about 95% identical to the amino acid sequence of SEQ ID NO: 39 across the entire length of SEQ ID NO: 39. In some embodiments, the abbreviated version of a dystrophin protein comprises, or consists essentially of, or yet further consists of an amino acid sequence that is at least about 100% identical to the amino acid sequence of SEQ ID NO: 39 across the entire length of SEQ ID NO: 39. In some embodiments, the polynucleotide encoding the abbreviated version of a dystrophin protein is a codon-optimized.

Disclosed herein are methods of increasing or restoring expression of dystrophin in a subject in need thereof, e.g. a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein.

In some embodiments, the expression level of dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of dystrophin prior to administering a first dose of the polynucleotide encoding SGCA, SGCG, SGCB, or SGCD. In some embodiments, the expression level of dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of dystrophin prior to administering one or more subsequent doses of the polynucleotide encoding SGCA, SGCG, SGCB, or SGCD. In some embodiments, the expression level of dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of dystrophin in a reference sample, wherein the reference sample is from one or more subjects suffering from a muscular dystrophy caused by a mutation in a sarcoglycan gene (e.g., SGCA, SGCB, SGCG, or SGCD). In some embodiments, the expression level of dystrophin is at least 50%, 60%, 70%, 80%, 90%, 100%, 110%, 120%, 130%, 140%, or 150% of the expression level of dystrophin in a reference sample, wherein the reference sample is from one or more healthy subjects (e.g., a subject not suffering from a muscular dystrophy caused by a mutation in a sarcoglycan gene). The expression level of dystrophin may be detected by any known methods for detecting or quantifying protein or mRNA levels. For instance, such methods may include immunofluorescence staining, Western blot, or polymerase chain reaction (PCR). In some embodiments, PCR is quantitative reverse transcription PCR (qRT-PCR). In some embodiments, dystrophin expression is detected in a sample from the subject. In some embodiments, the sample comprises, or consists essentially of, or yet further consists of a skeletal muscle cell or cardiac muscle cell.

Disclosed herein are methods of localizing dystrophin to a cell membrane in a subject suffering from a muscular dystrophy. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the cell membrane is a muscle cell membrane or sarcolemma. In some embodiments, the cell membrane is a skeletal muscle cell membrane or sarcolemma. In some embodiments, the cell membrane is a cardiac muscle cell membrane or sarcolemma.

Sarcoglycan Complex

In some embodiments, a method of repairing a sarcoglycan complex in a subject suffering from a muscular dystrophy, comprises, consists of, or yet further consists essentially of administering to the subject a polynucleotide encoding a sarcoglycan protein or abbreviated version of a dystrophin protein. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD OR BMD and the method comprises, or consists essentially of, or yet further consists of administering to the subject a polynucleotide encoding a dystrophin protein or an abbreviated version of a dystrophin protein. In some embodiments, the method further comprises, or consists essentially of, or yet further consists of detecting the expression level of at least one protein selected from sarcoglycan, dystrophin, and sarcospan. In some embodiments, the sarcoglycan is selected from α-sarcoglycan (SGCA), β-sarcoglycan (SGCB), δ-sarcoglycan (SGCD), or γ-sarcoglycan (SGCG). In some embodiments, said detecting comprises, or consists essentially of, or yet further consists of a method comprising, or consisting essentially of, or yet further consisting of at least one of immunofluorescence staining, Western blot, or polymerase chain reaction (PCR). In some embodiments, PCR is quantitative reverse transcription PCR (qRT-PCR). In some embodiments, expression level of the sarcoglycan, dystrophin, or sarcospan is determined from a sample from the subject. In some embodiments, the sample comprises, or consists essentially of, or yet further consists of a skeletal muscle cell or cardiac muscle cell. In some embodiments, the sarcoglycan complex is repaired when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the sarcoglycan complex is repaired when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the sarcoglycan complex is repaired when the expression level of the sarcoglycan, dystrophin, or sarcospan protein is increased as compared to the expression level of the sarcoglycan, dystrophin, or sarcospan in a reference sample, wherein the reference sample is from one or more subjects suffering from a muscular dystrophy caused by a mutation in a dystrophin or sarcoglycan gene. In some embodiments, the sarcoglycan complex is repaired when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin prior to administering the polynucleotide encoding the sarcoglycan protein or abbreviated version of the dystrophin protein. In some embodiments, the sarcoglycan complex is repaired when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin in a reference sample, wherein the reference sample is from one or more subjects suffering from a muscular dystrophy caused by a mutation in a dystrophin or sarcoglycan gene. In some embodiments, the sarcoglycan complex is repaired when the expression level of the sarcoglycan, sarcospan, or dystrophin is at least 50%, 60%, 70%, 80%, 90%, 100%, 110%, 120%, 130%, 140%, or 150% of the expression level of the sarcoglycan, sarcospan, or dystrophin in a reference sample, wherein the reference sample is from one or more healthy subjects (e.g., a subject not suffering from a muscular dystrophy caused by a mutation in a dystrophin or sarcoglycan gene).

In some embodiments, a method of restoring or stabilizing a dystrophin-associated protein complex (DAPC) in a subject suffering from a muscular dystrophy, comprises, consists of, or yet further consists essentially of administering to the subject a polynucleotide encoding a sarcoglycan protein or abbreviated version of a dystrophin protein. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD OR BMD and the method comprises, or consists essentially of, or yet further consists of administering to the subject a polynucleotide encoding a dystrophin protein or an abbreviated version of a dystrophin protein. In some embodiments, the method further comprises, or consists essentially of, or yet further consists of detecting the expression level of at least one protein selected from sarcoglycan, dystrophin, and sarcospan. In some embodiments, the sarcoglycan is selected from α-sarcoglycan (SGCA), β-sarcoglycan (SGCB), δ-sarcoglycan (SGCD), or γ-sarcoglycan (SGCG). In some embodiments, said detecting comprises, or consists essentially of, or yet further consists of a method comprising, or consisting essentially of, or yet further consisting of at least one of immunofluorescence staining, Western blot, or polymerase chain reaction (PCR). In some embodiments, PCR is quantitative reverse transcription PCR (qRT-PCR). In some embodiments, expression level of the sarcoglycan, dystrophin, or sarcospan is determined from a sample from the subject. In some embodiments, the sample comprises, or consists essentially of, or yet further consists of a skeletal muscle cell or cardiac muscle cell. In some embodiments, the DAPC is restored or stabilized when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the DAPC is restored or stabilized when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the DAPC is restored or stabilized when the expression level of the sarcoglycan, dystrophin, or sarcospan protein is increased as compared to the expression level of the sarcoglycan, dystrophin, or sarcospan in a reference sample, wherein the reference sample is from one or more subjects suffering from a muscular dystrophy caused by a mutation in a dystrophin or sarcoglycan gene. In some embodiments, the DAPC is restored or stabilized when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin prior to administering the polynucleotide encoding the sarcoglycan protein or abbreviated version of the dystrophin protein. In some embodiments, the DAPC is restored or stabilized when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin in a reference sample, wherein the reference sample is from one or more subjects suffering from a muscular dystrophy caused by a mutation in a dystrophin or sarcoglycan gene. In some embodiments, the DAPC is restored or stabilized when the expression level of the sarcoglycan, sarcospan, or dystrophin is at least 50%, 60%, 70%, 80%, 90%, 100%, 110%, 120%, 130%, 140%, or 150% of the expression level of the sarcoglycan, sarcospan, or dystrophin in a reference sample, wherein the reference sample is from one or more healthy subjects (e.g., a subject not suffering from a muscular dystrophy caused by a mutation in a dystrophin or sarcoglycan gene).

In some embodiments, a method of restoring or increasing the function of a dystrophin-associated protein complex (DAPC), in a subject suffering from a muscular dystrophy, comprises, consists of, or yet further consists essentially of administering to the subject a polynucleotide encoding a sarcoglycan protein or abbreviated version of a dystrophin protein. In some embodiments, the muscular dystrophy is LGMD2C and the method comprises, or consists essentially of, or yet further consists of administering to the subject a polynucleotide encoding a SGCG protein. In some embodiments, the muscular dystrophy is LGMD2D and the method comprises, or consists essentially of, or yet further consists of administering to the subject a polynucleotide encoding a SGCA protein. In some embodiments, the muscular dystrophy is LGMD2E and the method comprises, or consists essentially of, or yet further consists of administering to the subject a polynucleotide encoding a SGCB protein. In some embodiments, the muscular dystrophy is LGMD2F and the method comprises, or consists essentially of, or yet further consists of administering to the subject a polynucleotide encoding a SGCD protein. In some embodiments, the muscular dystrophy is DMD OR BMD and the method comprises, or consists essentially of, or yet further consists of administering to the subject a polynucleotide encoding a dystrophin protein or an abbreviated version of a dystrophin protein. In some embodiments, the method further comprises, or consists essentially of, or yet further consists of detecting the expression level of at least one protein selected from sarcoglycan, dystrophin, and sarcospan. In some embodiments, the sarcoglycan is selected from α-sarcoglycan (SGCA), β-sarcoglycan (SGCB), δ-sarcoglycan (SGCD), or γ-sarcoglycan (SGCG). In some embodiments, said detecting comprises, or consists essentially of, or yet further consists of a method comprising, or consisting essentially of, or yet further consisting of at least one of immunofluorescence staining, Western blot, or polymerase chain reaction (PCR). In some embodiments, PCR is quantitative reverse transcription PCR (qRT-PCR). In some embodiments, expression level of the sarcoglycan, dystrophin, or sarcospan is determined from a sample from the subject. In some embodiments, the sample comprises, or consists essentially of, or yet further consists of a skeletal muscle cell or cardiac muscle cell. In some embodiments, the DAPC is function is increased or restored when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the DAPC is function is increased or restored when the expression level of the sarcoglycan, dystrophin, or sarcospan protein is increased as compared to the expression level of the sarcoglycan, dystrophin, or sarcospan prior to administering the polynucleotide encoding the sarcoglycan protein or abbreviated version of the dystrophin protein. In some embodiments, the DAPC is function is increased or restored when the expression level of the sarcoglycan, dystrophin, or sarcospan protein is increased as compared to the expression level of the sarcoglycan, dystrophin, or sarcospan in a reference sample, wherein the reference sample is from one or more subjects suffering from a muscular dystrophy caused by a mutation in a dystrophin or sarcoglycan gene. In some embodiments, the DAPC is function is increased or restored when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin prior to administering the polynucleotide encoding the sarcoglycan protein or abbreviated version of the dystrophin protein. In some embodiments, the DAPC is function is increased or restored when the expression level of the sarcoglycan, sarcospan, or dystrophin is increased by at least 10%, 20%, 30%, 40%, 50%, 60%, 70%, 80%, 90%, 100%, 110%, 120%, 130%, 140%, 150%, 175%, 200%, 225%, 250%, 275%, or 300% or more as compared to the expression level of the sarcoglycan, sarcospan, or dystrophin in a reference sample, wherein the reference sample is from one or more subjects suffering from a muscular dystrophy caused by a mutation in a dystrophin or sarcoglycan gene. In some embodiments, the DAPC is function is increased or restored when the expression level of the sarcoglycan, sarcospan, or dystrophin is at least 50%, 60%, 70%, 80%, 90%, 100%, 110%, 120%, 130%, 140%, or 150% of the expression level of the sarcoglycan, sarcospan, or dystrophin in a reference sample, wherein the reference sample is from one or more healthy subjects (e.g., a subject not suffering from a muscular dystrophy caused by a mutation in a dystrophin or sarcoglycan gene).

Modes for Administration

In some embodiments, the polynucleotides encoding the sarcoglycan protein or abbreviated version of the dystrophin protein are administered orally, parenterally (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.). In some embodiments, the polynucleotide is administered intramuscularly or intravenously.

In some embodiments, the polynucleotides are administered in a nanoparticle, liposome, or encapsidated within a viral vector (e.g., viral vector particle). Alternatively, or additionally, the polynucleotide is comprised within a vector, e.g., a plasmid or viral vector. In some embodiments, the viral vector is a vector of a retrovirus, adenovirus, adeno-associated virus (AAV), lentivirus, alphavirus, flavivirus, rhabdovirus, measles virus, poxvirus, picornavirus, or herpes simplex virus. In some embodiments, the viral vector is a recombinant viral vector. In some embodiments, the viral vector is a recombinant AAV vector.

In some embodiments, the polynucleotides are administered in an adeno-associated viral (AAV) expression cassette, AAV genome, or AAV vector. In some embodiments, the AAV is AAVrh.20, AAV-1, AAV-2, AAV-3, AAV-4, AAV-5, AAV-6, AAV-7, AAVrh.74, AAV-8, AAV-9, AAV-10, AAVrh.10, AAV-11, AAV-12 and AAV-13. In some embodiments, the AAV is AAVrh.74. In some embodiments, the AAV genome is a single-stranded (ss) AAV genome. In some embodiments, the AAV genome is a self-complementary (sc) AAV genome. In some embodiments, the AAV is a self-complementary AAVrh.74. In some embodiments, the AAV is a scAAVrh.74 vector comprising a MHCK7 promoter. In some embodiments, the AAV genome comprises a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

In another aspect, the polynucleotides or recombinant AAV vectors described herein may be operably linked to a muscle-specific control element and/or an enhancer element. For example the muscle-specific control element is human skeletal actin gene element, cardiac actin gene element, myocyte-specific enhancer binding factor MEF element, muscle creatine kinase (MCK) promoter, tMCK (truncated MCK) element, myosin heavy chain (MHC) element, MHCK7 (a hybrid version of MHC and MCK) promoter, C5-12 (synthetic promoter), murine creatine kinase enhancer element, skeletal fast-twitch troponin C gene element, slow-twitch cardiac troponin C gene element, the slow-twitch troponin I gene element, hypoxia-inducible nuclear factors element, steroid-inducible element or glucocorticoid response element (GRE).

In some embodiments, the muscle-specific promoter is MHCK7 promoter. In some embodiments, the MHCK7 promoter comprises, consists essentially of, or further yet consist of a nucleotide sequence that is at least 70%, 80%, 85%, 90%, 95%, 97%, 98%, 99%, or 100% to the nucleotide sequence of SEQ ID NO: 4 across the full length of SEQ ID NO: 4. In some embodiments, the MHCK7 promoter comprises, consists essentially of, or further yet consist of a nucleotide sequence that is at least about 80% to the nucleotide sequence of SEQ ID NO: 4 across the full length of SEQ ID NO: 4. In some embodiments, the MHCK7 promoter comprises, consists essentially of, or further yet consist of a nucleotide sequence that is at least about 85% to the nucleotide sequence of SEQ ID NO: 4 across the full length of SEQ ID NO: 4. In some embodiments, the MHCK7 promoter comprises, consists essentially of, or further yet consist of a nucleotide sequence that is at least about 90% to the nucleotide sequence of SEQ ID NO: 4 across the full length of SEQ ID NO: 4. In some embodiments, the MHCK7 promoter comprises, consists essentially of, or further yet consist of a nucleotide sequence that is at least about 95% to the nucleotide sequence of SEQ ID NO: 4 across the full length of SEQ ID NO: 4. In some embodiments, the MHCK7 promoter comprises, consists essentially of, or further yet consist of a nucleotide sequence that is at least about 100% to the nucleotide sequence of SEQ ID NO: 4 across the full length of SEQ ID NO: 4.

In some embodiments, the muscle-specific promoter is tMCK promoter. In some embodiments, the tMCK promoter comprises, consists essentially of, or further yet consist of a nucleotide sequence that is at least 70%, 80%, 85%, 90%, 95%, 97%, 98%, 99%, or 100% to the nucleotide sequence of SEQ ID NO: 6 across the full length of SEQ ID NO: 6. In some embodiments, the tMCK promoter comprises, consists essentially of, or further yet consist of a nucleotide sequence that is at least about 80% to the nucleotide sequence of SEQ ID NO: 6 across the full length of SEQ ID NO: 6. In some embodiments, the tMCK promoter comprises, consists essentially of, or further yet consist of a nucleotide sequence that is at least about 85% to the nucleotide sequence of SEQ ID NO: 6 across the full length of SEQ ID NO: 6. In some embodiments, the tMCK promoter comprises, consists essentially of, or further yet consist of a nucleotide sequence that is at least about 90% to the nucleotide sequence of SEQ ID NO: 6 across the full length of SEQ ID NO: 6. In some embodiments, the tMCK promoter comprises, consists essentially of, or further yet consist of a nucleotide sequence that is at least about 95% to the nucleotide sequence of SEQ ID NO: 6 across the full length of SEQ ID NO: 6. In some embodiments, the tMCK promoter comprises, consists essentially of, or further yet consist of a nucleotide sequence that is at least about 100% to the nucleotide sequence of SEQ ID NO: 6 across the full length of SEQ ID NO: 6.

An exemplary rAAV described herein is pAAV.MHCK7.hSGCB which comprises the nucleotide sequence of SEQ ID NO: 3. Within the nucleotide sequence of SEQ ID NO: 3, the MCHK7 promoter spans nucleotides 130-921, a SV40 chimeric intron (SEQ ID NO: 9) spans nucleotides 931-1078, the SGCB sequence (SEQ ID NO: 1) spans nucleotides 1091-2047 and the poly A (SEQ ID NO: 10) spans nucleotides 2054-2106. In some embodiments, the rAAV pAAV.MHCK7.hSGCB comprises a nucleotide sequence of SEQ ID NO: 7. Within the nucleotide sequence of SEQ ID NO: 7, the MCHK7 promoter spans nucleotides 128-919, a SV40 chimeric intron spans nucleotides 929-1076, the SGCB sequence spans nucleotides 1086-2042 and the poly A spans nucleotides 2049-2101.

In some embodiments, the rAAV comprises a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence set forth in SEQ ID NO: 3 or SEQ ID NO: 7, or a nucleotide sequence that encodes a polypeptide that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to SEQ ID NO: 2.

1. An exemplary rAAV vector described herein is pAAV.MHCK7.hSGCG, which comprises the nucleotide sequence of SEQ ID NO: 19; wherein the MCHK7 promoter spans nucleotides 136-927, an intron spans nucleotides 937-1084, the SGCG sequence spans nucleotides 1094-1969 and the polyA spans nucleotides 1976-2028. In some cases, the only viral sequences included in a rAAV vector are the inverted terminal repeats, which are required for viral DNA replication and packaging. In some embodiments, pAAV.MHCK7.hSGCG is packaged in an AAV rh.74 capsid. In one embodiment, the AAV vector was administered to the subject at a dosage of 1.85e13 vg/kg or 7.41e13 vg/kg, wherein the dosage is quantified by a linearized PCR standard.

In some embodiments, the rAAV comprises a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence set forth in SEQ ID NO: 19.

An exemplary rAAV is scAAVrh74.tMCK.hSGCA (SEQ ID NO: 47). In some embodiments, the rAAV comprises a nucleotide sequence that is at least 65%, at least 70%, at least 75%, at least 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% identical to the nucleotide sequence set forth in SEQ ID NO: 47.

In one embodiment, the AAV is a pAAV.tMCK.hSGCA.KAN plasmid (SEQ ID NO: 48). In another embodiment, the rAAV comprises a nucleotide sequence that is at least about 65%, about 70%, about 75%, about 80%, about 81%, about 82%, about 83%, about 84%, about 85%, about 86%, about 87%, about 88%, or about 89%, more typically about 90%, about 91%, about 92%, about 93%, about 94%, about 95%, about 96%, about 97%, about 98%, or about 99% or more identical to SEQ ID NO: 48.

Titers of AAV vectors to be administered in methods of the invention will vary depending, for example, on the particular AAV, the mode of administration, the treatment goal, the individual, and the cell type(s) being targeted, and may be determined by methods standard in the art. Titers of AAV may range from at least about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). For instance, dosages of AAV may range from at least about 1×10⁶, about 1×10⁷, about 1×10⁸, about 1×10⁹, about 1×10¹⁰, about 1×10¹¹, about 1×10¹², about 2×10¹² about 3×10¹², about 4×10¹², about 5×10¹², about 6×10¹², about 7×10¹², about 8×10¹², about 9×10¹², about 1×10¹³ to about 1×10¹⁴ viral genomes. In some embodiments, dosages may be expressed in the units of viral genomes/kilogram of subject mass (vg/kg). For example, dosages of AAV is about 1×10⁶-1×10¹⁶ vg/kg, about 1×10⁸-1×10¹⁵ vg/kg, about 1×10¹⁰-1×10¹⁴ vg/kg, about 1×10¹²-1×10¹⁴ vg/kg, about 1×10¹³-1×10¹⁴ vg/kg, or about 1.80×10¹³-7.5×10¹³ vg/kg. In another embodiment, the dosages is about at least 1×10⁶, about 1×10⁷, about 1×10⁸, about 1×10⁹, about 1×10¹⁰, about 1×10¹¹, about 1×10¹², about 2×10¹², about 4×10¹², about 6×10¹², about 8×10¹², about 1×10¹³, about 2×10¹³, about 2.4×10¹³, about 3×10¹³, about 4×10¹³, about 5×10¹³, about 6×10¹³, about 7×10¹³, about 8×10¹³, about 9×10¹³, about 1×10¹⁴, about 1×10¹⁵, or at least about 1×10¹⁶ vg/kg. In one embodiment, the dosage is at least 2×10¹², 4×10¹², 6×10¹², 8×10¹², 1×10¹³, 2×10¹³, 2.4×10¹³, 3×10¹³, 4×10¹³, 5×10¹³, 6×10¹³, 7×10¹³, or 8×10¹³ vg/kg. In some embodiments, the dosage is at least about 1.85×10¹³ vg/kg. In some embodiments, the dosage is at least about 7.41×10¹³ vg/kg. In some embodiments, the dosage is quantified by linearized PCR standard. Alternatively, in some embodiments, the dosage is quantified by supercoiled PCR standard.

A therapeutically effective amount of the rAAV vector is a dose of rAAV ranging from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or 1e13 vg/kg to about 3e14 vg/kg, or about 1e13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg, 6e14 vg/kg, 7e14 vg/kg, 8e14 vg/kg, or 9e14 vg/kg. The invention also comprises compositions comprising these ranges of rAAV vector.

For example, a therapeutically effective amount of rAAV vector is a dose of 1e13 vg/kg, about 2e13 vg/kg, about 3e13 vg/kg, about 4e13 vg/kg, about 5e13 vg/kg, about 6e13 vg/kg, about 7e13 vg/kg, about 7.4e13 vg/kg, about 8e13 vg/kg, about 9e13 vg/kg, about 1e14 vg/kg, about 2e14 vg/kg, about 3e14 vg/kg, about 4e14 vg/kg and 5e14 vg/kg. The titer or dosage of AAV vectors can vary based on the physical forms of plasmid DNA as a quantitation standard. For example, the value of titer or dosage may vary based off of a supercoiled standard qPCR titering method or a linear standard qPCR tittering method. In one embodiment, a therapeutically effective amount of rAAV is a dose of 5e13 vg/kg based on a supercoiled plasmid as the quantitation standard or a dose of 1.85e13 vg/kg based on a linearized plasmid as the quantitation standard. In another embodiment, a therapeutically effective amount of rAAV is a dose of 2e14 vg/kg based on the supercoiled plasmid as the quantitation standard or a dose of 7.41e13 vg/kg based on the linearized plasmid as the quantitation standard. In another embodiment, the therapeutically effective amount of scAAVrh74.MHCK7.hSGCB is a dose ranging from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or 1e13 vg/kg to about 3e14 vg/kg, or about 1e13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg, 6e14 vg/kg, 7e14 vg/kg, 8e14 vg/kg, or 9e14 vg/kg, based on the supercoiled plasmid as the quantitation standard. The invention also comprises compositions comprising these doses of rAAV vector.

In some embodiments, the methods disclosed herein comprise, or consist essentially of, or yet further consist of administering at least about 1×10⁶, about 1×10⁷, about 1×10⁸, about 1×10⁹, about 1×10¹⁰, about 1×10¹¹, about 1×10¹², about 2×10¹², about 3×10¹², about 4×10¹², about 5×10¹², about 6×10¹², about 7×10¹², about 8×10¹², about 9×10¹², about 1×10¹³ vg in a total volume of 1.5 ml per injection. In some embodiments, the methods disclosed herein comprise, or consist essentially of, or yet further consist of administering a total daily dose of at least about 1×10⁶, about 1×10⁷, about 1×10⁸, about 1×10⁹, about 1×10¹⁰, about 1×10¹¹, about 1×10¹², about 2×10¹², about 3×10¹², about 4×10¹², about 5×10¹², about 6×10¹², about 7×10¹², about 8×10¹², about 9×10¹², about 1×10¹³, about 2×10¹³, about 5×10¹³, about 7×10¹³, about 1×10¹⁴ vg. One exemplary method of determining encapsidated vector genome titer uses quantitative PCR, such as the methods described in Pozsgai et al., Mol. Ther. 25(4): 855-869, 2017, which is incorporated by reference in its entirety.

In some embodiments, any of the methods disclosed herein comprise, consist essentially of, or yet further consist of administering to the subject an rAAV intravenous infusion over approximately 1 to 2 hours at a dose of about 5.0×10¹³ vg/kg or about 2.0×10¹⁴ vg/kg based on a supercoiled plasmid as the quantitation standard, or about 1.85×10¹³ vg/kg or 7.41×10¹³ vg/kg based on a linearized plasmid as the quantitation standard.

In some embodiments, the dose of rAAV administered using an intravenous route and the dose is about 1.0×10¹³ vg/kg to about 5×10¹⁴ based on a supercoiled plasmid as the quantitation standard or about 1.0×10¹³ vg/kg to about 1.0×10¹⁴ vg/kg based on a linearized plasmid as the quantitation standard.

In addition, the dose of the rAAV administered is about 1.5×10¹³ vg to about 2×10¹⁶ vg, or 1.5×10¹³ vg to 1×10¹⁶ vg, or about 1.5×10¹³ vg to about 2×10¹⁵ vg, or about 1.5×10¹³ vg to about 1×10¹⁵ vg. In addition, in any of the methods, compositions and uses, the dose of rAAV is administered at a concentration of about 10 mL/kg.

In some embodiments, any of the polynucleotides encoding the sarcoglycan protein or abbreviated version of the dystrophin protein are administered to the subject at least 1, 2, 3, 4, 5, 6, 7, 8, 9, or 10 times a day. In some embodiments, any of the polynucleotides or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the polynucleotides or compositions disclosed herein are administered to the subject at least every 1, 2, 3, 4, 5, 6, 7, 8, 9, or 10 days. In some embodiments, any of the polynucleotides or compositions disclosed herein are administered to the subject at least every 1, 2, 3, 4, 5, 6, 7, 8, 9, 10, 11, 12, 13, or 14 weeks. In some embodiments, any of the polynucleotides or compositions disclosed herein are administered to the subject for a period of at least 1, 2, 3, 4, 5, 6, 7, 8, 9, or 10 days. In some embodiments, any of the polynucleotides or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the polynucleotides or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the methods disclosed herein comprise, or consist essentially of, or yet further consist of administering any of the polynucleotides or compositions disclosed herein systemically. For example, systemic administration is administration into the circulatory system so that the entire body is affected. Systemic administration includes enteral administration such as absorption through the gastrointestinal tract and parenteral administration through injection, infusion or implantation.

In some embodiments, the methods disclosed herein comprise, or consist essentially of, or yet further consist of administering any of the polynucleotides or compositions disclosed herein locally. In some embodiments, the methods disclosed herein comprise, or consist essentially of, or yet further consist of administering any of the polynucleotides or compositions disclosed herein to one or more tissues. In some embodiments, the tissue is selected from muscle, epithelial, connective, and nervous tissue. In some embodiments, the tissue is a muscle tissue.

Combination therapies are also contemplated by the invention. Combination as used herein includes both simultaneous treatment and sequential treatments. Combinations of methods of the invention with standard medical treatments (e.g., corticosteroids) are specifically contemplated, as are combinations with novel therapies.

In some embodiments, the methods disclosed herein further comprise, or consist essentially of, or yet further consist of detecting the presence or absence of a mutation in a sarcoglycan gene or dystrophin gene in the subject prior to or subsequent to administering any of the polynucleotides or compositions disclosed herein to the subject. In some embodiments, any of the polynucleotides or compositions disclosed herein are administered to the subject upon detection of the presence of the mutation in the sarcoglycan gene or dystrophin gene.

In some embodiments, the methods disclosed herein further comprise, or consist essentially of, or yet further consist of detecting levels of sarcoglycan, sarcospan, or dystrophin protein in the subject prior to administering or subsequent to any of the polynucleotides or compositions disclosed herein to the subject. In some embodiments, the methods disclosed herein further comprise, or consist essentially of, or yet further consist of detecting levels of sarcoglycan, sarcospan, or dystrophin protein in the subject after administering any of the polynucleotides or compositions disclosed herein to the subject. In some embodiments, detecting the levels of sarcoglycan, sarcospan, or dystrophin comprises, or consists essentially of, or yet further consists of detecting expression of the sarcoglycan, sarcospan, or dystrophin gene. Detecting expression of the sarcoglycan, sarcospan, or dystrophin gene may comprise, or consist essentially of, or yet further consist of quantifying sarcoglycan, sarcospan, or dystrophin DNA or RNA levels. Alternatively, or additionally, detecting the levels of sarcoglycan, sarcospan, or dystrophin protein comprises, or consists essentially of, or yet further consists of quantifying the levels of sarcoglycan, sarcospan, or dystrophin protein. In some embodiments, the levels of sarcoglycan, sarcospan, or dystrophin protein are detected in a sample from the subject. In some embodiments, the sample is a body fluid sample. Examples of body fluid samples include, but are not limited to, blood, urine, sweat, saliva, stool, and synovial fluid. In some embodiments, the blood sample is a plasma or serum sample.

In some embodiments, the methods disclosed herein further comprise, or consist essentially of, or yet further consist of modifying the dose or dosing frequency of any of the polynucleotides or compositions that is administered to the subject. In some embodiments, modifying the dose or dosing frequency is based on the detection of sarcoglycan, sarcospan, and/or sarcoglycan, sarcospan, or dystrophin protein levels. In some embodiments, the dose or dosing frequency is reduced when sarcoglycan, sarcospan, and/or sarcoglycan, sarcospan, or dystrophin protein levels in the subject increase as compared to the sarcoglycan, sarcospan, and/or sarcoglycan, sarcospan, or dystrophin protein levels in the subject from an earlier time point (e.g., prior to administering the polynucleotides or compositions, or after administering an initial dose of the polynucleotides or compositions, but prior to administering a subsequent dose of the polynucleotides or compositions).

Promoters, Introns, PolyA Sequence, and Inverted Terminal Repeats

In some embodiments, any of the polynucleotides, viral genomes, or expression cassettes disclosed herein comprise, or consist essentially of, or yet further consist of (a) a first polynucleotide encoding a sarcoglycan, dystrophin, or abbreviated version of dystrophin; and (b) one or more of a promoter, intron, polyA sequence, and inverted terminal repeat.

In some embodiments, the polynucleotide, viral genome, or expression cassette comprises, or consists essentially of, or yet further consists of a promoter. In some embodiments, the promoter is a muscle-specific promoter. In some embodiments, the muscle-specific promoter is selected from an MHCK7 promoter and tMCK promoter. In some embodiments, the promoter comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 4 or 6 across the entire length of SEQ ID NO: 4 or 6.

In some embodiments, the polynucleotide, viral genome, or expression cassette comprises, or consists essentially of, or yet further consists of an intron. In some embodiments, the intron is a SV40 chimeric intron. In some embodiments, the intron comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 9 across the entire length of SEQ ID NO: 9.

In some embodiments, the polynucleotide, viral genome, or expression cassette comprises, or consists essentially of, or yet further consists of a polyA sequence. In some embodiments, the polyA sequence comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 10 across the entire length of SEQ ID NO: 10.

In some embodiments, the polynucleotide, viral genome, or expression cassette comprises, or consists essentially of, or yet further consists of an inverted terminal repeat (ITR). In some embodiments, the ITR comprises, or consists essentially of, or yet further consists of a nucleotide sequence that is at least 70%, 80%, 85%, 90%, 95%, 97%, 98%, 99%, or 100% identical to the nucleotide sequence of SEQ ID NO: 11 or 12 across the entire length of SEQ ID NO: 11 or 12.

Compositions

In a yet further aspect, a composition is provided that comprises, or alternatively consists essentially of, or yet further consisting of, any of one or more of the polynucleotides disclosed herein. In some embodiments, the composition comprises, consists of, or yet further consists essentially of any of the polynucleotides disclosed herein. In some embodiments, the composition comprises, consists of, or yet further consists of 1, 2, 3, 4, or 5 or more polynucleotides encoding 1, 2, 3, 4, or 5 or more proteins selected from sarcoglycan, dystrophin, or sarcospan. In some embodiments, the sarcoglycan is selected from α-sarcoglycan (SGCA), β-sarcoglycan (SGCB), δ-sarcoglycan (SGCD), or γ-sarcoglycan (SGCG). In some embodiments, the composition comprises, consists of, or yet further consists essentially of a polynucleotide encoding SGCB. In some embodiments, the composition comprises, consists of, or yet further consists essentially of 1, 2, 3, 4, or 5 or more nanoparticles, liposomes, or viral vectors comprising, or consisting essentially of, or yet further consisting of 1, 2, 3, 4, or 5 or more polynucleotides encoding 1, 2, 3, 4, or 5 or more proteins selected from sarcoglycan, dystrophin, or sarcospan.

In some aspects, the compositions provide one or more dosage units vg/kg, as described above and incorporated herein by reference.

Kits

In a yet further aspect, a kit is provided that comprises, or alternatively consists essentially of, or yet further consisting of, any of one or more of the polynucleotides or compositions, and instructions for use. In one aspect, any of one or more of the polynucleotides or compositions are detectably labeled or further comprise, or consist essentially of, or yet further consist of a purification or detectable marker.

In some embodiments, the kit comprises, consists of, or yet further consists essentially of any of the polynucleotides or compositions disclosed herein and instructions for use in vitro or in vivo. In some embodiments, the kit comprises, consists of, or yet further consists of 1, 2, 3, 4, or 5 or more polynucleotides encoding 1, 2, 3, 4, or 5 or more proteins selected from sarcoglycan, dystrophin, or sarcospan. In some embodiments, the kit comprises, consists of, or yet further consists of 1, 2, 3, 4, or 5 or more compositions comprising, or consisting essentially of, or yet further consisting of one or more polynucleotides encoding 1, 2, 3, 4, or 5 or more proteins selected from sarcoglycan, dystrophin, or sarcospan. In some embodiments, the sarcoglycan is selected from α-sarcoglycan (SGCA), β-sarcoglycan (SGCB), δ-sarcoglycan (SGCD), or γ-sarcoglycan (SGCG). In some embodiments, the kit comprises, consists of, or yet further consists essentially of a polynucleotide encoding SGCB or a composition comprising, or consisting essentially of, or yet further consisting of a polynucleotide encoding SGCB. In some embodiments, the kit comprises, consists of, or yet further consists essentially of 1, 2, 3, 4, or 5 or more nanoparticles, liposomes, or viral vectors comprising, or consisting essentially of, or yet further consisting of 1, 2, 3, 4, or 5 or more polynucleotides encoding 1, 2, 3, 4, or 5 or more proteins selected from sarcoglycan, dystrophin, or sarcospan. In some aspects, the compositions provide one or more dosage units vg/kg, as described above and incorporated herein by reference.

In some embodiments, the kit further comprises, or consists essentially of, or yet further consists of instructions for detecting the expression level of at least one protein selected from sarcoglycan, dystrophin, or sarcospan. In some embodiments, the sarcoglycan is selected from α-sarcoglycan (SGCA), β-sarcoglycan (SGCB), δ-sarcoglycan (SGCD), or γ-sarcoglycan (SGCG).

### EXAMPLES

### Example 1: SGCB Gene Transfer Restores DAPC

This example investigates whether DAPC function is restored following SGCB gene transfer in SGCB−/− mice and whether other sarcoglycan and sarcospan expression can serve as a surrogate marker for functional restoration of DAPC. This example also shows that there is an expression-functional correlation to define a dose response/expression level threshold for clinical (functional) benefit by characterizing SGCB+/− mice. Specifically, the objectives were to: (a) assess the ability of a SGCB transgene to restore sarcoglycan and sarcospan expression in SGCB−/− mice; and (b) test their utility as a surrogate marker for DAPC restoration

Limb-girdle muscular dystrophy type 2E (LGMD2E) is an autosomal recessive disease caused by mutations in β-sarcoglycan (SGCB) leading to protein deficiency, loss of formation of the sarcoglycan complex, and loss of stabilization of the dystrophin-associated protein complex (DAPC).

Individuals who have a single pathogenic variant are asymptomatic (carriers), and therefore able to compensate for the defective gene copy.

Sarcoglycanopathies present as progressive muscular dystrophies starting in the girdle muscles before extending to lower and upper extremity muscles, and can also present in the diaphragm and heart, resulting in respiratory and cardiac failure in specific patient subtypes.

The sarcoglycans and sarcospan are integral proteins critical for stabilizing the DAPC and providing mechanical support to the sarcolemma.

Adeno-associated virus (AAV)-mediated gene replacement therapy has shown early signs of potential to treat sarcoglycanopathies. Key considerations include a systematic and stepwise evaluation of safety, transduction, expression, localization, cellular impact, and clinical function.

With these considerations in mind, the self-complementary (sc) AAV.MHCK7.hSGCB construct was designed to restore functional β-sarcoglycan to muscles. The scAAV.MHCK7.hSGCB construct comprises (a) an AAVrh74 vector, which displays robust muscle (skeletal and cardiac) tissue tropism and has relatively low level of pre-existing immunity; (b) an MHCK7 promoter, which regulates and drives transgene expression selectively in skeletal and cardiac muscle; includes an alpha myosin heavy chain enhancer to drive especially strong expression in cardiac muscle; and (c) a hSGCB transgene, which carries full-length β-sarcoglycan cDNA.

SGCB−/− mice have been shown to concurrently display loss of additional sarcoglycans (α, γ, and δ). Evidence suggests sarcospan may also be lost in the absence of SGCB.

Methods

Transcriptional and translational regulation of SGCB along with functional outputs were assessed in normal wild-type (WT) mice, heterozygous SGCB+/− mice, and homozygous knockout (KO) SGCB−/− mice.

Transcript levels of SGCB in skeletal muscle of mice were measured using quantitative reverse transcription PCR (qRT-PCR).

Sarcoglycan and sarcospan protein expression in skeletal and cardiac muscle from untreated and vector-dosed SGCB−/− mice was evaluated by immunofluorescence staining and western blot.

Histological evaluations included hematoxylin and eosin staining of skeletal muscle (tibialis anterior [TA] and gastrocnemius [GAS]) and quantification of central nucleation.

Functional assessments included measurement of force production and resistance to contraction-induced injury in the TA muscle along with laser monitoring of open-field cage activity to assess overall ambulation (movement around the cage) and vertical activity (rearing onto hind limbs).

Animal models: C57BL6 wild-type (WT), SGCB+/− (SGCB het), and SGCB−/− (SGCB KO) mice were maintained under standardized conditions on a 12:12-hour light:dark cycle, with food and water provided ad libitum.

Results

Expression-Function Correlation:

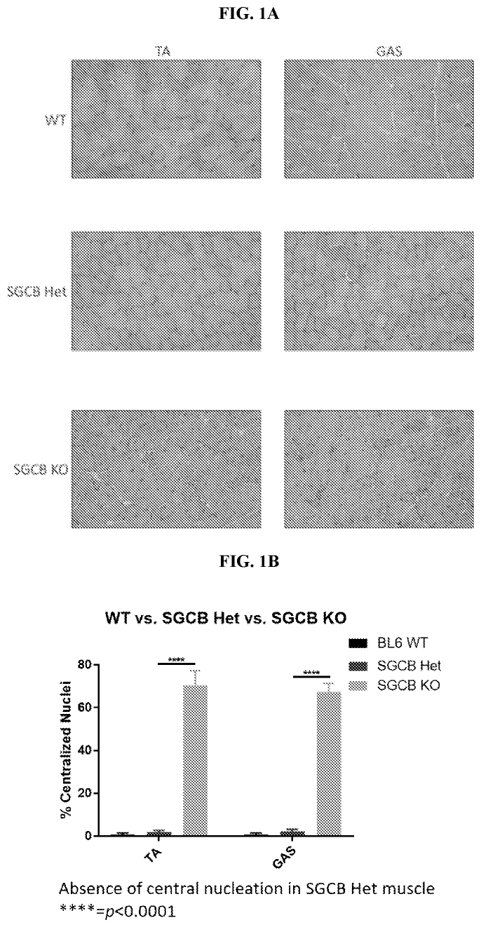

SGCB+/− mice were not found to have any significant dystrophic phenotype as shown by histopathology (FIG. 1A) and quantification of central nucleation in the TA and GAS (FIG. 1B) as compared to wild type mice. As shown in FIG. 1B, levels of central nucleation for SGCB het mice are similar to WT and dramatically different to SGCB KO mice.

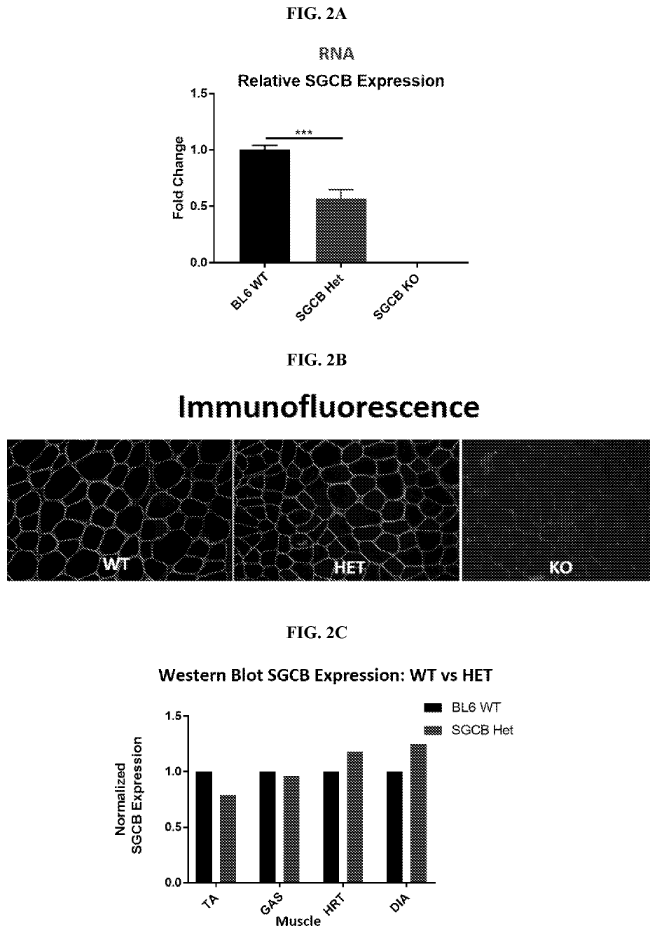

Sarcoglycan expression in SGCB+/−, SGCB−/−, and WT mice was determined at the transcript level and protein level. Transcript mRNA levels were measured by qRT-PCR (FIG. 2A), and protein production was measured by western blot (FIG. 2C) (immunofluorescence images also shown in FIG. 2B). SGCB expression is significantly reduced for the SGCB−/− mice as expected. SGCB mRNA levels are reduced for the SGCB+/− mice compared to WT mice, but no detectable differences are observed in protein production.

Analysis of Functional Outputs in SGCB Het Mice

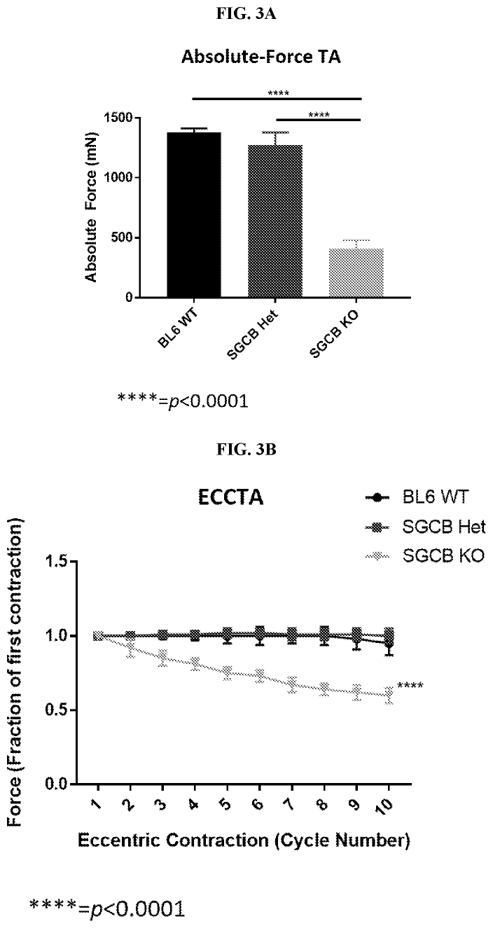

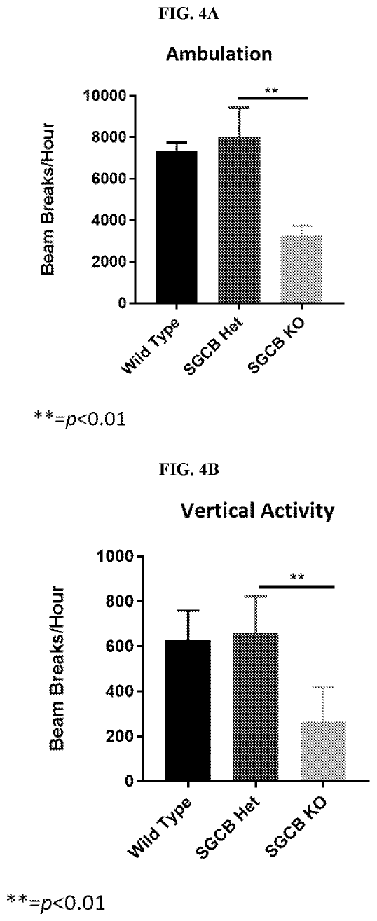

Absolute force and resistance to eccentric contraction were similar to WT mice in SGCB+/− TA muscle and significantly different compared to SGCB−/− mice (FIGS. 3A-3B). Analysis of open-field cage activity shows that both ambulation and vertical activity is not affected in the SGCB+/− mice compared to WT mice (FIGS. 4A-4B).

DAPC Restoration

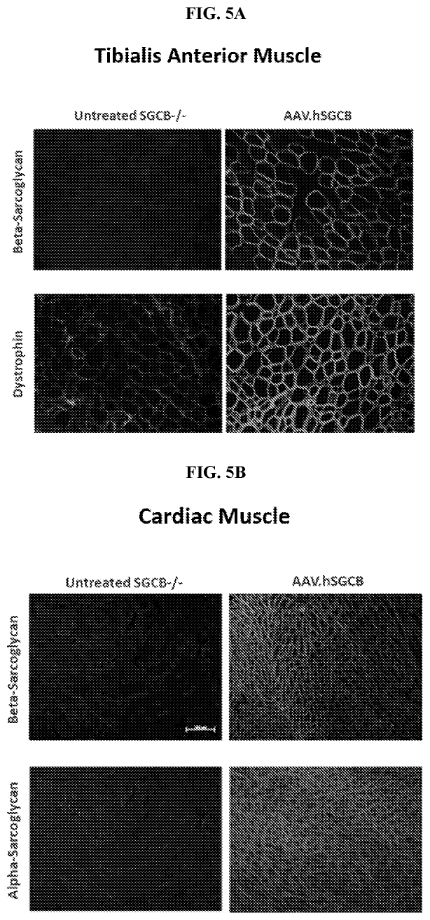

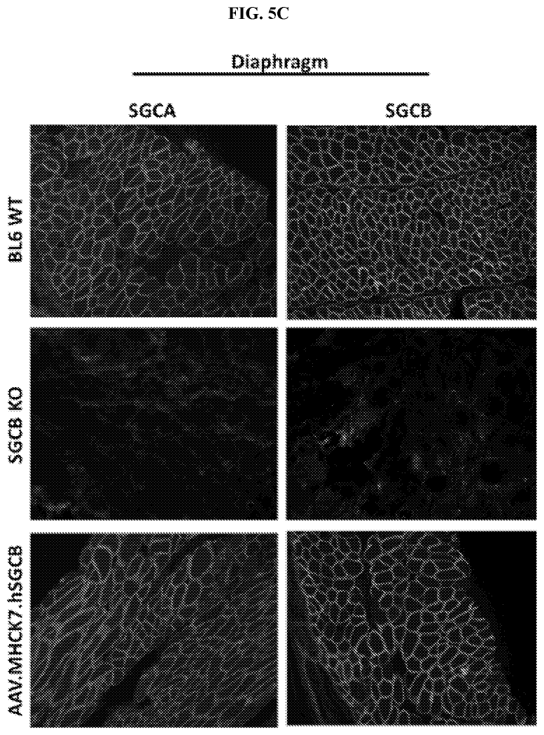

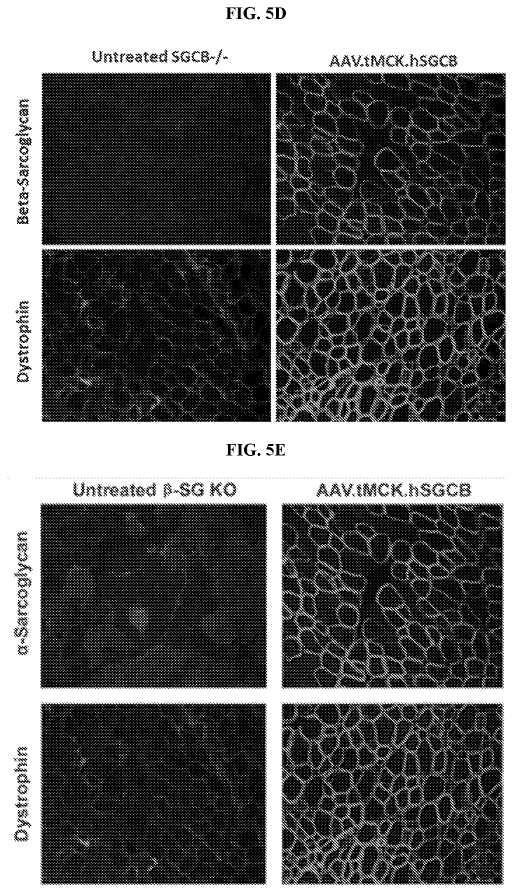

Dystrophin and SGCA expression were restored following aav.hSGCB gene transfer in SGCB−/− mice in both TA and cardiac muscle (FIGS. 5A-5C). As shown in FIG. 5A, loss of SGCB leads to reduction of dystrophin in the TA muscle. Restoring SGCB protein levels in the TA results in restoring dystrophin in the TA muscle. As shown in FIG. 5B, loss of SGCB leads to reduction of SGCA in cardiac muscle. Restoring SGCB protein levels in cardiac muscle results in restoring SGCA in cardiac muscle. As shown in FIG. 5C, loss of SGCB leads to reduction of SGCA in the diaphragm. Restoring SGCB protein levels in the diaphragm also resulted in restoring SGCA in the diaphragm. As shown in FIGS. 5D-5E, the restoration of SGCB protein levels, and subsequent restoration of dystrophin and SGCA, is not limited to the specific promoter as similar results were observed using an scAAV genome comprising a tMCK promoter and a human SGCB polynucleotide sequence.

As shown in FIG. 5D, loss of SGCB leads to reduction of dystrophin. Restoring SGCB protein levels at the sarcolemma results in restoring dystrophin at the sarcolemma. As shown in FIG. 5E, loss of SGCB leads to loss of SGCA and dystrophin, which are restored following AAV.hSGCB gene transfer.

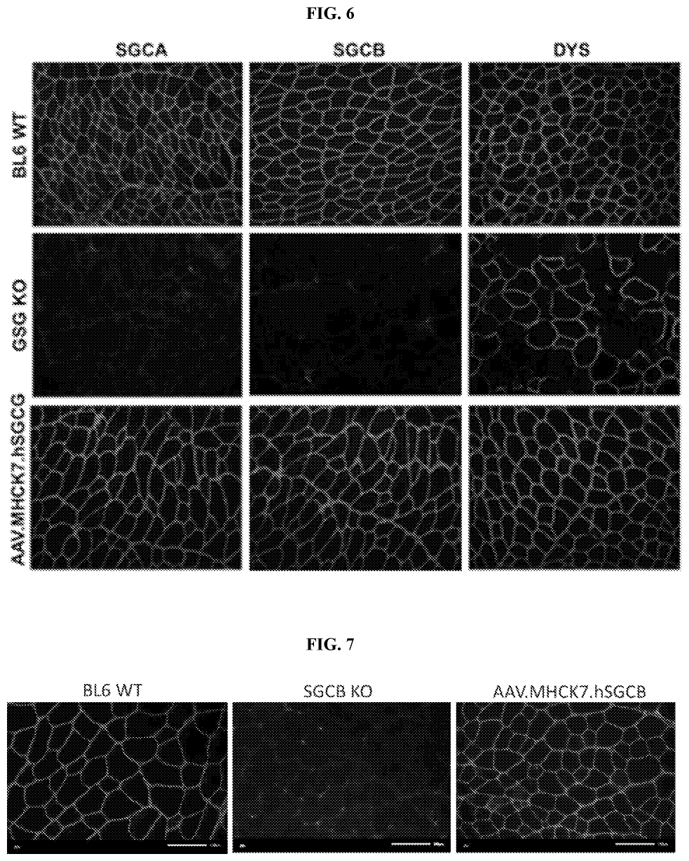

Alpha-sarcoglycan, β-sarcoglycan, and dystrophin expression were restored after aav.hSGCG gene transfer in the TA muscle of SGCG−/− mice (FIG. 6). As shown in FIG. 6, loss of SGCG leads to a reduction in SGCA and dystrophin (DYS) in the TA muscle (middle row). Restoring SGCG protein levels in the TA muscle results in restoring SGCA and DYS in the TA muscle (bottom row).

Sarcospan as Surrogate Biomarker for Restoration of DAPC

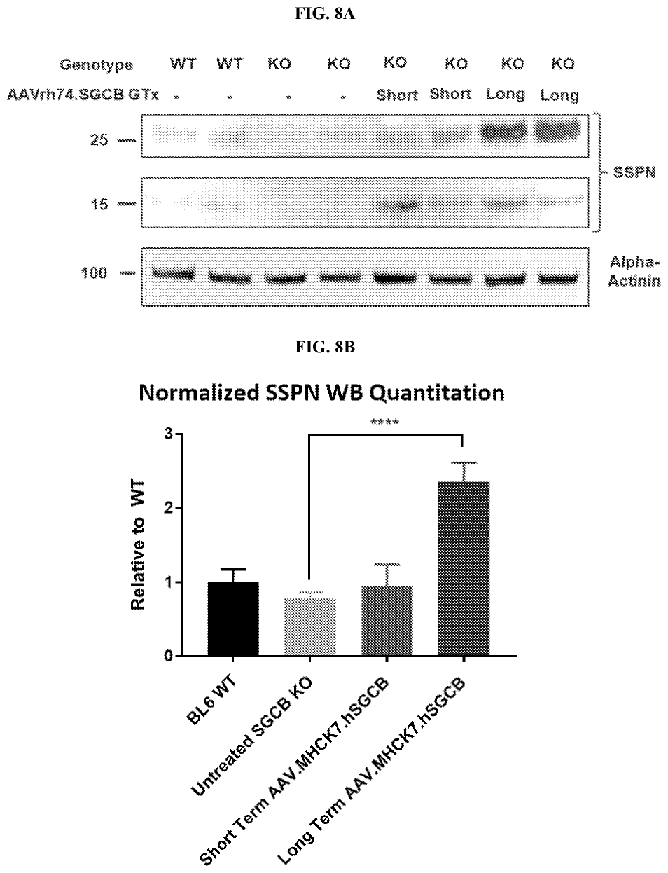

Sarcospan was reduced or absent in the TA muscle of SGCB−/− mice and restored in SGCB−/− mice following aav.hSGCB gene transfer as measured by immunofluorescence (FIG. 7) and western blot (FIGS. 8A-8B).

Conclusions

Overall SGCB+/− mice present with normal muscle phenotype similar to WT mice and do not develop any dystrophic histopathology.

RNA transcript levels of SGCB in het mice were found to be about half the level of WT mice as expected, however protein levels were normal and similar to WT mice.

Co-localization studies confirmed restoration of the DAPC following SGCB or SGCG gene therapy. This data demonstrates that additional sarcoglycans and sarcospan can serve as a surrogate marker for functional restoration of the DAPC.

### Example 2: SGCB Gene Transfer Restores DAPC

This example shows that DAPC function was restored following SGCB gene transfer in SGCB−/− mice at two dosages and that other sarcoglycan and sarcospan expression can serve as a surrogate marker for functional restoration of DAPC.

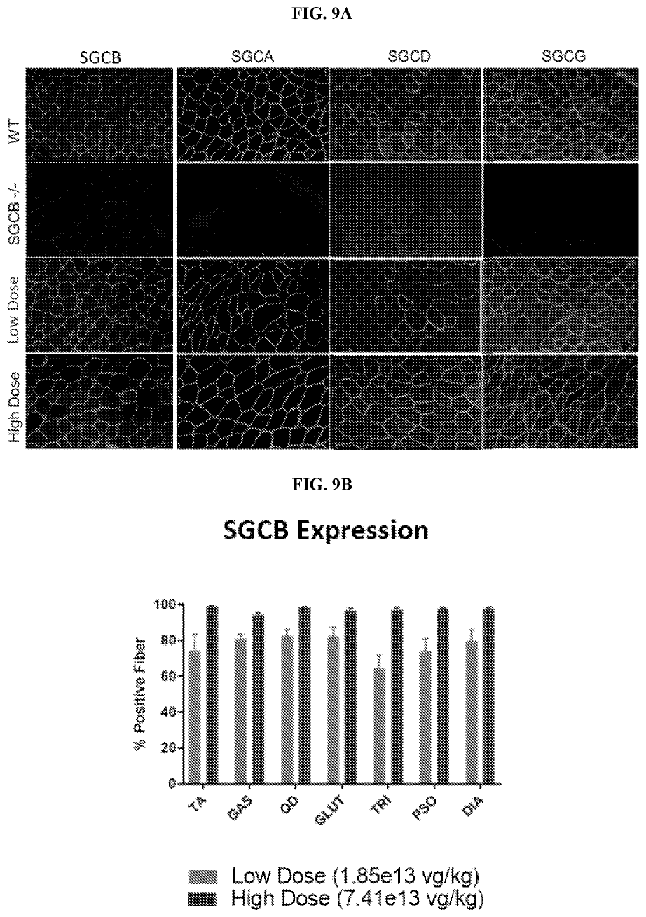

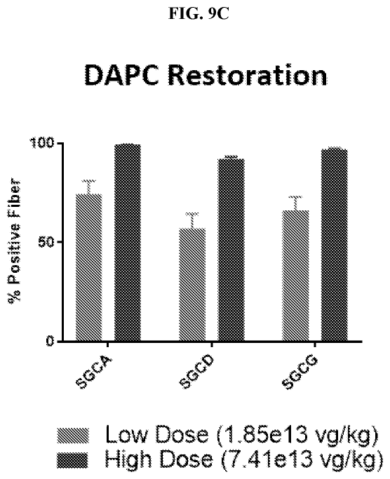

FIGS. 9A-9C show restoration of DAPC proteins in SGCB−/− mice after administration of scAAV.MHCK7.hSGCB at 1.85e13 vg/kg and 7.41e13 vg/kg based on a linearized PCR standard.

Muscle cells from SGCB−/− mice show absent or reduced sarcolemma expression of α-sarcoglycan (SGCA), β-sarcoglycan (SGCB), 7-sarcoglycan (SGCG) and δ-sarcoglycan (SGCD), components of the dystrophin-associated protein complex (DAPC) (FIG. 9A). Systemic, IV administration of scAAV.MHCK7.hSGCB at 1.85e13 vg/kg and 7.41e13 vg/kg (quantified by linearized PCR standard) to SGCB−/− mice not only increased SGCB expression but also SGCA, SGCG, and SGCD subunit expressions at the sarcolemma in SGCB−/− mice, demonstrating a dose-dependent restoration of DAPC proteins with scAAV.MHCK7.hSGCB (FIGS. 9B-9C). For FIG. 9B, TA refers to tibialis anterior; GAS refers to gastrocnemius; QD refers to quadricep; TRI refers to tricep; GLUT refers to gluteus; PSO refers to psoas major; and DIA refers to diaphragm.

Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

List of Sequences

| SEQ | | |
|---|---|---|
| ID | | |
| NO | Description | Sequence |
| 1 | SGCB nucleotide | atggcagcagcagccgccgcagccgccgagcagcagtcaagcaat |
| | sequence (homo | ggaccagtgaaaaaatcaatgagagaaaaagccgtcgagaggaga |
| | sapiens | tcagtgaataaggagcacaacagcaatttcaaagccggctacatc |
| | | cctattgacgaagatcgcctgcataagacaggcctgagggggcgc |
| | | aaaggaaacctggcaatctgcgtcatcattctgctgtttatcctg |
| | | gccgtgattaatctgatcattactctggtgatttgggctgtcatc |
| | | cgcattggcccaaacgggtgtgactctatggagttccacgaaagt |
| | | ggcctgctgcgatttaagcaggtgtccgatatgggggtcatccat |
| | | ccactgtacaaatctactgtcggcgggcggagaaacgagaatctg |
| | | gtgatcaccgggaacaatcagcccattgtgttccagcagggaacc |
| | | acaaagctgtctgtggaaaacaataaaacatcaatcactagcgac |
| | | attggcatgcagttctttgatccccggacccagaatatcctgttc |
| | | agtaccgactatgagacacacgaatttcatctgccttccggggtg |
| | | aagtctctgaacgtccagaaagccagcactgagagaatcaccagt |
| | | aacgctacatcagacctgaatatcaaggtggatggacgagctatt |
| | | gtccggggaaatgagggcgtgttcatcatgggcaagacaattgaa |
| | | tttcacatgggaggcaacatggagctgaaagcagaaaacagcatc |
| | | attctgaatgggagcgtgatggtctccactaccagactgcccagc |
| | | tcctctagtggagaccagctggggtccggagattgggtcaggtat |
| | | aagctgtgcatgtgtgccgatggcaccctgtttaaagtgcaggtc |
| | | accagccagaatatgggatgtcagattagcgataacccttgtggg |
| | | aatactcattaa |
| 2 | SGCB amino acid | MetAlaAlaAlaAlaAlaAlaAlaAlaGluGlnGlnSerSerAsn |
| | sequence (homo | GlyProValLysLysSerMetArgGluLysAlaValGluArgArg |
| | sapiens) | SerValAsnLysGluHisAsnSerAsnPheLysAlaGlyTyrIle |
| | | ProIleAspGluAspArgLeuHisLysThrGlyLeuArgGlyArg |
| | | LysGlyAsnLeuAlaIleCysValIleIleLeuLeuPheIleLeu |
| | | AlaValIleAsnLeuIleIleThrLeuValIleTrpAlaValIle |
| | | ArgIleGlyProAsnGlyCysAspSerMetGluPheHisGluSer |
| | | GlyLeuLeuArgPheLysGlnValSerAspMetGlyValIleHis |
| | | ProLeuTyrLysSerThrValGlyGlyArgArgAsnGluAsnLeu |
| | | ValIleThrGlyAsnAsnGlnProIleValPheGlnGlnGlyThr |
| | | ThrLysLeuSerValGluAsnAsnLysThrSerIleThrSerAsp |
| | | IleGlyMetGlnPhePheAspProArgThrGlnAsnIleLeuPhe |
| | | SerThrAspTyrGluThrHisGluPheHisLeuProSerGlyVal |
| | | LysSerLeuAsnValGlnLysAlaSerThrGluArgIleThrSer |
| | | AsnAlaThrSerAspLeuAsnIleLysValAspGlyArgAlaIle |
| | | ValArgGlyAsnGluGlyValPheIleMetGlyLysThrIleGlu |
| | | PheHisMetGlyGlyAsnMetGluLeuLysAlaGluAsnSerIle |
| | | IleLeuAsnGlySerValMetValSerThrThrArgLeuProSer |
| | | SerSerSerGlyAspGlnLeuGlySerGlyAspTrpValArgTyr |
| | | LysLeuCysMetCysAlaAspGlyThrLeuPheLysValGlnVal |
| | | ThrSerGlnAsnMetGlyCysGlnIleSerAspAsnProCysGly |
| | | AsnThrHis |
| 3 | pAAV.MHCK7.h | ctgcgcgctcgctcgctcactgaggccgcccgggcaaagcccggg |
| | SCGB (artificial | cgtcgggcgacctttggtcgcccggcctcagtgagcgagcgagcg |
| | sequence) | cgcagagagggagtggggttaaccaattggcgcggccgcaagctt |
| | | gcatgtctaagctagacccttcagattaaaaataactgaggtaag |
| | | ggcctgggtaggggaggtggtgtgagacgctcctgtctctcctct |
| | | atctgcccatcggccctttggggaggaggaatgtgcccaaggact |
| | | aaaaaaaggccatggagccagaggggcgagggcaacagacctttc |
| | | atgggcaaaccttggggccctgctgtctagcatgccccactacgg |
| | | gtctaggctgcccatgtaaggaggcaaggcctggggacacccgag |
| | | atgcctggttataattaacccagacatgtggctgccccccccccc |
| | | ccaacacctgctgcctctaaaaataaccctgtccctggtggatcc |
| | | cctgcatgcgaagatcttcgaacaaggctgtgggggactgagggc |
| | | aggctgtaacaggcttgggggccagggcttatacgtgcctgggac |
| | | tcccaaagtattactgttccatgttcccggcgaagggccagctgt |
| | | cccccgccagctagactcagcacttagtttaggaaccagtgagca |
| | | agtcagcccttggggcagcccatacaaggccatggggctgggcaa |
| | | gctgcacgcctgggtccggggtgggcacggtgcccgggcaacgag |
| | | ctgaaagctcatctgctctcaggggcccctccctggggacagccc |
| | | ctcctggctagtcacaccctgtaggctcctctatataacccaggg |
| | | gcacaggggctgccctcattctaccaccacctccacagcacagac |
| | | agacactcaggagcagccagcggcgcgcccaggtaagtttagtct |
| | | ttttgtcttttatttcaggtcccggatccggtggtggtgcaaatc |
| | | aaagaactgctcctcagtggatgttgcctttacttctaggcctgt |
| | | acggaagtgttacttctgctctaaaagctgcggaattgtacccgg |
| | | taccgccaccatggcagcagcagccgccgcagccgccgagcagca |
| | | gtcaagcaatggaccagtgaaaaaatcaatgagagaaaaagccgt |
| | | cgagaggagatcagtgaataaggagcacaacagcaatttcaaagc |
| | | cggctacatccctattgacgaagatcgcctgcataagacaggcct |
| | | gagggggcgcaaaggaaacctggcaatctgcgtcatcattctgct |
| | | gtttatcctggccgtgattaatctgatcattactctggtgatttg |
| | | ggctgtcatccgcattggcccaaacgggtgtgactctatggagtt |
| | | ccacgaaagtggcctgctgcgatttaagcaggtgtccgatatggg |
| | | ggtcatccatccactgtacaaatctactgtcggcgggcggagaaa |
| | | cgagaatctggtgatcaccgggaacaatcagcccattgtgttcca |
| | | gcagggaaccacaaagctgtctgtggaaaacaataaaacatcaat |
| | | cactagcgacattggcatgcagttctttgatccccggacccagaa |
| | | tatcctgttcagtaccgactatgagacacacgaatttcatctgcc |
| | | ttccggggtgaagtctctgaacgtccagaaagccagcactgagag |
| | | aatcaccagtaacgctacatcagacctgaatatcaaggtggatgg |
| | | acgagctattgtccggggaaatgagggcgtgttcatcatgggcaa |
| | | gacaattgaatttcacatgggaggcaacatggagctgaaagcaga |
| | | aaacagcatcattctgaatgggagcgtgatggtctccactaccag |
| | | actgcccagctcctctagtggagaccagctggggtccggagattg |
| | | ggtcaggtataagctgtgcatgtgtgccgatggcaccctgtttaa |
| | | agtgcaggtcaccagccagaatatgggatgtcagattagcgataa |
| | | cccttgtgggaatactcattaaaagcttggccgcaataaaagatc |
| | | tttattttcattagatctgtgtgttggttttttgtgtgtcctgca |
| | | ggggcgcgcctctagagcatggctacgtagataagtagcatggcg |
| | | ggttaatcattaactacaaggaacccctagtgatggagttggcca |
| | | ctccctctctgcgcgctcgctcgctcactgaggccgggcgaccaa |
| | | aggtcgcccgacgcccgggctttgcccgggcggcctcagtgagcg |
| | | agcgagcgcgc |
| 4 | MHCK7 promoter | aagcttgcatgtctaagctagacccttcagattaaaaataactga |
| | (artificial | ggtaagggcctgggtaggggaggtggtgtgagacgctcctgtctc |
| | sequence) | tcctctatctgcccatcggccctttggggaggaggaatgtgccca |
| | | aggactaaaaaaaggccatggagccagaggggcgagggcaacaga |
| | | cctttcatgggcaaaccttggggccctgctgtctagcatgcccca |
| | | ctacgggtctaggctgcccatgtaaggaggcaaggcctggggaca |
| | | cccgagatgcctggttataattaacccagacatgtggctgccccc |
| | | ccccccccaacacctgctgcctctaaaaataaccctgtccctggt |
| | | ggatcccctgcatgcgaagatcttcgaacaaggctgtgggggact |
| | | gagggcaggctgtaacaggcttgggggccagggcttatacgtgcc |
| | | tgggactcccaaagtattactgttccatgttcccggcgaagggcc |
| | | agctgtcccccgccagctagactcagcacttagtttaggaaccag |
| | | tgagcaagtcagcccttggggcagcccatacaaggccatggggct |
| | | gggcaagctgcacgcctgggtccggggtgggcacggtgcccgggc |
| | | aacgagctgaaagctcatctgctctcaggggcccctccctgggga |
| | | cagcccctcctggctagtcacaccctgtaggctcctctatataac |
| | | ccaggggcacaggggctgccctcattctaccaccacctccacagc |
| | | acagacagacactcaggagcagccagc |
| 5 | pAAV.tMCK.hS | ctgcgcgctcgctcgctcactgaggccgcccgggcaaagcccggg |
| | GCB (artificial | cgtcgggcgacctttggtcgcccggcctcagtgagcgagcgagcg |
| | sequence) | cgcagagagggagtggggttaaccaattggcggccgcaaacttgc |
| | | atgccccactacgggtctaggctgcccatgtaaggaggcaaggcc |
| | | tggggacacccgagatgcctggttataattaaccccaacacctgc |
| | | tgcccccccccccccaacacctgctgcctgagcctgagcggttac |
| | | cccaccccggtgcctgggtcttaggctctgtacaccatggaggag |
| | | aagctcgctctaaaaataaccctgtccctggtggatccactacgg |
| | | gtctatgctgcccatgtaaggaggcaaggcctggggacacccgag |
| | | atgcctggttataattaaccccaacacctgctgcccccccccccc |
| | | caacacctgctgcctgagcctgagcggttaccccaccccggtgcc |
| | | tgggtcttaggctctgtacaccatggaggagaagctcgctctaaa |
| | | aataaccctgtccctggtggaccactacgggtctaggctgcccat |
| | | gtaaggaggcaaggcctggggacacccgagatgcctggttataat |
| | | taaccccaacacctgctgccccccccccccaacacctgctgcctg |
| | | agcctgagcggttaccccaccccggtgcctgggtcttaggctctg |
| | | tacaccatggaggagaagctcgctctaaaaataaccctgtccctg |
| | | gtcctccctggggacagcccctcctggctagtcacaccctgtagg |
| | | ctcctctatataacccaggggcacaggggctgcccccgggtcacc |
| | | tgcagaagttggtcgtgaggcactgggcaggtaagtatcaaggtt |
| | | acaagacaggtttaaggagaccaatagaaactgggcttgtcgaga |
| | | cagagaagactcttgcgtttctgataggcacctattggtcttact |
| | | gacatccactttgcctttctctccacaggtgtccactcccagttc |
| | | aattacagcgcgtggtaccaccatggcagcagcagccgccgcagc |
| | | cgccgagcagcagtcaagcaatggaccagtgaaaaaatcaatgag |
| | | agaaaaagccgtcgagaggagatcagtgaataaggagcacaacag |
| | | caatttcaaagccggctacatccctattgacgaagatcgcctgca |
| | | taagacaggcctgagggggcgcaaaggaaacctggcaatctgcgt |
| | | catcattctgctgtttatcctggccgtgattaatctgatcattac |
| | | tctggtgatttgggctgtcatccgcattggcccaaacgggtgtga |
| | | ctctatggagttccacgaaagtggcctgctgcgatttaagcaggt |
| | | gtccgatatgggggtcatccatccactgtacaaatctactgtcgg |
| | | cgggcggagaaacgagaatctggtgatcaccgggaacaatcagcc |
| | | cattgtgttccagcagggaaccacaaagctgtctgtggaaaacaa |
| | | taaaacatcaatcactagcgacattggcatgcagttctttgatcc |
| | | ccggacccagaatatcctgttcagtaccgactatgagacacacga |
| | | atttcatctgccttccggggtgaagtctctgaacgtccagaaagc |
| | | cagcactgagagaatcaccagtaacgctacatcagacctgaatat |
| | | caaggtggatggacgagctattgtccggggaaatgagggcgtgtt |
| | | catcatgggcaagacaattgaatttcacatgggaggcaacatgga |
| | | gctgaaagcagaaaacagcatcattctgaatgggagcgtgatggt |
| | | ctccactaccagactgcccagctcctctagtggagaccagctggg |
| | | gtccggagattgggtcaggtataagctgtgcatgtgtgccgatgg |
| | | caccctgtttaaagtgcaggtcaccagccagaatatgggatgtca |
| | | gattagcgataacccttgtgggaatactcattaaaagcttggccg |
| | | caataaaagatctttattttcattagatctgtgtgttggtttttt |
| | | gtgtgtcctgcaggggcgcgcctctagagcatggctacgtagata |
| | | agtagcatggcgggttaatcattaactacaaggaacccctagtga |
| | | tggagttggccactccctctctgcgcgctcgctcgctcactgagg |
| | | ccgggcgaccaaaggtcgcccgacgcccgggctttgccc |
| 6 | tMCK promoter | ccactacgggtctaggctgcccatgtaaggaggcaaggcctgggg |
| | (artificial | acacccgagatgcctggttataattaaccccaacacctgctgccc |
| | sequence) | ccccccccccaacacctgctgcctgagcctgagcggttaccccac |
| | | cccggtgcctgggtcttaggctctgtacaccatggaggagaagct |
| | | cgctctaaaaataaccctgtccctggtggatccactacgggtcta |
| | | tgctgcccatgtaaggaggcaaggcctggggacacccgagatgcc |
| | | tggttataattaaccccaacacctgctgcccccccccccccaaca |
| | | cctgctgcctgagcctgagcggttaccccaccccggtgcctgggt |
| | | cttaggctctgtacaccatggaggagaagctcgctctaaaaataa |
| | | ccctgtccctggtggaccactacgggtctaggctgcccatgtaag |
| | | gaggcaaggcctggggacacccgagatgcctggttataattaacc |
| | | ccaacacctgctgccccccccccccaacacctgctgcctgagcct |
| | | gagcggttaccccaccccggtgcctgggtcttaggctctgtacac |
| | | catggaggagaagctcgctctaaaaataaccctgtccctggtcct |
| | | ccctggggacagcccctcctggctagtcacaccctgtaggctcct |
| | | ctatataacccaggggcacaggggctgcccccgggtcac |
| 7 | scAAVrh74.MHC | ctgcgcgctcgctcgctcactgaggccgcccgggcaaagcccggg |
| | K7.hSGCB | cgtcgggcgacctttggtcgcccggcctcagtgagcgagcgagcg |
| | (artificial | cgcagagagggagtggggttaaccaattggcggccgcaagcttgc |
| | sequence) | atgtctaagctagacccttcagattaaaaataactgaggtaaggg |
| | | cctgggtaggggaggtggtgtgagacgctcctgtctctcctctat |
| | | ctgcccatcggccctttggggaggaggaatgtgcccaaggactaa |
| | | aaaaaggccatggagccagaggggcgagggcaacagacctttcat |
| | | gggcaaaccttggggccctgctgtctagcatgccccactacgggt |
| | | ctaggctgcccatgtaaggaggcaaggcctggggacacccgagat |
| | | gcctggttataattaacccagacatgtggctgccccccccccccc |
| | | aacacctgctgcctctaaaaataaccctgtccctggtggatcccc |
| | | tgcatgcgaagatcttcgaacaaggctgtgggggactgagggcag |
| | | gctgtaacaggcttgggggccagggcttatacgtgcctgggactc |
| | | ccaaagtattactgttccatgttcccggcgaagggccagctgtcc |
| | | cccgccagctagactcagcacttagtttaggaaccagtgagcaag |
| | | tcagcccttggggcagcccatacaaggccatggggctgggcaagc |
| | | tgcacgcctgggtccggggtgggcacggtgcccgggcaacgagct |
| | | gaaagctcatctgctctcaggggcccctccctggggacagcccct |
| | | cctggctagtcacaccctgtaggctcctctatataacccaggggc |
| | | acaggggctgccctcattctaccaccacctccacagcacagacag |
| | | acactcaggagcagccagcggcgcgcccaggtaagtttagtcttt |
| | | ttgtcttttatttcaggtcccggatccggtggtggtgcaaatcaa |
| | | agaactgctcctcagtggatgttgcctttacttctaggcctgtac |
| | | ggaagtgttacttctgctctaaaagctgcggaattgtacccggta |
| | | ccaccatggcagcagcagccgccgcagccgccgagcagcagtcaa |
| | | gcaatggaccagtgaaaaaatcaatgagagaaaaagccgtcgaga |
| | | ggagatcagtgaataaggagcacaacagcaatttcaaagccggct |
| | | acatccctattgacgaagatcgcctgcataagacaggcctgaggg |
| | | ggcgcaaaggaaacctggcaatctgcgtcatcattctgctgttta |
| | | tcctggccgtgattaatctgatcattactctggtgatttgggctg |
| | | tcatccgcattggcccaaacgggtgtgactctatggagttccacg |
| | | aaagtggcctgctgcgatttaagcaggtgtccgatatgggggtca |
| | | tccatccactgtacaaatctactgtcggcgggcggagaaacgaga |
| | | atctggtgatcaccgggaacaatcagcccattgtgttccagcagg |
| | | gaaccacaaagctgtctgtggaaaacaataaaacatcaatcacta |
| | | gcgacattggcatgcagttctttgatccccggacccagaatatcc |
| | | tgttcagtaccgactatgagacacacgaatttcatctgccttccg |
| | | gggtgaagtctctgaacgtccagaaagccagcactgagagaatca |
| | | ccagtaacgctacatcagacctgaatatcaaggtggatggacgag |
| | | ctattgtccggggaaatgagggcgtgttcatcatgggcaagacaa |
| | | ttgaatttcacatgggaggcaacatggagctgaaagcagaaaaca |
| | | gcatcattctgaatgggagcgtgatggtctccactaccagactgc |
| | | ccagctcctctagtggagaccagctggggtccggagattgggtca |
| | | ggtataagctgtgcatgtgtgccgatggcaccctgtttaaagtgc |
| | | aggtcaccagccagaatatgggatgtcagattagcgataaccctt |
| | | gtgggaatactcattaaaagcttggccgcaataaaagatctttat |
| | | tttcattagatctgtgtgttggttttttgtgtgtcctgcaggggc |
| | | gcgcctctagagcatggctacgtagataagtagcatggcgggtta |
| | | atcattaactacaaggaacccctagtgatggagttggccactccc |
| | | tctctgcgcgctcgctcgctcactgaggccgggcgaccaaaggtc |
| | | gcccgacgcccgggctttgcccgggcggcctcagtgagcgagcga |
| | | gcgcgcag |
| 8 | ScAAVrh74.MHC | gcagctgcgcgctcgctcgctcactgaggccgcccgggcaaagcc |
| | K7.hSGCB | cgggcgtcgggcgacctttggtcgcccggcctcagtgagcgagcg |
| | (artificial | agcgcgcagagagggagtggggttaaccaattggcggccgcaagc |
| | sequence) | ttgcatgtctaagctagacccttcagattaaaaataactgaggta |
| | | agggcctgggtaggggaggtggtgtgagacgctcctgtctctcct |
| | | ctatctgcccatcggccctttggggaggaggaatgtgcccaagga |
| | | ctaaaaaaaggccatggagccagaggggcgagggcaacagacctt |
| | | tcatgggcaaaccttggggccctgctgtctagcatgccccactac |
| | | gggtctaggctgcccatgtaaggaggcaaggcctggggacacccg |
| | | agatgcctggttataattaacccagacatgtggctgccccccccc |
| | | ccccaacacctgctgcctctaaaaataaccctgtccctggtggat |
| | | cccctgcatgcgaagatcttcgaacaaggctgtgggggactgagg |
| | | gcaggctgtaacaggcttgggggccagggcttatacgtgcctggg |
| | | actcccaaagtattactgttccatgttcccggcgaagggccagct |
| | | gtcccccgccagctagactcagcacttagtttaggaaccagtgag |
| | | caagtcagcccttggggcagcccatacaaggccatggggctgggc |
| | | aagctgcacgcctgggtccggggtgggcacggtgcccgggcaacg |
| | | agctgaaagctcatctgctctcaggggcccctccctggggacagc |
| | | ccctcctggctagtcacaccctgtaggctcctctatataacccag |
| | | gggcacaggggctgccctcattctaccaccacctccacagcacag |
| | | acagacactcaggagcagccagcggcgcgcccaggtaagtttagt |
| | | ctttttgtcttttatttcaggtcccggatccggtggtggtgcaaa |
| | | tcaaagaactgctcctcagtggatgttgcctttacttctaggcct |
| | | gtacggaagtgttacttctgctctaaaagctgcggaattgtaccc |
| | | ggtaccaccatggcagcagcagccgccgcagccgccgagcagcag |
| | | tcaagcaatggaccagtgaaaaaatcaatgagagaaaaagccgtc |
| | | gagaggagatcagtgaataaggagcacaacagcaatttcaaagcc |
| | | ggctacatccctattgacgaagatcgcctgcataagacaggcctg |
| | | agggggcgcaaaggaaacctggcaatctgcgtcatcattctgctg |
| | | tttatcctggccgtgattaatctgatcattactctggtgatttgg |
| | | gctgtcatccgcattggcccaaacgggtgtgactctatggagttc |
| | | cacgaaagtggcctgctgcgatttaagcaggtgtccgatatgggg |
| | | gtcatccatccactgtacaaatctactgtcggcgggcggagaaac |
| | | gagaatctggtgatcaccgggaacaatcagcccattgtgttccag |
| | | cagggaaccacaaagctgtctgtggaaaacaataaaacatcaatc |
| | | actagcgacattggcatgcagttctttgatccccggacccagaat |
| | | atcctgttcagtaccgactatgagacacacgaatttcatctgcct |
| | | tccggggtgaagtctctgaacgtccagaaagccagcactgagaga |
| | | atcaccagtaacgctacatcagacctgaatatcaaggtggatgga |
| | | cgagctattgtccggggaaatgagggcgtgttcatcatgggcaag |
| | | acaattgaatttcacatgggaggcaacatggagctgaaagcagaa |
| | | aacagcatcattctgaatgggagcgtgatggtctccactaccaga |
| | | ctgcccagctcctctagtggagaccagctggggtccggagattgg |
| | | gtcaggtataagctgtgcatgtgtgccgatggcaccctgtttaaa |
| | | gtgcaggtcaccagccagaatatgggatgtcagattagcgataac |
| | | ccttgtgggaatactcattaaaagcttggccgcaataaaagatct |
| | | ttattttcattagatctgtgtgttggttttttgtgtgtcctgcag |
| | | gggcgcgcctctagagcatggctacgtagataagtagcatggcgg |
| | | gttaatcattaactacaaggaacccctagtgatggagttggccac |
| | | tccctctctgcgcgctcgctcgctcactgaggccgggcgaccaaa |
| | | ggtcgcccgacgcccgggctttgcccgggcggcctcagtgagcga |
| | | gcgagcgcgcagctggcgtaatagcgaagaggcccgcaccgatcg |
| | | cccttcccaacagttgcgcagcctgaatggcgaatggcgattccg |
| | | ttgcaatggctggcggtaatattgttctggatattaccagcaagg |
| | | ccgatagtttgagttcttctactcaggcaagtgatgttattacta |
| | | atcaaagaagtattgcgacaacggttaatttgcgtgatggacaga |
| | | ctcttttactcggtggcctcactgattataaaaacacttctcagg |
| | | attctggcgtaccgttcctgtctaaaatccctttaatcggcctcc |
| | | tgtttagctcccgctctgattctaacgaggaaagcacgttatacg |
| | | tgctcgtcaaagcaaccatagtacgcgccctgtagcggcgcatta |
| | | agcgcggcgggtgtggtggttacgcgcagcgtgaccgctacactt |
| | | gccagcgccctagcgcccgctcctttcgctttcttcccttccttt |
| | | ctcgccacgttcgccatcttcaaatatgtatccgctcatgagaca |
| | | ataaccctgataaatgcttcaataatattgaaaaaggaagagtcc |
| | | tgaggcggaaagaaccagctgtggaatgtgtgtcagttagggtgt |
| | | ggaaagtccccaggctccccagcaggcagaagtatgcaaagcatg |
| | | catctcaattagtcagcaaccaggtgtggaaagtccccaggctcc |
| | | ccagcaggcagaagtatgcaaagcatgcatctcaattagtcagca |
| | | accatagtcccgcccctaactccgccccatggctgactaattttt |
| | | tttatttatgcagaggccgaggccgcctcggcctctgagctattc |
| | | cagaagtagtgaggaggcttttttggaggcctaggcttttgcaaa |
| | | gatcgatcaagagacaggatgaggatcgtttcgcatgattgaaca |
| | | agatggattgcacgcaggttctccggccgcttgggtggagaggct |
| | | attcggctatgactgggcacaacagacaatcggctgctctgatgc |
| | | cgccgtgttccggctgtcagcgcaggggcgcccggttctttttgt |
| | | caagaccgacctgtccggtgccctgaatgaactgcaagacgaggc |
| | | agcgcggctatcgtggctggccacgacgggcgttccttgcgcagc |
| | | tgtgctcgacgttgtcactgaagcgggaagggactggctgctatt |
| | | gggcgaagtgccggggcaggatctcctgtcatctcaccttgctcc |
| | | tgccgagaaagtatccatcatggctgatgcaatgcggcggctgca |
| | | tacgcttgatccggctacctgcccattcgaccaccaagcgaaaca |
| | | tcgcatcgagcgagcacgtactcggatggaagccggtcttgtcga |
| | | tcaggatgatctggacgaagagcatcaggggctcgcgccagccga |
| | | actgttcgccaggctcaaggcgagcatgcccgacggcgaggatct |
| | | cgtcgtgacccatggcgatgcctgcttgccgaatatcatggtgga |
| | | aaatggccgcttttctggattcatcgactgtggccggctgggtgt |
| | | ggcggaccgctatcaggacatagcgttggctacccgtgatattgc |
| | | tgaagagcttggcggcgaatgggctgaccgcttcctcgtgcttta |
| | | cggtatcgccgctcccgattcgcagcgcatcgccttctatcgcct |
| | | tcttgacgagttcttctgagcgggactctggggttcgaaatgacc |
| | | gaccaagcgacgcccaacctgccatcacgagatttcgattccacc |
| | | gccgccttctatgaaaggttgggcttcggaatcgttttccgggac |
| | | gccggctggatgatcctccagcgcggggatctcatgctggagttc |
| | | ttcgcccaccctagggggaggctaactgaaacacggaaggagaca |
| | | ataccggaaggaacccgcgctatgacggcaataaaaagacagaat |
| | | aaaaacgttgcgcaaactattaactggcgaactacttactctagc |
| | | ttcccggcaacaattaatagactggatggaggcggataaagttgc |
| | | aggaccacttctgcgctcggcccttccggctggctggtttattgc |
| | | tgataaatctggagccggtgagcgtgggtctcgcggtatcattgc |
| | | agcactggggccagatggtaagccctcccgtatcgtagttatcta |
| | | cacgacggggagtcaggcaactatggatgaacgaaatagacagat |
| | | cgctgagataggtgcctcactgattaagcattggtaactgtcaga |
| | | ccaagtttactcatatatactttagattgatttaaaacttcattt |
| | | ttaatttaaaaggatctaggtgaagatcctttttgataatctcat |
| | | gaccaaaatcccttaacgtgagttttcgttccactgagcgtcaga |
| | | ccccgtagaaaagatcaaaggatcttcttgagatcctttttttct |
| | | gcgcgtaatctgctgcttgcaaacaaaaaaaccaccgctaccagc |
| | | ggtggtttgtttgccggatcaagagctaccaactctttttccgaa |
| | | ggtaactggcttcagcagagcgcagataccaaatactgttcttct |
| | | agtgtagccgtagttaggccaccacttcaagaactctgtagcacc |
| | | gcctacatacctcgctctgctaatcctgttaccagtggctgctgc |
| | | cagtggcgataagtcgtgtcttaccgggttggactcaagacgata |
| | | gttaccggataaggcgcagcggtcgggctgaacggggggttcgtg |
| | | cacacagcccagcttggagcgaacgacctacaccgaactgagata |
| | | cctacagcgtgagctatgagaaagcgccacgcttcccgaagggag |
| | | aaaggcggacaggtatccggtaagcggcagggtcggaacaggaga |
| | | gcgcacgagggagcttccagggggaaacgcctggtatctttatag |
| | | tcctgtcgggtttcgccacctctgacttgagcgtcgatttttgtg |
| | | atgctcgtcaggggggcggagcctatggaaaaacgccagcaacgc |
| | | ggcctttttacggttcctggccttttgctggccttttgctcacat |
| | | gttctttcctgcgttatcccctgattctgtggataaccgtattac |
| | | cgcctttgagtgagctgataccgctcgccgcagccgaacgaccga |
| | | gcgcagcgagtcagtgagcgaggaagcggaagagcgcccaatacg |
| | | caaaccgcctctccccgcgcgttggccgattcattaat |
| 9 | SV40 | aggtaagtttagtctttttgtcttttatttcaggtcccggatccg |
| | Chimeric | gtggtggtgcaaatcaaagaactgctcctcagtggatgttgcctt |
| | Intron | tacttctaggcctgtacggaagtgttacttctgctctaaaagctg |
| | (artificial | cggaattgtaccc |
| | sequence) | |
| 10 | polyA sequence | ggccgcaataaaagatctttattttcattagatctgtgtgttggt |
| | (artificial | tttttgtg |
| | sequence) | |
| 11 | 5′ ITR | ctgcgcgctcgctcgctcactgaggccgcccgggcaaagcccggg |
| | (artificial | cgtcgggcgacctttggtcgcccggcctcagtgagcgagcgagcg |
| | sequence) | cgcagagagggagtggggtt |
| 12 | 3′ ITR | aggaacccctagtgatggagttggccactccctctctgcgcgctc |
| | (artificial | gctcgctcactgaggccgggcgaccaaagg |
| | sequence) | tcgcccgacgcccgggctttgcccgggcggcctcagtgagcgagc |
| | SGCA (Genbank | gagcgcgcagctctgtcactcaccgggcgggccaggccgggcagc |
| | Accession No. | catggctgagacactcttctggactcctctcctcgtggttctcct |
| | NM_000023.4) | ggcagggctgggggacaccgaggcccagcagaccacgctacaccc |
| | | acttgtgggccgtgtctttgtgcacaccttggaccatgagacgtt |
| | | tctgagccttcctgagcatgtcgctgtcccacccgctgtccacat |
| | | cacctaccacgcccacctccagggacacccagacctgccccggtg |
| | | gctccgctacacccagcgcagcccccaccaccctggcttcctcta |
| | | cggctctgccaccccagaagatcgtgggctccaggtcattgaggt |
| | | cacagcctacaatcgggacagctttgataccactcggcagaggct |
| | | ggtgctggagattggggacccagaaggccccctgctgccatacca |
| | | agccgagttcctggtgcgcagccacgatgcggaggaggtgctgcc |
| | | ctcaacacctgccagccgcttcctctcagccttggggggactctg |
| | | ggagcccggagagcttcagctgctcaacgtcacctctgccttgga |
| 13 | | ccgtgggggccgtgtcccccttcccattgagggccgaaaagaagg |
| | | ggtatacattaaggtgggttctgcctcacctttttctacttgcct |
| | | gaagatggtggcatcccccgatagccacgcccgctgtgcccaggg |
| | | ccagcctccacttctgtcttgctacgacaccttggcaccccactt |
| | | ccgcgttgactggtgcaatgtgaccctggtggataagtcagtgcc |
| | | ggagcctgcagatgaggtgcccaccccaggtgatgggatcctgga |
| | | gcatgacccgttcttctgcccacccactgaggccccagaccgtga |
| | | cttcttggtggatgctctggtcaccctcctggtgcccctgctggt |
| | | ggccctgcttctcaccttgctgctggcctatgtcatgtgctgccg |
| | | gcgggagggaaggctgaagagagacctggctacctccgacatcca |
| | | gatggtccaccactgcaccatccacgggaacacagaggagctgcg |
| | | gcagatggcggccagccgcgaggtgccccggccactctccaccct |
| | | gcccatgttcaatgtgcacacaggtgagcggctgcctccccgcgt |
| | | ggacagcgcccaggtgcccctcattctggaccagcactgacagcc |
| | | tagccagtggttccaggtccagccctgacttcatcctcccttctc |
| | | tgtccacaccacgagtggcacatcccacctgctgattccagctcc |
| | | tggccctcctggaacccaggctctaaacaagcagggagagggggt |
| | | ggggtggggtgagagtgtgtggagtaaggacattcagaataaata |
| | | tctgctgctctgctcaccaattgctgctggcagcctctcccgtc |
| 14 | SGCA (Genbank | ctctgtcactcaccgggcgggccaggccgggcagccatggctgag |
| | Accession No. | acactcttctggactcctctcctcgtggttctcctggcagggctg |
| | NM_001135697.3) | ggggacaccgaggcccagcagaccacgctacacccacttgtgggc |
| | | cgtgtctttgtgcacaccttggaccatgagacgtttctgagcctt |
| | | cctgagcatgtcgctgtcccacccgctgtccacatcacctaccac |
| | | gcccacctccagggacacccagacctgccccggtggctccgctac |
| | | acccagcgcagcccccaccaccctggcttcctctacggctctgcc |
| | | accccagaagatcgtgggctccaggtcattgaggtcacagcctac |
| | | aatcgggacagctttgataccactcggcagaggctggtgctggag |
| | | attggggacccagaaggccccctgctgccataccaagccgagttc |
| | | ctggtgcgcagccacgatgcggaggaggtgctgccctcaacacct |
| | | gccagccgcttcctctcagccttggggggactctgggagcccgga |
| | | gagcttcagctgctcaacgtcacctctgccttggaccgtgggggc |
| | | cgtgtcccccttcccattgagggccgaaaagaagggctgaagaga |
| | | gacctggctacctccgacatccagatggtccaccactgcaccatc |
| | | cacgggaacacagaggagctgcggcagatggcggccagccgcgag |
| | | gtgccccggccactctccaccctgcccatgttcaatgtgcacaca |
| | | ggtgagcggctgcctccccgcgtggacagcgcccaggtgcccctc |
| | | attctggaccagcactgacagcctagccagtggttccaggtccag |
| | | ccctgacttcatcctcccttctctgtccacaccacgagtggcaca |
| | | tcccacctgctgattccagctcctggccctcctggaacccaggct |
| | | ctaaacaagcagggagagggggtggggtggggtgagagtgtgtgg |
| | | agtaaggacattcagaataaatatctgctgctctgctcaccaatt |
| | | gctgctggcagcctctcccgtc |
| 15 | SGCA (Genbank | MetAlaGluThrLeuPheTrpThrProLeuLeuValValLeuLeu |
| | Accession No. | AlaGlyLeuGlyAspThrGluAlaGlnGlnThrThrLeuHisPro |
| | NP_000014.1) | LeuValGlyArgValPheValHisThrLeuAspHisGluThrPhe |
| | | LeuSerLeuProGluHisValAlaValProProAlaValHisIle |
| | | ThrTyrHisAlaHisLeuGlnGlyHisProAspLeuProArgTrp |
| | | LeuArgTyrThrGlnArgSerProHisHisProGlyPheLeuTyr |
| | | GlySerAlaThrProGluAspArgGlyLeuGlnValIleGluVal |
| | | ThrAlaTyrAsnArgAspSerPheAspThrThrArgGlnArgLeu |
| | | ValLeuGluIleGlyAspProGluGlyProLeuLeuProTyrGln |
| | | AlaGluPheLeuValArgSerHisAspAlaGluGluValLeuPro |
| | | SerThrProAlaSerArgPheLeuSerAlaLeuGlyGlyLeuTrp |
| | | GluProGlyGluLeuGlnLeuLeuAsnValThrSerAlaLeuAsp |
| | | ArgGlyGlyArgValProLeuProIleGluGlyArgLysGluGly |
| | | ValTyrIleLysValGlySerAlaSerProPheSerThrCysLeu |
| | | LysMetValAlaSerProAspSerHisAlaArgCysAlaGlnGly |
| | | GlnProProLeuLeuSerCysTyrAspThrLeuAlaProHisPhe |
| | | ArgValAspTrpCysAsnValThrLeuValAspLysSerValPro |
| | | GluProAlaAspGluValProThrProGlyAspGlyIleLeuGlu |
| | | HisAspProPhePheCysProProThrGluAlaProAspArgAsp |
| | | PheLeuValAspAlaLeuValThrLeuLeuValProLeuLeuVal |
| | | AlaLeuLeuLeuThrLeuLeuLeuAlaTyrValMetCysCysArg |
| | | ArgGluGlyArgLeuLysArgAspLeuAlaThrSerAspIleGln |
| | | MetValHisHisCysThrIleHisGlyAsnThrGluGluLeuArg |
| | | GlnMetAlaAlaSerArgGluVaIProArgProLenSerThrLeu |
| | | ProMetPheAsnValHisThrGlyGluArgLeuProProArgVal |
| | | AspSerAlaGlnValProLenIleLeuAspGlnHis |
| 16 | SGCA (NCBI | MetAlaGluThrLeuPheTrpThrProLeuLeuValValLeuLeu |
| | Reference | AlaGlyLeuGlyAspThrGluAlaGlnGlnThrThrLeuHisPro |
| | Sequence | LeuValGlyArgValPheValHisThrLeuAspHisGluThrPhe |
| | NP_0011292169. | LeuSerLeuProGluHisValAlaValProProAlaValHisIle |
| | 1) | ThrTyrHisAlaHisLeuGlnGlyHisProAspLeuProArgTrp |
| | | LeuArgTyrThrGlnArgSerProHisHisProGlyPheLeuTyr |
| | | GlySerAlaThrProGluAspArgGlyLeuGlnValIleGluVal |
| | | ThrAlaTyrAsnArgAspSerPheAspThrThrArgGlnArgLeu |
| | | ValLeuGluIleGlyAspProGluGlyProLeuLeuProTyrGln |
| | | AlaGluPheLeuValArgSerHisAspAlaGluGluValLeuPro |
| | | SerThrProAlaSerArgPheLeuSerAlaLeuGlyGlyLeuTrp |
| | | GluProGlyGluLeuGlnLeuLeuAsnValThrSerAlaLeuAsp |
| | | ArgGlyGlyArgValProLeuProIleGluGlyArgLysGluGly |
| | | LeuLysArgAspLeuAlaThrSerAspIleGlnMetValHisHis |
| | | CysThrIleHisGlyAsnThrGluGluLeuArgGlnMetAlaAla |
| | | SerArgGluValProArgProLeuSerThrLeuProMetPheAsn |
| | | ValHisThrGlyGluArgLeuProProArgValAspSerAlaGln |
| | | ValProLeuIleLeuAspGlnHis |
| 17 | SGCB (Genbank | acagtcgggcggggagctcggcggcggcgggcgcgggaagatggc |
| | Accession No. | ggcagcggcggcggcggctgcagaacagcaaagttccaatggtcc |
| | NM_000232.5) | tgtaaagaagtccatgcgtgagaaggctgttgagagaaggagtgt |
| | | caataaagagcacaacagtaactttaaagctggatacattccgat |
| | | tgatgaagatcgtctccacaaaacagggttgagaggaagaaaggg |
| | | caatttagccatctgtgtgattatcctcttgtttatcctggctgt |
| | | catcaatttaataataacacttgttatttgggccgtgattcgcat |
| | | tggaccaaatggctgtgatagtatggagtttcatgaaagtggcct |
| | | gcttcgatttaagcaagtatctgacatgggagtgatccaccctct |
| | | ttataaaagcacagtaggaggaaggcgaaatgaaaatttggtcat |
| | | cactggcaacaaccagcctattgtttttcagcaagggacaacaaa |
| | | gctcagtgtagaaaacaacaaaacttctattacaagtgacatcgg |
| | | catgcagttttttgacccgaggactcaaaatatcttattcagcac |
| | | agactatgaaactcatgagtttcatttgccaagtggagtgaaaag |
| | | tttgaatgttcaaaaggcatctactgaaaggattaccagcaatgc |
| | | taccagtgatttaaatataaaagttgatgggcgtgctattgtgcg |
| | | tggaaatgaaggtgtattcattatgggcaaaaccattgaatttca |
| | | catgggtggtaatatggagttaaaggcggaaaacagtatcatcct |
| | | aaatggatctgtgatggtcagcaccacccgcctacccagttcctc |
| | | cagtggagaccagttgggtagtggtgactgggtacgctacaagct |
| | | ctgcatgtgtgctgatgggacgctcttcaaggtgcaagtaaccag |
| | | ccagaacatgggctgccaaatctcagacaacccctgtggaaacac |
| | | tcattaaaagaaccccagaggtcaccaacatgtttatatcttgac |
| | | ttgacttttttatgcatgcaaatcattgtttttacagagtttgtg |
| | | ataactcataattattttaatggcagagcactgctgtatctgttt |
| | | tatggtctacatagttaaaatcttctcagagagcctaaattctaa |
| | | tacattttattaatttatactaatcttcatatttactgttctcta |
| | | aaataattatgagaagcaaataaaatcaaaagtcatgtttaaaga |
| | | cgtgtttttaaaattccactatcccttttctaaaggttaaaggtc |
| | | tgaagcagctgtttagattcactgtaagtaaactttggtaactct |
| | | aatggggatagacccacttaagatatttaaaaaggtatggcatca |
| | | gcgtttcatgctctgccttttagcttctaaaaggaaagatgcaga |
| | | tttctagtgcattaagcctgagccatattctcacatgcaagtgaa |
| | | gtcattaaagaactttacatatgtgagatagaaacaatggttcct |
| | | tagttttgcactgggaagaaaatattttgtaaaagaatgtttatt |
| | | tgaaataatgataactatcaattgttcacaatgtggtggaaatta |
| | | aaacaccatctcagctttaacttttaaataataatgataactatc |
| | | tttattgagcatcttctacatcctaggcattgtcctaggcattgc |
| | | atgtttatatccccaattctcaccacaaccctgcaagtaggtggt |
| | | attatccaagttttacccattaagaaactgaagatcagagaagtt |
| | | aagaaacttgctcaacatcatatagtaagtagcagagttgggatt |
| | | ggaattcaggcatgactcaaacctggatgtacttgattccaaatg |
| | | ccatgttgttttcactctctgcactgactttttaattatttaaaa |
| | | ctctagaaagatgaacaaaggttaatttaaacttacctaagaaga |
| | | tgagaatcaaacaaaacagatatgcttactctagttaaaaagaaa |
| | | ataaatctcatgtcagacccagaaaggaccaatcactgtccgatt |
| | | gtaagctatgttgggccaattccaaaatattatacgatggagagg |
| | | tcaaatttacctacttctgagttacctcagtttcccaacaatgga |
| | | ccttggcacactggagtaacaatacataacagagttgccaagata |
| | | tttataccctcagcactcggggcaacacagtggaaagtggggagg |
| | | ccatagacccaaacaagttctttgggccaggcatggcctagtaag |
| | | tacaccatgcctcgaaaataagtccagaagcactggactaaagag |
| | | tgctaatgcaggaaataatacacataatttttaggtaaggataat |
| | | aatttatctctgctcctaatattactatcccattgtaattattta |
| | | taaccctcaagccagttgatttttaatatatttgattggaaaaga |
| | | actctctggtattattaagactcacacagaatcagggacagggcc |
| | | cccaaaggagtttgctgtaaaataggcagtagagttgtggcatgg |
| | | gcccaaccctgcattcaagtgtaacagcattctgtcagggtcact |
| | | ttgaattgtgcacataagaaaaccaatacaaaaaacaatttgtat |
| | | tcaatattgtcacatttctctctggtagaaaaatcaaatacctta |
| | | gagattatgaagtcattaatttatactgaaattggattgacttac |
| | | tacctaacactgagcgctgtttttaaatgaaaagaatgagattta |
| | | taaccacttgagtgttattgcagtgatatttgaactcatttgaat |
| | | atattcagtatcatttaatgtctgaattcagaaaaaaatgccgaa |
| | | atttttattcagatggtccataaattaaattgcatattcattact |
| | | tatctgctctatttagatttattttaaaagtttatttaagtaaat |
| | | atttttataaaaaccagaaaacactgtattacaaaatattattta |
| | | ttaaatgtagttcaggaaataatctatttttactcttttttggga |
| | | aatacttgtgtttttgatacatctccatgaagttcttttgagagg |
| | | agaggctattttgatgtttttatacaactgaaggttaacagccat |
| | | agcattttatgatactttacaggtagtcctggctttttccctgaa |
| | | acataagcttggaaaatctattagaaagcagaacagggcaaactc |
| | | tccattttacttatggcttttctaatttttaattaattaatttat |
| | | ttttttgagacagagtcttgttctgtcgccaggctggagtgcagt |
| | | ggcacaatctcggctctcggctcactgcaacctctgcctcccagg |
| | | ttcaagcgattctcctgcctcagcctcccgagtacctgggactac |
| | | aggcatgtgccacagttcccggctaatttttgtatttttagtaga |
| | | gacggggtttcaccatgttggccaggatggtctcaatctcttgac |
| | | ctcgtgatctgcctgccttggcctcccaaagtgctgggattacag |
| | | gtgtgagccaccacgcctggccggcttatttttatccacagtaaa |
| | | tcttcagcaactcattgtctccaccagatagtatttttctgtaaa |
| | | tgaaatgctgacttcgcctcttcctgctgtatgctcatccctgca |
| | | ctgagcacagatatgacaagcagtagccatgggggaggtgggtga |
| | | caaagataggaccccgggagggggcgcaggtacatgctagtttca |
| | | attaccacagtattctagagacgggttgcaatgacaaggggggca |
| | | aatgaaatcaatgcaagatttcttaataatgggcagacagaaaaa |
| | | tgtaaaaccacacaaaacggactgctgataatattttaaaatata |
| | | cttatttgtcttctttttgcattgtgaaaaaaacaaaataaattt |
| | | tgtgtgataattttgatgatgaaaggtggaagttctacctagatt |
| | | tgaatgagtgtttttttaagggaatgagaatgtcatggtgctaaa |
| | | cctgacaaataagagatcattgaaatgctgaaaattttaacagtc |
| | | ttcttaaaagtattgagggggcaaaaattaccaattatggtatac |
| | | aaaaataagcctataaatgtgtttcacattgctaacttgagtttc |
| | | agttgattcagtttgtaataactagtaatgagcttctgtttacaa |
| | | taaaaattctgtaaattg |
| 18 | SGCB (Genbank | MetAlaAlaAlaAlaAlaAlaAlaAlaGluGlnGlnSerSerAsn |
| | Accession No. | GlyProValLysLysSerMetArgGluLysAlaValGluArgArg |
| | NP_000223.1) | SerValAsnLysGluHisAsnSerAsnPheLysAlaGlyTyrIle |
| | | ProIleAspGluAspArgLeuHisLysThrGlyLeuArgGlyArg |
| | | LysGlyAsnLeuAlaIleCysValIleIleLeuLeuPheIleLeu |
| | | AlaValIleAsnLeuIleIleThrLeuValIleTrpAlaValIle |
| | | ArgIleGlyProAsnGlyCysAspSerMetGluPheHisGluSer |
| | | GlyLeuLeuArgPheLysGlnValSerAspMetGlyValIleHis |
| | | ProLeuTyrLysSerThrValGlyGlyArgArgAsnGluAsnLeu |
| | | ValIleThrGlyAsnAsnGlnProIleValPheGlnGlnGlyThr |
| | | ThrLysLeuSerValGluAsnAsnLysThrSerIleThrSerAsp |
| | | IleGlyMetGlnPhePheAspProArgThrGlnAsnIleLeuPhe |
| | | SerThrAspTyrGluThrHisGluPheHisLeuProSerGlyVal |
| | | LysSerLeuAsnValGlnLysAlaSerThrGluArgIleThrSer |
| | | AsnAlaThrSerAspLeuAsnIleLysValAspGlyArgAlaIle |
| | | ValArgGlyAsnGluGlyValPheIleMetGlyLysThrIleGlu |
| | | PheHisMetGlyGlyAsnMetGluLeuLysAlaGluAsnSerIle |
| | | IleLeuAsnGlySerValMetValSerThrThrArgLeuProSer |
| | | SerSerSerGlyAspGlnLeuGlySerGlyAspTrpValArgTyr |
| | | LysLeuCysMetCysAlaAspGlyThrLeuPheLysValGlnVal |
| | | ThrSerGlnAsnMetGlyCysGlnIleSerAspAsnProCysGly |
| | | AsnThrHis |
| 19 | pAAV.MHCK7.h | ctgcgcgctcgctcgctcactgaggccgcccgggcaaagcccggg |
| | SGCG (artificial | cgtcgggcgacctttggtcgcccggcctcagtgagcgagcgagcg |
| | sequence) | cgcagagagggagtggggttaaccaattggcgcggccgcaagctt |
| | | gcatgtctaagctagacccttcagattaaaaataactgaggtaag |
| | | ggcctgggtaggggaggtggtgtgagacgctcctgtctctcctct |
| | | atctgcccatcggccctttggggaggaggaatgtgcccaaggact |
| | | aaaaaaaggccatggagccagaggggcgagggcaacagacctttc |
| | | atgggcaaaccttggggccctgctgtctagcatgccccactacgg |
| | | gtctaggctgcccatgtaaggaggcaaggcctggggacacccgag |
| | | atgcctggttataattaacccagacatgtggctgccccccccccc |
| | | ccaacacctgctgcctctaaaaataaccctgtccctggtggatcc |
| | | cctgcatgcgaagatcttcgaacaaggctgtgggggactgagggc |
| | | aggctgtaacaggcttgggggccagggcttatacgtgcctgggac |
| | | tcccaaagtattactgttccatgttcccggcgaagggccagctgt |
| | | cccccgccagctagactcagcacttagtttaggaaccagtgagca |
| | | agtcagcccttggggcagcccatacaaggccatggggctgggcaa |
| | | gctgcacgcctgggtccggggtgggcacggtgcccgggcaacgag |
| | | ctgaaagctcatctgctctcaggggcccctccctggggacagccc |
| | | ctcctggctagtcacaccctgtaggctcctctatataacccaggg |
| | | gcacaggggctgccctcattctaccaccacctccacagcacagac |
| | | agacactcaggagcagccagcggcgcgcccaggtaagtttagtct |
| | | ttttgtcttttatttcaggtcccggatccggtggtggtgcaaatc |
| | | aaagaactgctcctcagtggatgttgcctttacttctaggcctgt |
| | | acggaagtgttacttctgctctaaaagctgcggaattgtacccgg |
| | | taccaccatggtgagggagcagtacaccacagcaaccgagggaat |
| | | ctgcatcgagaggccagagaaccagtacgtgtataagatcggcat |
| | | ctacggctggcggaagagatgtctgtatctgttcgtgctgctgct |
| | | gctgatcatcctggtggtgaatctggccctgaccatctggatcct |
| | | gaaagtgatgtggttttccccagcaggaatgggacacctgtgcgt |
| | | gacaaaggacggactgcggctggagggagagtctgagttcctgtt |
| | | tcccctgtatgccaaggagatccacagcagagtggatagctccct |
| | | gctgctgcagtccacccagaacgtgacagtgaacgcaaggaatag |
| | | cgagggagaggtgaccggcagactgaaggtcggccccaagatggt |
| | | ggaggtgcagaatcagcagttccagatcaactccaatgacggcaa |
| | | gcctctgtttacagtggatgagaaggaggtggtggtgggcaccga |
| | | caagctgagggtgacaggacctgagggcgccctgttcgagcactc |
| | | tgtggagaccccactggtgcgcgcagacccttttcaggatctgag |
| | | gctggagagcccaacacgcagcctgtccatggacgcacccagagg |
| | | cgtgcacatccaggcacacgcaggcaagatcgaggccctgagcca |
| | | gatggatatcctgttccactctagcgacggcatgctggtgctgga |
| | | tgccgagaccgtgtgcctgcctaagctggtgcagggcacatgggg |
| | | cccatctggctcctctcagagcctgtacgagatctgcgtgtgccc |
| | | agatggcaagctgtatctgtccgtggccggcgtgtctaccacatg |
| | | ccaggagcacaaccacatctgtctgtgactcgagggccgcaataa |
| | | aagatctttattttcattagatctgtgtgttggttttttgtgtgt |
| | | cctgcaggggcgcgcctaatctagagcatggctacgtagataagt |
| | | agcatggcgggttaatcattaactacaaggaacccctagtgatgg |
| | | agttggccactccctctctgcgcgctcgctcgctcactgaggccg |
| | | ggcgaccaaaggtcgcccgacgcccgggctttgcccgggcggcct |
| | | cagtgagcgagcgagcgcgc |
| 20 | SGCG nucleotide | atggtgagggagcagtacaccacagcaaccgagggaatctgcatc |
| | sequence | gagaggccagagaaccagtacgtgtataagatcggcatctacggc |
| | (artificial | tggcggaagagatgtctgtatctgttcgtgctgctgctgctgatc |
| | sequence) | atcctggtggtgaatctggccctgaccatctggatcctgaaagtg |
| | | atgtggttttccccagcaggaatgggacacctgtgcgtgacaaag |
| | | gacggactgcggctggagggagagtctgagttcctgtttcccctg |
| | | tatgccaaggagatccacagcagagtggatagctccctgctgctg |
| | | cagtccacccagaacgtgacagtgaacgcaaggaatagcgaggga |
| | | gaggtgaccggcagactgaaggtcggccccaagatggtggaggtg |
| | | cagaatcagcagttccagatcaactccaatgacggcaagcctctg |
| | | tttacagtggatgagaaggaggtggtggtgggcaccgacaagctg |
| | | agggtgacaggacctgagggcgccctgttcgagcactctgtggag |
| | | accccactggtgcgcgcagacccttttcaggatctgaggctggag |
| | | agcccaacacgcagcctgtccatggacgcacccagaggcgtgcac |
| | | atccaggcacacgcaggcaagatcgaggccctgagccagatggat |
| | | atcctgttccactctagcgacggcatgctggtgctggatgccgag |
| | | accgtgtgcctgcctaagctggtgcagggcacatggggcccatct |
| | | ggctcctctcagagcctgtacgagatctgcgtgtgcccagatggc |
| | | aagctgtatctgtccgtggccggcgtgtctaccacatgccaggag |
| | | cacaaccacatctgtctgtga |
| 21 | SGCG (Genbank | attctgtaagtcatagaaaagtttgaaacattctgtctgtggtag |
| | Accession No. | agctcgggccagctgtagttcattcgccagtgtgcttttcttaat |
| | NM_000231.3) | atctaagatggtgcgtgagcagtacactacagccacagaaggcat |
| | | ctgcatagagaggccagagaatcagtatgtctacaaaattggcat |
| | | ttatggctggagaaagcgctgtctctacttgtttgttcttctttt |
| | | actcatcatcctcgttgtgaatttagctcttacaatttggattct |
| | | taaagtgatgtggttttctccagcaggaatgggccacttgtgtgt |
| | | aacaaaagatggactgcgcttggaaggggaatcagaatttttatt |
| | | cccattgtatgccaaagaaatacactccagagtggactcatctct |
| | | gcttctacaatcaacccagaatgtgactgtaaatgcgcgcaactc |
| | | agaaggggaggtcacaggcaggttaaaagtcggtcccaaaatggt |
| | | agaagtccagaatcaacagtttcagatcaactccaacgacggcaa |
| | | gccactatttactgtagatgagaaggaagttgtggttggtacaga |
| | | taaacttcgagtaactgggcctgaaggggctctttttgaacattc |
| | | agtggagacaccccttgtcagagccgacccgtttcaagaccttag |
| | | attagaatcccccactcggagtctaagcatggatgccccaagggg |
| | | tgtgcatattcaagctcacgctgggaaaattgaggcgctttctca |
| | | aatggatattctttttcatagtagtgatggaatgcttgtgcttga |
| | | tgctgaaactgtgtgcttacccaagctggtgcaggggacgtgggg |
| | | tccctctggcagctcacagagcctctacgaaatctgtgtgtgtcc |
| | | agatgggaagctgtacctgtctgtggccggtgtgagcaccacgtg |
| | | ccaggagcacaaccacatctgcctctgagctgcctgcgtcctctc |
| | | ggtgagctgtgcagtgccggccccagatcctcacacccagggagc |
| | | agctgcacatcgtgaaagactgaggcagcgtggatgggaagtaaa |
| | | cgcttccagaggaactcagaaaaaattatgtgccagtgaaagtgt |
| | | ttggacaaaaactacatgatctcaaaatgcacgtggatgtgagac |
| | | acaaaagttgacaaaatggaaaagcaatgtgtttttccactggat |
| | | taattttcaccggaacaattgcgaattctctctgcctcgcctccc |
| | | cctatcttgtccgtgtgggcacacactgagtgttgagttgccgtg |
| | | tggagttaatgtatgacgctccactgtggatatctaatgccctgt |
| | | tgagagtagccttgctcagtactaaaatgccccaaagttctatac |
| | | agcatttcctttatagcattcaaacctcacatcctcccttcagtt |
| | | taatgcaagtaagtcaggtttcacaagaaaattttcaagttttga |
| | | agggaatttgaggttgatctggttttcaagatgtagttaaaggaa |
| | | taaatcactcaaaattaaactttctgtatatagtcaataagcaat |
| | | aaaaacctcatttttcaga |
| 22 | SGCG (Genbank | agagtcgtcgctgcggtcgctgaggaaggacggagcagaggcccg |
| | Accession No. | cgctgtctggggagaagactgtggtgtcatcccgtcgggaatgaa |
| | NM_001378244.1) | gggaaatgcagcggctgtttgcgccccgggactccaagaagtaca |
| | | gcagatggtgcgtgagcagtacactacagccacagaaggcatctg |
| | | catagagaggccagagaatcagtatgtctacaaaattggcattta |
| | | tggctggagaaagcgctgtctctacttgtttgttcttcttttact |
| | | catcatcctcgttgtgaatttagctcttacaatttggattcttaa |
| | | agtgatgtggttttctccagcaggaatgggccacttgtgtgtaac |
| | | aaaagatggactgcgcttggaaggggaatcagaatttttattccc |
| | | attgtatgccaaagaaatacactccagagtggactcatctctgct |
| | | tctacaatcaacccagaatgtgactgtaaatgcgcgcaactcaga |
| | | aggggaggtcacaggcaggttaaaagtcggtcccaaaatggtaga |
| | | agtccagaatcaacagtttcagatcaactccaacgacggcaagcc |
| | | actatttactgtagatgagaaggaagttgtggttggtacagataa |
| | | acttcgagtaactgggcctgaaggggctctttttgaacattcagt |
| | | ggagacaccccttgtcagagccgacccgtttcaagaccttagatt |
| | | agaatcccccactcggagtctaagcatggatgccccaaggggtgt |
| | | gcatattcaagctcacgctgggaaaattgaggcgctttctcaaat |
| | | ggatattctttttcatagtagtgatggaatgcttgtgcttgatgc |
| | | tgaaactgtgtgcttacccaagctggtgcaggggacgtggggtcc |
| | | ctctggcagctcacagagcctctacgaaatctgtgtgtgtccaga |
| | | tgggaagctgtacctgtctgtggccggtgtgagcaccacgtgcca |
| | | ggagcacaaccacatctgcctctgagctgcctgcgtcctctcggt |
| | | gagctgtgcagtgccggccccagatcctcacacccagggagcagc |
| | | tgcacatcgtgaaagactgaggcagcgtggatgggaagtaaacgc |
| | | ttccagaggaactcagaaaaaattatgtgccagtgaaagtgtttg |
| | | gacaaaaactacatgatctcaaaatgcacgtggatgtgagacaca |
| | | aaagttgacaaaatggaaaagcaatgtgtttttccactggattaa |
| | | ttttcaccggaacaattgcgaattctctctgcctcgcctccccct |
| | | atcttgtccgtgtgggcacacactgagtgttgagttgccgtgtgg |
| | | agttaatgtatgacgctccactgtggatatctaatgccctgttga |
| | | gagtagccttgctcagtactaaaatgccccaaagttctatacagc |
| | | atttcctttatagcattcaaacctcacatcctcccttcagtttaa |
| | | tgcaagtaagtcaggtttcacaagaaaattttcaagttttgaagg |
| | | gaatttgaggttgatctggttttcaagatgtagttaaaggaataa |
| | | atcactcaaaattaaactttctgtatatagtcaataagcaataaa |
| | | aacctcatttttcaga |
| 23 | SGCG (Genbank | cctttctccagggacagttgctgaagcttcatcctttgctctcat |
| | Accession No. | tcttcttattttcagaaactatgaataaatcttctacaccatctt |
| | NM_001378245.1) | cccgagagctttagtaagacctcagactggagtagagttgaagga |
| | | gggaatgccagtctgaagaaggaaaggaagaggagagacagcaag |
| | | gagaacttcagttgtcaagatggtgcgtgagcagtacactacagc |
| | | cacagaaggcatctgcatagagaggccagagaatcagtatgtcta |
| | | caaaattggcatttatggctggagaaagcgctgtctctacttgtt |
| | | tgttcttcttttactcatcatcctcgttgtgaatttagctcttac |
| | | aatttggattcttaaagtgatgtggttttctccagcaggaatggg |
| | | ccacttgtgtgtaacaaaagatggactgcgcttggaaggggaatc |
| | | agaatttttattcccattgtatgccaaagaaatacactccagagt |
| | | ggactcatctctgcttctacaatcaacccagaatgtgactgtaaa |
| | | tgcgcgcaactcagaaggggaggtcacaggcaggttaaaagtcgg |
| | | tcccaaaatggtagaagtccagaatcaacagtttcagatcaactc |
| | | caacgacggcaagccactatttactgtagatgagaaggaagttgt |
| | | ggttggtacagataaacttcgagtaactgggcctgaaggggctct |
| | | ttttgaacattcagtggagacaccccttgtcagagccgacccgtt |
| | | tcaagaccttagattagaatcccccactcggagtctaagcatgga |
| | | tgccccaaggggtgtgcatattcaagctcacgctgggaaaattga |
| | | ggcgctttctcaaatggatattctttttcatagtagtgatggaat |
| | | gcttgtgcttgatgctgaaactgtgtgcttacccaagctggtgca |
| | | ggggacgtggggtccctctggcagctcacagagcctctacgaaat |
| | | ctgtgtgtgtccagatgggaagctgtacctgtctgtggccggtgt |
| | | gagcaccacgtgccaggagcacaaccacatctgcctctgagctgc |
| | | ctgcgtcctctcggtgagctgtgcagtgccggccccagatcctca |
| | | cacccagggagcagctgcacatcgtgaaagactgaggcagcgtgg |
| | | atgggaagtaaacgcttccagaggaactcagaaaaaattatgtgc |
| | | cagtgaaagtgtttggacaaaaactacatgatctcaaaatgcacg |
| | | tggatgtgagacacaaaagttgacaaaatggaaaagcaatgtgtt |
| | | tttccactggattaattttcaccggaacaattgcgaattctctct |
| | | gcctcgcctccccctatcttgtccgtgtgggcacacactgagtgt |
| | | tgagttgccgtgtggagttaatgtatgacgctccactgtggatat |
| | | ctaatgccctgttgagagtagccttgctcagtactaaaatgcccc |
| | | aaagttctatacagcatttcctttatagcattcaaacctcacatc |
| | | ctcccttcagtttaatgcaagtaagtcaggtttcacaagaaaatt |
| | | ttcaagttttgaagggaatttgaggttgatctggttttcaagatg |
| | | tagttaaaggaataaatcactcaaaattaaactttctgtatatag |
| | | tcaataagcaataaaaacctcatttttcaga |
| 24 | SGCG (Genbank | attctgtaagtcatagaaaagtttgaaacattctgtctgtggtag |
| | Accession No. | agctcgggccagctgtagttcattcgccagtgtgcttttcttaat |
| | NM_001378246.1) | atctaagtcttattttcagaaactatgaataaatcttctacacca |
| | | tcttcccgagagctttagtaagacctcagactggagtagagttga |
| | | aggagggaatgccagtctgaagaaggaaaggaagaggagagacag |
| | | caaggagaacttcagttgtcaagatggtgcgtgagcagtacacta |
| | | cagccacagaaggcatctgcatagagaggccagagaatcagtatg |
| | | tctacaaaattggcatttatggctggagaaagcgctgtctctact |
| | | tgtttgttcttcttttactcatcatcctcgttgtgaatttagctc |
| | | ttacaatttggattcttaaagtgatgtggttttctccagcaggaa |
| | | tgggccacttgtgtgtaacaaaagatggactgcgcttggaagggg |
| | | aatcagaatttttattcccattgtatgccaaagaaatacactcca |
| | | gagtggactcatctctgcttctacaatcaacccagaatgtgactg |
| | | taaatgcgcgcaactcagaaggggaggtcacaggcaggttaaaag |
| | | tcggtcccaaaatggtagaagtccagaatcaacagtttcagatca |
| | | actccaacgacggcaagccactatttactgtagatgagaaggaag |
| | | ttgtggttggtacagataaacttcgagtaactgggcctgaagggg |
| | | ctctttttgaacattcagtggagacaccccttgtcagagccgacc |
| | | cgtttcaagaccttagattagaatcccccactcggagtctaagca |
| | | tggatgccccaaggggtgtgcatattcaagctcacgctgggaaaa |
| | | ttgaggcgctttctcaaatggatattctttttcatagtagtgatg |
| | | gaatgcttgtgcttgatgctgaaactgtgtgcttacccaagctgg |
| | | tgcaggggacgtggggtccctctggcagctcacagagcctctacg |
| | | aaatctgtgtgtgtccagatgggaagctgtacctgtctgtggccg |
| | | gtgtgagcaccacgtgccaggagcacaaccacatctgcctctgag |
| | | ctgcctgcgtcctctcggtgagctgtgcagtgccggccccagatc |
| | | ctcacacccagggagcagctgcacatcgtgaaagactgaggcagc |
| | | gtggatgggaagtaaacgcttccagaggaactcagaaaaaattat |
| | | gtgccagtgaaagtgtttggacaaaaactacatgatctcaaaatg |
| | | cacgtggatgtgagacacaaaagttgacaaaatggaaaagcaatg |
| | | tgtttttccactggattaattttcaccggaacaattgcgaattct |
| | | ctctgcctcgcctccccctatcttgtccgtgtgggcacacactga |
| | | gtgttgagttgccgtgtggagttaatgtatgacgctccactgtgg |
| | | atatctaatgccctgttgagagtagccttgctcagtactaaaatg |
| | | ccccaaagttctatacagcatttcctttatagcattcaaacctca |
| | | catcctcccttcagtttaatgcaagtaagtcaggtttcacaagaa |
| | | aattttcaagttttgaagggaatttgaggttgatctggttttcaa |
| | | gatgtagttaaaggaataaatcactcaaaattaaactttctgtat |
| | | atagtcaataagcaataaaaacctcatttttcaga |
| 25 | SGCG amino acid | MetValArgGluGlnTyrThrThrAlaThrGluGlyIleCysIle |
| | sequence (homo | GluArgProGluAsnGlnTyrValTyrLysIlcGIyllcTyrGly |
| | sapiens) | TrpArgLysArgCysLcuTyrLcuPhcValLcuLcuLcuLcuIle |
| | | IleLeuValValAsnLeuAlaLeuThrIleTrpIleLeuLysVal |
| | | MetTrpPheSerProAlaGlyMetGlyHisLeuCysValThrLys |
| | | AspGlyLeuArgLeuGluGlyGluSerGluPheLeuPheProLeu |
| | | TyrAlaLysGlnIleHisSerArgValAspSerSerLeuLeuLeu |
| | | GlnSerThrGlnAsnValThrValAsnAlaArgAsnSerGluGly |
| | | GluValThrGlyArgLeuLysValGlyProLysMetVaIGluVal |
| | | GlnAsnGlnGlnPheGlnIleAsnSerAsnAspGlyLysProLeu |
| | | PheThrValAspGluLysGluValValValGlyThrAspLysLeu |
| | | ArgValThrGlyProGluGlyAlaLeuPheGluHisSerValGlu |
| | | ThrProLeuValArgAlaAspProPheGlnAspLeuArgLeuGlu |
| | | SerProThrArgSerLeuSerMetAspAlaProArgGlyValHis |
| | | IleGlnAlaHisAlaGlyLysIleGluAlaLeuSerGlnMetAsp |
| | | IleLeuPheHisSerSerAspGlyMetLeuValLeuAspAlaGlu |
| | | ThrValCysLcuProLysLcuValGInGlyThrTrpGlyProScr |
| | | GlyScrScrGInScrLcuTyrGluIlcCysValCysProAspGly |
| | | LysLeuTyrLeuSerValAlaGlyValSerThrThrCysGlnGlu |
| | | HisAsnHisIleCysLeu |
| 26 | SGCG (Genbank | MetValArgGluGlnTyrThrThrAlaThrGluGlyIleCysIle |
| | Accession No. | GluArgProGluAsnGlnTyrValTyrLysIleGlyIleTyrGly |
| | NP_000222.2) | TrpArgLysArgCysLeuTyrLeuPheValLeuLeuLeuLeuIle |
| | | IleLeuValValAsnLeuAlaLeuThrIleTrpIleLeuLysVal |
| | | MetTrpPheSerProAlaGlyMetGlyHisLeuCysValThrLys |
| | | AspGlyLeuArgLeuGluGlyGluSerGluPheLeuPheProLeu |
| | | TyrAlaLysGlnIleHisSerArgValAspSerSerLeuLeuLeu |
| | | GlnSerThrGlnAsnValThrValAsnAlaArgAsnSerGluGly |
| | | GluValThrGlyArgLeuLysValGlyProLysMetVaIGluVal |
| | | GlnAsnGlnGlnPheGlnIleAsnSerAsnAspGlyLysProLeu |
| | | PheThrValAspGluLysGluValValValGlyThrAspLysLeu |
| | | ArgValThrGlyProGluGlyAlaLeuPheGluHisSerValGlu |
| | | ThrProLeuValArgAlaAspProPheGlnAspLeuArgLeuGlu |
| | | SerProThrArgSerLeuSerMetAspAlaProArgGlyValHis |
| | | IleGlnAlaHisAlaGlyLysIleGluAlaLeuSerGlnMetAsp |
| | | IleLeuPheHisSerSerAspGlyMetLeuValLeuAspAlaGlu |
| | | ThrValCysLeuProLysLeuValGlnGlyThrTrpGlyProSer |
| | | GlySerSerGlnSerLeuTyrGluIleCysValCysProAspGly |
| | | LysLeuTyrLeuSerValAlaGlyValSerThrThrCysGlnGlu |
| | | HisAsnHisIleCysLeu |
| 27 | SGCG (Genbank | MetLysGlyAsnAlaAlaAlaValCysAlaProGlyLeuGlnGlu |
| | Accession No. | ValGlnGlnMetValArgGluGlnTyrThrThrAlaThrGluGly |
| | NP_001365173.1) | IleCysIleGluArgProGluAsnGlnTyrValTyrLysIleGly |
| | | IleTyrGlyTrpArgLysArgCysLeuTyrLeuPheValLeuLeu |
| | | LeuLeuIleIleLeuValValAsnLeuAlaLeuThrIleTrpIle |
| | | LeuLysValMetTrpPheSerProAlaGlyMetGlyHisLeuCys |
| | | ValThrLysAspGlyLeuArgLeuGluGlyGluSerGluPheLeu |
| | | PheProLeuTyrAlaLysGluIleHisSerArgValAspSerSer |
| | | LeuLeuLeuGlnSerThrGlnAsnValThrValAsnAlaArgAsn |
| | | SerGluGlyGluValThrGlyArgLeuLysValGlyProLysMet |
| | | ValGluValGlnAsnGlnGlnPheGlnIleAsnSerAsnAspGly |
| | | LysProLeuPheThrValAspGluLysGluValValValGlyThr |
| | | AspLysLeuArgValThrGlyProGluGlyAlaLeuPheGluHis |
| | | SerValGluThrProLeuValArgAlaAspProPheGlnAspLeu |
| | | ArgLeuGluSerProThrArgSerLeuSerMetAspAlaProArg |
| | | GlyValHisIleGlnAlaHisAlaGlyLysIleGluAlaLeuSer |
| | | GlnMetAspIleLeuPheHisSerSerAspGlyMetLeuValLeu |
| | | AspAlaGluThrValCysLeuProLysLeuValGlnGlyThrTrp |
| | | GlyProSerGlySerSerGlnSerLeuTyrGlnIleCysValCys |
| | | ProAspGlyLysLeuTyrLeuSerValAlaGlyValSerThrThr |
| | | CysGlnGluHisAsnHisIleCysLeu |
| 28 | SGCG (Genbank | MetValArgGluGlnTyrThrThrAlaThrGluGlyIleCysIle |
| | Accession No. | GluArgProGluAsnGlnTyrValTyrLysIleGlyIleTyrGly |
| | NP_001365174.1) | TrpArgLysArgCysLeuTyrLeuPheValLeuLeuLeuLeuIle |
| | | IleLeuValValAsnLeuAlaLeuThrIleTrpIleLeuLysVal |
| | | MetTrpPheSerProAlaGlyMetGlyHisLeuCysValThrLys |
| | | AspGlyLeuArgLeuGluGlyGluSerGluPheLeuPheProLeu |
| | | TyrAlaLysGlnIleHisSerArgValAspSerSerLeuLeuLeu |
| | | GlnSerThrGlnAsnValThrValAsnAlaArgAsnSerGluGly |
| | | GluValThrGlyArgLeuLysValGlyProLysMetVaIGluVal |
| | | GlnAsnGlnGlnPheGlnIleAsnSerAsnAspGlyLysProLeu |
| | | PheThrValAspGluLysGluValValValGlyThrAspLysLeu |
| | | ArgValThrGlyProGluGlyAlaLeuPheGluHisSerValGlu |
| | | ThrProLeuValArgAlaAspProPheGlnAspLeuArgLeuGlu |
| | | SerProThrArgSerLeuSerMetAspAlaProArgGlyValHis |
| | | IleGlnAlaHisAlaGlyLysIleGluAlaLeuSerGlnMetAsp |
| | | IleLeuPheHisSerSerAspGlyMetLeuValLeuAspAlaGlu |
| | | ThrValCysLeuProLysLeuValGlnGlyThrTrpGlyProSer |
| | | GlySerSerGlnSerLeuTyrGluIleCysValCysProAspGly |
| | | LysLeuTyrLeuSerValAlaGlyValSerThrThrCysGlnGlu |
| | | HisAsnHisIleCysLeu |
| 29 | SGCG (Genbank | MetValArgGluGlnTyrThrThrAlaThrGluGlyIleCysIle |
| | Accession No. | GluArgProGluAsnGlnTyrValTyrLysIleGlyIleTyrGly |
| | NP_001365175.1) | TrpArgLysArgCysLeuTyrLeuPheValLeuLeuLeuLeuIle |
| | | IleLeuValValAsnLeuAlaLeuThrIleTrpIleLeuLysVal |
| | | MetTrpPheSerProAlaGlyMetGlyHisLeuCysValThrLys |
| | | AspGlyLeuArgLeuGluGlyGluSerGluPheLeuPheProLeu |
| | | TyrAlaLysGlnIleHisSerArgValAspSerSerLeuLeuLeu |
| | | GlnSerThrGlnAsnValThrValAsnAlaArgAsnSerGluGly |
| | | GluValThrGlyArgLeuLysValGlyProLysMetVaIGluVal |
| | | GlnAsnGlnGlnPheGlnIleAsnSerAsnAspGlyLysProLeu |
| | | PheThrValAspGluLysGluValValValGlyThrAspLysLeu |
| | | ArgValThrGlyProGluGlyAlaLeuPheGluHisSerValGlu |
| | | ThrProLeuValArgAlaAspProPheGlnAspLeuArgLeuGlu |
| | | SerProThrArgSerLeuSerMetAspAlaProArgGlyValHis |
| | | IleGlnAlaHisAlaGlyLysIleGluAlaLeuSerGlnMetAsp |
| | | IleLeuPheHisSerSerAspGlyMetLeuValLeuAspAlaGlu |
| | | ThrValCysLeuProLysLeuValGlnGlyThrTrpGlyProSer |
| | | GlySerSerGlnSerLeuTyrGluIleCysValCysProAspGly |
| | | LysLeuTyrLeuSerValAlaGlyValSerThrThrCysGlnGlu |
| | | HisAsnHisIleCysLeu |
| 30 | SGCD (Genbank | agagaggacttatctccagcatctagcactacagagcagaggctg |
| | Accession No. | tgtggagaatggctgaaaaatcagaattggttgtagaagcagttt |
| | NM_000337.5) | tctttctggttgtgagtatgagcccggcagacaccatgagcgctg |
| | | ttgcaggggagtcggcctgtgcttgacacatgtgtttcccattga |
| | | tagctggagacagcccagtagctgtgagtcggtctgacaaagcca |
| | | tattgaagtacggagtacggtttcaaagcagtcagaaaaagaacg |
| | | ggaatgctgttcaggaaattcttcaggcatgggcagggacttggc |
| | | tgcagttctgcagttggaaaatctgactggggcagcttctgagcg |
| | | caggctgggcctgcacacactcagcgggccgagtggccacctcct |
| | | tcagagctgctcagcacgccctgggatcgcgggcggttttcatcg |
| | | gccggtttgtgaaacggacaagagagagacattactgccgggagt |
| | | gttgagtgaagggaccaggtggagatgatgcctcaggagcagtac |
| | | actcaccaccggagcaccatgcctggctctgtggggccacaggta |
| | | tacaaggtggggatttatggctggcggaaacgatgcctgtatttc |
| | | tttgtcctgctcctcatgattttaatactggtgaacttggccatg |
| | | accatctggattctcaaagtcatgaacttcacaattgatggaatg |
| | | ggaaacctgaggatcacagaaaaaggtctaaagctagaaggagac |
| | | tctgaattcttacaacctctctacgccaaagaaatccagtcccga |
| | | ccaggtaatgccctgtacttcaagtctgccagaaatgttacagtg |
| | | aacattctcaatgaccagactaaagtgctaactcagcttataaca |
| | | ggtccaaaagccgtagaagcttatggtaaaaaatttgaggtaaaa |
| | | actgtttctggaaaattgctcttctctgcagacaataatgaagtg |
| | | gtagtaggagctgaaagattacgagttttaggagcggagggcaca |
| | | gtgttccctaaatctatagaaacacctaatgtcagggcagacccc |
| | | ttcaaagaactaaggttggagtccccaacccggtctctagtgatg |
| | | gaggccccaaaaggagtggaaatcaatgcagaagctggcaatatg |
| | | gaagccacctgcaggacagagctgagactggaatccaaagatgga |
| | | gagattaagttagatgctgcgaaaatcaggctacctagactgcct |
| | | catggatcctacacgcctacaggaacgaggcagaaggtcttcgag |
| | | atctgcgtctgcgccaatgggagattattcctgtctcaggcagga |
| | | gctgggtccacttgtcagataaacacaagtgtctgcctctgaaag |
| | | actatccatagtggacattgttggcagcataaaggccttttttgg |
| | | ctttagacactggctgccagctatttttactagaacacagaaagc |
| | | ctatcaaagaccttgtgtgtatgtgtacgtgtgtgtgcgtgcttg |
| | | agtgtgttcgcgtgtgtgggtggatataaatatatataaatatat |
| | | ataaataaatatatatatcctctgtataaaatgaggtttcagtac |
| | | aaaaggaaccatgggtgaccctgcgatatccactgtcattttcca |
| | | tcccatccccaccacctgagtgacagaaatctaaacacacatccg |
| | | tcccaacattccccagaccattcagaatcacacagcgtattaaac |
| | | actgacagaatcttcatctagatattcgagtagcagcatatcttc |
| | | tcttttagtgtcattacgagggagtgatggcgggaatctcagtcg |
| | | cactcaagctctgagacctttgtatcaaaaataggcatttgattt |
| | | cctgttttagctttagtaaggctggctaacttccccctcttcaag |
| | | ctaggaactggcaatgctgtagaagtcagccgtaggaattcaaaa |
| | | tggctggcctaccttggctaccagacatattggggtttttgtagt |
| | | tgaatgaatgaggaggatgaatttcagcaaattttgaactgctca |
| | | cccaacttctgctatcttgctccctccaaactcacagattctcct |
| | | acagtcaaattaggagctgtaaatcagcacaaaataagataacag |
| | | ctgttcctcagtgagctggaagctacttaatggcctgatgggcaa |
| | | tgaacaaacgggtgatatgtctctgtttaagggaaaaatggctta |
| | | aaagctgttctgttctcacttctgactttaaccaaaagatttcaa |
| | | cccacaatgatcaggtcaatcaaaatccctaagagcagaactcct |
| | | accccaaaagaagcctggaagtctaattaagagtagcataaggaa |
| | | cctaattatgtttaccatgtttcttgggattggtgggaaatgtca |
| | | aaacatgccctttatttttaaaggcattcacaaactctctgactt |
| | | tgttcttcttatatattttttcagtgccgggatattcatattcct |
| | | aaagccactattgtgttttctctaagaagcactacatgccaccag |
| | | aattgtgcactgaaagataatacaaactgagtgtctttatgagaa |
| | | tcactgtgtcccctgaggcccagcagtacctgcttccctgtatgt |
| | | ggaagcagcacctcattcccgccatcagctacctctcatcacccc |
| | | accttcatcatcatgctccagggtcaccctggccagctcttgtgc |
| | | tgggacaggggattacaccatctctgttcaaagagggggaaatgt |
| | | gcctatgccttaagtcaatgcactcagcaaggagaagcacatctt |
| | | atttatcttgttacctatagtttactttgggtgattggaggggaa |
| | | tgacttagttatgactggacatcttaaaagctgatagacaagcca |
| | | aatggctggcagatgatgtggatttcaaagagcccagaatgaact |
| | | catcactggcttagacagtcctggatgccatttggaaagtagtgg |
| | | ccctgcaagcctaaatgaagtagtatttgtaggcctgggtggcat |
| | | ttggatttttcttttccctcaacaaggttttactttttcttactt |
| | | tacaagcaagggaagttttgtgataggagagaaataaaagatttg |
| | | atattttttgagatgacactcaagcatcaggctgagatttgcaca |
| | | catgggatgtaaaagcaagctgtgtgttgcttagtcacttactta |
| | | gaagtagatggtgggggacagcggcgtgggtcctagcctggccag |
| | | tgatgctgctggcgtccagaccccagactcactccaagcactctt |
| | | gttcaatatctcatgcagaagagttgggctggtcactcttagggg |
| | | tgagaccccgtgattggttggtttgtagcactaaggtctaaaaag |
| | | gaaaaccataaaagacatcagattacgctggatcagtataattaa |
| | | tattccatagggccatgttgccagaatctgtatgtatcaatacag |
| | | ggtttttccaagccaggaaacgccctccttggctactaggagcac |
| | | ctatcccatatcattcagataaacaatgatagctacaaagtcatt |
| | | tgtggctagataggttaagacaggtgattttttaaagtagactgt |
| | | ctttgcattttgccatctgaagttcattaatctttagatgacaaa |
| | | aaagcaaaaagttcccagaacgtttttgcttagattttgttctaa |
| | | tcaccacgtgaaggaatgagattagcaccacaagtttcatgccaa |
| | | taaaagagactggtgtgatcccacatgcaaaatttaatcctaagg |
| | | gtagtgagatcacaaacagaataaaaataagagcaatcaaccata |
| | | taaatcaagtacctattgggaacagacataacattcaatttttca |
| | | tttatgctaagtgaccacagtatacaaagtaataagcaggaaatt |
| | | tgatatgggttaaattatgcattttgttcagattttggaaattgg |
| | | tatgcattataagtttctcaatgtacatctttttatcccaacacc |
| | | ctcaaactagaatatttgtcagtggtcaagagaaaaaattttacc |
| | | tgaattcttgggggccggcggggagcttttacattaaaaatctac |
| | | taacgcctactttttaaaaaatgagattctttctaatctttatat |
| | | atgacattttctagacaatcgcacctttgggtatattaaacagct |
| | | ggtatcataacaaagaatccaaatgaaccttcaatatactagaag |
| | | ttctagtaggttaatattgttcagaagatttttacaaattaaaaa |
| | | ctgatttccaaatatgttcaacatttacttctatttgatatctgc |
| | | tcaagaagtcatagaagtcttgggaaactattcgagtatcacaga |
| | | ggttttcaaaagcccttatggtgacatctacctaggtaaaagcct |
| | | gacatgtggctttataattgttatgttacccaagggataaacttg |
| | | aactggctttgaacatcctttaggtcattttctctttggataatt |
| | | ttcatcgcatatccagcaactatagaccaaagtttgcttaaggtt |
| | | tgaccctagagcagaggtgggcaaactatgacctggaaaccaaat |
| | | ctgacttaccaccttttcctgtagttaaagttttattgtcacata |
| | | gccacatacattcatttacatgttttctgattttacactacagca |
| | | gcaggatggagtagttgtgtcagagactatgtatcccacaaagca |
| | | tagattacttactatttagtcctttacaggcaaagtttcctcacc |
| | | cctgacctagaggtttttgtggtatgcattggatatagcaggaaa |
| | | gaaagcacatttccaaacagcagggagtaagcttacatttctgtg |
| | | taggtttgggaatatagttacttggcaaagtctttcaggaaggaa |
| | | gcccttccttatgttacatgtggaaagcctgccttccaagacatg |
| | | tggaagtaattgatccacctgccagagaaacacaggctcagagga |
| | | tgcctgggaacagggaggatgggattagtggaagcttaatggaaa |
| | | aggaaagttatgatcctccaagacccttaattgatagaccatacc |
| | | aggttctgcaggtcagcatcattgtgtaatgagagtgaagtaggg |
| | | gaccctgtggttcaaccttagaatctgtttcctgtaggctctttc |
| | | tgctgtctatattcattaaagttttccacttcaccctcccatagt |
| | | ctagagggatgcccattccatggtcctccagagaatagttttgac |
| | | ttaacatgtctgtttagcccacatcacgtcagttatcaacaccgc |
| | | cactgtgcttactgttcctacagccacaccaggcttgaagagtta |
| | | gtgagaccaacaaataattggaagtattggaaaaagcaaaataca |
| | | tggggacaaaaaaaatacagtgaaattctttttatcaaactgatg |
| | | ctgtgagaaaccagatgaatgccagtttggctttatttctaagaa |
| | | tctgggtcttcattctctggtgtagaaggaatgcaaaaaactata |
| | | acaacaacaacaaaaacatattttgaaaagacatattctgacatc |
| | | tctgcttgtgtgtggtaaggcaggttcctatcagacatttatccc |
| | | tttggtcaagatcccttttgctcatccagggtttcatactcaata |
| | | tcgcttaaaaaaaaaaaagtatcagctagggatgactctggaagt |
| | | atgagtatcatggtggggaggaaggaattttttttaaatgtaaat |
| | | gacccccatttaccagaccctaatcaaagtcacttaagggaatcc |
| | | ctcagccttttatttggaaacagttgaaataaactggcagcagct |
| | | agatcagagtatcttgctttattttataaaggccaaaggtagtat |
| | | gaagtttggaccaaaaaggtaaatagatccattccagcacctgat |
| | | actgatttttcaaggctctatgaaaggtcaaaaatttcattaaac |
| | | aagaccagttctccctcttccccctgtcccaagaaatcttaggca |
| | | tgaaaaggataaggaaacagctcctggaatgatacatttgcatag |
| | | tgccctagtagcaggttgggaaaaagttataatataagaaacaac |
| | | cttcgaaaacaggccttttatctcaaagataaaatgtctttcttg |
| | | tggtctttcatcactatctccgtggtggaaggttcccctagttcc |
| | | aacatatttccattaaaatagtcaaagccacggcattgggatgtc |
| | | agatgcctctctttctttgtggtaatcggaatttaaaattataca |
| | | gttgcctctgaatttctcatgcacaaagccaaaccactgataaga |
| | | gataaagcagtctgaagcctgctgcttcagccagcacagcacacc |
| | | acacacgctcgcactttcaaaagcaatgtgatttctatggttctt |
| | | aaaagctttcttcataagggagtccctgaaatttctcaaggcagg |
| | | tttgaatggcaaagggaaaataattacttgtgggaggtcctcctt |
| | | ttgagtattgttagagcatacatgtaaaagaaaataacctttttg |
| | | gggcaactcatgctcacacatgctgttttctttggttctccccct |
| | | accttccttttgtagatattgacagaataggaggaaatgagcatc |
| | | cttatttgagaaagagcaagaatgtcatgagcccttgatgcaata |
| | | gtaagtgtgatgtcatcatacagtgttaatgatgctatcaaatcc |
| | | atcaataaacagtctcaaaccttccaacaacagtgctcactgctg |
| | | ctcctcaacttcagcccagcaagcagtaatatcatcacccatttt |
| | | gagatatgcaggtggaatagaacaaagaaacagccactgtaatcg |
| | | agaagcatgtttactgtctaaatccacctgttgcagtaggaagcc |
| | | agagtggggttccaaatgcctcattaagtatgtggacagcctcac |
| | | tagtaagtgagtgaatttggcttcatcactgaacattagctaagg |
| | | tcagcttaataacacaaatatgaggccgacttctttgcgagaaga |
| | | gaaaagaaaacatctgcttgatttaaaatccaccccacatgccta |
| | | gagttgtctaatagtccctcactttccaatggcttcacatcctac |
| | | ttctacatttggggttttttggtgaaatcagagatagctcaggat |
| | | ttcataaaacgagaaactccaaactggtctattaggttccatggg |
| | | aacacttgtagccaaaggattgtctgagggcaggaagacgacact |
| | | tgtcaacaaggaagacagtgtttctttagttcccatattcatcta |
| | | attcatggggttctaacattttggggggccatagattcttttgac |
| | | aatctgagccaatctatgaaacttctccccaaaaagaaccacccc |
| | | acaaaatcttgcaaaacgtaagagattttcctggatctgaagcta |
| | | cccgaagacatgggaagagttgtattctattattcaattttaaga |
| | | atatttattaatattcactgagctattgctagccactgtgctaaa |
| | | cattttacatacattctcctatttcatcctcaaaacaatcctttg |
| | | agttgggttttaatatagttccaatattcggatgtgaaaactgag |
| | | gcttatagtggctaagaaacttgcccaaagccactacctagaaaa |
| | | tggcagagctggaatgtaggtttaactcctgaccactatgctata |
| | | aagtcaccacatgtcaaactaattttcaagttgttgggacatgtc |
| | | ccctactaggttttaaactagatcttccttggtagatgaagctat |
| | | agaacttctattttccctgctttctgtagtctctcacagtgacag |
| | | catctatactaaagtatagatacctaaggggaaaatatagaaagc |
| | | ctgcctgaataatagaatctagaacaacaacaaaaatattaattt |
| | | tttctgtgtatgcttaggtcaagcttaaaaaaaaaaaaaaaagac |
| | | cggaaaatacctgggttgttagcctcacatttaggaaaaaattgt |
| | | aatactcagttatctgtgtgtgtggctaaacaagtcagcatttct |
| | | gcacacatacatctctttcctttatacttcccttcaaaagacaaa |
| | | tatcttacttttgatctttgacactatttggtcagtattcttctt |
| | | tacctttacttgtggcaaaactcaaggaagcgatcaaatagaggg |
| | | aagctcatttctatcattgtctctgtttccctataagaaagaact |
| | | accagggactcactgactgcattaggcatacaatgtcagagctga |
| | | gctgaccactctggtcctgtaatgtctttggcctcacaccttggc |
| | | agccatcattaatgggccatacccttccccaggtgcagaattctc |
| | | ctccccagagcactcaggccgttactaccaatttatctgagttgg |
| | | aaataagactcatttgccagttcttatttttaaagtggcaccctt |
| | | taactttgaacctgtgtattttacactggcatcctagattcagca |
| | | atgaggtttggtggtgtttcaactaggaagggagaaaatgagtgc |
| | | atctgaagttccttacagcttggtttctttggaatgctttcatct |
| | | tctaagcaaagggatcagggtttgatctgtaagagttaaaaagac |
| | | aaagtcattttgaagaattaactcagccagggatcatgcaaaaag |
| | | attagaaaccataatgcccttgttaaagccctgctgtcaacctgc |
| | | cttcacccagagcttagagggccacagcagcaaagaggttggggt |
| | | ccatccctctctgatgtgctttttccacaacacatatctggtcct |
| | | ctggcaggattgtggatagagctcctcaccatacccaaaagactc |
| | | agccccagtgccagtgctttcctggttcaacaacccaccacaaaa |
| | | ccttagtaaaaggatgagccaaaaatgaaaaagactcgactctac |
| | | agtaagtcagtcagggatttcctttttaatggtttaagacatcca |
| | | aatggcaagccaggaatagataccattaaagggtctcataggact |
| | | aaccttaccagagccagaaatctagctctctggaagagatgcaag |
| | | attctagaaaagtaaagggaagtgtcggcacatctaaatttagtg |
| | | aacacaaaattaatttttatctagtctgtgacggagggaataaag |
| | | tttttcatgtatcaaccacctcccccagtcaggtttctccctttt |
| | | tgagattatgaagaagctgagacatacttcttaaggaggtcgtgt |
| | | tttagaaggaaaaggcagaggctatccatcattatgctggctaga |
| | | tgcgcttctgaagaagccggattctgatgttcttaaccaaaatgg |
| | | tgaggtcatggaagtcccatttgcttggagattttgaaaaaaaaa |
| | | aaaaaaaaaaacccattcccataaagtaattgagttcagcctttg |
| | | gattatttttggtttggtttttctctggttttgggtgtgatgtaa |
| | | gaagagctttttagttttgttttgaataacatcaatccttgcaca |
| | | ctctatgcaaaaattttgtaagcatttcaataatgctatgaatta |
| | | caaggaactattttaactttattacactttctgtataaaaaattt |
| | | gtatttaatattatttcgaccacagtcttgtaaaatatattaata |
| | | aaaataatgattggtaagaaggaaaaaaaaaaaaaaaaaa |
| 31 | SGCD (Genbank | agagctgctcagcacgccctgggatcgcgggcggttttcatcggc |
| | Accession No. | cggtttgtgaaacggacaagagagatgcctcaggagcagtacact |
| | NM_001128209.2) | caccaccggagcaccatgcctggctctgtggggccacaggtatac |
| | | aaggtggggatttatggctggcggaaacgatgcctgtatttcttt |
| | | gtcctgctcctcatgattttaatactggtgaacttggccatgacc |
| | | atctggattctcaaagtcatgaacttcacaattgatggaatggga |
| | | aacctgaggatcacagaaaaaggtctaaagctagaaggagactct |
| | | gaattcttacaacctctctacgccaaagaaatccagtcccgacca |
| | | ggtaatgccctgtacttcaagtctgccagaaatgttacagtgaac |
| | | attctcaatgaccagactaaagtgctaactcagcttataacaggt |
| | | ccaaaagccgtagaagcttatggtaaaaaatttgaggtaaaaact |
| | | gtttctggaaaattgctcttctctgcagacaataatgaagtggta |
| | | gtaggagctgaaagattacgagttttaggagcggagggcacagtg |
| | | ttccctaaatctatagaaacacctaatgtcagggcagaccccttc |
| | | aaagaactaaggttggagtccccaacccggtctctagtgatggag |
| | | gccccaaaaggagtggaaatcaatgcagaagctggcaatatggaa |
| | | gccacctgcaggacagagctgagactggaatccaaagatggagag |
| | | attaagttagatgctgcgaaaatcaggctacctagactgcctcat |
| | | ggatcctacacgcctacaggaacgaggcagaaggtcttcgagatc |
| | | tgcgtctgcgccaatgggagattattcctgtctcaggcaggagct |
| | | gggtccacttgtcagataaacacaagtgtctgcctctgaaagact |
| | | atccatagtggacattgttggcagcataaaggccttttttggctt |
| | | tagacactggctgccagctatttttactagaacacagaaagccta |
| | | tcaaagaccttgtgtgtatgtgtacgtgtgtgtgcgtgcttgagt |
| | | gtgttcgcgtgtgtgggtggatataaatatatataaatatatata |
| | | aataaatatatatatcctctgtataaaatgaggtttcagtacaaa |
| | | aggaaccatgggtgaccctgcgatatccactgtcattttccatcc |
| | | catccccaccacctgagtgacagaaatctaaacacacatccgtcc |
| | | caacattccccagaccattcagaatcacacagcgtattaaacact |
| | | gacagaatcttcatctagatattcgagtagcagcatatcttctct |
| | | tttagtgtcattacgagggagtgatggcgggaatctcagtcgcac |
| | | tcaagctctgagacctttgtatcaaaaataggcatttgatttcct |
| | | gttttagctttagtaaggctggctaacttccccctcttcaagcta |
| | | ggaactggcaatgctgtagaagtcagccgtaggaattcaaaatgg |
| | | ctggcctaccttggctaccagacatattggggtttttgtagttga |
| | | atgaatgaggaggatgaatttcagcaaattttgaactgctcaccc |
| | | aacttctgctatcttgctccctccaaactcacagattctcctaca |
| | | gtcaaattaggagctgtaaatcagcacaaaataagataacagctg |
| | | ttcctcagtgagctggaagctacttaatggcctgatgggcaatga |
| | | acaaacgggtgatatgtctctgtttaagggaaaaatggcttaaaa |
| | | gctgttctgttctcacttctgactttaaccaaaagatttcaaccc |
| | | acaatgatcaggtcaatcaaaatccctaagagcagaactcctacc |
| | | ccaaaagaagcctggaagtctaattaagagtagcataaggaacct |
| | | aattatgtttaccatgtttcttgggattggtgggaaatgtcaaaa |
| | | catgccctttatttttaaaggcattcacaaactctctgactttgt |
| | | tcttcttatatattttttcagtgccgggatattcatattcctaaa |
| | | gccactattgtgttttctctaagaagcactacatgccaccagaat |
| | | tgtgcactgaaagataatacaaactgagtgtctttatgagaatca |
| | | ctgtgtcccctgaggcccagcagtacctgcttccctgtatgtgga |
| | | agcagcacctcattcccgccatcagctacctctcatcaccccacc |
| | | ttcatcatcatgctccagggtcaccctggccagctcttgtgctgg |
| | | gacaggggattacaccatctctgttcaaagagggggaaatgtgcc |
| | | tatgccttaagtcaatgcactcagcaaggagaagcacatcttatt |
| | | tatcttgttacctatagtttactttgggtgattggaggggaatga |
| | | cttagttatgactggacatcttaaaagctgatagacaagccaaat |
| | | ggctggcagatgatgtggatttcaaagagcccagaatgaactcat |
| | | cactggcttagacagtcctggatgccatttggaaagtagtggccc |
| | | tgcaagcctaaatgaagtagtatttgtaggcctgggtggcatttg |
| | | gatttttcttttccctcaacaaggttttactttttcttactttac |
| | | aagcaagggaagttttgtgataggagagaaataaaagatttgata |
| | | ttttttgagatgacactcaagcatcaggctgagatttgcacacat |
| | | gggatgtaaaagcaagctgtgtgttgcttagtcacttacttagaa |
| | | gtagatggtgggggacagcggcgtgggtcctagcctggccagtga |
| | | tgctgctggcgtccagaccccagactcactccaagcactcttgtt |
| | | caatatctcatgcagaagagttgggctggtcactcttaggggtga |
| | | gaccccgtgattggttggtttgtagcactaaggtctaaaaaggaa |
| | | aaccataaaagacatcagattacgctggatcagtataattaatat |
| | | tccatagggccatgttgccagaatctgtatgtatcaatacagggt |
| | | ttttccaagccaggaaacgccctccttggctactaggagcaccta |
| | | tcccatatcattcagataaacaatgatagctacaaagtcatttgt |
| | | ggctagataggttaagacaggtgattttttaaagtagactgtctt |
| | | tgcattttgccatctgaagttcattaatctttagatgacaaaaaa |
| | | gcaaaaagttcccagaacgtttttgcttagattttgttctaatca |
| | | ccacgtgaaggaatgagattagcaccacaagtttcatgccaataa |
| | | aagagactggtgtgatcccacatgcaaaatttaatcctaagggta |
| | | gtgagatcacaaacagaataaaaataagagcaatcaaccatataa |
| | | atcaagtacctattgggaacagacataacattcaatttttcattt |
| | | atgctaagtgaccacagtatacaaagtaataagcaggaaatttga |
| | | tatgggttaaattatgcattttgttcagattttggaaattggtat |
| | | gcattataagtttctcaatgtacatctttttatcccaacaccctc |
| | | aaactagaatatttgtcagtggtcaagagaaaaaattttacctga |
| | | attcttgggggccggcggggagcttttacattaaaaatctactaa |
| | | cgcctactttttaaaaaatgagattctttctaatctttatatatg |
| | | acattttctagacaatcgcacctttgggtatattaaacagctggt |
| | | atcataacaaagaatccaaatgaaccttcaatatactagaagttc |
| | | tagtaggttaatattgttcagaagatttttacaaattaaaaactg |
| | | atttccaaatatgttcaacatttacttctatttgatatctgctca |
| | | agaagtcatagaagtcttgggaaactattcgagtatcacagaggt |
| | | tttcaaaagcccttatggtgacatctacctaggtaaaagcctgac |
| | | atgtggctttataattgttatgttacccaagggataaacttgaac |
| | | tggctttgaacatcctttaggtcattttctctttggataattttc |
| | | atcgcatatccagcaactatagaccaaagtttgcttaaggtttga |
| | | ccctagagcagaggtgggcaaactatgacctggaaaccaaatctg |
| | | acttaccaccttttcctgtagttaaagttttattgtcacatagcc |
| | | acatacattcatttacatgttttctgattttacactacagcagca |
| | | ggatggagtagttgtgtcagagactatgtatcccacaaagcatag |
| | | attacttactatttagtcctttacaggcaaagtttcctcacccct |
| | | gacctagaggtttttgtggtatgcattggatatagcaggaaagaa |
| | | agcacatttccaaacagcagggagtaagcttacatttctgtgtag |
| | | gtttgggaatatagttacttggcaaagtctttcaggaaggaagcc |
| | | cttccttatgttacatgtggaaagcctgccttccaagacatgtgg |
| | | aagtaattgatccacctgccagagaaacacaggctcagaggatgc |
| | | ctgggaacagggaggatgggattagtggaagcttaatggaaaagg |
| | | aaagttatgatcctccaagacccttaattgatagaccataccagg |
| | | ttctgcaggtcagcatcattgtgtaatgagagtgaagtaggggac |
| | | cctgtggttcaaccttagaatctgtttcctgtaggctctttctgc |
| | | tgtctatattcattaaagttttccacttcaccctcccatagtcta |
| | | gagggatgcccattccatggtcctccagagaatagttttgactta |
| | | acatgtctgtttagcccacatcacgtcagttatcaacaccgccac |
| | | tgtgcttactgttcctacagccacaccaggcttgaagagttagtg |
| | | agaccaacaaataattggaagtattggaaaaagcaaaatacatgg |
| | | ggacaaaaaaaatacagtgaaattctttttatcaaactgatgctg |
| | | tgagaaaccagatgaatgccagtttggctttatttctaagaatct |
| | | gggtcttcattctctggtgtagaaggaatgcaaaaaactataaca |
| | | acaacaacaaaaacatattttgaaaagacatattctgacatctct |
| | | gcttgtgtgtggtaaggcaggttcctatcagacatttatcccttt |
| | | ggtcaagatcccttttgctcatccagggtttcatactcaatatcg |
| | | cttaaaaaaaaaaaagtatcagctagggatgactctggaagtatg |
| | | agtatcatggtggggaggaaggaattttttttaaatgtaaatgac |
| | | ccccatttaccagaccctaatcaaagtcacttaagggaatccctc |
| | | agccttttatttggaaacagttgaaataaactggcagcagctaga |
| | | tcagagtatcttgctttattttataaaggccaaaggtagtatgaa |
| | | gtttggaccaaaaaggtaaatagatccattccagcacctgatact |
| | | gatttttcaaggctctatgaaaggtcaaaaatttcattaaacaag |
| | | accagttctccctcttccccctgtcccaagaaatcttaggcatga |
| | | aaaggataaggaaacagctcctggaatgatacatttgcatagtgc |
| | | cctagtagcaggttgggaaaaagttataatataagaaacaacctt |
| | | cgaaaacaggccttttatctcaaagataaaatgtctttcttgtgg |
| | | tctttcatcactatctccgtggtggaaggttcccctagttccaac |
| | | atatttccattaaaatagtcaaagccacggcattgggatgtcaga |
| | | tgcctctctttctttgtggtaatcggaatttaaaattatacagtt |
| | | gcctctgaatttctcatgcacaaagccaaaccactgataagagat |
| | | aaagcagtctgaagcctgctgcttcagccagcacagcacaccaca |
| | | cacgctcgcactttcaaaagcaatgtgatttctatggttcttaaa |
| | | agctttcttcataagggagtccctgaaatttctcaaggcaggttt |
| | | gaatggcaaagggaaaataattacttgtgggaggtcctccttttg |
| | | agtattgttagagcatacatgtaaaagaaaataacctttttgggg |
| | | caactcatgctcacacatgctgttttctttggttctccccctacc |
| | | ttccttttgtagatattgacagaataggaggaaatgagcatcctt |
| | | atttgagaaagagcaagaatgtcatgagcccttgatgcaatagta |
| | | agtgtgatgtcatcatacagtgttaatgatgctatcaaatccatc |
| | | aataaacagtctcaaaccttccaacaacagtgctcactgctgctc |
| | | ctcaacttcagcccagcaagcagtaatatcatcacccattttgag |
| | | atatgcaggtggaatagaacaaagaaacagccactgtaatcgaga |
| | | agcatgtttactgtctaaatccacctgttgcagtaggaagccaga |
| | | gtggggttccaaatgcctcattaagtatgtggacagcctcactag |
| | | taagtgagtgaatttggcttcatcactgaacattagctaaggtca |
| | | gcttaataacacaaatatgaggccgacttctttgcgagaagagaa |
| | | aagaaaacatctgcttgatttaaaatccaccccacatgcctagag |
| | | ttgtctaatagtccctcactttccaatggcttcacatcctacttc |
| | | tacatttggggttttttggtgaaatcagagatagctcaggatttc |
| | | ataaaacgagaaactccaaactggtctattaggttccatgggaac |
| | | acttgtagccaaaggattgtctgagggcaggaagacgacacttgt |
| | | caacaaggaagacagtgtttctttagttcccatattcatctaatt |
| | | catggggttctaacattttggggggccatagattcttttgacaat |
| | | ctgagccaatctatgaaacttctccccaaaaagaaccaccccaca |
| | | aaatcttgcaaaacgtaagagattttcctggatctgaagctaccc |
| | | gaagacatgggaagagttgtattctattattcaattttaagaata |
| | | tttattaatattcactgagctattgctagccactgtgctaaacat |
| | | tttacatacattctcctatttcatcctcaaaacaatcctttgagt |
| | | tgggttttaatatagttccaatattcggatgtgaaaactgaggct |
| | | tatagtggctaagaaacttgcccaaagccactacctagaaaatgg |
| | | cagagctggaatgtaggtttaactcctgaccactatgctataaag |
| | | tcaccacatgtcaaactaattttcaagttgttgggacatgtcccc |
| | | tactaggttttaaactagatcttccttggtagatgaagctataga |
| | | acttctattttccctgctttctgtagtctctcacagtgacagcat |
| | | ctatactaaagtatagatacctaaggggaaaatatagaaagcctg |
| | | cctgaataatagaatctagaacaacaacaaaaatattaatttttt |
| | | ctgtgtatgcttaggtcaagcttaaaaaaaaaaaaaaaagaccgg |
| | | aaaatacctgggttgttagcctcacatttaggaaaaaattgtaat |
| | | actcagttatctgtgtgtgtggctaaacaagtcagcatttctgca |
| | | cacatacatctctttcctttatacttcccttcaaaagacaaatat |
| | | cttacttttgatctttgacactatttggtcagtattcttctttac |
| | | ctttacttgtggcaaaactcaaggaagcgatcaaatagagggaag |
| | | ctcatttctatcattgtctctgtttccctataagaaagaactacc |
| | | agggactcactgactgcattaggcatacaatgtcagagctgagct |
| | | gaccactctggtcctgtaatgtctttggcctcacaccttggcagc |
| | | catcattaatgggccatacccttccccaggtgcagaattctcctc |
| | | cccagagcactcaggccgttactaccaatttatctgagttggaaa |
| | | taagactcatttgccagttcttatttttaaagtggcaccctttaa |
| | | ctttgaacctgtgtattttacactggcatcctagattcagcaatg |
| | | aggtttggtggtgtttcaactaggaagggagaaaatgagtgcatc |
| | | tgaagttccttacagcttggtttctttggaatgctttcatcttct |
| | | aagcaaagggatcagggtttgatctgtaagagttaaaaagacaaa |
| | | gtcattttgaagaattaactcagccagggatcatgcaaaaagatt |
| | | agaaaccataatgcccttgttaaagccctgctgtcaacctgcctt |
| | | cacccagagcttagagggccacagcagcaaagaggttggggtcca |
| | | tccctctctgatgtgctttttccacaacacatatctggtcctctg |
| | | gcaggattgtggatagagctcctcaccatacccaaaagactcagc |
| | | cccagtgccagtgctttcctggttcaacaacccaccacaaaacct |
| | | tagtaaaaggatgagccaaaaatgaaaaagactcgactctacagt |
| | | aagtcagtcagggatttcctttttaatggtttaagacatccaaat |
| | | ggcaagccaggaatagataccattaaagggtctcataggactaac |
| | | cttaccagagccagaaatctagctctctggaagagatgcaagatt |
| | | ctagaaaagtaaagggaagtgtcggcacatctaaatttagtgaac |
| | | acaaaattaatttttatctagtctgtgacggagggaataaagttt |
| | | ttcatgtatcaaccacctcccccagtcaggtttctccctttttga |
| | | gattatgaagaagctgagacatacttcttaaggaggtcgtgtttt |
| | | agaaggaaaaggcagaggctatccatcattatgctggctagatgc |
| | | gcttctgaagaagccggattctgatgttcttaaccaaaatggtga |
| | | ggtcatggaagtcccatttgcttggagattttgaaaaaaaaaaaa |
| | | aaaaaaaacccattcccataaagtaattgagttcagcctttggat |
| | | tatttttggtttggtttttctctggttttgggtgtgatgtaagaa |
| | | gagctttttagttttgttttgaataacatcaatccttgcacactc |
| | | tatgcaaaaattttgtaagcatttcaataatgctatgaattacaa |
| | | ggaactattttaactttattacactttctgtataaaaaatttgta |
| | | tttaatattatttcgaccacagtcttgtaaaatatattaataaaa |
| | | ataatgattggtaagaaggaaa |
| 32 | SGCD (Genbank | agagctgctcagcacgccctgggatcgcgggcggttttcatcggc |
| | Accession No. | cggtttgtgaaacggacaagagagagacattactgccgggagtgt |
| | NM_172244.3) | tgagtgaagggaccaggtggagatgatgcctcaggagcagtacac |
| | | tcaccaccggagcaccatgcctggctctgtggggccacaggtata |
| | | caaggtggggatttatggctggcggaaacgatgcctgtatttctt |
| | | tgtcctgctcctcatgattttaatactggtgaacttggccatgac |
| | | catctggattctcaaagtcatgaacttcacaattgatggaatggg |
| | | aaacctgaggatcacagaaaaaggtctaaagctagaaggagactc |
| | | tgaattcttacaacctctctacgccaaagaaatccagtcccgacc |
| | | aggtaatgccctgtacttcaagtctgccagaaatgttacagtgaa |
| | | cattctcaatgaccagactaaagtgctaactcagcttataacagg |
| | | tccaaaagccgtagaagcttatggtaaaaaatttgaggtaaaaac |
| | | tgtttctggaaaattgctcttctctgcagacaataatgaagtggt |
| | | agtaggagctgaaagattacgagttttaggagcggagggcacagt |
| | | gttccctaaatctatagaaacacctaatgtcagggcagacccctt |
| | | caaagaactaaggttggagtccccaacccggtctctagtgatgga |
| | | ggccccaaaaggagtggaaatcaatgcagaagctggcaatatgga |
| | | agccacctgcaggacagagctgagactggaatccaaagatggaga |
| | | ggtgagggatgagaaggacagaagttcaaagagctacagcttcaa |
| | | caggccaacccttcccataactggttgacctcggagttggatcct |
| | | acagtgtatcaacaaaaggagccaagcaggttttatttctgaaac |
| | | aattaattgagcagcatgattataagccaaacccacaatccatca |
| | | aagtgatgatttcttatttgtaaaatgcagagataatggcatgta |
| | | ttccaagtacagaattatatgaccatgaaaatgaatgctattttc |
| | | aaattctctcttgtcaccttaaaataagattttgttagccaacat |
| | | aattaagctgtatatattatacacatctggctcaagatgaa |
| 33 | SGCD (Genbank | MetMetProGlnGluGlnTyrThrHisHisArgSerThrMetPro |
| | Accession No. | GlySerValGlyProGlnValTyrLysValGlyIleTyrGlyTrp |
| | NP_000328.2) | ArgLysArgCysLeuTyrPhePheValLeuLeuLeuMetIleLeu |
| | | IleLeuValAsnLeuAlaMetThrIleTrpIleLeuLysValMet |
| | | AsnPheThrIleAspGlyMetGlyAsnLeuArgIleThrGluLys |
| | | GlyLeuLysLeuGluGlyAspSerGluPheLeuGlnProLeuTyr |
| | | AlaLysGlnIleGlnSerArgProGlyAsnAlaLeuTyrPheLys |
| | | SerAlaArgAsnValThrValAsnIleLeuAsnAspGlnThrLys |
| | | ValLeuThrGlnLenIleThrGlyProLysAlaValGluAlaTyr |
| | | GlyLysLysPheGluValLysThrValSerGlyLysLeuLeuPhe |
| | | SerAlaAspAsnAsnGluValValValGlyAlaGluArgLeuArg |
| | | ValLeuGlyAlaGluGlyThrValPheProLysSerIleGluThr |
| | | ProAsnValArgAlaAspProPheLysGluLeuArgLeuGluSer |
| | | ProThrArgSerLeuValMetGluAlaProLysGlyValGluIle |
| | | AsnAlaGluAlaGlyAsnMetGluAlaThrCysArgThrGluLeu |
| | | ArgLeuGluSerLysAspGlyGlnIleLysLeuAspAlaAlaLys |
| | | IleArgLeuProArgLeuProHisGlySerTyrThrProThrGly |
| | | ThrArgGlnLysValPheGlnIleCysValCysAlaAsnGlyArg |
| | | LeuPheLeuSerGlnAlaGlyAlaGlySerThrCysGlnIleAsn |
| | | ThrSerValCysLeu |
| 34 | SGCD (Genbank | MetProGlnGluGlnTyrThrHisHisArgSerThrMetProGly |
| | Accession No. | SerValGlyProGlnValTyrLysValGlyIleTyrGlyTrpArg |
| | NP_001121681.1) | LysArgCysLeuTyrPhePheValLeuLeuLeuMetIleLeuIle |
| | | LeuValAsnLeuAlaMetThrIleTrpIleLeuLysValMetAsn |
| | | PheThrIleAspGlyMetGlyAsnLeuArgIleThrGluLysGly |
| | | LeuLysLeuGluGlyAspSerGluPheLeuGlnProLeuTyrAla |
| | | LysGlnIleGlnSerArgProGlyAsnAlaLeuTyrPheLysSer |
| | | AlaArgAsnValThrValAsnIleLeuAsnAspGlnThrLysVal |
| | | LeuThrGlnLeuIleThrGlyProLysAlaValGluAlaTyrGly |
| | | LysLysPheGluValLysThrValSerGlyLysLeuLeuPheSer |
| | | AlaAspAsnAsnGluValValValGlyAlaGluArgLeuArgVal |
| | | LeuGlyAlaGluGlyThrValPheProLysSerIleGluThrPro |
| | | AsnValArgAlaAspProPheLysGluLeuArgLeuGluSerPro |
| | | ThrArgSerLeuValMetGluAlaProLysGlyValGluIleAsn |
| | | AlaGluAlaGlyAsnMetGluAlaThrCysArgThrGluLeuArg |
| | | LeuGluSerLysAspGlyGluIleLysLeuAspAlaAlaLysIle |
| | | ArgLeuProArgLeuProHisGlySerTyrThrProThrGlyThr |
| | | ArgGlnLysValPheGluIleCysValCysAlaAsnGlyArgLeu |
| | | PheLeuSerGlnAlaGlyAlaGlySerThrCysGlnIleAsnThr |
| | | SerValCysLeu |
| 35 | SGCD (Genbank | MetMetProGlnGluGlnTyrThrHisHisArgSerThrMetPro |
| | Accession No. | GlySerValGlyProGlnValTyrLysValGlyIleTyrGlyTrp |
| | NP_758447.1) | ArgLysArgCysLeuTyrPhePheValLeuLeuLeuMetIleLeu |
| | | IleLeuValAsnLeuAlaMetThrIleTrpIleLeuLysValMet |
| | | AsnPheThrIleAspGlyMetGlyAsnLeuArgIleThrGluLys |
| | | GlyLeuLysLeuGluGlyAspSerGluPheLeuGlnProLeuTyr |
| | | AlaLysGlnIleGlnSerArgProGlyAsnAlaLeuTyrPheLys |
| | | SerAlaArgAsnValThrValAsnIleLeuAsnAspGlnThrLys |
| | | ValLeuThrGlnLenIleThrGlyProLysAlaValGluAlaTyr |
| | | GlyLysLysPheGluValLysThrValSerGlyLysLeuLeuPhe |
| | | SerAlaAspAsnAsnGluValValValGlyAlaGluArgLeuArg |
| | | ValLeuGlyAlaGluGlyThrValPheProLysSerIleGluThr |
| | | ProAsnValArgAlaAspProPheLysGluLeuArgLeuGluSer |
| | | ProThrArgSerLeuValMetGluAlaProLysGlyValGluIle |
| | | AsnAlaGluAlaGlyAsnMetGluAlaThrCysArgThrGluLeu |
| | | ArgLeuGluSerLysAspGlyGluValArgAspGluLysAspArg |
| | | SerSerLysSerTyrSerPheAsnArgProThrLeuProIleThr |
| | | Gly |
| 36 | Dystrophin | gaagtcataactgatagaagatcatctactttgtttacatgtttg |
| | (Genbank | aatcatatagattcaagtcagttaatttcactaaaactcatcaat |
| | Accession No. | tattattcatcaattagggtaaatgtatttaaaaaattgtttttt |
| | AH003182.2) | aggctttacaaagttctctgcaagagcaacaaagtggcctatact |
| | | atctcagcaccactgtgaaagagatgtcgaagaaagcgccctctg |
| | | aaattagccggaaatatcaatcagaatttgaagaaattgagggac |
| | | gctggaagaagctctcctcccagctggttgagcattgtcaaaagc |
| | | tagaggagcaaatgaataaactccgaaaaattcaggtaattcagg |
| | | attttactttctaccctcatttttatttacttgttttttccctaa |
| | | cgatacactgtaaactgtaaaggtacnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnataaaaatg |
| | | cagtmttagtaaaaatattctttgcctmaagaactacttagagac |
| | | atcctttaaacatgggaattgtttttgggcctgtgtttagacata |
| | | acacaatgatgaattgtgtnaaaagtaatcagcacaccagtaatg |
| | | ccttataacgggtctcgtttcagaatcacatacaaaccctgaaga |
| | | aatggatggctgaagttgatgtttttctgaaggaggaatggcctg |
| | | cccttggggattcagaaattctaaaaaagcagctgaaacagtgca |
| | | gagtaagatttttatatgatgcctttaatatgaataattttgtat |
| | | gaatatnatttggttagatcagtgttttacagctggggtggattt |
| | | tgctctcctctccccnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnaagactgttaggcagtcatc |
| | | tatatcaaatatctctgtatctcaagatcccaaaagacaaaaatc |
| | | caatatgcaatgccatcagttcccaattttctagctatgtttcat |
| | | atctatatgtggcagtaatttttttcagctggcttaaattgattt |
| | | attttcttagcttttagtcagtgatattcagacaattcagcccag |
| | | tctaaacagtgtcaatgaaggtgggcagaagataaagaatgaagc |
| | | agagccagagtttgcttcgagacttgagacagaactcaaagaact |
| | | taacactcagtgggatcacatgtgccaacaggtatagacaatctc |
| | | tttcactgaggcttgcctcaacgtacttaactaagatttcctaat |
| | | gtctcccttcaccgttacttttggttaaggctttgttcctatgtt |
| | | tttgctttaaagcacnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnctcatgcatataagcttttt |
| | | acttctttatcattactatctaagctttctattctttacatactg |
| | | atgaaataatataataataatgtttcatcactgtcaataatcgtg |
| | | ttttgtttgtttgttttgtggaaggtctatgccagaaaggaggcc |
| | | ttgaagggaggtttggagaaaactgtaagcctccagaaagatcta |
| | | tcagagatgcacgaatggatgacacaagctgaagaagagtatctt |
| | | gagagagattttgaatataaaactccagatgaattacagaaagca |
| | | gttgaagagatgaaggtaaaaaaaaaaaagaaaaactaagtaaaa |
| | | caaaggaaataaatggaaaaagaaagaaatgcaacaatgcttgaa |
| | | gtcgtatacagtctgctctttcctggttctaagagaagaggttga |
| | | ttcttcattnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnagagctaaagaagaggcccaacaaaa |
| | | agaagcgaaagtgaaactccttactgagtctgtaaatagtgtcat |
| | | agctcaagctccacctgtagcacaagaggccttaaaaaaggaact |
| | | tgaaactctaaccaccaactaccagtggctctgcactaggctgaa |
| | | tgggaaatgcaagactttggaagtcagttgcttttcttggtcttt |
| | | gtcaattatatgtcaatacatggtcatagttannnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnngaa |
| | | gttttaataatgaaatggcaaaatttcacatttacttttctacca |
| | | taatatttaatctgtgatatatatttctttcttaggaagtttggg |
| | | catgttggcatgagttattgtcatacttggagaaagcaaacaagt |
| | | ggctaaatgaagtagaatttaaacttaaaaccactgaaaacattc |
| | | ctggcggagctgaggaaatctctgaggtgctagatgtaagttgta |
| | | aattaagccaaatgatgatgatttatatgcagtattaaaagaggt |
| | | acnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnntttgctttaagattatgancttaatgatatgta |
| | | aatcagaagatactgagcatttgctgataatccaatgtatttaga |
| | | aaaaaaaggagaaatngtaattattgcaaatgtgtttcagtcact |
| | | tgaaaatttgatgcgacattcagaggataacccaaatcagattcg |
| | | catattggcacagaccctaacagatggcggagtcatggatgagct |
| | | aatcaatgaggaacttgagacatttaattctcgttggagggaact |
| | | acatgaagaggtattaagataagtgaaaatctctttaatctaatt |
| | | tgcattaatgtatagcagatacagctctcagatatannnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nagctatgaagggtaatcgttttacctgatacagtyttattyctt |
| | | ctttttaggctgtaaggaggcaaaagttgcttgaacagagcatcc |
| | | agtctgcccaggagactgaaaaatccttacacttaatccaggagt |
| | | ccctcacattcattgacaagcagttggcagcttatattgcagaca |
| | | aggtggacgcagctcaaatgcctcaggaagcccaggcaagtacat |
| | | ctgggaatcagcttccattctttttgtttgtatgacctcacgnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnaacgtgtaagaaaatagttattttaaataacagaaata |
| | | taaaagttccaaataagtggttataacgaaatttgaattaaagag |
| | | taaactaaattacatttcataataattcttttcaggtaacagaaa |
| | | gaaagcaacagttggagaaatgcttgaaattgtcccgtaagatgc |
| | | gaaaggaaatgaatgtcttgacagaatggctggcagctacagata |
| | | tggaattgacaaagagatcagcagttgaaggaatgcctagtaatt |
| | | tggattctgaagttgcctggggaaaggtaaaacctatatcactga |
| | | aggttattttgaacatacgtgaaaacacataatatgattttgtaa |
| | | ggaagtattaacatgtagcaataatagcatcataaatattaatat |
| | | tgtttcatattccctttcttaaaattaannnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnaataaat |
| | | gtatttcttttggtttatgtttcttataaaaagtaattttgattt |
| | | aaagtagractacctttttttttaggcctccattcctttgaagga |
| | | attggagcagtttaactcagatatacaaaaattgcttgaaccact |
| | | ggaggctgaaattcagcagggggtgaatctgaaagaggaagactt |
| | | caataaagatatggtaaattggttgtgaatgaactaggagtggaa |
| | | ataaatatttggaaagaacttagataagnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnggattaa |
| | | agtgtgtgtttaaataacatgtctaattatctctgttaacaatgt |
| | | acagctttttaaaaaccaaaatgaagactgtacttgttgtttttg |
| | | atcagaatgaagacaatgagggtactgtaaaagaattgttgcaaa |
| | | gaggagacaacttacaacaaagaatcacagatgagagaaagcgag |
| | | aggaaataaagataaaacagcagctgttacagacaaaacataatg |
| | | ctctcaaggtattagagctaaaattataatataccttgcctgtgg |
| | | tttttttttaatatatagggtaatatataatgtgcattaataaaa |
| | | tctgcttcagactcttagtcatcagaaactcactttttctgttca |
| | | atgtgtatgcttatttaacatttttgaggtggtatttnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnaacctaagttgtacaaaaagatgagggacgcaagttattttga |
| | | taaataactgcagccagaagtgcactatacatatatattgatatt |
| | | ttaataatgtctgcaccatgaacaggatttgaggtctcaaagaag |
| | | aaaaaaggctctagaaatttctcatcagtggtatcagtacaagag |
| | | gcaggctgatgatctcctgaaatgcttggatgacattgaaaaaaa |
| | | attagccagcctacctgagcccagagatgaaaggaaaataaaggt |
| | | aatgttgtgttttagaatgtcaataccagattttattatacagtt |
| | | taattaacctgtgaagatcatatttaaaatgttgatgttcttgtt |
| | | tctattaacgttctctttgagggatgcatttccttgtattgtgtg |
| | | ttgttttcagtaaatgtattgtattaaggtgttattcaattgacn |
| | | nnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnaatnacattcatagcttgtttcttttctttrgtaat |
| | | tctgcacatattcttcttcctgctgtcctgtaggacctccaaggt |
| | | gaaattgaagctcacacagatgtttatcacaacctggatgaaaac |
| | | agccaaaaaatcctgagatccctggaaggttccgatgatgcagtc |
| | | ctgttacaaagacgtttggataacatgaacttcaagtggagtgaa |
| | | cttcggaaaaagtctctcaacattaggtaggaaaagatgtggagc |
| | | aaaaaggccacaaataaatnaaaatggccaaattttcctcattgt |
| | | cttagcacaagtaactggtatctcacatgtctacgtaaatcatcc |
| | | caatttcagnnnnnnnnnnnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnntaaccatcagtgtataaataaaagag |
| | | atagaaattgacctggagtttcataaacaagttctgagcacccag |
| | | gattaattttgagaagaatgccacaagcctttcttagcacttctt |
| | | ttcatctcatttcacaggccttcaagagggaattgaaaactaaag |
| | | aacctgtaatcatgagtactcttgagactgtacgaatatttctga |
| | | cagagcagcctttggaaggactagagaaactctaccaggagccca |
| | | gaggtaattgaatgtggaactataataacatattgatagaaggat |
| | | cagtggtgacggagcagcccatccattcttgctgccagggtctgg |
| | | atagctctcatattttcttnnnnnnnnnnnnnnnnnnnnnnnnnn |
| | | nnnnnnnnnnnnnnnnnnnnnnnnnnnnnnnnnnnnnnnnnnnnn |
| | | nnnnnnnnnnnnnnnnnnnnnnnnnnnnnaaacgtcggattgata |
| | | taaggtagaaactcaggaagatatactttagatgttctgggctgt |
| | | atcaaaatttatgccaaagtataaaaaagccgttaatcagtaggt |
| | | taccctcttgttcaactgtactctttctttcttccagtatgacct |
| | | ttttgacaatgtttaaaaaaaaagaatgtggcctaaaaccttgtc |
| | | atattgccaatttagagctgcctcctgaggagagagcccagaatg |
| | | tcactcggcttctacgaaagcaggctgaggaggtcaatactgagt |
| | | gggaaaaattgaacctgcactccgctgactggcagagaaaaatag |
| | | atgagacccttgaaagactccaggaacttcaagaggccacggatg |
| | | agctggacctcaagctgcgccaagctgaggtgatcaagggatcct |
| | | ggcagcccgtgggcgatctcctcattgactctctccaagatcacc |
| | | tcgagaaagtcaaggtacggtctacttctttact |
| 37 | Dystrophin | AlaLeuGlnSerSerLeuGlnGluGlnGlnSerGlyLeuTyrTyr |
| | (Genbank | LeuSerThrThrValLysGluMetSerLysLysAlaProSerGlu |
| | Accession No. | IleSerArgLysTyrGlnSerGluPheGluGluIleGluGlyArg |
| | AAA74506.1) | TrpLysLysLeuSerSerGlnLeuValGluHisCysGlnLysLeu |
| | | GluGluGlnMetAsnLysLeuArgLysIleGlnAsnHisIleGln |
| | | ThrLeuLysLysTrpMetAlaGluValAspValPheLeuLysGlu |
| | | GluTrpProAlaLeuGlyAspSerGlnIleLeuLysLysGlnLeu |
| | | LysGlnCysArgLeuLeuValSerAspIleGlnThrIleGlnPro |
| | | SerLeuAsnSerValAsnGluGlyGlyGlnLysIleLysAsnGlu |
| | | AlaGluProGluPheAlaSerArgLeuGluThrGluLeuLysGlu |
| | | LeuAsnThrGlnTrpAspHisMetCysGlnGlnValTyrAlaArg |
| | | LysGluAlaLeuLysGlyGlyLeuGluLysThrValSerLeuGln |
| | | LysAspLeuSerGluMetHisGluTrpMetThrGlnAlaGluGlu |
| | | GluTyrLeuGluArgAspPheGluTyrLysThrProAspGluLeu |
| | | GlnLysAlaValGluGluMetLysArgAlaLysGluGluAlaGln |
| | | GlnLysGluAlaLysValLysLeuLeuThrGluSerValAsnSer |
| | | ValIleAlaGlnAlaProProValAlaGlnGluAlaLeuLysLys |
| | | GluLeuGluThrLeuThrThrAsnTyrGlnTrpLeuCysThrArg |
| | | LeuAsnGlyLysCysLysThrLeuGluGluValTrpAlaCysTrp |
| | | HisGluLeuLeuSerTyrLeuGluLysAlaAsnLysTrpLeuAsn |
| | | GluValGluPheLysLeuLysThrThrGluAsnIleProGlyGly |
| | | AlaGluGlnIleSerGlnValLeuAspSerLeuGluAsnLeuMet |
| | | ArgHisSerGluAspAsnProAsnGlnIleArgIleLeuAlaGln |
| | | ThrLeuThrAspGlyGlyValMetAspGluLenIleAsnGluGlu |
| | | LeuGluThrPheAsnSerArgTrpArgGluLeuHisGInGluAla |
| | | ValArgArgGlnLysLeuLeuGluGlnSerIleGlnSerAlaGln |
| | | GluThrGluLysSerLeuHisLeuIleGlnGluSerLeuThrPhe |
| | | IleAspLysGlnLeuAlaAlaTyrIleAlaAspLysValAspAla |
| | | AlaGlnMetProGlnGluAlaGln |
| 38 | Microdystrophin | atgctgtggtgggaggaggtggaggattgttatgaaagggaggac |
| | nucleotide | gtgcagaagaagacttttaccaagtgggtgaacgctcagttcagc |
| | sequence | aaatttgggaagcagcacatcgagaatctgttttccgacctgcag |
| | (artificial | gatgggagacggctgctggatctgctggaaggactgactggccag |
| | sequence) | aagctgcccaaagagaaggggagcactagggtgcacgccctgaac |
| | | aacgtgaacaaagctctgagagtgctgcagaacaacaacgtggat |
| | | ctggtgaatattggcagtactgatatcgtggacgggaaccacaaa |
| | | ctgacactgggcctgatctggaacattattctgcactggcaggtg |
| | | aaaaatgtgatgaagaacatcatggccgggctgcagcagaccaat |
| | | tccgagaagatcctgctgtcttgggtgcggcagagcacccgcaac |
| | | tatccccaggtgaacgtgattaacttcactacatcctggagcgac |
| | | gggctggccctgaatgctctgattcacagccacaggcctgatctg |
| | | ttcgactggaatagcgtggtgtgccagcagtctgccacacagcgc |
| | | ctggaacatgccttcaatatcgctcggtaccagctggggatcgaa |
| | | aaactgctggacccagaggatgtggacactacatacccagataaa |
| | | aagtctattctgatgtacattactagcctgttccaggtgctgcca |
| | | cagcaggtgtctattgaagccattcaggaggtggaaatgctgccc |
| | | cgcccccccaaagtgactaaagaggagcattttcagctgcatcat |
| | | cagatgcattacagccagcagattaccgtgagcctggctcaggga |
| | | tatgagcgcaccagtagtccaaaaccacggttcaagtcctacgct |
| | | tatacccaggctgcctacgtgacaactagcgaccctactagatcc |
| | | ccctttccatcccagcacctggaggccccagaggacaagagcttt |
| | | gggtccagcctgatggaaagcgaggtgaatctggatcggtaccag |
| | | acagccctggaggaggtgctgagctggctgctgagtgctgaagac |
| | | acactgcaggcccagggcgaaatttccaatgacgtggaagtggtg |
| | | aaggatcagttccacacacacgagggctatatgatggacctgaca |
| | | gctcaccaggggcgcgtgggcaatatcctgcagctgggctctaaa |
| | | ctgatcggcaccgggaaactgagtgaggacgaggaaacagaagtg |
| | | caggagcagatgaacctgctgaacagccgctgggagtgtctgaga |
| | | gtggctagtatggagaagcagtccaacctgcaccgggtgctgatg |
| | | gacctgcagaaccagaaactgaaagagctgaacgactggctgaca |
| | | aagactgaggaacgcacaaggaagatggaggaggagccactggga |
| | | cccgacctggaggatctgaagagacaggtgcagcagcataaggtg |
| | | ctgcaggaggatctggaacaggagcaggtgcgggtgaactccctg |
| | | acacatatggtggtggtggtggacgaatctagtggagatcacgcc |
| | | accgccgccctggaggaacagctgaaggtgctgggggaccggtgg |
| | | gccaacatttgccggtggaccgaggacaggtgggtgctgctgcag |
| | | gacatcctgctgaaatggcagaggctgaccgaggagcagtgtctg |
| | | tttagtgcttggctgagcgagaaagaggacgccgtgaacaagatc |
| | | cacacaaccggctttaaggatcagaacgaaatgctgtctagcctg |
| | | cagaaactggctgtgctgaaggccgatctggagaaaaagaagcag |
| | | agcatgggcaaactgtatagcctgaaacaggacctgctgagcacc |
| | | ctgaagaacaagagcgtgacccagaagacagaagcctggctggat |
| | | aactttgcccgctgctgggacaacctggtgcagaaactggagaaa |
| | | agtacagctcagatctctcaggctgtgaccacaacccagcctagc |
| | | ctgacccagacaaccgtgatggaaaccgtgaccaccgtgacaacc |
| | | cgcgaacagatcctggtgaaacatgcccaggaagagctgccacct |
| | | ccacctccccagaagaagagaaccctggagcggctgcaggagctg |
| | | caggaagccactgacgaactggacctgaagctgaggcaggccgaa |
| | | gtgattaaggggtcttggcagcctgtgggcgatctgctgattgat |
| | | tccctgcaggaccacctggaaaaggtgaaggctctgagaggcgaa |
| | | attgctccactgaaggagaacgtgagtcatgtgaacgatctggct |
| | | agacagctgacaacactgggcatccagctgagcccatacaatctg |
| | | agcacactggaggacctgaataccaggtggaagctgctgcaggtg |
| | | gctgtggaagaccgggtgcggcagctgcatgaggcccatcgcgac |
| | | ttcggaccagccagccagcactttctgagcacatccgtgcagggg |
| | | ccctgggagagggccatttctcccaacaaggtgccctactatatt |
| | | aatcacgagacccagaccacttgttgggaccatcccaagatgaca |
| | | gaactgtaccagtccctggccgatctgaacaacgtgaggtttagc |
| | | gcttacagaaccgctatgaagctgagacggctgcagaaggccctg |
| | | tgcctggatctgctgtccctgtccgccgcctgcgatgccctggat |
| | | cagcataatctgaagcagaacgatcagccaatggatatcctgcag |
| | | atcatcaactgcctgaccactatctacgacaggctggagcaggag |
| | | cacaacaacctggtgaacgtgcctctgtgcgtggatatgtgcctg |
| | | aactggctgctgaacgtgtatgacactgggcgcaccggccggatc |
| | | agagtgctgagttttaaaactgggattatctccctgtgtaaggcc |
| | | cacctggaggacaagtacaggtacctgttcaagcaggtggctagt |
| | | agcactggattttgtgaccagcgccgcctgggactgctgctgcat |
| | | gatagtatccagattcctagacagctgggagaggtggctagtttc |
| | | ggaggatctaacatcgaacccagcgtgcgcagctgtttccagttt |
| | | gccaataacaaacctgaaatcgaggctgctctgttcctggattgg |
| | | atgcgcctggaaccacagagcatggtgtggctgcctgtgctgcac |
| | | agagtggctgccgccgaaactgccaagcaccaggctaaatgcaac |
| | | atctgcaaggaatgtcccattatcggctttcgctacaggagtctg |
| | | aaacattttaactacgatatttgccagagctgcttcttttccgga |
| | | agagtggccaaaggacacaagatgcactaccctatggtggaatat |
| | | tgcaccccaactacatctggcgaagatgtgcgcgattttgccaag |
| | | gtgctgaagaataagtttcggactaagaggtacttcgccaagcac |
| | | ccccgcatggggtatctgccagtgcagacagtgctggaaggagac |
| | | aatatggagaccgatacaatgtgagc |
| 39 | Microdystrophin | MetLeuTrpTrpGluGluValGluAspCysTyrGluArgGluAsp |
| | amino acid | ValGlnLysLysThrPheThrLysTrpValAsnAlaGlnPheSer |
| | sequence | LysPheGlyLysGlnHisIleGluAsnLeuPheSerAspLeuGln |
| | (artificial | AspGlyArgArgLeuLeuAspLeuLeuGluGlyLeuThrGlyGln |
| | sequence) | LysLeuProLysGluLysGlySerThrArgValHisAlaLeuAsn |
| | | AsnValAsnLysAlaLeuArgValLeuGlnAsnAsnAsnValAsp |
| | | LeuValAsnIleGlySerThrAspIleValAspGlyAsnHisLys |
| | | LeuThrLeuGlyLenIleTrpAsnIleIleLeuHisTrpGlnVal |
| | | LysAsnValMetLysAsnIleMetAlaGlyLeuGlnGlnThrAsn |
| | | SerGluLysIleLeuLeuSerTrpValArgGlnSerThrArgAsn |
| | | TyrProGlnValAsnValIleAsnPheThrThrSerTrpSerAsp |
| | | GlyLeuAlaLeuAsnAlaLeuIleHisSerHisArgProAspLeu |
| | | PheAspTrpAsnSerValValCysGlnGlnSerAlaThrGlnArg |
| | | LeuGluHisAlaPheAsnIleAlaArgTyrGlnLeuGlyIleGlu |
| | | LysLeuLeuAspProGluAspValAspThrThrTyrProAspLys |
| | | LysSerIleLeuMetTyrIleThrSerLeuPheGlnValLeuPro |
| | | GlnGlnValSerIleGluAlaIleGlnGluValGluMetLeuPro |
| | | ArgProProLysValThrLysGluGluHisPheGlnLeuHisHis |
| | | GlnMetHisTyrSerGlnGlnIleThrValSerLeuAlaGlnGly |
| | | TyrGInArgThrSerSerProLysProArgPheLysSerTyrAla |
| | | TyrThrGlnAlaAlaTyrValThrThrSerAspProThrArgSer |
| | | ProPheProSerGlnHisLeuGluAlaProGluAspLysSerPhe |
| | | GlySerSerLeuMetGluSerGluValAsnLeuAspArgTyrGln |
| | | ThrAlaLeuGluGluValLeuSerTrpLeuLeuSerAlaGluAsp |
| | | ThrLeuGlnAlaGlnGlyGluIleSerAsnAspValGluValVal |
| | | LysAspGlnPheHisThrHisGluGlyTyrMetMetAspLeuThr |
| | | AlaHisGlnGlyArgValGlyAsnIleLeuGlnLeuGlySerLys |
| | | LenIleGlyThrGlyLysLeuSerGluAspGluGluThrGluVal |
| | | GlnGluGlnMetAsnLeuLeuAsnSerArgTrpGluCysLeuArg |
| | | ValAlaSerMetGluLysGlnSerAsnLeuHisArgValLeuMet |
| | | AspLeuGlnAsnGlnLysLeuLysGluLeuAsnAspTrpLeuThr |
| | | LysThrGluGluArgThrArgLysMetGluGluGluProLeuGly |
| | | ProAspLeuGluAspLeuLysArgGlnValGlnGlnHisLysVal |
| | | LeuGlnGluAspLeuGluGlnGluGlnValArgValAsnSerLeu |
| | | ThrHisMetValValValValAspGluSerSerGlyAspHisAla |
| | | ThrAlaAlaLeuGluGluGlnLeuLysValLeuGlyAspArgTrp |
| | | AlaAsnIleCysArgTrpThrGluAspArgTrpValLeuLeuGln |
| | | AspIleLeuLeuLysTrpGlnArgLeuThrGluGluGlnCysLeu |
| | | PheSerAlaTrpLeuSerGluLysGluAspAlaValAsnLysIle |
| | | HisThrThrGlyPheLysAspGlnAsnGluMetLeuSerSerLeu |
| | | GlnLysLeuAlaValLeuLysAlaAspLeuGluLysLysLysGln |
| | | SerMetGlyLysLeuTyrSerLeuLysGlnAspLeuLeuSerThr |
| | | LeuLysAsnLysSerValThrGlnLysThrGluAlaTrpLeuAsp |
| | | AsnPheAlaArgCysTrpAspAsnLeuValGlnLysLeuGluLys |
| | | SerThrAlaGlnIleSerGlnAlaValThrThrThrGlnProSer |
| | | LeuThrGlnThrThrValMetGluThrValThrThrValThrThr |
| | | ArgGluGlnIleLeuValLysHisAlaGlnGluGluLeuProPro |
| | | ProProProGlnLysLysArgThrLeuGluArgLeuGlnGluLeu |
| | | GlnGluAlaThrAspGluLeuAspLeuLysLeuArgGlnAlaGlu |
| | | ValIleLysGlySerTrpGlnProValGlyAspLeuLenIleAsp |
| | | SerLeuGlnAspHisLeuGluLysValLysAlaLeuArgGlyGlu |
| | | IleAlaProLeuLysGluAsnValSerHisValAsnAspLeuAla |
| | | ArgGlnLeuThrThrLeuGlyIleGlnLeuSerProTyrAsnLeu |
| | | SerThrLeuGluAspLeuAsnThrArgTrpLysLeuLeuGlnVal |
| | | AlaValGluAspArgValArgGlnLeuHisGluAlaHisArgAsp |
| | | PheGlyProAlaSerGlnHisPheLeuSerThrSerValGInGly |
| | | ProTrpGluArgAlaIleSerProAsnLysValProTyrTyrIle |
| | | AsnHisGluThrGlnThrThrCysTrpAspHisProLysMetThr |
| | | GluLeuTyrGlnSerLeuAlaAspLeuAsnAsnValArgPheSer |
| | | AlaTyrArgThrAlaMetLysLeuArgArgLeuGlnLysAlaLeu |
| | | CysLeuAspLeuLeuSerLeuSerAlaAlaCysAspAlaLeuAsp |
| | | GlnHisAsnLeuLysGInAsnAspGlnProMetAspIleLeuGln |
| | | IleIleAsnCysLeuThrThrIleTyrAspArgLeuGluGlnGlu |
| | | HisAsnAsnLeuValAsnValProLeuCysValAspMetCysLeu |
| | | AsnTrpLeuLeuAsnValTyrAspThrGlyArgThrGlyArgIle |
| | | ArgValLeuSerPheLysThrGlyIleIleSerLeuCysLysAla |
| | | HisLeuGluAspLysTyrArgTyrLeuPheLysGlnValAlaSer |
| | | SerThrGlyPheCysAspGlnArgArgLeuGlyLeuLeuLeuHis |
| | | AspSerIleGlnIleProArgGlnLeuGlyGluValAlaSerPhe |
| | | GlyGlySerAsnIleGluProSerValArgSerCysPheGlnPhe |
| | | AlaAsnAsnLysProGluIleGluAlaAlaLeuPheLeuAspTrp |
| | | MetArgLeuGluProGlnSerMetValTrpLeuProValLeuHis |
| | | ArgValAlaAlaAlaGluThrAlaLysHisGlnAlaLysCysAsn |
| | | IleCysLysGluCysProIleIleGlyPheArgTyrArgSerLeu |
| | | LysHisPheAsnTyrAspIleCysGlnSerCysPhePheSerGly |
| | | ArgValAlaLysGlyHisLysMetHisTyrProMetValGluTyr |
| | | CysThrProThrThrSerGlyGluAspValArgAspPheAlaLys |
| | | ValLeuLysAsnLysPheArgThrLysArgTyrPheAlaLysHis |
| | | ProArgMetGlyTyrLeuProValGlnThrValLeuGluGlyAsp |
| | | AsnMetGluThrAspThrMet |
| 40 | Mini dystrophin | GGGATTCCCTCACTTTCCCCCTACAGGACTCAGATCTGGGAGGCA |
| | AR4-R23 | ATTACCTTCGGAGAAAAACGAATAGGAAAAACTGAAGTGTTACTT |
| | (artificial | TTTTTAAAGCTGCTGAAGTTTGTTGGTTTCTCATTGTTTTTAAGC |
| | sequence) | CTACTGGAGCAATAAAGTTTGAAGAACTTTTACCAGGTTTTTTTT |
| | | ATCGCTGCCTTGATATACACTTTTCAAAATGCTTTGGTGGGAAGA |
| | | AGTAGAGGACTGTTATGAAAGAGAAGATGTTCAAAAGAAAACATT |
| | | CACAAAATGGGTAAATGCACAATTTTCTAAGTTTGGGAAGCAGCA |
| | | TATTGAGAACCTCTTCAGTGACCTACAGGATGGGAGGCGCCTCCT |
| | | AGACCTCCTCGAAGGCCTGACAGGGCAAAAACTGCCAAAAGAAAA |
| | | AGGATCCACAAGAGTTCATGCCCTGAACAATGTCAACAAGGCACT |
| | | GCGGGTTTTGCAGAACAATAATGTTGATTTAGTGAATATTGGAAG |
| | | TACTGACATCGTAGATGGAAATCATAAACTGACTCTTGGTTTGAT |
| | | TTGGAATATAATCCTCCACTGGCAGGTCAAAAATGTAATGAAAAA |
| | | TATCATGGCTGGATTGCAACAAACCAACAGTGAAAAGATTCTCCT |
| | | GAGCTGGGTCCGACAATCAACTCGTAATTATCCACAGGTTAATGT |
| | | AATCAACTTCACCACCAGCTGGTCTGATGGCCTGGCTTTGAATGC |
| | | TCTCATCCATAGTCATAGGCCAGACCTATTTGACTGGAATAGTGT |
| | | GGTTTGCCAGCAGTCAGCCACACAACGACTGGAACATGCATTCAA |
| | | CATCGCCAGATATCAATTAGGCATAGAGAAACTACTCGATCCTGA |
| | | AGATGTTGATACCACCTATCCAGATAAGAAGTCCATCTTAATGTA |
| | | CATCACATCACTCTTCCAAGTTTTGCCTCAACAAGTGAGCATTGA |
| | | AGCCATCCAGGAAGTGGAAATGTTGCCAAGGCCACCTAAAGTGAC |
| | | TAAAGAAGAACATTTTCAGTTACATCATCAAATGCACTATTCTCA |
| | | ACAGATCACGGTCAGTCTAGCACAGGGATATGAGAGAACTTCTTC |
| | | CCCTAAGCCTCGATTCAAGAGCTATGCCTACACACAGGCTGCTTA |
| | | TGTCACCACCTCTGACCCTACACGGAGCCCATTTCCTTCACAGCA |
| | | TTTGGAAGCTCCTGAAGACAAGTCATTTGGCAGTTCATTGATGGA |
| | | GAGTGAAGTAAACCTGGACCGTTATCAAACAGCTTTAGAAGAAGT |
| | | ATTATCGTGGCTTCTTTCTGCTGAGGACACATTGCAAGCACAAGG |
| | | AGAGATTTCTAATGATGTGGAAGTGGTGAAAGACCAGTTTCATAC |
| | | TCATGAGGGGTACATGATGGATTTGACAGCCCATCAGGGCCGGGT |
| | | TGGTAATATTCTACAATTGGGAAGTAAGCTGATTGGAACAGGAAA |
| | | ATTATCAGAAGATGAAGAAACTGAAGTACAAGAGCAGATGAATCT |
| | | CCTAAATTCAAGATGGGAATGCCTCAGGGTAGCTAGCATGGAAAA |
| | | ACAAAGCAATTTACATAGAGTTTTAATGGATCTCCAGAATCAGAA |
| | | ACTGAAAGAGTTGAATGACTGGCTAACAAAAACAGAAGAAAGAAC |
| | | AAGGAAAATGGAGGAAGAGCCTCTTGGACCTGATCTTGAAGACCT |
| | | AAAACGCCAAGTACAACAACATAAGGTGCTTCAAGAAGATCTAGA |
| | | ACAAGAACAAGTCAGGGTCAATTCTCTCACTCACATGGTGGTGGT |
| | | AGTTGATGAATCTAGTGGAGATCACGCAACTGCTGCTTTGGAAGA |
| | | ACAACTTAAGGTATTGGGAGATCGATGGGCAAACATCTGTAGATG |
| | | GACAGAAGACCGCTGGGTTCTTTTACAAGACATCCTTCTCAAATG |
| | | GCAACGTCTTACTGAAGAACAGTGCCTTTTTAGTGCATGGCTTTC |
| | | AGAAAAAGAAGATGCAGTGAACAAGATTCACACAACTGGCTTTAA |
| | | AGATCAAAATGAAATGTTATCAAGTCTTCAAAAACTGGCCGTTTT |
| | | AAAAGCGGATCTAGAAAAGAAAAAGCAATCCATGGGCAAACTGTA |
| | | TTCACTCAAACAAGATCTTCTTTCAACACTGAAGAATAAGTCAGT |
| | | GACCCAGAAGACGGAAGCATGGCTGGATAACTTTGCCCGGTGTTG |
| | | GGATAATTTAGTCCAAAAACTTGAAAAGAGTACAGCACAGATTTC |
| | | ACAGGCTGTCACCACCACTCAGCCATCACTAACACAGACAACTGT |
| | | AATGGAAACAGTAACTACGGTGACCACAAGGGAACAGATCCTGGT |
| | | AAAGCATGCTCAAGAGGAACTTCCACCACCACCTCCCCAAAAGAA |
| | | GAGGCAGATTACTGTGGATCTTGAAAGACTCCAGGAACTTCAAGA |
| | | GGCCACGGATGAGCTGGACCTCAAGCTGCGCCAAGCTGAGGTGAT |
| | | CAAGGGATCCTGGCAGCCCGTGGGCGATCTCCTCATTGACTCTCT |
| | | CCAAGATCACCTCGAGAAAGTCAAGGCACTTCGAGGAGAAATTGC |
| | | GCCTCTGAAAGAGAACGTGAGCCACGTCAATGACCTTGCTCGCCA |
| | | GCTTACCACTTTGGGCATTCAGCTCTCACCGTATAACCTCAGCAC |
| | | TCTGGAAGACCTGAACACCAGATGGAAGCTTCTGCAGGTGGCCGT |
| | | CGAGGACCGAGTCAGGCAGCTGCATGAAGCCCACAGGGACTTTGG |
| | | TCCAGCATCTCAGCACTTTCTTTCCACGTCTGTCCAGGGTCCCTG |
| | | GGAGAGAGCCATCTCGCCAAACAAAGTGCCCTACTATATCAACCA |
| | | CGAGACTCAAACAACTTGCTGGGACCATCCCAAAATGACAGAGCT |
| | | CTACCAGTCTTTAGCTGACCTGAATAATGTCAGATTCTCAGCTTA |
| | | TAGGACTGCCATGAAACTCCGAAGACTGCAGAAGGCCCTTTGCTT |
| | | GGATCTCTTGAGCCTGTCAGCTGCATGTGATGCCTTGGACCAGCA |
| | | CAACCTCAAGCAAAATGACCAGCCCATGGATATCCTGCAGATTAT |
| | | TAATTGTTTGACCACTATTTATGACCGCCTGGAGCAAGAGCACAA |
| | | CAATTTGGTCAACGTCCCTCTCTGCGTGGATATGTGTCTGAACTG |
| | | GCTGCTGAATGTTTATGATACGGGACGAACAGGGAGGATCCGTGT |
| | | CCTGTCTTTTAAAACTGGCATCATTTCCCTGTGTAAAGCACATTT |
| | | GGAAGACAAGTACAGATACCTTTTCAAGCAAGTGGCAAGTTCAAC |
| | | AGGATTTTGTGACCAGCGCAGGCTGGGCCTCCTTCTGCATGATTC |
| | | TATCCAAATTCCAAGACAGTTGGGTGAAGTTGCATCCTTTGGGGG |
| | | CAGTAACATTGAGCCAAGTGTCCGGAGCTGCTTCCAATTTGCTAA |
| | | TAATAAGCCAGAGATCGAAGCGGCCCTCTTCCTAGACTGGATGAG |
| | | ACTGGAACCCCAGTCCATGGTGTGGCTGCCCGTCCTGCACAGAGT |
| | | GGCTGCTGCAGAAACTGCCAAGCATCAGGCCAAATGTAACATCTG |
| | | CAAAGAGTGTCCAATCATTGGATTCAGGTACAGGAGTCTAAAGCA |
| | | CTTTAATTATGACATCTGCCAAAGCTGCTTTTTTTCTGGTCGAGT |
| | | TGCAAAAGGCCATAAAATGCACTATCCCATGGTGGAATATTGCAC |
| | | TCCGACTACATCAGGAGAAGATGTTCGAGACTTTGCCAAGGTACT |
| | | AAAAAACAAATTTCGAACCAAAAGGTATTTTGCGAAGCATCCCCG |
| | | AATGGGCTACCTGCCAGTGCAGACTGTCTTAGAGGGGGACAACAT |
| | | GGAAACGCCTGCCTCGTCCCCTCAGCTTTCACACGATGATACTCA |
| | | TTCACGCATTGAACATTATGCTAGCAGGCTAGCAGAAATGGAAAA |
| | | CAGCAATGGATCTTATCTAAATGATAGCATCTCTCCTAATGAGAG |
| | | CATAGATGATGAACATTTGTTAATCCAGCATTACTGCCAAAGTTT |
| | | GAACCAGGACTCCCCCCTGAGCCAGCCTCGTAGTCCTGCCCAGAT |
| | | CTTGATTTCCTTAGAGAGTGAGGAAAGAGGGGAGCTAGAGAGAAT |
| | | CCTAGCAGATCTTGAGGAAGAAAACAGGAATCTGCAAGCAGAATA |
| | | TGACCGTCTAAAGCAGCAGCACGAACATAAAGGCCTGTCCCCACT |
| | | GCCGTCCCCTCCTGAAATGATGCCCACCTCTCCCCAGAGTCCCCG |
| | | GGATGCTGAGCTCATTGCTGAGGCCAAGCTACTGCGTCAACACAA |
| | | AGGCCGCCTGGAAGCCAGGATGCAAATCCTGGAAGACCACAATAA |
| | | ACAGCTGGAGTCACAGTTACACAGGCTAAGGCAGCTGCTGGAGCA |
| | | ACCCCAGGCAGAGGCCAAAGTGAATGGCACAACGGTGTCCTCTCC |
| | | TTCTACCTCTCTACAGAGGTCCGACAGCAGTCAGCCTATGCTGCT |
| | | CCGAGTGGTTGGCAGTCAAACTTCGGACTCCATGGGTGAGGAAGA |
| | | TCTTCTCAGTCCTCCCCAGGACACAAGCACAGGGTTAGAGGAGGT |
| | | GATGGAGCAACTCAACAACTCCTTCCCTAGTTCAAGAGGAAGAAA |
| | | TACCCCTGGAAAGCCAATGAGAGAGGACACAATGTAGGAAGTCTT |
| | | TTCCACATGGCAGATGATTTGGGCAGAGCGATGGAGTCCTTAGTA |
| | | TCAGTCATGACAGATGAAGAAGGAGCAGAATAAATGTTTTACAAC |
| | | TCCTGATTCCCGCATGGTTTTTATAATATTCATACAACAAAGAGG |
| | | ATTAGACAGTAAGAGTTTACAAGAAATAAATCTATATTTTTGTGA |
| | | AGGGTAGTGGTATTATACTGTAGATTTCAGTAGTTTCTAAGTCTG |
| | | TTATTGTTTTGTTAACAATGGCAGGTTTTACACGTCTATGCAATT |
| | | GTACAAAAAAGTTATAAGAAAACTACATGTAAAATCTTGATAGCT |
| | | AAATAACTTGCCATTTCTTTATATGGAACGCATTTTGGGTTGTTT |
| | | AAAAATTTATAACAGTTATAAAGAAAGATTGTAAACTAAAGTGTG |
| | | CTTTATAAAAAAAAGTTGTTTATAAAAACCCCTAAAAACAAAACA |
| | | AACACACACACACACACATACACACACACACACAAAACTTTGAGG |
| | | CAGCGCATTGTTTTGCATCCTTTTGGCGTGATATCCATATGAAAT |
| | | TCATGGCTTTTTCTTTTTTTGCATATTAAAGATAAGACTTCCTCT |
| | | ACCACCACACCAAATGACTACTACACACTGCTCATTTGAGAACTG |
| | | TCAGCTGAGTGGGGCAGGCTTGAGTTTTCATTTCATATATCTATA |
| | | TGTCTATAAGTATATAAATACTATAGTTATATAGATAAAGAGATA |
| | | CGAATTTCTATAGACTGACTTTTTCCATTTTTTAAATGTTCATGT |
| | | CACATCCTAATAGAAAGAAATTACTTCTAGTCAGTCATCCAGGCT |
| | | TACCTGCTTGGTCTAGA |
| 41 | Mini dystrophin | GGGATTCCCTCACTTTCCCCCTACAGGACTCAGATCTGGGAGGCA |
| | AR2-R21 | ATTACCTTCGGAGAAAAACGAATAGGAAAAACTGAAGTGTTACTT |
| | (artificial | TTTTTAAAGCTGCTGAAGTTTGTTGGTTTCTCATTGTTTTTAAGC |
| | sequence) | CTACTGGAGCAATAAAGTTTGAAGAACTTTTACCAGGTTTTTTTT |
| | | ATCGCTGCCTTGATATACACTTTTCAAAATGCTTTGGTGGGAAGA |
| | | AGTAGAGGACTGTTATGAAAGAGAAGATGTTCAAAAGAAAACATT |
| | | CACAAAATGGGTAAATGCACAATTTTCTAAGTTTGGGAAGCAGCA |
| | | TATTGAGAACCTCTTCAGTGACCTACAGGATGGGAGGCGCCTCCT |
| | | AGACCTCCTCGAAGGCCTGACAGGGCAAAAACTGCCAAAAGAAAA |
| | | AGGATCCACAAGAGTTCATGCCCTGAACAATGTCAACAAGGCACT |
| | | GCGGGTTTTGCAGAACAATAATGTTGATTTAGTGAATATTGGAAG |
| | | TACTGACATCGTAGATGGAAATCATAAACTGACTCTTGGTTTGAT |
| | | TTGGAATATAATCCTCCACTGGCAGGTCAAAAATGTAATGAAAAA |
| | | TATCATGGCTGGATTGCAACAAACCAACAGTGAAAAGATTCTCCT |
| | | GAGCTGGGTCCGACAATCAACTCGTAATTATCCACAGGTTAATGT |
| | | AATCAACTTCACCACCAGCTGGTCTGATGGCCTGGCTTTGAATGC |
| | | TCTCATCCATAGTCATAGGCCAGACCTATTTGACTGGAATAGTGT |
| | | GGTTTGCCAGCAGTCAGCCACACAACGACTGGAACATGCATTCAA |
| | | CATCGCCAGATATCAATTAGGCATAGAGAAACTACTCGATCCTGA |
| | | AGATGTTGATACCACCTATCCAGATAAGAAGTCCATCTTAATGTA |
| | | CATCACATCACTCTTCCAAGTTTTGCCTCAACAAGTGAGCATTGA |
| | | AGCCATCCAGGAAGTGGAAATGTTGCCAAGGCCACCTAAAGTGAC |
| | | TAAAGAAGAACATTTTCAGTTACATCATCAAATGCACTATTCTCA |
| | | ACAGATCACGGTCAGTCTAGCACAGGGATATGAGAGAACTTCTTC |
| | | CCCTAAGCCTCGATTCAAGAGCTATGCCTACACACAGGCTGCTTA |
| | | TGTCACCACCTCTGACCCTACACGGAGCCCATTTCCTTCACAGCA |
| | | TTTGGAAGCTCCTGAAGACAAGTCATTTGGCAGTTCATTGATGGA |
| | | GAGTGAAGTAAACCTGGACCGTTATCAAACAGCTTTAGAAGAAGT |
| | | ATTATCGTGGCTTCTTTCTGCTGAGGACACATTGCAAGCACAAGG |
| | | AGAGATTTCTAATGATGTGGAAGTGGTGAAAGACCAGTTTCATAC |
| | | TCATGAGGGGTACATGATGGATTTGACAGCCCATCAGGGCCGGGT |
| | | TGGTAATATTCTACAATTGGGAAGTAAGCTGATTGGAACAGGAAA |
| | | ATTATCAGAAGATGAAGAAACTGAAGTACAAGAGCAGATGAATCT |
| | | CCTAAATTCAAGATGGGAATGCCTCAGGGTAGCTAGCATGGAAAA |
| | | ACAAAGCAATTTACATCATAGATTACTGCAACAGTTCCCCCTGGA |
| | | CCTGGAAAAGTTTCTTGCCTGGCTTACAGAAGCTGAAACAACTGC |
| | | CAATGTCCTACAGGATGCTACCCGTAAGGAAAGGCTCCTAGAAGA |
| | | CTCCAAGGGAGTAAAAGAGCTGATGAAACAATGGCAAGACCTCCA |
| | | AGGTGAAATTGAAGCTCACACAGATGTTTATCACAACCTGGATGA |
| | | AAACAGCCAAAAAATCCTGAGATCCCTGGAAGGTTCCGATGATGC |
| | | AGTCCTGTTACAAAGACGTTTGGATAACATGAACTTCAAGTGGAG |
| | | TGAACTTCGGAAAAAGTCTCTCAACATTAGGTCCCATTTGGAAGC |
| | | CAGTTCTGACCAGTGGAAGCGTCTGCACCTTTCTCTGCAGGAACT |
| | | TCTGGTGTGGCTACAGCTGAAAGATGATGAATTAAGCCGGCAGGC |
| | | ACCTATTGGAGGCGACTTTCCAGCAGTTCAGAAGCAGAACGATGT |
| | | ACATAGGGCCTTCAAGAGGGAATTGAAAACTAAAGAACCTGTAAT |
| | | CATGAGTACTCTTGAGACTGTACGAATATTTCTGACAGAGCAGCC |
| | | TTTGGAAGGACTAGAGAAACTCTACCAGGAGCCCAGAGAGCTGCC |
| | | TCCTGAGGAGAGAGCCCAGAATGTCACTCGGCTTCTACGAAAGCA |
| | | GGCTGAGGAGGTCAATACTGAGTGGGAAAAATTGAACCTGCACTC |
| | | CGCTGACTGGCAGAGAAAAATAGATGAGACCCTTGAAAGACTCCA |
| | | GGAACTTCAAGAGGCCACGGATGAGCTGGACCTCAAGCTGCGCCA |
| | | AGCTGAGGTGATCAAGGGATCCTGGCAGCCCGTGGGCGATCTCCT |
| | | CATTGACTCTCTCCAAGATCACCTCGAGAAAGTCAAGGCACTTCG |
| | | AGGAGAAATTGCGCCTCTGAAAGAGAACGTGAGCCACGTCAATGA |
| | | CCTTGCTCGCCAGCTTACCACTTTGGGCATTCAGCTCTCACCGTA |
| | | TAACCTCAGCACTCTGGAAGACCTGAACACCAGATGGAAGCTTCT |
| | | GCAGGTGGCCGTCGAGGACCGAGTCAGGCAGCTGCATGAAGCCCA |
| | | CAGGGACTTTGGTCCAGCATCTCAGCACTTTCTTTCCACGTCTGT |
| | | CCAGGGTCCCTGGGAGAGAGCCATCTCGCCAAACAAAGTGCCCTA |
| | | CTATATCAACCACGAGACTCAAACAACTTGCTGGGACCATCCCAA |
| | | AATGACAGAGCTCTACCAGTCTTTAGCTGACCTGAATAATGTCAG |
| | | ATTCTCAGCTTATAGGACTGCCATGAAACTCCGAAGACTGCAGAA |
| | | GGCCCTTTGCTTGGATCTCTTGAGCCTGTCAGCTGCATGTGATGC |
| | | CTTGGACCAGCACAACCTCAAGCAAAATGACCAGCCCATGGATAT |
| | | CCTGCAGATTATTAATTGTTTGACCACTATTTATGACCGCCTGGA |
| | | GCAAGAGCACAACAATTTGGTCAACGTCCCTCTCTGCGTGGATAT |
| | | GTGTCTGAACTGGCTGCTGAATGTTTATGATACGGGACGAACAGG |
| | | GAGGATCCGTGTCCTGTCTTTTAAAACTGGCATCATTTCCCTGTG |
| | | TAAAGCACATTTGGAAGACAAGTACAGATACCTTTTCAAGCAAGT |
| | | GGCAAGTTCAACAGGATTTTGTGACCAGCGCAGGCTGGGCCTCCT |
| | | TCTGCATGATTCTATCCAAATTCCAAGACAGTTGGGTGAAGTTGC |
| | | ATCCTTTGGGGGCAGTAACATTGAGCCAAGTGTCCGGAGCTGCTT |
| | | CCAATTTGCTAATAATAAGCCAGAGATCGAAGCGGCCCTCTTCCT |
| | | AGACTGGATGAGACTGGAACCCCAGTCCATGGTGTGGCTGCCCGT |
| | | CCTGCACAGAGTGGCTGCTGCAGAAACTGCCAAGCATCAGGCCAA |
| | | ATGTAACATCTGCAAAGAGTGTCCAATCATTGGATTCAGGTACAG |
| | | GAGTCTAAAGCACTTTAATTATGACATCTGCCAAAGCTGCTTTTT |
| | | TTCTGGTCGAGTTGCAAAAGGCCATAAAATGCACTATCCCATGGT |
| | | GGAATATTGCACTCCGACTACATCAGGAGAAGATGTTCGAGACTT |
| | | TGCCAAGGTACTAAAAAACAAATTTCGAACCAAAAGGTATTTTGC |
| | | GAAGCATCCCCGAATGGGCTACCTGCCAGTGCAGACTGTCTTAGA |
| | | GGGGGACAACATGGAAACGCCTGCCTCGTCCCCTCAGCTTTCACA |
| | | CGATGATACTCATTCACGCATTGAACATTATGCTAGCAGGCTAGC |
| | | AGAAATGGAAAACAGCAATGGATCTTATCTAAATGATAGCATCTC |
| | | TCCTAATGAGAGCATAGATGATGAACATTTGTTAATCCAGCATTA |
| | | CTGCCAAAGTTTGAACCAGGACTCCCCCCTGAGCCAGCCTCGTAG |
| | | TCCTGCCCAGATCTTGATTTCCTTAGAGAGTGAGGAAAGAGGGGA |
| | | GCTAGAGAGAATCCTAGCAGATCTTGAGGAAGAAAACAGGAATCT |
| | | GCAAGCAGAATATGACCGTCTAAAGCAGCAGCACGAACATAAAGG |
| | | CCTGTCCCCACTGCCGTCCCCTCCTGAAATGATGCCCACCTCTCC |
| | | CCAGAGTCCCCGGGATGCTGAGCTCATTGCTGAGGCCAAGCTACT |
| | | GCGTCAACACAAAGGCCGCCTGGAAGCCAGGATGCAAATCCTGGA |
| | | AGACCACAATAAACAGCTGGAGTCACAGTTACACAGGCTAAGGCA |
| | | GCTGCTGGAGCAACCCCAGGCAGAGGCCAAAGTGAATGGCACAAC |
| | | GGTGTCCTCTCCTTCTACCTCTCTACAGAGGTCCGACAGCAGTCA |
| | | GCCTATGCTGCTCCGAGTGGTTGGCAGTCAAACTTCGGACTCCAT |
| | | GGGTGAGGAAGATCTTCTCAGTCCTCCCCAGGACACAAGCACAGG |
| | | GTTAGAGGAGGTGATGGAGCAACTCAACAACTCCTTCCCTAGTTC |
| | | AAGAGGAAGAAATACCCCTGGAAAGCCAATGAGAGAGGACACAAT |
| | | GTAGGAAGTCTTTTCCACATGGCAGATGATTTGGGCAGAGCGATG |
| | | GAGTCCTTAGTATCAGTCATGACAGATGAAGAAGGAGCAGAATAA |
| | | ATGTTTTACAACTCCTGATTCCCGCATGGTTTTTATAATATTCAT |
| | | ACAACAAAGAGGATTAGACAGTAAGAGTTTACAAGAAATAAATCT |
| | | ATATTTTTGTGAAGGGTAGTGGTATTATACTGTAGATTTCAGTAG |
| | | TTTCTAAGTCTGTTATTGTTTTGTTAACAATGGCAGGTTTTACAC |
| | | GTCTATGCAATTGTACAAAAAAGTTATAAGAAAACTACATGTAAA |
| | | ATCTTGATAGCTAAATAACTTGCCATTTCTTTATATGGAACGCAT |
| | | TTTGGGTTGTTTAAAAATTTATAACAGTTATAAAGAAAGATTGTA |
| | | AACTAAAGTGTGCTTTATAAAAAAAAGTTGTTTATAAAAACCCCT |
| | | AAAAACAAAACAAACACACACACACACACATACACACACACACAC |
| | | AAAACTTTGAGGCAGCGCATTGTTTTGCATCCTTTTGGCGTGATA |
| | | TCCATATGAAATTCATGGCTTTTTCTTTTTTTGCATATTAAAGAT |
| | | AAGACTTCCTCTACCACCACACCAAATGACTACTACACACTGCTC |
| | | ATTTGAGAACTGTCAGCTGAGTGGGGCAGGCTTGAGTTTTCATTT |
| | | CATATATCTATATGTCTATAAGTATATAAATACTATAGTTATATA |
| | | GATAAAGAGATACGAATTTCTATAGACTGACTTTTTCCATTTTTT |
| | | AAATGTTCATGTCACATCCTAATAGAAAGAAATTACTTCTAGTCA |
| | | GTCATCCAGGCTTACCTGCTTGGTCTAGA |
| 42 | Mini dystrophin | GGGATTCCCTCACTTTCCCCCTACAGGACTCAGATCTGGGAGGCA |
| | AR2-R21 + H3 | ATTACCTTCGGAGAAAAACGAATAGGAAAAACTGAAGTGTTACTT |
| | (artificial | TTTTTAAAGCTGCTGAAGTTTGTTGGTTTCTCATTGTTTTTAAGC |
| | sequence) | CTACTGGAGCAATAAAGTTTGAAGAACTTTTACCAGGTTTTTTTT |
| | | ATCGCTGCCTTGATATACACTTTTCAAAATGCTTTGGTGGGAAGA |
| | | AGTAGAGGACTGTTATGAAAGAGAAGATGTTCAAAAGAAAACATT |
| | | CACAAAATGGGTAAATGCACAATTTTCTAAGTTTGGGAAGCAGCA |
| | | TATTGAGAACCTCTTCAGTGACCTACAGGATGGGAGGCGCCTCCT |
| | | AGACCTCCTCGAAGGCCTGACAGGGCAAAAACTGCCAAAAGAAAA |
| | | AGGATCCACAAGAGTTCATGCCCTGAACAATGTCAACAAGGCACT |
| | | GCGGGTTTTGCAGAACAATAATGTTGATTTAGTGAATATTGGAAG |
| | | TACTGACATCGTAGATGGAAATCATAAACTGACTCTTGGTTTGAT |
| | | TTGGAATATAATCCTCCACTGGCAGGTCAAAAATGTAATGAAAAA |
| | | TATCATGGCTGGATTGCAACAAACCAACAGTGAAAAGATTCTCCT |
| | | GAGCTGGGTCCGACAATCAACTCGTAATTATCCACAGGTTAATGT |
| | | AATCAACTTCACCACCAGCTGGTCTGATGGCCTGGCTTTGAATGC |
| | | TCTCATCCATAGTCATAGGCCAGACCTATTTGACTGGAATAGTGT |
| | | GGTTTGCCAGCAGTCAGCCACACAACGACTGGAACATGCATTCAA |
| | | CATCGCCAGATATCAATTAGGCATAGAGAAACTACTCGATCCTGA |
| | | AGATGTTGATACCACCTATCCAGATAAGAAGTCCATCTTAATGTA |
| | | CATCACATCACTCTTCCAAGTTTTGCCTCAACAAGTGAGCATTGA |
| | | AGCCATCCAGGAAGTGGAAATGTTGCCAAGGCCACCTAAAGTGAC |
| | | TAAAGAAGAACATTTTCAGTTACATCATCAAATGCACTATTCTCA |
| | | ACAGATCACGGTCAGTCTAGCACAGGGATATGAGAGAACTTCTTC |
| | | CCCTAAGCCTCGATTCAAGAGCTATGCCTACACACAGGCTGCTTA |
| | | TGTCACCACCTCTGACCCTACACGGAGCCCATTTCCTTCACAGCA |
| | | TTTGGAAGCTCCTGAAGACAAGTCATTTGGCAGTTCATTGATGGA |
| | | GAGTGAAGTAAACCTGGACCGTTATCAAACAGCTTTAGAAGAAGT |
| | | ATTATCGTGGCTTCTTTCTGCTGAGGACACATTGCAAGCACAAGG |
| | | AGAGATTTCTAATGATGTGGAAGTGGTGAAAGACCAGTTTCATAC |
| | | TCATGAGGGGTACATGATGGATTTGACAGCCCATCAGGGCCGGGT |
| | | TGGTAATATTCTACAATTGGGAAGTAAGCTGATTGGAACAGGAAA |
| | | ATTATCAGAAGATGAAGAAACTGAAGTACAAGAGCAGATGAATCT |
| | | CCTAAATTCAAGATGGGAATGCCTCAGGGTAGCTAGCATGGAAAA |
| | | ACAAAGCAATTTACATGCTCCTGGACTGACCACTATTGGAGCCTC |
| | | TCCTACTCAGACTGTTACTCTGGTGACACAACCTGTGGTTACTAA |
| | | GGAAACTGCCATCTCCAAACTAGAAATGCCATCTTCCTTGATGTT |
| | | GGAGCATAGATTACTGCAACAGTTCCCCCTGGACCTGGAAAAGTT |
| | | TCTTGCCTGGCTTACAGAAGCTGAAACAACTGCCAATGTCCTACA |
| | | GGATGCTACCCGTAAGGAAAGGCTCCTAGAAGACTCCAAGGGAGT |
| | | AAAAGAGCTGATGAAACAATGGCAAGACCTCCAAGGTGAAATTGA |
| | | AGCTCACACAGATGTTTATCACAACCTGGATGAAAACAGCCAAAA |
| | | AATCCTGAGATCCCTGGAAGGTTCCGATGATGCAGTCCTGTTACA |
| | | AAGACGTTTGGATAACATGAACTTCAAGTGGAGTGAACTTCGGAA |
| | | AAAGTCTCTCAACATTAGGTCCCATTTGGAAGCCAGTTCTGACCA |
| | | GTGGAAGCGTCTGCACCTTTCTCTGCAGGAACTTCTGGTGTGGCT |
| | | ACAGCTGAAAGATGATGAATTAAGCCGGCAGGCACCTATTGGAGG |
| | | CGACTTTCCAGCAGTTCAGAAGCAGAACGATGTACATAGGGCCTT |
| | | CAAGAGGGAATTGAAAACTAAAGAACCTGTAATCATGAGTACTCT |
| | | TGAGACTGTACGAATATTTCTGACAGAGCAGCCTTTGGAAGGACT |
| | | AGAGAAACTCTACCAGGAGCCCAGAGAGCTGCCTCCTGAGGAGAG |
| | | AGCCCAGAATGTCACTCGGCTTCTACGAAAGCAGGCTGAGGAGGT |
| | | CAATACTGAGTGGGAAAAATTGAACCTGCACTCCGCTGACTGGCA |
| | | GAGAAAAATAGATGAGACCCTTGAAAGACTCCAGGAACTTCAAGA |
| | | GGCCACGGATGAGCTGGACCTCAAGCTGCGCCAAGCTGAGGTGAT |
| | | CAAGGGATCCTGGCAGCCCGTGGGCGATCTCCTCATTGACTCTCT |
| | | CCAAGATCACCTCGAGAAAGTCAAGGCACTTCGAGGAGAAATTGC |
| | | GCCTCTGAAAGAGAACGTGAGCCACGTCAATGACCTTGCTCGCCA |
| | | GCTTACCACTTTGGGCATTCAGCTCTCACCGTATAACCTCAGCAC |
| | | TCTGGAAGACCTGAACACCAGATGGAAGCTTCTGCAGGTGGCCGT |
| | | CGAGGACCGAGTCAGGCAGCTGCATGAAGCCCACAGGGACTTTGG |
| | | TCCAGCATCTCAGCACTTTCTTTCCACGTCTGTCCAGGGTCCCTG |
| | | GGAGAGAGCCATCTCGCCAAACAAAGTGCCCTACTATATCAACCA |
| | | CGAGACTCAAACAACTTGCTGGGACCATCCCAAAATGACAGAGCT |
| | | CTACCAGTCTTTAGCTGACCTGAATAATGTCAGATTCTCAGCTTA |
| | | TAGGACTGCCATGAAACTCCGAAGACTGCAGAAGGCCCTTTGCTT |
| | | GGATCTCTTGAGCCTGTCAGCTGCATGTGATGCCTTGGACCAGCA |
| | | CAACCTCAAGCAAAATGACCAGCCCATGGATATCCTGCAGATTAT |
| | | TAATTGTTTGACCACTATTTATGACCGCCTGGAGCAAGAGCACAA |
| | | CAATTTGGTCAACGTCCCTCTCTGCGTGGATATGTGTCTGAACTG |
| | | GCTGCTGAATGTTTATGATACGGGACGAACAGGGAGGATCCGTGT |
| | | CCTGTCTTTTAAAACTGGCATCATTTCCCTGTGTAAAGCACATTT |
| | | GGAAGACAAGTACAGATACCTTTTCAAGCAAGTGGCAAGTTCAAC |
| | | AGGATTTTGTGACCAGCGCAGGCTGGGCCTCCTTCTGCATGATTC |
| | | TATCCAAATTCCAAGACAGTTGGGTGAAGTTGCATCCTTTGGGGG |
| | | CAGTAACATTGAGCCAAGTGTCCGGAGCTGCTTCCAATTTGCTAA |
| | | TAATAAGCCAGAGATCGAAGCGGCCCTCTTCCTAGACTGGATGAG |
| | | ACTGGAACCCCAGTCCATGGTGTGGCTGCCCGTCCTGCACAGAGT |
| | | GGCTGCTGCAGAAACTGCCAAGCATCAGGCCAAATGTAACATCTG |
| | | CAAAGAGTGTCCAATCATTGGATTCAGGTACAGGAGTCTAAAGCA |
| | | CTTTAATTATGACATCTGCCAAAGCTGCTTTTTTTCTGGTCGAGT |
| | | TGCAAAAGGCCATAAAATGCACTATCCCATGGTGGAATATTGCAC |
| | | TCCGACTACATCAGGAGAAGATGTTCGAGACTTTGCCAAGGTACT |
| | | AAAAAACAAATTTCGAACCAAAAGGTATTTTGCGAAGCATCCCCG |
| | | AATGGGCTACCTGCCAGTGCAGACTGTCTTAGAGGGGGACAACAT |
| | | GGAAACGCCTGCCTCGTCCCCTCAGCTTTCACACGATGATACTCA |
| | | TTCACGCATTGAACATTATGCTAGCAGGCTAGCAGAAATGGAAAA |
| | | CAGCAATGGATCTTATCTAAATGATAGCATCTCTCCTAATGAGAG |
| | | CATAGATGATGAACATTTGTTAATCCAGCATTACTGCCAAAGTTT |
| | | GAACCAGGACTCCCCCCTGAGCCAGCCTCGTAGTCCTGCCCAGAT |
| | | CTTGATTTCCTTAGAGAGTGAGGAAAGAGGGGAGCTAGAGAGAAT |
| | | CCTAGCAGATCTTGAGGAAGAAAACAGGAATCTGCAAGCAGAATA |
| | | TGACCGTCTAAAGCAGCAGCACGAACATAAAGGCCTGTCCCCACT |
| | | GCCGTCCCCTCCTGAAATGATGCCCACCTCTCCCCAGAGTCCCCG |
| | | GGATGCTGAGCTCATTGCTGAGGCCAAGCTACTGCGTCAACACAA |
| | | AGGCCGCCTGGAAGCCAGGATGCAAATCCTGGAAGACCACAATAA |
| | | ACAGCTGGAGTCACAGTTACACAGGCTAAGGCAGCTGCTGGAGCA |
| | | ACCCCAGGCAGAGGCCAAAGTGAATGGCACAACGGTGTCCTCTCC |
| | | TTCTACCTCTCTACAGAGGTCCGACAGCAGTCAGCCTATGCTGCT |
| | | CCGAGTGGTTGGCAGTCAAACTTCGGACTCCATGGGTGAGGAAGA |
| | | TCTTCTCAGTCCTCCCCAGGACACAAGCACAGGGTTAGAGGAGGT |
| | | GATGGAGCAACTCAACAACTCCTTCCCTAGTTCAAGAGGAAGAAA |
| | | TACCCCTGGAAAGCCAATGAGAGAGGACACAATGTAGGAAGTCTT |
| | | TTCCACATGGCAGATGATTTGGGCAGAGCGATGGAGTCCTTAGTA |
| | | TCAGTCATGACAGATGAAGAAGGAGCAGAATAAATGTTTTACAAC |
| | | TCCTGATTCCCGCATGGTTTTTATAATATTCATACAACAAAGAGG |
| | | ATTAGACAGTAAGAGTTTACAAGAAATAAATCTATATTTTTGTGA |
| | | AGGGTAGTGGTATTATACTGTAGATTTCAGTAGTTTCTAAGTCTG |
| | | TTATTGTTTTGTTAACAATGGCAGGTTTTACACGTCTATGCAATT |
| | | GTACAAAAAAGTTATAAGAAAACTACATGTAAAATCTTGATAGCT |
| | | AAATAACTTGCCATTTCTTTATATGGAACGCATTTTGGGTTGTTT |
| | | AAAAATTTATAACAGTTATAAAGAAAGATTGTAAACTAAAGTGTG |
| | | CTTTATAAAAAAAAGTTGTTTATAAAAACCCCTAAAAACAAAACA |
| | | AACACACACACACACACATACACACACACACACAAAACTTTGAGG |
| | | CAGCGCATTGTTTTGCATCCTTTTGGCGTGATATCCATATGAAAT |
| | | TCATGGCTTTTTCTTTTTTTGCATATTAAAGATAAGACTTCCTCT |
| | | ACCACCACACCAAATGACTACTACACACTGCTCATTTGAGAACTG |
| | | TCAGCTGAGTGGGGCAGGCTTGAGTTTTCATTTCATATATCTATA |
| | | TGTCTATAAGTATATAAATACTATAGTTATATAGATAAAGAGATA |
| | | CGAATTTCTATAGACTGACTTTTTCCATTTTTTAAATGTTCATGT |
| | | CACATCCTAATAGAAAGAAATTACTTCTAGTCAGTCATCCAGGCT |
| | | TACCTGCTTGGTCTAGA |
| 43 | Mini dystrophin | GGGATTCCCTCACTTTCCCCCTACAGGACTCAGATCTGGGAGGCA |
| | AH2-R19 | ATTACCTTCGGAGAAAAACGAATAGGAAAAACTGAAGTGTTACTT |
| | (artificial | TTTTTAAAGCTGCTGAAGTTTGTTGGTTTCTCATTGTTTTTAAGC |
| | sequence) | CTACTGGAGCAATAAAGTTTGAAGAACTTTTACCAGGTTTTTTTT |
| | | ATCGCTGCCTTGATATACACTTTTCAAAATGCTTTGGTGGGAAGA |
| | | AGTAGAGGACTGTTATGAAAGAGAAGATGTTCAAAAGAAAACATT |
| | | CACAAAATGGGTAAATGCACAATTTTCTAAGTTTGGGAAGCAGCA |
| | | TATTGAGAACCTCTTCAGTGACCTACAGGATGGGAGGCGCCTCCT |
| | | AGACCTCCTCGAAGGCCTGACAGGGCAAAAACTGCCAAAAGAAAA |
| | | AGGATCCACAAGAGTTCATGCCCTGAACAATGTCAACAAGGCACT |
| | | GCGGGTTTTGCAGAACAATAATGTTGATTTAGTGAATATTGGAAG |
| | | TACTGACATCGTAGATGGAAATCATAAACTGACTCTTGGTTTGAT |
| | | TTGGAATATAATCCTCCACTGGCAGGTCAAAAATGTAATGAAAAA |
| | | TATCATGGCTGGATTGCAACAAACCAACAGTGAAAAGATTCTCCT |
| | | GAGCTGGGTCCGACAATCAACTCGTAATTATCCACAGGTTAATGT |
| | | AATCAACTTCACCACCAGCTGGTCTGATGGCCTGGCTTTGAATGC |
| | | TCTCATCCATAGTCATAGGCCAGACCTATTTGACTGGAATAGTGT |
| | | GGTTTGCCAGCAGTCAGCCACACAACGACTGGAACATGCATTCAA |
| | | CATCGCCAGATATCAATTAGGCATAGAGAAACTACTCGATCCTGA |
| | | AGATGTTGATACCACCTATCCAGATAAGAAGTCCATCTTAATGTA |
| | | CATCACATCACTCTTCCAAGTTTTGCCTCAACAAGTGAGCATTGA |
| | | AGCCATCCAGGAAGTGGAAATGTTGCCAAGGCCACCTAAAGTGAC |
| | | TAAAGAAGAACATTTTCAGTTACATCATCAAATGCACTATTCTCA |
| | | ACAGATCACGGTCAGTCTAGCACAGGGATATGAGAGAACTTCTTC |
| | | CCCTAAGCCTCGATTCAAGAGCTATGCCTACACACAGGCTGCTTA |
| | | TGTCACCACCTCTGACCCTACACGGAGCCCATTTCCTTCACAGCA |
| | | TTTGGAAGCTCCTGAAGACAAGTCATTTGGCAGTTCATTGATGGA |
| | | GAGTGAAGTAAACCTGGACCGTTATCAAACAGCTTTAGAAGAAGT |
| | | ATTATCGTGGCTTCTTTCTGCTGAGGACACATTGCAAGCACAAGG |
| | | AGAGATTTCTAATGATGTGGAAGTGGTGAAAGACCAGTTTCATAC |
| | | TCATGAGGGGTACATGATGGATTTGACAGCCCATCAGGGCCGGGT |
| | | TGGTAATATTCTACAATTGGGAAGTAAGCTGATTGGAACAGGAAA |
| | | ATTATCAGAAGATGAAGAAACTGAAGTACAAGAGCAGATGAATCT |
| | | CCTAAATTCAAGATGGGAATGCCTCAGGGTAGCTAGCATGGAAAA |
| | | ACAAAGCAATTTACATAGAGTTTTAATGGATCTCCAGAATCAGAA |
| | | ACTGAAAGAGTTGAATGACTGGCTAACAAAAACAGAAGAAAGAAC |
| | | AAGGAAAATGGAGGAAGAGCCTCTTGGACCTGATCTTGAAGACCT |
| | | AAAACGCCAAGTACAACAACATAAGGTGCTTCAAGAAGATCTAGA |
| | | ACAAGAACAAGTCAGGGTCAATTCTCTCACTCACATGGTGGTGGT |
| | | AGTTGATGAATCTAGTGGAGATCACGCAACTGCTGCTTTGGAAGA |
| | | ACAACTTAAGGTATTGGGAGATCGATGGGCAAACATCTGTAGATG |
| | | GACAGAAGACCGCTGGGTTCTTTTACAAGACATCCTTCTCAAATG |
| | | GCAACGTCTTACTGAAGAACAGTGCCTTTTTAGTGCATGGCTTTC |
| | | AGAAAAAGAAGATGCAGTGAACAAGATTCACACAACTGGCTTTAA |
| | | AGATCAAAATGAAATGTTATCAAGTCTTCAAAAACTGGCCGTTTT |
| | | AAAAGCGGATCTAGAAAAGAAAAAGCAATCCATGGGCAAACTGTA |
| | | TTCACTCAAACAAGATCTTCTTTCAACACTGAAGAATAAGTCAGT |
| | | GACCCAGAAGACGGAAGCATGGCTGGATAACTTTGCCCGGTGTTG |
| | | GGATAATTTAGTCCAAAAACTTGAAAAGAGTACAGCACAGATTTC |
| | | ACAGCAGCCTGACCTAGCTCCTGGACTGACCACTATTGGAGCCTC |
| | | TCCTACTCAGACTGTTACTCTGGTGACACAACCTGTGGTTACTAA |
| | | GGAAACTGCCATCTCCAAACTAGAAATGCCATCTTCCTTGATGTT |
| | | GGAGGTACCTGCTCTGGCAGATTTCAACCGGGCTTGGACAGAACT |
| | | TACCGACTGGCTTTCTCTGCTTGATCAAGTTATAAAATCACAGAG |
| | | GGTGATGGTGGGTGACCTTGAGGATATCAACGAGATGATCATCAA |
| | | GCAGAAGGCAACAATGCAGGATTTGGAACAGAGGCGTCCCCAGTT |
| | | GGAAGAACTCATTACCGCTGCCCAAAATTTGAAAAACAAGACCAG |
| | | CAATCAAGAGGCTAGAACAATCATTACGGATCGAATTGAAAGAAT |
| | | TCAGAATCAGTGGGATGAAGTACAAGAACACCTTCAGAACCGGAG |
| | | GCAACAGTTGAATGAAATGTTAAAGGATTCAACACAATGGCTGGA |
| | | AGCTAAGGAAGAAGCTGAGCAGGTCTTAGGACAGGCCAGAGCCAA |
| | | GCTTGAGTCATGGAAGGAGGGTCCCTATACAGTAGATGCAATCCA |
| | | AAAGAAAATCACAGAAACCAAGCAGTTGGCCAAAGACCTCCGCCA |
| | | GTGGCAGACAAATGTAGATGTGGCAAATGACTTGGCCCTGAAACT |
| | | TCTCCGGGATTATTCTGCAGATGATACCAGAAAAGTCCACATGAT |
| | | AACAGAGAATATCAATGCCTCTTGGAGAAGCATTCATAAAAGGGT |
| | | GAGTGAGCGAGAGGCTGCTTTGGAAGAAACTCATAGATTACTGCA |
| | | ACAGTTCCCCCTGGACCTGGAAAAGTTTCTTGCCTGGCTTACAGA |
| | | AGCTGAAACAACTGCCAATGTCCTACAGGATGCTACCCGTAAGGA |
| | | AAGGCTCCTAGAAGACTCCAAGGGAGTAAAAGAGCTGATGAAACA |
| | | ATGGCAAGACCTCCAAGGTGAAATTGAAGCTCACACAGATGTTTA |
| | | TCACAACCTGGATGAAAACAGCCAAAAAATCCTGAGATCCCTGGA |
| | | AGGTTCCGATGATGCAGTCCTGTTACAAAGACGTTTGGATAACAT |
| | | GAACTTCAAGTGGAGTGAACTTCGGAAAAAGTCTCTCAACATTAG |
| | | GTCCCATTTGGAAGCCAGTTCTGACCAGTGGAAGCGTCTGCACCT |
| | | TTCTCTGCAGGAACTTCTGGTGTGGCTACAGCTGAAAGATGATGA |
| | | ATTAAGCCGGCAGGCACCTATTGGAGGCGACTTTCCAGCAGTTCA |
| | | GAAGCAGAACGATGTACATAGGGCCTTCAAGAGGGAATTGAAAAC |
| | | TAAAGAACCTGTAATCATGAGTACTCTTGAGACTGTACGAATATT |
| | | TCTGACAGAGCAGCCTTTGGAAGGACTAGAGAAACTCTACCAGGA |
| | | GCCCAGAGAGCTGCCTCCTGAGGAGAGAGCCCAGAATGTCACTCG |
| | | GCTTCTACGAAAGCAGGCTGAGGAGGTCAATACTGAGTGGGAAAA |
| | | ATTGAACCTGCACTCCGCTGACTGGCAGAGAAAAATAGATGAGAC |
| | | CCTTGAAAGACTCCAGGAACTTCAAGAGGCCACGGATGAGCTGGA |
| | | CCTCAAGCTGCGCCAAGCTGAGGTGATCAAGGGATCCTGGCAGCC |
| | | CGTGGGCGATCTCCTCATTGACTCTCTCCAAGATCACCTCGAGAA |
| | | AGTCAAGGCACTTCGAGGAGAAATTGCGCCTCTGAAAGAGAACGT |
| | | GAGCCACGTCAATGACCTTGCTCGCCAGCTTACCACTTTGGGCAT |
| | | TCAGCTCTCACCGTATAACCTCAGCACTCTGGAAGACCTGAACAC |
| | | CAGATGGAAGCTTCTGCAGGTGGCCGTCGAGGACCGAGTCAGGCA |
| | | GCTGCATGAAGCCCACAGGGACTTTGGTCCAGCATCTCAGCACTT |
| | | TCTTTCCACGTCTGTCCAGGGTCCCTGGGAGAGAGCCATCTCGCC |
| | | AAACAAAGTGCCCTACTATATCAACCACGAGACTCAAACAACTTG |
| | | CTGGGACCATCCCAAAATGACAGAGCTCTACCAGTCTTTAGCTGA |
| | | CCTGAATAATGTCAGATTCTCAGCTTATAGGACTGCCATGAAACT |
| | | CCGAAGACTGCAGAAGGCCCTTTGCTTGGATCTCTTGAGCCTGTC |
| | | AGCTGCATGTGATGCCTTGGACCAGCACAACCTCAAGCAAAATGA |
| | | CCAGCCCATGGATATCCTGCAGATTATTAATTGTTTGACCACTAT |
| | | TTATGACCGCCTGGAGCAAGAGCACAACAATTTGGTCAACGTCCC |
| | | TCTCTGCGTGGATATGTGTCTGAACTGGCTGCTGAATGTTTATGA |
| | | TACGGGACGAACAGGGAGGATCCGTGTCCTGTCTTTTAAAACTGG |
| | | CATCATTTCCCTGTGTAAAGCACATTTGGAAGACAAGTACAGATA |
| | | CCTTTTCAAGCAAGTGGCAAGTTCAACAGGATTTTGTGACCAGCG |
| | | CAGGCTGGGCCTCCTTCTGCATGATTCTATCCAAATTCCAAGACA |
| | | GTTGGGTGAAGTTGCATCCTTTGGGGGCAGTAACATTGAGCCAAG |
| | | TGTCCGGAGCTGCTTCCAATTTGCTAATAATAAGCCAGAGATCGA |
| | | AGCGGCCCTCTTCCTAGACTGGATGAGACTGGAACCCCAGTCCAT |
| | | GGTGTGGCTGCCCGTCCTGCACAGAGTGGCTGCTGCAGAAACTGC |
| | | CAAGCATCAGGCCAAATGTAACATCTGCAAAGAGTGTCCAATCAT |
| | | TGGATTCAGGTACAGGAGTCTAAAGCACTTTAATTATGACATCTG |
| | | CCAAAGCTGCTTTTTTTCTGGTCGAGTTGCAAAAGGCCATAAAAT |
| | | GCACTATCCCATGGTGGAATATTGCACTCCGACTACATCAGGAGA |
| | | AGATGTTCGAGACTTTGCCAAGGTACTAAAAAACAAATTTCGAAC |
| | | CAAAAGGTATTTTGCGAAGCATCCCCGAATGGGCTACCTGCCAGT |
| | | GCAGACTGTCTTAGAGGGGGACAACATGGAAACTCCCGTTACTCT |
| | | GATCAACTTCTGGCCAGTAGATTCTGCGCCTGCCTCGTCCCCTCA |
| | | GCTTTCACACGATGATACTCATTCACGCATTGAACATTATGCTAG |
| | | CAGGCTAGCAGAAATGGAAAACAGCAATGGATCTTATCTAAATGA |
| | | TAGCATCTCTCCTAATGAGAGCATAGATGATGAACATTTGTTAAT |
| | | CCAGCATTACTGCCAAAGTTTGAACCAGGACTCCCCCCTGAGCCA |
| | | GCCTCGTAGTCCTGCCCAGATCTTGATTTCCTTAGAGAGTGAGGA |
| | | AAGAGGGGAGCTAGAGAGAATCCTAGCAGATCTTGAGGAAGAAAA |
| | | CAGGAATCTGCAAGCAGAATATGACCGTCTAAAGCAGCAGCACGA |
| | | ACATAAAGGCCTGTCCCCACTGCCGTCCCCTCCTGAAATGATGCC |
| | | CACCTCTCCCCAGAGTCCCCGGGATGCTGAGCTCATTGCTGAGGC |
| | | CAAGCTACTGCGTCAACACAAAGGCCGCCTGGAAGCCAGGATGCA |
| | | AATCCTGGAAGACCACAATAAACAGCTGGAGTCACAGTTACACAG |
| | | GCTAAGGCAGCTGCTGGAGCAACCCCAGGCAGAGGCCAAAGTGAA |
| | | TGGCACAACGGTGTCCTCTCCTTCTACCTCTCTACAGAGGTCCGA |
| | | CAGCAGTCAGCCTATGCTGCTCCGAGTGGTTGGCAGTCAAACTTC |
| | | GGACTCCATGGGTGAGGAAGATCTTCTCAGTCCTCCCCAGGACAC |
| | | AAGCACAGGGTTAGAGGAGGTGATGGAGCAACTCAACAACTCCTT |
| | | CCCTAGTTCAAGAGGAAGAAATACCCCTGGAAAGCCAATGAGAGA |
| | | GGACACAATGTAGGAAGTCTTTTCCACATGGCAGATGATTTGGGC |
| | | AGAGCGATGGAGTCCTTAGTATCAGTCATGACAGATGAAGAAGGA |
| | | GCAGAATAAATGTTTTACAACTCCTGATTCCCGCATGGTTTTTAT |
| | | AATATTCATACAACAAAGAGGATTAGACAGTAAGAGTTTACAAGA |
| | | AATAAATCTATATTTTTGTGAAGGGTAGTGGTATTATACTGTAGA |
| | | TTTCAGTAGTTTCTAAGTCTGTTATTGTTTTGTTAACAATGGCAG |
| | | GTTTTACACGTCTATGCAATTGTACAAAAAAGTTATAAGAAAACT |
| | | ACATGTAAAATCTTGATAGCTAAATAACTTGCCATTTCTTTATAT |
| | | GGAACGCATTTTGGGTTGTTTAAAAATTTATAACAGTTATAAAGA |
| | | AAGATTGTAAACTAAAGTGTGCTTTATAAAAAAAAGTTGTTTATA |
| | | AAAACCCCTAAAAACAAAACAAACACACACACACACACATACACA |
| | | CACACACACAAAACTTTGAGGCAGCGCATTGTTTTGCATCCTTTT |
| | | GGCGTGATATCCATATGAAATTCATGGCTTTTTCTTTTTTTGCAT |
| | | ATTAAAGATAAGACTTCCTCTACCACCACACCAAATGACTACTAC |
| | | ACACTGCTCATTTGAGAACTGTCAGCTGAGTGGGGCAGGCTTGAG |
| | | TTTTCATTTCATATATCTATATGTCTATAAGTATATAAATACTAT |
| | | AGTTATATAGATAAAGAGATACGAATTTCTATAGACTGACTTTTT |
| | | CCATTTTTTAAATGTTCATGTCACATCCTAATAGAAAGAAATTAC |
| | | TTCTAGTCAGTCATCCAGGCTTACCTGCTTGGTCTAGAATGGATT |
| | | TTTCCCGGAGCCGGAAGCCAGGAGGAAACTACACCACACTAAAAC |
| | | ATTGTCTACAGCTCCAGATGTTTCTCATTTTAAACAACTTTCCAC |
| | | TGACAACGAAAGTAAAGTAAAGTATTGGATTTTTTTAAAGGGAAC |
| | | ATGTGAATGAATACACAGGACTTATTATATCAGAGTGAGTAATCG |
| | | GTTGGTTGGTTGATTGATTGATTGATTGATACATTCAGCTTCCTG |
| | | CTGCTAGCAATGCCACGATTTAGATTTAATGATGCTTCAGTGGAA |
| | | ATCAATCAGAAGGTATTCTGACCTTGTGAACATCAGAAGGTATTT |
| | | TTTAACTCCCAAGCAGTAGCAGGACGATGATAGGGCTGGAGGGCT |
| | | ATGGATTCCCAGCCCATCCCTGTGAAGGAGTAGGCCACTCTTTAA |
| | | GTGAAGGATTGGATGATTGTTCATAATACATAAAGTTCTCTGTAA |
| | | TTACAACTAAATTATTATGCCCTCTTCTCACAGTCAAAAGGAACT |
| | | GGGTGGTTTGGTTTTTGTTGCTTTTTTAGATTTATTGTCCCATGT |
| | | GGGATGAGTTTTTAAATGCCACAAGACATAATTTAAAATAAATAA |
| | | ACTTTGGGAAAAGGTGTAAGACAGTAGCCCCATCACATTTGTGAT |
| | | ACTGACAGGTATCAACCCAGAAGCCCATGAACTGTGTTTCCATCC |
| | | TTTGCATTTCTCTGCGAGTAGTTCCACACAGGTTTGTAAGTAAGT |
| | | AAGAAAGAAGGCAAATTGATTCAAATGTTACAAAAAAACCCTTCT |
| | | TGGTGGATTAGACAGGTTAAATATATAAACAAACAAACAAAAATT |
| | | GCTCAAAAAAGAGGAGAAAAGCTCAAGAGGAAAAGCTAAGGACTG |
| | | GTAGGAAAAAGCTTTACTCTTTCATGCCATTTTATTTCTTTTTGA |
| | | TTTTTAAATCATTCATTCAATAGATACCACCGTGTGACCTATAAT |
| | | TTTGCAAATCTGTTACCTCTGACATCAAGTGTAATTAGCTTTTGG |
| | | AGAGTGGGCTGACATCAAGTGTAATTAGCTTTTGGAGAGTGGGTT |
| | | TTGTCCATTATTAATAATTAATTAATTAACATCAAACACGGCTTC |
| | | TCATGCTATTTCTACCTCACTTTGGTTTTGGGGTGTTCCTGATAA |
| | | TTGTGCACACCTGAGTTCACAGCTTCACCACTTGTCCATTGCGTT |
| | | ATTTTCTTTTTCCTTTATAATTCTTTCTTTTTCCTTCATAATTTT |
| | | CAAAAGAAAACCCAAAGCTCTAAGGTAACAAATTACCAAATTACA |
| | | TGAAGATTTGGTTTTTGTCTTGCATTTTTTTCCTTTATGTGACGC |
| | | TGGACCTTTTCTTTACCCAAGGATTTTTAAAACTCAGATTTAAAA |
| | | CAAGGGGTTACTTTACATCCTACTAAGAAGTTTAAGTAAGTAAGT |
| | | TTCATTCTAAAATCAGAGGTAAATAGAGTGCATAAATAATTTTGT |
| | | TTTAATCTTTTTGTTTTTCTTTTAGACACATTAGCTCTGGAGTGA |
| | | GTCTGTCATAATATTTGAACAAAAATTGAGAGCTTTATTGCTGCA |
| | | TTTTAAGCATAATTAATTTGGACATTATTTCGTGTTGTGTTCTTT |
| | | ATAACCACCGAGTATTAAACTGTAAATCATAATGTAACTGAAGCA |
| | | TAAACATCACATGGCATGTTTTGTCATTGTTTTCAGGTACTGAGT |
| | | TCTTACTTGAGTATCATAATATATTGTGTTTTAACACCAACACTG |
| | | TAACATTTACGAATTATTTTTTTAAACTTCAGTTTTACTGCATTT |
| | | TCACAACATATCAGACTTCACCAAATATATGCCTTACTATTGTAT |
| | | TATAGTACTGCTTTACTGTGTATCTCAATAAAGCACGCAGTTATG |
| | | TTAC |
| 44 | Mini dystrophin | GGGATTCCCTCACTTTCCCCCTACAGGACTCAGATCTGGGAGGCA |
| | AR9-R16 | ATTACCTTCGGAGAAAAACGAATAGGAAAAACTGAAGTGTTACTT |
| | (artificial | TTTTTAAAGCTGCTGAAGTTTGTTGGTTTCTCATTGTTTTTAAGC |
| | sequence) | CTACTGGAGCAATAAAGTTTGAAGAACTTTTACCAGGTTTTTTTT |
| | | ATCGCTGCCTTGATATACACTTTTCAAAATGCTTTGGTGGGAAGA |
| | | AGTAGAGGACTGTTATGAAAGAGAAGATGTTCAAAAGAAAACATT |
| | | CACAAAATGGGTAAATGCACAATTTTCTAAGTTTGGGAAGCAGCA |
| | | TATTGAGAACCTCTTCAGTGACCTACAGGATGGGAGGCGCCTCCT |
| | | AGACCTCCTCGAAGGCCTGACAGGGCAAAAACTGCCAAAAGAAAA |
| | | AGGATCCACAAGAGTTCATGCCCTGAACAATGTCAACAAGGCACT |
| | | GCGGGTTTTGCAGAACAATAATGTTGATTTAGTGAATATTGGAAG |
| | | TACTGACATCGTAGATGGAAATCATAAACTGACTCTTGGTTTGAT |
| | | TTGGAATATAATCCTCCACTGGCAGGTCAAAAATGTAATGAAAAA |
| | | TATCATGGCTGGATTGCAACAAACCAACAGTGAAAAGATTCTCCT |
| | | GAGCTGGGTCCGACAATCAACTCGTAATTATCCACAGGTTAATGT |
| | | AATCAACTTCACCACCAGCTGGTCTGATGGCCTGGCTTTGAATGC |
| | | TCTCATCCATAGTCATAGGCCAGACCTATTTGACTGGAATAGTGT |
| | | GGTTTGCCAGCAGTCAGCCACACAACGACTGGAACATGCATTCAA |
| | | CATCGCCAGATATCAATTAGGCATAGAGAAACTACTCGATCCTGA |
| | | AGATGTTGATACCACCTATCCAGATAAGAAGTCCATCTTAATGTA |
| | | CATCACATCACTCTTCCAAGTTTTGCCTCAACAAGTGAGCATTGA |
| | | AGCCATCCAGGAAGTGGAAATGTTGCCAAGGCCACCTAAAGTGAC |
| | | TAAAGAAGAACATTTTCAGTTACATCATCAAATGCACTATTCTCA |
| | | ACAGATCACGGTCAGTCTAGCACAGGGATATGAGAGAACTTCTTC |
| | | CCCTAAGCCTCGATTCAAGAGCTATGCCTACACACAGGCTGCTTA |
| | | TGTCACCACCTCTGACCCTACACGGAGCCCATTTCCTTCACAGCA |
| | | TTTGGAAGCTCCTGAAGACAAGTCATTTGGCAGTTCATTGATGGA |
| | | GAGTGAAGTAAACCTGGACCGTTATCAAACAGCTTTAGAAGAAGT |
| | | ATTATCGTGGCTTCTTTCTGCTGAGGACACATTGCAAGCACAAGG |
| | | AGAGATTTCTAATGATGTGGAAGTGGTGAAAGACCAGTTTCATAC |
| | | TCATGAGGGGTACATGATGGATTTGACAGCCCATCAGGGCCGGGT |
| | | TGGTAATATTCTACAATTGGGAAGTAAGCTGATTGGAACAGGAAA |
| | | ATTATCAGAAGATGAAGAAACTGAAGTACAAGAGCAGATGAATCT |
| | | CCTAAATTCAAGATGGGAATGCCTCAGGGTAGCTAGCATGGAAAA |
| | | ACAAAGCAATTTACATAGAGTTTTAATGGATCTCCAGAATCAGAA |
| | | ACTGAAAGAGTTGAATGACTGGCTAACAAAAACAGAAGAAAGAAC |
| | | AAGGAAAATGGAGGAAGAGCCTCTTGGACCTGATCTTGAAGACCT |
| | | AAAACGCCAAGTACAACAACATAAGGTGCTTCAAGAAGATCTAGA |
| | | ACAAGAACAAGTCAGGGTCAATTCTCTCACTCACATGGTGGTGGT |
| | | AGTTGATGAATCTAGTGGAGATCACGCAACTGCTGCTTTGGAAGA |
| | | ACAACTTAAGGTATTGGGAGATCGATGGGCAAACATCTGTAGATG |
| | | GACAGAAGACCGCTGGGTTCTTTTACAAGACATCCTTCTCAAATG |
| | | GCAACGTCTTACTGAAGAACAGTGCCTTTTTAGTGCATGGCTTTC |
| | | AGAAAAAGAAGATGCAGTGAACAAGATTCACACAACTGGCTTTAA |
| | | AGATCAAAATGAAATGTTATCAAGTCTTCAAAAACTGGCCGTTTT |
| | | AAAAGCGGATCTAGAAAAGAAAAAGCAATCCATGGGCAAACTGTA |
| | | TTCACTCAAACAAGATCTTCTTTCAACACTGAAGAATAAGTCAGT |
| | | GACCCAGAAGACGGAAGCATGGCTGGATAACTTTGCCCGGTGTTG |
| | | GGATAATTTAGTCCAAAAACTTGAAAAGAGTACAGCACAGATTTC |
| | | ACAGGCTGTCACCACCACTCAGCCATCACTAACACAGACAACTGT |
| | | AATGGAAACAGTAACTACGGTGACCACAAGGGAACAGATCCTGGT |
| | | AAAGCATGCTCAAGAGGAACTTCCACCACCACCTCCCCAAAAGAA |
| | | GAGGCAGATTACTGTGGATTCTGAAATTAGGAAAAGGTTGGATGT |
| | | TGATATAACTGAACTTCACAGCTGGATTACTCGCTCAGAAGCTGT |
| | | GTTGCAGAGTCCTGAATTTGCAATCTTTCGGAAGGAAGGCAACTT |
| | | CTCAGACTTAAAAGAAAAAGTCAATGCCATAGAGCGAGAAAAAGC |
| | | TGAGAAGTTCAGAAAACTGCAAGATGCCAGCAGATCAGCTCAGGC |
| | | CCTGGTGGAACAGATGGTGAATGAGGGTGTTAATGCAGATAGCAT |
| | | CAAACAAGCCTCAGAACAACTGAACAGCCGGTGGATCGAATTCTG |
| | | CCAGTTGCTAAGTGAGAGACTTAACTGGCTGGAGTATCAGAACAA |
| | | CATCATCGCTTTCTATAATCAGCTACAACAATTGGAGCAGATGAC |
| | | AACTACTGCTGAAAACTGGTTGAAAATCCAACCCACCACCCCATC |
| | | AGAGCCAACAGCAATTAAAAGTCAGTTAAAAATTTGTAAGGATGA |
| | | AGTCAACCGGCTATCAGGTCTTCAACCTCAAATTGAACGATTAAA |
| | | AATTCAAAGCATAGCCCTGAAAGAGAAAGGACAAGGACCCATGTT |
| | | CCTGGATGCAGACTTTGTGGCCTTTACAAATCATTTTAAGCAAGT |
| | | CTTTTCTGATGTGCAGGCCAGAGAGAAAGAGCTACAGACAATTTT |
| | | TGACACTTTGCCACCAATGCGCTATCAGGAGACCATGAGTGCCAT |
| | | CAGGACATGGGTCCAGCAGTCAGAAACCAAACTCTCCATACCTCA |
| | | ACTTAGTGTCACCGACTATGAAATCATGGAGCAGAGACTCGGGGA |
| | | ATTGCAGGCTTTACAAAGTTCTCTGCAAGAGCAACAAAGTGGCCT |
| | | ATACTATCTCAGCACCACTGTGAAAGAGATGTCGAAGAAAGCGCC |
| | | CTCTGAAATTAGCCGGAAATATCAATCAGAATTTGAAGAAATTGA |
| | | GGGACGCTGGAAGAAGCTCTCCTCCCAGCTGGTTGAGCATTGTCA |
| | | AAAGCTAGAGGAGCAAATGAATAAACTCCGAAAAATTCAGAATCA |
| | | CATACAAACCCTGAAGAAATGGATGGCTGAAGTTGATGTTTTTCT |
| | | GAAGGAGGAATGGCCTGCCCTTGGGGATTCAGAAATTCTAAAAAA |
| | | GCAGCTGAAACAGTGCAGACTTTTAGTCAGTGATATTCAGACAAT |
| | | TCAGCCCAGTCTAAACAGTGTCAATGAAGGTGGGCAGAAGATAAA |
| | | GAATGAAGCAGAGCCAGAGTTTGCTTCGAGACTTGAGACAGAACT |
| | | CAAAGAACTTAACACTCAGTGGGATCACATGTGCCAACAGGTCTA |
| | | TGCCAGAAAGGAGGCCTTGAAGGGAGGTTTGGAGAAAACTGTAAG |
| | | CCTCCAGAAAGATCTATCAGAGATGCACGAATGGATGACACAAGC |
| | | TGAAGAAGAGTATCTTGAGAGAGATTTTGAATATAAAACTCCAGA |
| | | TGAATTACAGAAAGCAGTTGAAGAGATGAAGAGAGCTAAAGAAGA |
| | | GGCCCAACAAAAAGAAGCGAAAGTGAAACTCCTTACTGAGTCTGT |
| | | AAATAGTGTCATAGCTCAAGCTCCACCTGTAGCACAAGAGGCCTT |
| | | AAAAAAGGAACTTGAAACTCTAACCACCAACTACCAGTGGCTCTG |
| | | CACTAGGCTGAATGGGAAATGCAAGACTTTGGAAGAATCTGTTGA |
| | | GAAATGGCGGCGTTTTCATTATGATATAAAGATATTTAATCAGTG |
| | | GCTAACAGAAGCTGAACAGTTTCTCAGAAAGACACAAATTCCTGA |
| | | GAATTGGGAACATGCTAAATACAAATGGTATCTTAAGGAACTCCA |
| | | GGATGGCATTGGGCAGCGGCAAACTGTTGTCAGAACATTGAATGC |
| | | AACTGGGGAAGAAATAATTCAGCAATCCTCAAAAACAGATGCCAG |
| | | TATTCTACAGGAAAAATTGGGAAGCCTGAATCTGCGGTGGCAGGA |
| | | GGTCTGCAAACAGCTGTCAGACAGAAAAAAGAGGCTAGAAGAACA |
| | | AAAGAATATCTTGTCAGAATTTCAAAGAGATTTAAATGAATTTGT |
| | | TTTATGGTTGGAGGAAGCAGATAACATTGCTAGTATCCCACTTGA |
| | | ACCTGGAAAAGAGCAGCAACTAAAAGAAAAGCTTGAGCAAGTCAA |
| | | GTTACTGGTGGAAGAGTTGCCCCTGCGCCAGGGAATTCTCAAACA |
| | | ATTAAATGAAACTGGAGGACCCGTGCTTGTAAGTGCTCCCATAAG |
| | | CCCAGAAGAGCAAGATAAACTTGAAAATAAGCTCAAGCAGACAAA |
| | | TCTCCAGTGGATAAAGGTTTCCAGAGCTTTACCTGAGAAACAAGG |
| | | AGAAATTGAAGCTCAAATAAAAGACCTTGGGCAGCTTGAAAAAAA |
| | | GCTTGAAGACCTTGAAGAGCAGTTAAATCATCTGCTGCTGTGGTT |
| | | ATCTCCTATTAGGAATCAGTTGGAAATTTATAACCAACCAAACCA |
| | | AGAAGGACCATTTGACGTTCAGGAAACTGAAATAGCAGTTCAAGC |
| | | TAAACAACCGGATGTGGAAGAGATTTTGTCTAAAGGGCAGCATTT |
| | | GTACAAGGAAAAACCAGCCACTCAGCCAGTGAAGAGGAAGTTAGA |
| | | AGATCTGAGCTCTGAGTGGAAGGCGGTAAACCGTTTACTTCAAGA |
| | | GCTGAGGGCAAAGCAGCCTGACCTAGCTCCTGGACTGACCACTAT |
| | | TGGAGCCTCTCCTACTCAGACTGTTACTCTGGTGACACAACCTGT |
| | | GGTTACTAAGGAAACTGCCATCTCCAAACTAGAAATGCCATCTTC |
| | | CTTGATGTTGGAGGTACCTGCTCTGGCAGATTTCAACCGGGCTTG |
| | | GACAGAACTTACCGACTGGCTTTCTCTGCTTGATCAAGTTATAAA |
| | | ATCACAGAGGGTGATGGTGGGTGACCTTGAGGATATCAACGAGAT |
| | | GATCATCAAGCAGAAGGCAACAATGCAGGATTTGGAACAGAGGCG |
| | | TCCCCAGTTGGAAGAACTCATTACCGCTGCCCAAAATTTGAAAAA |
| | | CAAGACCAGCAATCAAGAGGCTAGAACAATCATTACGGATCGAAT |
| | | TGAAAGAATTCAGAATCAGTGGGATGAAGTACAAGAACACCTTCA |
| | | GAACCGGAGGCAACAGTTGAATGAAATGTTAAAGGATTCAACACA |
| | | ATGGCTGGAAGCTAAGGAAGAAGCTGAGCAGGTCTTAGGACAGGC |
| | | CAGAGCCAAGCTTGAGTCATGGAAGGAGGGTCCCTATACAGTAGA |
| | | TGCAATCCAAAAGAAAATCACAGAAACCAAGCAGTTGGCCAAAGA |
| | | CCTCCGCCAGTGGCAGACAAATGTAGATGTGGCAAATGACTTGGC |
| | | CCTGAAACTTCTCCGGGATTATTCTGCAGATGATACCAGAAAAGT |
| | | CCACATGATAACAGAGAATATCAATGCCTCTTGGAGAAGCATTCA |
| | | TAAAAGGGTGAGTGAGCGAGAGGCTGCTTTGGAAGAAACTCATAG |
| | | ATTACTGCAACAGTTCCCCCTGGACCTGGAAAAGTTTCTTGCCTG |
| | | GCTTACAGAAGCTGAAACAACTGCCAATGTCCTACAGGATGCTAC |
| | | CCGTAAGGAAAGGCTCCTAGAAGACTCCAAGGGAGTAAAAGAGCT |
| | | GATGAAACAATGGCAAGACCTCCAAGGTGAAATTGAAGCTCACAC |
| | | AGATGTTTATCACAACCTGGATGAAAACAGCCAAAAAATCCTGAG |
| | | ATCCCTGGAAGGTTCCGATGATGCAGTCCTGTTACAAAGACGTTT |
| | | GGATAACATGAACTTCAAGTGGAGTGAACTTCGGAAAAAGTCTCT |
| | | CAACATTAGGTCCCATTTGGAAGCCAGTTCTGACCAGTGGAAGCG |
| | | TCTGCACCTTTCTCTGCAGGAACTTCTGGTGTGGCTACAGCTGAA |
| | | AGATGATGAATTAAGCCGGCAGGCACCTATTGGAGGCGACTTTCC |
| | | AGCAGTTCAGAAGCAGAACGATGTACATAGGGCCTTCAAGAGGGA |
| | | ATTGAAAACTAAAGAACCTGTAATCATGAGTACTCTTGAGACTGT |
| | | ACGAATATTTCTGACAGAGCAGCCTTTGGAAGGACTAGAGAAACT |
| | | CTACCAGGAGCCCAGAGAGCTGCCTCCTGAGGAGAGAGCCCAGAA |
| | | TGTCACTCGGCTTCTACGAAAGCAGGCTGAGGAGGTCAATACTGA |
| | | GTGGGAAAAATTGAACCTGCACTCCGCTGACTGGCAGAGAAAAAT |
| | | AGATGAGACCCTTGAAAGACTCCAGGAACTTCAAGAGGCCACGGA |
| | | TGAGCTGGACCTCAAGCTGCGCCAAGCTGAGGTGATCAAGGGATC |
| | | CTGGCAGCCCGTGGGCGATCTCCTCATTGACTCTCTCCAAGATCA |
| | | CCTCGAGAAAGTCAAGGCACTTCGAGGAGAAATTGCGCCTCTGAA |
| | | AGAGAACGTGAGCCACGTCAATGACCTTGCTCGCCAGCTTACCAC |
| | | TTTGGGCATTCAGCTCTCACCGTATAACCTCAGCACTCTGGAAGA |
| | | CCTGAACACCAGATGGAAGCTTCTGCAGGTGGCCGTCGAGGACCG |
| | | AGTCAGGCAGCTGCATGAAGCCCACAGGGACTTTGGTCCAGCATC |
| | | TCAGCACTTTCTTTCCACGTCTGTCCAGGGTCCCTGGGAGAGAGC |
| | | CATCTCGCCAAACAAAGTGCCCTACTATATCAACCACGAGACTCA |
| | | AACAACTTGCTGGGACCATCCCAAAATGACAGAGCTCTACCAGTC |
| | | TTTAGCTGACCTGAATAATGTCAGATTCTCAGCTTATAGGACTGC |
| | | CATGAAACTCCGAAGACTGCAGAAGGCCCTTTGCTTGGATCTCTT |
| | | GAGCCTGTCAGCTGCATGTGATGCCTTGGACCAGCACAACCTCAA |
| | | GCAAAATGACCAGCCCATGGATATCCTGCAGATTATTAATTGTTT |
| | | GACCACTATTTATGACCGCCTGGAGCAAGAGCACAACAATTTGGT |
| | | CAACGTCCCTCTCTGCGTGGATATGTGTCTGAACTGGCTGCTGAA |
| | | TGTTTATGATACGGGACGAACAGGGAGGATCCGTGTCCTGTCTTT |
| | | TAAAACTGGCATCATTTCCCTGTGTAAAGCACATTTGGAAGACAA |
| | | GTACAGATACCTTTTCAAGCAAGTGGCAAGTTCAACAGGATTTTG |
| | | TGACCAGCGCAGGCTGGGCCTCCTTCTGCATGATTCTATCCAAAT |
| | | TCCAAGACAGTTGGGTGAAGTTGCATCCTTTGGGGGCAGTAACAT |
| | | TGAGCCAAGTGTCCGGAGCTGCTTCCAATTTGCTAATAATAAGCC |
| | | AGAGATCGAAGCGGCCCTCTTCCTAGACTGGATGAGACTGGAACC |
| | | CCAGTCCATGGTGTGGCTGCCCGTCCTGCACAGAGTGGCTGCTGC |
| | | AGAAACTGCCAAGCATCAGGCCAAATGTAACATCTGCAAAGAGTG |
| | | TCCAATCATTGGATTCAGGTACAGGAGTCTAAAGCACTTTAATTA |
| | | TGACATCTGCCAAAGCTGCTTTTTTTCTGGTCGAGTTGCAAAAGG |
| | | CCATAAAATGCACTATCCCATGGTGGAATATTGCACTCCGACTAC |
| | | ATCAGGAGAAGATGTTCGAGACTTTGCCAAGGTACTAAAAAACAA |
| | | ATTTCGAACCAAAAGGTATTTTGCGAAGCATCCCCGAATGGGCTA |
| | | CCTGCCAGTGCAGACTGTCTTAGAGGGGGACAACATGGAAACTCC |
| | | CGTTACTCTGATCAACTTCTGGCCAGTAGATTCTGCGCCTGCCTC |
| | | GTCCCCTCAGCTTTCACACGATGATACTCATTCACGCATTGAACA |
| | | TTATGCTAGCAGGCTAGCAGAAATGGAAAACAGCAATGGATCTTA |
| | | TCTAAATGATAGCATCTCTCCTAATGAGAGCATAGATGATGAACA |
| | | TTTGTTAATCCAGCATTACTGCCAAAGTTTGAACCAGGACTCCCC |
| | | CCTGAGCCAGCCTCGTAGTCCTGCCCAGATCTTGATTTCCTTAGA |
| | | GAGTGAGGAAAGAGGGGAGCTAGAGAGAATCCTAGCAGATCTTGA |
| | | GGAAGAAAACAGGAATCTGCAAGCAGAATATGACCGTCTAAAGCA |
| | | GCAGCACGAACATAAAGGCCTGTCCCCACTGCCGTCCCCTCCTGA |
| | | AATGATGCCCACCTCTCCCCAGAGTCCCCGGGATGCTGAGCTCAT |
| | | TGCTGAGGCCAAGCTACTGCGTCAACACAAAGGCCGCCTGGAAGC |
| | | CAGGATGCAAATCCTGGAAGACCACAATAAACAGCTGGAGTCACA |
| | | GTTACACAGGCTAAGGCAGCTGCTGGAGCAACCCCAGGCAGAGGC |
| | | CAAAGTGAATGGCACAACGGTGTCCTCTCCTTCTACCTCTCTACA |
| | | GAGGTCCGACAGCAGTCAGCCTATGCTGCTCCGAGTGGTTGGCAG |
| | | TCAAACTTCGGACTCCATGGGTGAGGAAGATCTTCTCAGTCCTCC |
| | | CCAGGACACAAGCACAGGGTTAGAGGAGGTGATGGAGCAACTCAA |
| | | CAACTCCTTCCCTAGTTCAAGAGGAAGAAATACCCCTGGAAAGCC |
| | | AATGAGAGAGGACACAATGTAGGAAGTCTTTTCCACATGGCAGAT |
| | | GATTTGGGCAGAGCGATGGAGTCCTTAGTATCAGTCATGACAGAT |
| | | GAAGAAGGAGCAGAATAAATGTTTTACAACTCCTGATTCCCGCAT |
| | | GGTTTTTATAATATTCATACAACAAAGAGGATTAGACAGTAAGAG |
| | | TTTACAAGAAATAAATCTATATTTTTGTGAAGGGTAGTGGTATTA |
| | | TACTGTAGATTTCAGTAGTTTCTAAGTCTGTTATTGTTTTGTTAA |
| | | CAATGGCAGGTTTTACACGTCTATGCAATTGTACAAAAAAGTTAT |
| | | AAGAAAACTACATGTAAAATCTTGATAGCTAAATAACTTGCCATT |
| | | TCTTTATATGGAACGCATTTTGGGTTGTTTAAAAATTTATAACAG |
| | | TTATAAAGAAAGATTGTAAACTAAAGTGTGCTTTATAAAAAAAAG |
| | | TTGTTTATAAAAACCCCTAAAAACAAAACAAACACACACACACAC |
| | | ACATACACACACACACACAAAACTTTGAGGCAGCGCATTGTTTTG |
| | | CATCCTTTTGGCGTGATATCCATATGAAATTCATGGCTTTTTCTT |
| | | TTTTTGCATATTAAAGATAAGACTTCCTCTACCACCACACCAAAT |
| | | GACTACTACACACTGCTCATTTGAGAACTGTCAGCTGAGTGGGGC |
| | | AGGCTTGAGTTTTCATTTCATATATCTATATGTCTATAAGTATAT |
| | | AAATACTATAGTTATATAGATAAAGAGATACGAATTTCTATAGAC |
| | | TGACTTTTTCCATTTTTTAAATGTTCATGTCACATCCTAATAGAA |
| | | AGAAATTACTTCTAGTCAGTCATCCAGGCTTACCTGCTTGGTCTA |
| | | GAATGGATTTTTCCCGGAGCCGGAAGCCAGGAGGAAACTACACCA |
| | | CACTAAAACATTGTCTACAGCTCCAGATGTTTCTCATTTTAAACA |
| | | ACTTTCCACTGACAACGAAAGTAAAGTAAAGTATTGGATTTTTTT |
| | | AAAGGGAACATGTGAATGAATACACAGGACTTATTATATCAGAGT |
| | | GAGTAATCGGTTGGTTGGTTGATTGATTGATTGATTGATACATTC |
| | | AGCTTCCTGCTGCTAGCAATGCCACGATTTAGATTTAATGATGCT |
| | | TCAGTGGAAATCAATCAGAAGGTATTCTGACCTTGTGAACATCAG |
| | | AAGGTATTTTTTAACTCCCAAGCAGTAGCAGGACGATGATAGGGC |
| | | TGGAGGGCTATGGATTCCCAGCCCATCCCTGTGAAGGAGTAGGCC |
| | | ACTCTTTAAGTGAAGGATTGGATGATTGTTCATAATACATAAAGT |
| | | TCTCTGTAATTACAACTAAATTATTATGCCCTCTTCTCACAGTCA |
| | | AAAGGAACTGGGTGGTTTGGTTTTTGTTGCTTTTTTAGATTTATT |
| | | GTCCCATGTGGGATGAGTTTTTAAATGCCACAAGACATAATTTAA |
| | | AATAAATAAACTTTGGGAAAAGGTGTAAGACAGTAGCCCCATCAC |
| | | ATTTGTGATACTGACAGGTATCAACCCAGAAGCCCATGAACTGTG |
| | | TTTCCATCCTTTGCATTTCTCTGCGAGTAGTTCCACACAGGTTTG |
| | | TAAGTAAGTAAGAAAGAAGGCAAATTGATTCAAATGTTACAAAAA |
| | | AACCCTTCTTGGTGGATTAGACAGGTTAAATATATAAACAAACAA |
| | | ACAAAAATTGCTCAAAAAAGAGGAGAAAAGCTCAAGAGGAAAAGC |
| | | TAAGGACTGGTAGGAAAAAGCTTTACTCTTTCATGCCATTTTATT |
| | | TCTTTTTGATTTTTAAATCATTCATTCAATAGATACCACCGTGTG |
| | | ACCTATAATTTTGCAAATCTGTTACCTCTGACATCAAGTGTAATT |
| | | AGCTTTTGGAGAGTGGGCTGACATCAAGTGTAATTAGCTTTTGGA |
| | | GAGTGGGTTTTGTCCATTATTAATAATTAATTAATTAACATCAAA |
| | | CACGGCTTCTCATGCTATTTCTACCTCACTTTGGTTTTGGGGTGT |
| | | TCCTGATAATTGTGCACACCTGAGTTCACAGCTTCACCACTTGTC |
| | | CATTGCGTTATTTTCTTTTTCCTTTATAATTCTTTCTTTTTCCTT |
| | | CATAATTTTCAAAAGAAAACCCAAAGCTCTAAGGTAACAAATTAC |
| | | CAAATTACATGAAGATTTGGTTTTTGTCTTGCATTTTTTTCCTTT |
| | | ATGTGACGCTGGACCTTTTCTTTACCCAAGGATTTTTAAAACTCA |
| | | GATTTAAAACAAGGGGTTACTTTACATCCTACTAAGAAGTTTAAG |
| | | TAAGTAAGTTTCATTCTAAAATCAGAGGTAAATAGAGTGCATAAA |
| | | TAATTTTGTTTTAATCTTTTTGTTTTTCTTTTAGACACATTAGCT |
| | | CTGGAGTGAGTCTGTCATAATATTTGAACAAAAATTGAGAGCTTT |
| | | ATTGCTGCATTTTAAGCATAATTAATTTGGACATTATTTCGTGTT |
| | | GTGTTCTTTATAACCACCGAGTATTAAACTGTAAATCATAATGTA |
| | | ACTGAAGCATAAACATCACATGGCATGTTTTGTCATTGTTTTCAG |
| | | GTACTGAGTTCTTACTTGAGTATCATAATATATTGTGTTTTAACA |
| | | CCAACACTGTAACATTTACGAATTATTTTTTTAAACTTCAGTTTT |
| | | ACTGCATTTTCACAACATATCAGACTTCACCAAATATATGCCTTA |
| | | CTATTGTATTATAGTACTGCTTTACTGTGTATCTCAATAAAGCAC |
| | | GCAGTTATGTTAC |
| 45 | SGCA nucleotide | atggccgagacactgttctggactcctctgctggtggtgctgctg |
| | sequence (homo | gctggactgggagataccgaggctcagcagaccacactgcaccca |
| | sapiens) | ctggtgggccgggtgttcgtgcacaccctggaccatgagacattt |
| | | ctgagtctgccagaacacgtggctgtgccacctgctgtgcatatc |
| | | acttaccacgcccatctgcagggccatcctgatctgccacggtgg |
| | | ctgagatacacccagagatcaccccaccatcctggattcctgtat |
| | | ggaagcgctaccccagaggacaggggactgcaggtgatcgaagtg |
| | | acagcttacaaccgcgacagttttgatactaccaggcagcgcctg |
| | | gtgctggagattggggatccagaaggacccctgctgccttatcag |
| | | gccgagttcctggtgcggtcacacgacgctgaggaagtgctgcca |
| | | tcaacacccgccagcagatttctgtccgctctgggaggactgtgg |
| | | gagccaggagaactgcagctgctgaatgtgactagcgctctggat |
| | | aggggaggaagggtgccactgccaatcgagggaaggaaggaaggg |
| | | gtgtacattaaagtgggaagcgcttccccattctccacctgcctg |
| | | aagatggtggcttctcctgatagtcacgctaggtgcgctcaggga |
| | | cagccaccactgctgtcctgttatgacacactggccccccatttt |
| | | cgcgtggactggtgcaacgtgactctggtggataaatctgtgcct |
| | | gagccagctgacgaagtgccaacccctggagacggaatcctggag |
| | | cacgatcctttcttttgtcctccaacagaagccccagacagggat |
| | | ttcctggtggacgctctggtgactctgctggtgcctctgctggtg |
| | | gctctgctgctgaccctgctgctggcttatgtgatgtgctgtcgg |
| | | agagagggacggctgaagagagacctggccacatctgatatccag |
| | | atggtgcaccattgtactattcacggcaacaccgaggaactgcgc |
| | | cagatggctgcttctagggaggtgccaaggccactgagtacactg |
| | | cctatgtttaatgtgcacactggcgaacggctgccccctagagtg |
| | | gatagcgcccaggtgccactgattctggaccagcattga |
| 46 | SGCA amino acid | MetAlaGluThrLeuPheTrpThrProLeuLeuValValLeuLeu |
| | sequence (homo | AlaGlyLeuGlyAspThrGluAlaGlnGlnThrThrLeuHisPro |
| | sapiens) | LeuValGlyArgValPheValHisThrLeuAspHisGluThrPhe |
| | | LeuSerLeuProGluHisValAlaValProProAlaValHisIle |
| | | ThrTyrHisAlaHisLeuGlnGlyHisProAspLeuProArgTrp |
| | | LeuArgTyrThrGlnArgSerProHisHisProGlyPheLeuTyr |
| | | GlySerAlaThrProGluAspArgGlyLeuGlnValIleGluVal |
| | | ThrAlaTyrAsnArgAspSerPheAspThrThrArgGlnArgLeu |
| | | ValLeuGluIleGlyAspProGluGlyProLeuLeuProTyrGln |
| | | AlaGluPheLeuValArgSerHisAspAlaGluGluValLeuPro |
| | | SerThrProAlaSerArgPheLeuSerAlaLeuGlyGlyLeuTrp |
| | | GluProGlyGluLeuGlnLeuLeuAsnValThrSerAlaLeuAsp |
| | | ArgGlyGlyArgValProLeuProIleGluGlyArgLysGluGly |
| | | ValTyrIleLysValGlySerAlaSerProPheSerThrCysLeu |
| | | LysMetValAlaSerProAspSerHisAlaArgCysAlaGlnGly |
| | | GlnProProLeuLeuSerCysTyrAspThrLeuAlaProHisPhe |
| | | ArgValAspTrpCysAsnValThrLeuValAspLysSerValPro |
| | | GluProAlaAspGluValProThrProGlyAspGlyIleLeuGlu |
| | | HisAspProPhePheCysProProThrGluAlaProAspArgAsp |
| | | PheLeuValAspAlaLeuValThrLeuLeuValProLeuLeuVal |
| | | AlaLeuLeuLeuThrLeuLeuLeuAlaTyrValMetCysCysArg |
| | | ArgGluGlyArgLeuLysArgAspLeuAlaThrSerAspIleGln |
| | | MetValHisHisCysThrIleHisGlyAsnThrGluGluLeuArg |
| | | GlnMetAlaAlaSerArgGluVaIProArgProLenSerThrLeu |
| | | ProMetPheAsnValHisThrGlyGluArgLeuProProArgVal |
| | | AspSerAlaGlnValProLenIleLeuAspGlnHis |
| 47 | scAAVrh74.tMC | ctgcgcgctcgctcgctcactgaggccgcccgggcaaagcccggg |
| | K.hSGCA | cgtcgggcgacctttggtcgcccggcctcagtgagcgagcgagcg |
| | (artificial | cgcagagagggagtggggttaaccaattggcggccgcaaacttgc |
| | sequence) | atgccccactacgggtctaggctgcccatgtaaggaggcaaggcc |
| | | tggggacacccgagatgcctggttataattaaccccaacacctgc |
| | | tgcccccccccccccaacacctgctgcctgagcctgagcggttac |
| | | cccaccccggtgcctgggtcttaggctctgtacaccatggaggag |
| | | aagctcgctctaaaaataaccctgtccctggtggatccactacgg |
| | | gtctatgctgcccatgtaaggaggcaaggcctggggacacccgag |
| | | atgcctggttataattaaccccaacacctgctgcccccccccccc |
| | | caacacctgctgcctgagcctgagcggttaccccaccccggtgcc |
| | | tgggtcttaggctctgtacaccatggaggagaagctcgctctaaa |
| | | aataaccctgtccctggtggaccactacgggtctaggctgcccat |
| | | gtaaggaggcaaggcctggggacacccgagatgcctggttataat |
| | | taaccccaacacctgctgccccccccccccaacacctgctgcctg |
| | | agcctgagcggttaccccaccccggtgcctgggtcttaggctctg |
| | | tacaccatggaggagaagctcgctctaaaaataaccctgtccctg |
| | | gtcctccctggggacagcccctcctggctagtcacaccctgtagg |
| | | ctcctctatataacccaggggcacaggggctgcccccgggtcacc |
| | | tgcagaagttggtcgtgaggcactgggcaggtaagtatcaaggtt |
| | | acaagacaggtttaaggagaccaatagaaactgggcttgtcgaga |
| | | cagagaagactcttgcgtttctgataggcacctattggtcttact |
| | | gacatccactttgcctttctctccacaggtgtccactcccagttc |
| | | aattacagcgcgtggtaccaccatggccgagacactgttctggac |
| | | tcctctgctggtggtgctgctggctggactgggagataccgaggc |
| | | tcagcagaccacactgcacccactggtgggccgggtgttcgtgca |
| | | caccctggaccatgagacatttctgagtctgccagaacacgtggc |
| | | tgtgccacctgctgtgcatatcacttaccacgcccatctgcaggg |
| | | ccatcctgatctgccacggtggctgagatacacccagagatcacc |
| | | ccaccatcctggattcctgtatggaagcgctaccccagaggacag |
| | | gggactgcaggtgatcgaagtgacagcttacaaccgcgacagttt |
| | | tgatactaccaggcagcgcctggtgctggagattggggatccaga |
| | | aggacccctgctgccttatcaggccgagttcctggtgcggtcaca |
| | | cgacgctgaggaagtgctgccatcaacacccgccagcagatttct |
| | | gtccgctctgggaggactgtgggagccaggagaactgcagctgct |
| | | gaatgtgactagcgctctggataggggaggaagggtgccactgcc |
| | | aatcgagggaaggaaggaaggggtgtacattaaagtgggaagcgc |
| | | ttccccattctccacctgcctgaagatggtggcttctcctgatag |
| | | tcacgctaggtgcgctcagggacagccaccactgctgtcctgtta |
| | | tgacacactggccccccattttcgcgtggactggtgcaacgtgac |
| | | tctggtggataaatctgtgcctgagccagctgacgaagtgccaac |
| | | ccctggagacggaatcctggagcacgatcctttcttttgtcctcc |
| | | aacagaagccccagacagggatttcctggtggacgctctggtgac |
| | | tctgctggtgcctctgctggtggctctgctgctgaccctgctgct |
| | | ggcttatgtgatgtgctgtcggagagagggacggctgaagagaga |
| | | cctggccacatctgatatccagatggtgcaccattgtactattca |
| | | cggcaacaccgaggaactgcgccagatggctgcttctagggaggt |
| | | gccaaggccactgagtacactgcctatgtttaatgtgcacactgg |
| | | cgaacggctgccccctagagtggatagcgcccaggtgccactgat |
| | | tctggaccagcattgaggccgcaataaaagatctttattttcatt |
| | | agatctgtgtgttggttttttgtgtgtcctgcaggggcgcgcctc |
| | | tagagcatggctacgtagataagtagcatggcgggttaatcatta |
| | | actacaaggaacccctagtgatggagttggccactccctctctgc |
| | | gcgctcgctcgctcactgaggccgggcgaccaaaggtcgcccgac |
| | | gcccgggctttgcccgggcggcctcagtgagcgagcgagcgcgca |
| | | g |
| 48 | pAAV.tMCK.hS | atgcagctgcgcgctcgctcgctcactgaggccgcccgggcaaag |
| | GCA.KAN | cccgggcgtcgggcgacctttggtcgcccggcctcagtgagcgag |
| | (artificial | cgagcgcgcagagagggagtggggttaaccaattggcggccgcaa |
| | sequence) | acttgcatgccccactacgggtctaggctgcccatgtaaggaggc |
| | | aaggcctggggacacccgagatgcctggttataattaaccccaac |
| | | acctgctgcccccccccccccaacacctgctgcctgagcctgagc |
| | | ggttaccccaccccggtgcctgggtcttaggctctgtacaccatg |
| | | gaggagaagctcgctctaaaaataaccctgtccctggtggatcca |
| | | ctacgggtctatgctgcccatgtaaggaggcaaggcctggggaca |
| | | cccgagatgcctggttataattaaccccaacacctgctgcccccc |
| | | cccccccaacacctgctgcctgagcctgagcggttaccccacccc |
| | | ggtgcctgggtcttaggctctgtacaccatggaggagaagctcgc |
| | | tctaaaaataaccctgtccctggtggaccactacgggtctaggct |
| | | gcccatgtaaggaggcaaggcctggggacacccgagatgcctggt |
| | | tataattaaccccaacacctgctgccccccccccccaacacctgc |
| | | tgcctgagcctgagcggttaccccaccccggtgcctgggtcttag |
| | | gctctgtacaccatggaggagaagctcgctctaaaaataaccctg |
| | | tccctggtcctccctggggacagcccctcctggctagtcacaccc |
| | | tgtaggctcctctatataacccaggggcacaggggctgcccccgg |
| | | gtcacctgcagaagttggtcgtgaggcactgggcaggtaagtatc |
| | | aaggttacaagacaggtttaaggagaccaatagaaactgggcttg |
| | | tcgagacagagaagactcttgcgtttctgataggcacctattggt |
| | | cttactgacatccactttgcctttctctccacaggtgtccactcc |
| | | cagttcaattacagcgcgtggtaccaccatggccgagacactgtt |
| | | ctggactcctctgctggtggtgctgctggctggactgggagatac |
| | | cgaggctcagcagaccacactgcacccactggtgggccgggtgtt |
| | | cgtgcacaccctggaccatgagacatttctgagtctgccagaaca |
| | | cgtggctgtgccacctgctgtgcatatcacttaccacgcccatct |
| | | gcagggccatcctgatctgccacggtggctgagatacacccagag |
| | | atcaccccaccatcctggattcctgtatggaagcgctaccccaga |
| | | ggacaggggactgcaggtgatcgaagtgacagcttacaaccgcga |
| | | cagttttgatactaccaggcagcgcctggtgctggagattgggga |
| | | tccagaaggacccctgctgccttatcaggccgagttcctggtgcg |
| | | gtcacacgacgctgaggaagtgctgccatcaacacccgccagcag |
| | | atttctgtccgctctgggaggactgtgggagccaggagaactgca |
| | | gctgctgaatgtgactagcgctctggataggggaggaagggtgcc |
| | | actgccaatcgagggaaggaaggaaggggtgtacattaaagtggg |
| | | aagcgcttccccattctccacctgcctgaagatggtggcttctcc |
| | | tgatagtcacgctaggtgcgctcagggacagccaccactgctgtc |
| | | ctgttatgacacactggccccccattttcgcgtggactggtgcaa |
| | | cgtgactctggtggataaatctgtgcctgagccagctgacgaagt |
| | | gccaacccctggagacggaatcctggagcacgatcctttcttttg |
| | | tcctccaacagaagccccagacagggatttcctggtggacgctct |
| | | ggtgactctgctggtgcctctgctggtggctctgctgctgaccct |
| | | gctgctggcttatgtgatgtgctgtcggagagagggacggctgaa |
| | | gagagacctggccacatctgatatccagatggtgcaccattgtac |
| | | tattcacggcaacaccgaggaactgcgccagatggctgcttctag |
| | | ggaggtgccaaggccactgagtacactgcctatgtttaatgtgca |
| | | cactggcgaacggctgccccctagagtggatagcgcccaggtgcc |
| | | actgattctggaccagcattgaggccgcaataaaagatctttatt |
| | | ttcattagatctgtgtgttggttttttgtgtgtcctgcaggggcg |
| | | cgcctctagagcatggctacgtagataagtagcatggcgggttaa |
| | | tcattaactacaaggaacccctagtgatggagttggccactccct |
| | | ctctgcgcgctcgctcgctcactgaggccgggcgaccaaaggtcg |
| | | cccgacgcccgggctttgcccgggcggcctcagtgagcgagcgag |
| | | cgcgcagctggcgtaatagcgaagaggcccgcaccgatcgccctt |
| | | cccaacagttgcgcagcctgaatggcgaatggcgattccgttgca |
| | | atggctggcggtaatattgttctggatattaccagcaaggccgat |
| | | agtttgagttcttctactcaggcaagtgatgttattactaatcaa |
| | | agaagtattgcgacaacggttaatttgcgtgatggacagactctt |
| | | ttactcggtggcctcactgattataaaaacacttctcaggattct |
| | | ggcgtaccgttcctgtctaaaatccctttaatcggcctcctgttt |
| | | agctcccgctctgattctaacgaggaaagcacgttatacgtgctc |
| | | gtcaaagcaaccatagtacgcgccctgtagcggcgcattaagcgc |
| | | ggcgggtgtggtggttacgcgcagcgtgaccgctacacttgccag |
| | | cgccctagcgcccgctcctttcgctttcttcccttcctttctcgc |
| | | cacgttcgccatcttcaaatatgtatccgctcatgagacaataac |
| | | cctgataaatgcttcaataatattgaaaaaggaagagtcctgagg |
| | | cggaaagaaccagctgtggaatgtgtgtcagttagggtgtggaaa |
| | | gtccccaggctccccagcaggcagaagtatgcaaagcatgcatct |
| | | caattagtcagcaaccaggtgtggaaagtccccaggctccccagc |
| | | aggcagaagtatgcaaagcatgcatctcaattagtcagcaaccat |
| | | agtcccgcccctaactccgccccatggctgactaattttttttat |
| | | ttatgcagaggccgaggccgcctcggcctctgagctattccagaa |
| | | gtagtgaggaggcttttttggaggcctaggcttttgcaaagatcg |
| | | atcaagagacaggatgaggatcgtttcgcatgattgaacaagatg |
| | | gattgcacgcaggttctccggccgcttgggtggagaggctattcg |
| | | gctatgactgggcacaacagacaatcggctgctctgatgccgccg |
| | | tgttccggctgtcagcgcaggggcgcccggttctttttgtcaaga |
| | | ccgacctgtccggtgccctgaatgaactgcaagacgaggcagcgc |
| | | ggctatcgtggctggccacgacgggcgttccttgcgcagctgtgc |
| | | tcgacgttgtcactgaagcgggaagggactggctgctattgggcg |
| | | aagtgccggggcaggatctcctgtcatctcaccttgctcctgccg |
| | | agaaagtatccatcatggctgatgcaatgcggcggctgcatacgc |
| | | ttgatccggctacctgcccattcgaccaccaagcgaaacatcgca |
| | | tcgagcgagcacgtactcggatggaagccggtcttgtcgatcagg |
| | | atgatctggacgaagagcatcaggggctcgcgccagccgaactgt |
| | | tcgccaggctcaaggcgagcatgcccgacggcgaggatctcgtcg |
| | | tgacccatggcgatgcctgcttgccgaatatcatggtggaaaatg |
| | | gccgcttttctggattcatcgactgtggccggctgggtgtggcgg |
| | | accgctatcaggacatagcgttggctacccgtgatattgctgaag |
| | | agcttggcggcgaatgggctgaccgcttcctcgtgctttacggta |
| | | tcgccgctcccgattcgcagcgcatcgccttctatcgccttcttg |
| | | acgagttcttctgagcgggactctggggttcgaaatgaccgacca |
| | | agcgacgcccaacctgccatcacgagatttcgattccaccgccgc |
| | | cttctatgaaaggttgggcttcggaatcgttttccgggacgccgg |
| | | ctggatgatcctccagcgcggggatctcatgctggagttcttcgc |
| | | ccaccctagggggaggctaactgaaacacggaaggagacaatacc |
| | | ggaaggaacccgcgctatgacggcaataaaaagacagaataaaaa |
| | | cgttgcgcaaactattaactggcgaactacttactctagcttccc |
| | | ggcaacaattaatagactggatggaggcggataaagttgcaggac |
| | | cacttctgcgctcggcccttccggctggctggtttattgctgata |
| | | aatctggagccggtgagcgtgggtctcgcggtatcattgcagcac |
| | | tggggccagatggtaagccctcccgtatcgtagttatctacacga |
| | | cggggagtcaggcaactatggatgaacgaaatagacagatcgctg |
| | | agataggtgcctcactgattaagcattggtaactgtcagaccaag |
| | | tttactcatatatactttagattgatttaaaacttcatttttaat |
| | | ttaaaaggatctaggtgaagatcctttttgataatctcatgacca |
| | | aaatcccttaacgtgagttttcgttccactgagcgtcagaccccg |
| | | tagaaaagatcaaaggatcttcttgagatcctttttttctgcgcg |
| | | taatctgctgcttgcaaacaaaaaaaccaccgctaccagcggtgg |
| | | tttgtttgccggatcaagagctaccaactctttttccgaaggtaa |
| | | ctggcttcagcagagcgcagataccaaatactgttcttctagtgt |
| | | agccgtagttaggccaccacttcaagaactctgtagcaccgccta |
| | | catacctcgctctgctaatcctgttaccagtggctgctgccagtg |
| | | gcgataagtcgtgtcttaccgggttggactcaagacgatagttac |
| | | cggataaggcgcagcggtcgggctgaacggggggttcgtgcacac |
| | | agcccagcttggagcgaacgacctacaccgaactgagatacctac |
| | | agcgtgagctatgagaaagcgccacgcttcccgaagggagaaagg |
| | | cggacaggtatccggtaagcggcagggtcggaacaggagagcgca |
| | | cgagggagcttccagggggaaacgcctggtatctttatagtcctg |
| | | tcgggtttcgccacctctgacttgagcgtcgatttttgtgatgct |
| | | cgtcaggggggcggagcctatggaaaaacgccagcaacgcggcct |
| | | ttttacggttcctggccttttgctggccttttgctcacatgttct |
| | | ttcctgcgttatcccctgattctgtggataaccgtattaccgcct |
| | | ttgagtgagctgataccgctcgccgcagccgaacgaccgagcgca |
| | | gcgagtcagtgagcgaggaagcggaagagcgcccaatacgcaaac |
| | | cgcctctccccgcgcgttggccgattcattaatg |

1. A method of restoring or stabilizing a dystrophin-associated protein complex (DAPC) in a subject suffering from muscular dystrophy, comprising administering to the subject a polynucleotide sequence encoding (a) a sarcoglycan and/or (b) dystrophin or abbreviated version thereof, wherein the polynucleotide encoding the sarcoglycan comprises a nucleotide sequence that is at least 70% identical to a sequence of any one of SEQ ID NOs: 1, 3, 5, 7, 8, 13, 14, 17, 19-24, 30-32, 45, 47, or 48 across the entire length of SEQ ID NOs: 1, 3, 5, 7, 8, 13, 14, 17, 19-24, 30-32, 45, 47, or 48, respectively.

2. The method of claim 1, wherein the muscular dystrophy is Duchenne muscular dystrophy (DMD) or Becker muscular dystrophy (BMD).

3. The method of claim 2, wherein the method comprises administering to the subject a polynucleotide encoding dystrophin or an abbreviated version of dystrophin.

4. The method of claim 2, wherein the abbreviated version of dystrophin is a microdystrophin or mini dystrophin.

5. The method of claim 1, wherein when the muscular dystrophy is:
a. LGMD2C, the composition comprises a polynucleotide of one or more of SEQ ID NOs: 19-24;
b. LGMD2D, the composition comprises a polynucleotide of one or more of SEQ ID NOs: 13, 14, 45, 47, and 48;
c. LGMD2E, the composition comprises a polynucleotide of one or more of SEQ ID NOs: 1, 3, 5, 7, 8, and 17: or
d. LGMD2F, the composition comprises a polynucleotide of SEQ ID Nos: 30-32.

6-12. (canceled)

13. A method for one or both of:
localizing a first sarcoglycan, sarcospan, and/or dystrophin to a muscle cell membrane or sarcolemma in a subject suffering from muscular dystrophy; and
increasing or enhancing expression of a first sarcoglycan, sarcospan, and/or dystrophin to a muscle cell membrane or sarcolemma in a subject suffering from muscular dystrophy,
comprising administering to the subject a polynucleotide sequence encoding (a) a second sarcoglycan; and/or (b) dystrophin or abbreviated version thereof, wherein the first sarcoglycan is different from the second sarcoglycan, and wherein the polynucleotide encoding the second sarcoglycan comprises a nucleotide sequence that is at least 70% identical to a sequence of any one of SEQ ID NOs: 1, 3, 5, 7, 8, 13, 14, 17, 19-24, 30-32, 45, 47, or 48 across the entire length of SEQ ID NOs: 1, 3, 5, 7, 8, 13, 14, 17, 19-24, 30-32, 45, 47, or 48.

14. (canceled)

15. The method of claim 13, wherein the muscular dystrophy is DMD or BMD.

16. The method of claim 15, wherein the method comprises administering to the subject a polynucleotide encoding dystrophin or an abbreviated version of dystrophin.

17. The method of claim 16, wherein the polynucleotide encoding dystrophin comprises a nucleotide sequence that is at least 70%, 80%, 85%, 90%, 95%, 97%, 98%, 99%, or 100% identical to the nucleotide sequence of SEQ ID NO: 36 or 37 across the entire length of SEQ ID NO: 36 or 37; and/or
the polynucleotide encoding the abbreviated version of dystrophin comprises (a) a nucleotide sequence that is at least 70%, 80%, 85%, 90%, 95%, 97%, 98%, 99%, or 100% identical to the nucleotide sequence of any one of SEQ ID NOs: 38 and 40-44 across the entire length of SEQ ID NOs: 38 and 40-44: or (b) a nucleotide sequence that encodes an abbreviated version of a dystrophin protein comprising an amino acid sequence that is at least 70%, 80%, 85%, 90%, 95%, 97%, 98%, 99%, or 100% identical to the amino acid sequence of SEQ ID NO: 39 across the entire length of SEQ ID NO: 39.

18-19. (canceled)

20. The method of claim 13, wherein when the muscular dystrophy is:
a. LGMD2C, the composition comprises a polynucleotide of one or more of SEQ ID NOs: 19-24;
b. LGMD2D, the composition comprises a polynucleotide of one or more of SEQ ID NOs: 13, 14, 45, 47, and 48;
c. LGMD2E, the composition comprises a polynucleotide of one or more of SEQ ID NOs: 1, 3, 5, 7, 8, and 17: or
d. LGMID2F, the composition comprises a polynucleotide of SEQ ID Nos: 30-32.

21-31. (canceled)

32. The method of claim 13, wherein the first sarcoglycan comprises one or more of SGCD, SGCB, SGCA, and SGCG.

33-37. (canceled)

38. The method of claim 13, wherein the expression of the first sarcoglycan, sarcospan, or dystrophin to the muscle cell membrane or sarcolemma is increased by at least 10%, 20%, 30%, 40%, 50%, 60%, 70%, 80%, 90%, 100%, 110%, 120%, 130%, 140%, 150%, 160%, 170%, 180%, 190%, or 200% as compared to the expression of the first sarcoglycan, sarcospan, or dystrophin prior to administering one or more doses of the polynucleotide.

39-44. (canceled)

45. The method of claim 13, wherein the polynucleotide is encapsidated within a viral vector, wherein the polynucleotide comprises a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

46-92. (canceled)

93. A composition comprising a polynucleotide, wherein the polynucleotide is encapsidated within a viral vector, wherein the polynucleotide comprises a nucleotide sequence that is at least 70%, 80%, 85%, 90%, 95%, 97%, 98%, 99%, or 100% identical to the nucleotide sequence of any one of SEQ ID NOs: 3, 5, 7, 8, 19, 47, and 48 across the entire length of SEQ ID NOs: 3, 5, 7, 8, 19, 47, and 48.

94-107. (canceled)

108. The method of claim 1, wherein the polynucleotide further comprises one or more of a promoter, an intron, a polyA sequence, and an inverted terminal repeat (ITR).

109. The method of claim 108, wherein the promoter is a muscle-specific promoter.

110. The method of claim 109, wherein the muscle-specific promoter is selected from an MHCK7 promoter and tMCK promoter.

111. The method of claim 108, wherein:
the promoter comprises a nucleotide sequence that is at least 70%, 80%, 85%, 90%, 95%, 97%, 98%, 99%, or 100% identical to the nucleotide sequence of SEQ ID NO: 4 or 6 across the entire length of SEQ ID NO: 4 or 6;
the intron comprises a nucleotide sequence that is at least 70%, 80%, 85%, 90%, 95%, 97%, 98%, 99%, or 100% identical to the nucleotide sequence of SEQ ID NO: 9 across the entire length of SEQ ID NO: 9;
the polyA sequence comprises a nucleotide sequence that is at least 70%, 80%, 85%, 90%, 95%, 97%, 98%, 99%, or 100% identical to the nucleotide sequence of SEQ ID NO: 10 across the entire length of SEQ ID NO: 10; and/or
the ITR comprises a nucleotide sequence that is at least 70%, 80%, 85%, 90%, 95%, 97%, 98%, 99%, or 100% identical to the nucleotide sequence of SEQ ID NO: 11 or 12 across the entire length of SEQ ID NO: 11 or 12.

112-131. (canceled)

132. The method of claim 13, wherein the polynucleotide further comprises one or more of a promoter, an intron, a polyA sequence, and an inverted terminal repeat (ITR).

133. The method of claim 132, wherein the promoter is selected from an MHCK7 promoter and a tMCK promoter.

134. The method of claim 132, wherein:
the promoter comprises a nucleotide sequence that is at least 70%, 80%, 85%, 90%, 95%, 97%, 98%, 99%, or 100% identical to the nucleotide sequence of SEQ ID NO: 4 or 6 across the entire length of SEQ ID NO: 4 or 6;
the intron comprises a nucleotide sequence that is at least 70%, 80%, 85%, 90%, 95%, 97%, 98%, 99%, or 100% identical to the nucleotide sequence of SEQ ID NO: 9 across the entire length of SEQ ID NO: 9;
the polyA sequence comprises a nucleotide sequence that is at least 70%, 80%, 85%, 90%, 95%, 97%, 98%, 99%, or 100% identical to the nucleotide sequence of SEQ ID NO: 10 across the entire length of SEQ ID NO: 10; and/or
the ITR comprises a nucleotide sequence that is at least 70%, 80%, 85%, 90%, 95%, 97%, 98%, 99%, or 100% identical to the nucleotide sequence of SEQ ID NO: 11 or 12 across the entire length of SEQ ID NO: 11 or 12.

135. The method of claim 45, wherein the viral vector is an adeno-associated viral (AAV) vector.
